SEQUENCE LISTING (1136 sequences)

SEQ ID NO: 1 - DNA, 25 bp, synthetic construct
  misc_feature 1..25: note = Synthetic oligonucleotide
  source 1..25: mol_type = other DNA
caccggaccccctccaccccgcctc

SEQ ID NO: 2 - DNA, 25 bp, synthetic construct
  misc_feature 1..25: note = Synthetic oligonucleotide
  source 1..25: mol_type = other DNA
aaacgaggcggggtggagggggtcc

SEQ ID NO: 3 - DNA, 25 bp, synthetic construct
  misc_feature 1..25: note = Synthetic oligonucleotide
  source 1..25: mol_type = other DNA
caccgcttgccccacagggcagtaa

SEQ ID NO: 4 - DNA, 25 bp, synthetic construct
  misc_feature 1..25: note = Synthetic oligonucleotide
  source 1..25: mol_type = other DNA
aaacttactgccctgtggggcaagc

SEQ ID NO: 5 - DNA, 25 bp, synthetic construct
  misc_feature 1..25: note = Synthetic oligonucleotide
  source 1..25: mol_type = other DNA
caccggggcggtgctacaactgggc

SEQ ID NO: 6 - DNA, 25 bp, synthetic construct
  misc_feature 1..25: note = Synthetic oligonucleotide
  source 1..25: mol_type = other DNA
aaacgcccagttgtagcaccgcccc

SEQ ID NO: 7 - DNA, 25 bp, synthetic construct
  misc_feature 1..25: note = Synthetic oligonucleotide
  source 1..25: mol_type = other DNA
caccgcttcaagagcaacagtgctg

SEQ ID NO: 8 - DNA, 25 bp, synthetic construct
  misc_feature 1..25: note = Synthetic oligonucleotide
  source 1..25: mol_type = other DNA
aaaccagcactgttgctcttgaagc

SEQ ID NO: 9 - DNA, 25 bp, synthetic construct
  misc_feature 1..25: note = Synthetic oligonucleotide
  source 1..25: mol_type = other DNA
caccgggtgtaaaatcaacacccta

SEQ ID NO: 10 - DNA, 25 bp, synthetic construct
  misc_feature 1..25: note = Synthetic oligonucleotide
  source 1..25: mol_type = other DNA
aaactagggtgttgattttacaccc

SEQ ID NO: 11 - DNA, 25 bp, synthetic construct
  misc_feature 1..25: note = Synthetic oligonucleotide
  source 1..25: mol_type = other DNA
caccgagccccagcaagagcacaag

SEQ ID NO: 12 - DNA, 25 bp, synthetic construct
  misc_feature 1..25: note = Synthetic oligonucleotide
  source 1..25: mol_type = other DNA
aaaccttgtgctcttgctggggctc

SEQ ID NO: 13 - DNA, 70 bp, synthetic construct
  misc_feature 1..70: note = Synthetic oligonucleotide
  misc_difference 30..37: note = a, c, t, g, unknown or other
  source 1..70: mol_type = other DNA
aatgatacggcgaccaccgagatctacacnnnnnnnnacactctttccctacacgacgctcttccgatct

SEQ ID NO: 14 - DNA, 65 bp, synthetic construct
  misc_feature 1..65: note = Synthetic oligonucleotide
  misc_difference 25..32: note = a, c, t, g, unknown or other
  source 1..65: mol_type = other DNA
caagcagaagacggcatacgagatnnnnnnnngtgactggagttcagacgtgtgctcttccgatc

SEQ ID NO: 15 - DNA, 45 bp, synthetic construct
  misc_feature 1..45: note = Synthetic oligonucleotide
  source 1..45: mol_type = other DNA
ccccttagggataacagggtaatccaactgccatatgccctgggt

SEQ ID NO: 16 - DNA, 42 bp, synthetic construct
  misc_feature 1..42: note = Synthetic oligonucleotide
  source 1..42: mol_type = other DNA
ccccttagggataacagggtaatccggcgggtgggtcacaaa

SEQ ID NO: 17 - DNA, 43 bp, synthetic construct
  misc_feature 1..43: note = Synthetic oligonucleotide
  source 1..43: mol_type = other DNA
ccccttagggataacagggtaatccacaggacagggtcagcgt

SEQ ID NO: 18 - DNA, 50 bp, synthetic construct
  misc_feature 1..50: note = Synthetic oligonucleotide
  source 1..50: mol_type = other DNA
ccccttagggataacagggtaatcggcccctacaatactatcttgaccct

SEQ ID NO: 19 - DNA, 47 bp, synthetic construct
  misc_feature 1..47: note = Synthetic oligonucleotide
  source 1..47: mol_type = other DNA
ccccttagggataacagggtaatctgagagacagggtcttgctgttg

SEQ ID NO: 20 - DNA, 46 bp, synthetic construct
  misc_feature 1..46: note = Synthetic oligonucleotide
  source 1..46: mol_type = other DNA
ccccttagggataacagggtaatcccaacttggtgggggtagagtg

SEQ ID NO: 21 - DNA, 42 bp, synthetic construct
  misc_feature 1..42: note = Synthetic oligonucleotide
  source 1..42: mol_type = other DNA
ccccttagggataacagggtaatctcacgcagacgcccccat

SEQ ID NO: 22 - DNA, 47 bp, synthetic construct
  misc_feature 1..47: note = Synthetic oligonucleotide
  source 1..47: mol_type = other DNA
ccccttagggataacagggtaatcagcctggagttaagggtgtctcc

SEQ ID NO: 23 - DNA, 46 bp, synthetic construct
  misc_feature 1..46: note = Synthetic oligonucleotide
  source 1..46: mol_type = other DNA
ccccttagggataacagggtaatcccccaggagcgtggatgactac

SEQ ID NO: 24 - DNA, 42 bp, synthetic construct
  misc_feature 1..42: note = Synthetic oligonucleotide
  source 1..42: mol_type = other DNA
ccccttagggataacagggtaatcgcaggtgaggccgtgcag

SEQ ID NO: 25 - DNA, 49 bp, synthetic construct
  misc_feature 1..49: note = Synthetic oligonucleotide
  source 1..49: mol_type = other DNA
ccccttagggataacagggtaatctgagttatgtggtcccctctaggaa

SEQ ID NO: 26 - DNA, 44 bp, synthetic construct
  misc_feature 1..44: note = Synthetic oligonucleotide
  source 1..44: mol_type = other DNA
ccccttagggataacagggtaatcagcgtcgtctcctggagctc

SEQ ID NO: 27 - DNA, 50 bp, synthetic construct
  misc_feature 1..50: note = Synthetic oligonucleotide
  source 1..50: mol_type = other DNA
ccccttagggataacagggtaatctgatggcatcaaaatgtgtgtccagt

SEQ ID NO: 28 - DNA, 48 bp, synthetic construct
  misc_feature 1..48: note = Synthetic oligonucleotide
  source 1..48: mol_type = other DNA
ccccttagggataacagggtaatcggaggtggcttcacttaggaggtc

SEQ ID NO: 29 - DNA, 44 bp, synthetic construct
  misc_feature 1..44: note = Synthetic oligonucleotide
  source 1..44: mol_type = other DNA
ccccttagggataacagggtaatcagcaaggctgacaccaggtg

SEQ ID NO: 30 - DNA, 45 bp, synthetic construct
  misc_feature 1..45: note = Synthetic oligonucleotide
  source 1..45: mol_type = other DNA
ccccttagggataacagggtaatcggctgggatctggggagagag

SEQ ID NO: 31 - DNA, 49 bp, synthetic construct
  misc_feature 1..49: note = Synthetic oligonucleotide
  source 1..49: mol_type = other DNA
ccccttagggataacagggtaatcaatcatcaggtgcaaggcaagactg

SEQ ID NO: 32 - DNA, 47 bp, synthetic construct
  misc_feature 1..47: note = Synthetic oligonucleotide
  source 1..47: mol_type = other DNA
ccccttagggataacagggtaatctctgactggagttcccttcacca

SEQ ID NO: 33 - DNA, 45 bp, synthetic construct
  misc_feature 1..45: note = Synthetic oligonucleotide
  source 1..45: mol_type = other DNA
ccccttagggataacagggtaatcgaggagcctggacagacgaag

SEQ ID NO: 34 - DNA, 46 bp, synthetic construct
  misc_feature 1..46: note = Synthetic oligonucleotide
  source 1..46: mol_type = other DNA
ccccttagggataacagggtaatcgctgccagaagcctgtagagat

SEQ ID NO: 35 - DNA, 45 bp, synthetic construct
  misc_feature 1..45: note = Synthetic oligonucleotide
  source 1..45: mol_type = other DNA
ccccttagggataacagggtaatcagctcgggggtgattagttgc

SEQ ID NO: 36 - DNA, 42 bp, synthetic construct
  misc_feature 1..42: note = Synthetic oligonucleotide
  source 1..42: mol_type = other DNA
ccccttagggataacagggtaatcgccgtggcccgacaccta

SEQ ID NO: 37 - DNA, 48 bp, synthetic construct
  misc_feature 1..48: note = Synthetic oligonucleotide
  source 1..48: mol_type = other DNA
ccccttagggataacagggtaatctaagaagggccccttgatgaggtc

SEQ ID NO: 38 - DNA, 46 bp, synthetic construct
  misc_feature 1..46: note = Synthetic oligonucleotide
  source 1..46: mol_type = other DNA
ccccttagggataacagggtaatcgggagaggtgggtcaactttgg

SEQ ID NO: 39 - DNA, 45 bp, synthetic construct
  misc_feature 1..45: note = Synthetic oligonucleotide
  source 1..45: mol_type = other DNA
ccccttagggataacagggtaatcgagggtgggcgtggctatgta

SEQ ID NO: 40 - DNA, 46 bp, synthetic construct
  misc_feature 1..46: note = Synthetic oligonucleotide
  source 1..46: mol_type = other DNA
ccccttagggataacagggtaatcatgaagctggactgcaccatcg

SEQ ID NO: 41 - DNA, 42 bp, synthetic construct
  misc_feature 1..42: note = Synthetic oligonucleotide
  source 1..42: mol_type = other DNA
ccccttagggataacagggtaatctggggaggggcgaaggtc

SEQ ID NO: 42 - DNA, 42 bp, synthetic construct
  misc_feature 1..42: note = Synthetic oligonucleotide
  source 1..42: mol_type = other DNA
ccccttagggataacagggtaatcccgggtcctgcgtccctt

SEQ ID NO: 43 - DNA, 42 bp, synthetic construct
  misc_feature 1..42: note = Synthetic oligonucleotide
  source 1..42: mol_type = other DNA
ccccttagggataacagggtaatcccgcagcctccagatgca

SEQ ID NO: 44 - DNA, 50 bp, synthetic construct
  misc_feature 1..50: note = Synthetic oligonucleotide
  source 1..50: mol_type = other DNA
ccccttagggataacagggtaatcctgaggctcttatcaaacaactgcca

SEQ ID NO: 45 - DNA, 42 bp, synthetic construct
  misc_feature 1..42: note = Synthetic oligonucleotide
  source 1..42: mol_type = other DNA
ccccttagggataacagggtaatccgagtcccggcgctgtcc

SEQ ID NO: 46 - DNA, 42 bp, synthetic construct
  misc_feature 1..42: note = Synthetic oligonucleotide
  source 1..42: mol_type = other DNA
ccccttagggataacagggtaatccctcctcggtccgctgag

SEQ ID NO: 47 - DNA, 48 bp, synthetic construct
  misc_feature 1..48: note = Synthetic oligonucleotide
  source 1..48: mol_type = other DNA
ccccttagggataacagggtaatccaatctctgacttggacagctgca

SEQ ID NO: 48 - DNA, 49 bp, synthetic construct
  misc_feature 1..49: note = Synthetic oligonucleotide
  source 1..49: mol_type = other DNA
ccccttagggataacagggtaatctcgatgatggcctatgggttgaaaa

SEQ ID NO: 49 - DNA, 45 bp, synthetic construct
  misc_feature 1..45: note = Synthetic oligonucleotide
  source 1..45: mol_type = other DNA
ccccttagggataacagggtaatctgtgcttcttccaagctgcgt

SEQ ID NO: 50 - DNA, 49 bp, synthetic construct
  misc_feature 1..49: note = Synthetic oligonucleotide
  source 1..49: mol_type = other DNA
ccccttagggataacagggtaatctgtgattttcctggctttagcgcta

SEQ ID NO: 51 - DNA, 48 bp, synthetic construct
  misc_feature 1..48: note = Synthetic oligonucleotide
  source 1..48: mol_type = other DNA
ccccttagggataacagggtaatctctacaaccaagcccatttgtcca

SEQ ID NO: 52 - DNA, 47 bp, synthetic construct
  misc_feature 1..47: note = Synthetic oligonucleotide
  source 1..47: mol_type = other DNA
ccccttagggataacagggtaatcagcctctctaccatttgtgctga

SEQ ID NO: 53 - DNA, 43 bp, synthetic construct
  misc_feature 1..43: note = Synthetic oligonucleotide
  source 1..43: mol_type = other DNA
ccccttagggataacagggtaatccctgccagggttgcatggg

SEQ ID NO: 54 - DNA, 45 bp, synthetic construct
  misc_feature 1..45: note = Synthetic oligonucleotide
  source 1..45: mol_type = other DNA
ccccttagggataacagggtaatcttgtccccaccctcgtcactc

SEQ ID NO: 55 - DNA, 42 bp, synthetic construct
  misc_feature 1..42: note = Synthetic oligonucleotide
  source 1..42: mol_type = other DNA
ccccttagggataacagggtaatcggcagggacccggcgaca

SEQ ID NO: 56 - DNA, 43 bp, synthetic construct
  misc_feature 1..43: note = Synthetic oligonucleotide
  source 1..43: mol_type = other DNA
ccccttagggataacagggtaatcggaagggaggcgctaggca

SEQ ID NO: 57 - DNA, 42 bp, synthetic construct
  misc_feature 1..42: note = Synthetic oligonucleotide
  source 1..42: mol_type = other DNA
ccccttagggataacagggtaatcgctgggcctgcacgctca

SEQ ID NO: 58 - DNA, 43 bp, synthetic construct
  misc_feature 1..43: note = Synthetic oligonucleotide
  source 1..43: mol_type = other DNA
ccccttagggataacagggtaatcgacagccaccctgctccac

SEQ ID NO: 59 - DNA, 43 bp, synthetic construct
  misc_feature 1..43: note = Synthetic oligonucleotide
  source 1..43: mol_type = other DNA
ccccttagggataacagggtaatccctagcaacggccctggca

SEQ ID NO: 60 - DNA, 44 bp, synthetic construct
  misc_feature 1..44: note = Synthetic oligonucleotide
  source 1..44: mol_type = other DNA
ccccttagggataacagggtaatcccctaccctgccgcgctcct

SEQ ID NO: 61 - DNA, 43 bp, synthetic construct
  misc_feature 1..43: note = Synthetic oligonucleotide
  source 1..43: mol_type = other DNA
ccccttagggataacagggtaatcggcacctcattggggacgt

SEQ ID NO: 62 - DNA, 44 bp, synthetic construct
  misc_feature 1..44: note = Synthetic oligonucleotide
  source 1..44: mol_type = other DNA
ccccttagggataacagggtaatctgctctgaaccgaggccttg

SEQ ID NO: 63 - DNA, 42 bp, synthetic construct
  misc_feature 1..42: note = Synthetic oligonucleotide
  source 1..42: mol_type = other DNA
ccccttagggataacagggtaatcgcagggccccagagaggt

SEQ ID NO: 64 - DNA, 46 bp, synthetic construct
  misc_feature 1..46: note = Synthetic oligonucleotide
  source 1..46: mol_type = other DNA
ccccttagggataacagggtaatcgcagcgacgccactctttatct

SEQ ID NO: 65 - DNA, 42 bp, synthetic construct
  misc_feature 1..42: note = Synthetic oligonucleotide
  source 1..42: mol_type = other DNA
ccccttagggataacagggtaatccgcgctgacagctgttgc

SEQ ID NO: 66 - DNA, 48 bp, synthetic construct
  misc_feature 1..48: note = Synthetic oligonucleotide
  source 1..48: mol_type = other DNA
ccccttagggataacagggtaatctgagtttcatgctcagtccctgca

SEQ ID NO: 67 - DNA, 43 bp, synthetic construct
  misc_feature 1..43: note = Synthetic oligonucleotide
  source 1..43: mol_type = other DNA
ccccttagggataacagggtaatcagggctctggggtgactcc

SEQ ID NO: 68 - DNA, 50 bp, synthetic construct
  misc_feature 1..50: note = Synthetic oligonucleotide
  source 1..50: mol_type = other DNA
ccccttagggataacagggtaatcctccctactcaaccccgagccctcct

SEQ ID NO: 69 - DNA, 42 bp, synthetic construct
  misc_feature 1..42: note = Synthetic oligonucleotide
  source 1..42: mol_type = other DNA
ccccttagggataacagggtaatcgcgccagaccagctccga

SEQ ID NO: 70 - DNA, 42 bp, synthetic construct
  misc_feature 1..42: note = Synthetic oligonucleotide
  source 1..42: mol_type = other DNA
ccccttagggataacagggtaatcctccttgcccggggtagg

SEQ ID NO: 71 - DNA, 50 bp, synthetic construct
  misc_feature 1..50: note = Synthetic oligonucleotide
  source 1..50: mol_type = other DNA
ccccttagggataacagggtaatccttctcatccttgtatcagctgcctt

SEQ ID NO: 72 - DNA, 48 bp, synthetic construct
  misc_feature 1..48: note = Synthetic oligonucleotide
  source 1..48: mol_type = other DNA
ccccttagggataacagggtaatcgggagagtgccattctcagcctaa

SEQ ID NO: 73 - DNA, 43 bp, synthetic construct
  misc_feature 1..43: note = Synthetic oligonucleotide
  source 1..43: mol_type = other DNA
ccccttagggataacagggtaatcggtggatcacgcagtcgga

SEQ ID NO: 74 - DNA, 44 bp, synthetic construct
  misc_feature 1..44: note = Synthetic oligonucleotide
  source 1..44: mol_type = other DNA
ccccttagggataacagggtaatccatgagcacacccaccacca

SEQ ID NO: 75 - DNA, 45 bp, synthetic construct
  misc_feature 1..45: note = Synthetic oligonucleotide
  source 1..45: mol_type = other DNA
ccccttagggataacagggtaatcgccttgtcccacatcacagca

SEQ ID NO: 76 - DNA, 50 bp, synthetic construct
  misc_feature 1..50: note = Synthetic oligonucleotide
  source 1..50: mol_type = other DNA
ccccttagggataacagggtaatccgcctgggactacttctcgtttgaaa

SEQ ID NO: 77 - DNA, 42 bp, synthetic construct
  misc_feature 1..42: note = Synthetic oligonucleotide
  source 1..42: mol_type = other DNA
ccccttagggataacagggtaatccccccaacgtggcctcag

SEQ ID NO: 78 - DNA, 45 bp, synthetic construct
  misc_feature 1..45: note = Synthetic oligonucleotide
  source 1..45: mol_type = other DNA
ccccttagggataacagggtaatccacagctgtccaaacgaggct

SEQ ID NO: 79 - DNA, 46 bp, synthetic construct
  misc_feature 1..46: note = Synthetic oligonucleotide
  source 1..46: mol_type = other DNA
ccccttagggataacagggtaatcggatgcaactgagggctcctta

SEQ ID NO: 80 - DNA, 43 bp, synthetic construct
  misc_feature 1..43: note = Synthetic oligonucleotide
  source 1..43: mol_type = other DNA
ccccttagggataacagggtaatcggaaggtcgctgggaagcc

SEQ ID NO: 81 - DNA, 42 bp, synthetic construct
  misc_feature 1..42: note = Synthetic oligonucleotide
  source 1..42: mol_type = other DNA
ccccttagggataacagggtaatctgcccagcctccctgcag

SEQ ID NO: 82 - DNA, 45 bp, synthetic construct
  misc_feature 1..45: note = Synthetic oligonucleotide
  source 1..45: mol_type = other DNA
ccccttagggataacagggtaatcagctcctctgtgtgacatgcc

SEQ ID NO: 83 - DNA, 49 bp, synthetic construct
  misc_feature 1..49: note = Synthetic oligonucleotide
  source 1..49: mol_type = other DNA
ccccttagggataacagggtaatcaccaaccagctaacactgctatgca

SEQ ID NO: 84 - DNA, 46 bp, synthetic construct
  misc_feature 1..46: note = Synthetic oligonucleotide
  source 1..46: mol_type = other DNA
ccccttagggataacagggtaatcactcaggtgtgctggcactgat

SEQ ID NO: 85 - DNA, 49 bp, synthetic construct
  misc_feature 1..49: note = Synthetic oligonucleotide
  source 1..49: mol_type = other DNA
ccccttagggataacagggtaatcacatacaaccagttcacccagttac

SEQ ID NO: 86 - DNA, 49 bp, synthetic construct
  misc_feature 1..49: note = Synthetic oligonucleotide
  source 1..49: mol_type = other DNA
ccccttagggataacagggtaatcggtgtgtgtttgctgttaccgctta

SEQ ID NO: 87 - DNA, 44 bp, synthetic construct
  misc_feature 1..44: note = Synthetic oligonucleotide
  source 1..44: mol_type = other DNA
ccccttagggataacagggtaatccctggagcgaaggcctggag

SEQ ID NO: 88 - DNA, 45 bp, synthetic construct
  misc_feature 1..45: note = Synthetic oligonucleotide
  source 1..45: mol_type = other DNA
ccccttagggataacagggtaatctactgatgggggtgagctcca

SEQ ID NO: 89 - DNA, 47 bp, synthetic construct
  misc_feature 1..47: note = Synthetic oligonucleotide
  source 1..47: mol_type = other DNA
ccccttagggataacagggtaatctgtgtctctgctttctgttggca

SEQ ID NO: 90 - DNA, 46 bp, synthetic construct
  misc_feature 1..46: note = Synthetic oligonucleotide
  source 1..46: mol_type = other DNA
ccccttagggataacagggtaatctcttcaagcagcccaccttctg

SEQ ID NO: 91 - DNA, 42 bp, synthetic construct
  misc_feature 1..42: note = Synthetic oligonucleotide
  source 1..42: mol_type = other DNA
ccccttagggataacagggtaatcactcccgccggttccaag

SEQ ID NO: 92 - DNA, 44 bp, synthetic construct
  misc_feature 1..44: note = Synthetic oligonucleotide
  source 1..44: mol_type = other DNA
ccccttagggataacagggtaatcccaaagcacaggtggggact

SEQ ID NO: 93 - DNA, 44 bp, synthetic construct
  misc_feature 1..44: note = Synthetic oligonucleotide
  source 1..44: mol_type = other DNA
ccccttagggataacagggtaatccagctgcttgggctccgttg

SEQ ID NO: 94 - DNA, 43 bp, synthetic construct
  misc_feature 1..43: note = Synthetic oligonucleotide
  source 1..43: mol_type = other DNA
ccccttagggataacagggtaatcccccaggccacaggaaacc

SEQ ID NO: 95 - DNA, 45 bp, synthetic construct
  misc_feature 1..45: note = Synthetic oligonucleotide
  source 1..45: mol_type = other DNA
ccccttagggataacagggtaatcgctagggtggctgtgactcag

SEQ ID NO: 96 - DNA, 46 bp, synthetic construct
  misc_feature 1..46: note = Synthetic oligonucleotide
  source 1..46: mol_type = other DNA
ccccttagggataacagggtaatccctctggcttcccatgggtgag

SEQ ID NO: 97 - DNA, 49 bp, synthetic construct
  misc_feature 1..49: note = Synthetic oligonucleotide
  source 1..49: mol_type = other DNA
ccccttagggataacagggtaatcctccctgagaagagctgaacatagc

SEQ ID NO: 98 - DNA, 48 bp, synthetic construct
  misc_feature 1..48: note = Synthetic oligonucleotide
  source 1..48: mol_type = other DNA
ccccttagggataacagggtaatctcaacccttcccatgactgaggtg

SEQ ID NO: 99 - DNA, 42 bp, synthetic construct
  misc_feature 1..42: note = Synthetic oligonucleotide
  source 1..42: mol_type = other DNA
ccccttagggataacagggtaatccccaaccccctgcagctg

SEQ ID NO: 100 - DNA, 47 bp, synthetic construct
  misc_feature 1..47: note = Synthetic oligonucleotide
  source 1..47: mol_type = other DNA
ccccttagggataacagggtaatctcaaaatcccaagggcattgttc

SEQ ID NO: 101 - DNA, 50 bp, synthetic construct
  misc_feature 1..50: note = Synthetic oligonucleotide
  source 1..50: mol_type = other DNA
ccccttagggataacagggtaatccattgtgtcttcttggtacccttttt

SEQ ID NO: 102 - DNA, 46 bp, synthetic construct
  misc_feature 1..46: note = Synthetic oligonucleotide
  source 1..46: mol_type = other DNA
ccccttagggataacagggtaatcagatcacacgaggcagagggaa

SEQ ID NO: 103 - DNA, 46 bp, synthetic construct
  misc_feature 1..46: note = Synthetic oligonucleotide
  source 1..46: mol_type = other DNA
ccccttagggataacagggtaatcgcaatctcacctcctccctctc

SEQ ID NO: 104 - DNA, 43 bp, synthetic construct
  misc_feature 1..43: note = Synthetic oligonucleotide
  source 1..43: mol_type = other DNA
ccccttagggataacagggtaatcagccaaacacagaaaggcc

SEQ ID NO: 105 - DNA, 45 bp, synthetic construct
  misc_feature 1..45: note = Synthetic oligonucleotide
  source 1..45: mol_type = other DNA
ccccttagggataacagggtaatcgtgagccatgatcgtgcactc

SEQ ID NO: 106 - DNA, 47 bp, synthetic construct
  misc_feature 1..47: note = Synthetic oligonucleotide
  source 1..47: mol_type = other DNA
ccccttagggataacagggtaatcactacattggaggagtgtgtacc

SEQ ID NO: 107 - DNA, 45 bp, synthetic construct
  misc_feature 1..45: note = Synthetic oligonucleotide
  source 1..45: mol_type = other DNA
ccccttagggataacagggtaatcctctgctttcccctcccacct

SEQ ID NO: 108 - DNA, 47 bp, synthetic construct
  misc_feature 1..47: note = Synthetic oligonucleotide
  source 1..47: mol_type = other DNA
ccccttagggataacagggtaatcctgccctgttggataacccttct

SEQ ID NO: 109 - DNA, 50 bp, synthetic construct
  misc_feature 1..50: note = Synthetic oligonucleotide
  source 1..50: mol_type = other DNA
ccccttagggataacagggtaatccttggaaagggatgctctgaatacct

SEQ ID NO: 110 - DNA, 46 bp, synthetic construct
  misc_feature 1..46: note = Synthetic oligonucleotide
  source 1..46: mol_type = other DNA
ccccttagggataacagggtaatcagctgcactttctcccggacaa

SEQ ID NO: 111 - DNA, 49 bp, synthetic construct
  misc_feature 1..49: note = Synthetic oligonucleotide
  source 1..49: mol_type = other DNA
ccccttagggataacagggtaatctgttgttaaggctgttggcatctgt

SEQ ID NO: 112 - DNA, 47 bp, synthetic construct
  misc_feature 1..47: note = Synthetic oligonucleotide
  source 1..47: mol_type = other DNA
ccccttagggataacagggtaatcaggaaaacacggttgcatcctga

SEQ ID NO: 113 - DNA, 44 bp, synthetic construct
  misc_feature 1..44: note = Synthetic oligonucleotide
  source 1..44: mol_type = other DNA
ccccttagggataacagggtaatcgcaccagctcttcggccaag

SEQ ID NO: 114 - DNA, 47 bp, synthetic construct
  misc_feature 1..47: note = Synthetic oligonucleotide
  source 1..47: mol_type = other DNA
ccccttagggataacagggtaatctccgtgtgtttgactccctcaac

SEQ ID NO: 115 - DNA, 45 bp, synthetic construct
  misc_feature 1..45: note = Synthetic oligonucleotide
  source 1..45: mol_type = other DNA
ccccttagggataacagggtaatcggcggtgtcagcaaagctagg

SEQ ID NO: 116 - DNA, 43 bp, synthetic construct
  misc_feature 1..43: note = Synthetic oligonucleotide
  source 1..43: mol_type = other DNA
ccccttagggataacagggtaatcagcaccgatgaggcatggg

SEQ ID NO: 117 - DNA, 45 bp, synthetic construct
  misc_feature 1..45: note = Synthetic oligonucleotide
  source 1..45: mol_type = other DNA
ccccttagggataacagggtaatcgtgctgcctccccctctggta

SEQ ID NO: 118 - DNA, 45 bp, synthetic construct
  misc_feature 1..45: note = Synthetic oligonucleotide
  source 1..45: mol_type = other DNA
ccccttagggataacagggtaatcggagtgactggatgctgggtt

SEQ ID NO: 119 - DNA, 47 bp, synthetic construct
  misc_feature 1..47: note = Synthetic oligonucleotide
  source 1..47: mol_type = other DNA
ccccttagggataacagggtaatcggagaggggcctggaagattctc

SEQ ID NO: 120 - DNA, 47 bp, synthetic construct
  misc_feature 1..47: note = Synthetic oligonucleotide
  source 1..47: mol_type = other DNA
ccccttagggataacagggtaatcgggtagacttgacataagcacca

SEQ ID NO: 121 - DNA, 44 bp, synthetic construct
  misc_feature 1..44: note = Synthetic oligonucleotide
  source 1..44: mol_type = other DNA
ccccttagggataacagggtaatcaagaaaaggggctgctgggt

SEQ ID NO: 122 - DNA, 46 bp, synthetic construct
  misc_feature 1..46: note = Synthetic oligonucleotide
  source 1..46: mol_type = other DNA
ccccttagggataacagggtaatcgacgtgattcgagttcctggca

SEQ ID NO: 123 - DNA, 46 bp, synthetic construct
  misc_feature 1..46: note = Synthetic oligonucleotide
  source 1..46: mol_type = other DNA
ccccttagggataacagggtaatcgggtctggggtcctgaaatgac

SEQ ID NO: 124 - DNA, 49 bp, synthetic construct
  misc_feature 1..49: note = Synthetic oligonucleotide
  source 1..49: mol_type = other DNA
ccccttagggataacagggtaatccccactagatcctgtcacaattccc

SEQ ID NO: 125 - DNA, 44 bp, synthetic construct
  misc_feature 1..44: note = Synthetic oligonucleotide
  source 1..44: mol_type = other DNA
ccccttagggataacagggtaatctgtgctcccgctcccatgag

SEQ ID NO: 126 - DNA, 50 bp, synthetic construct
  misc_feature 1..50: note = Synthetic oligonucleotide
  source 1..50: mol_type = other DNA
ccccttagggataacagggtaatctgcacaagaacctgctgtctaaactt

SEQ ID NO: 127 - DNA, 42 bp, synthetic construct
  misc_feature 1..42: note = Synthetic oligonucleotide
  source 1..42: mol_type = other DNA
ccccttagggataacagggtaatcacgacggccgcacagtgg

SEQ ID NO: 128 - DNA, 44 bp, synthetic construct
  misc_feature 1..44: note = Synthetic oligonucleotide
  source 1..44: mol_type = other DNA
ccccttagggataacagggtaatccccgcccactctcgactctt

SEQ ID NO: 129 - DNA, 43 bp, synthetic construct
  misc_feature 1..43: note = Synthetic oligonucleotide
  source 1..43: mol_type = other DNA
ccccttagggataacagggtaatcccaggactccccaggtgct

SEQ ID NO: 130 - DNA, 46 bp, synthetic construct
  misc_feature 1..46: note = Synthetic oligonucleotide
  source 1..46: mol_type = other DNA
ccccttagggataacagggtaatcctctagcccgaaaagccaagct

SEQ ID NO: 131 - DNA, 44 bp, synthetic construct
  misc_feature 1..44: note = Synthetic oligonucleotide
  source 1..44: mol_type = other DNA
ccccttagggataacagggtaatcctctgccagatgctgctcgt

SEQ ID NO: 132 - DNA, 47 bp, synthetic construct
  misc_feature 1..47: note = Synthetic oligonucleotide
  source 1..47: mol_type = other DNA
ccccttagggataacagggtaatcgctgttgcaggtccagaggacac

SEQ ID NO: 133 - DNA, 45 bp, synthetic construct
  misc_feature 1..45: note = Synthetic oligonucleotide
  source 1..45: mol_type = other DNA
ccccttagggataacagggtaatcgaagacgtgggttctggcaga

SEQ ID NO: 134 - DNA, 42 bp, synthetic construct
  misc_feature 1..42: note = Synthetic oligonucleotide
  source 1..42: mol_type = other DNA
ccccttagggataacagggtaatccccccgtcccctctgcca

SEQ ID NO: 135 - DNA, 43 bp, synthetic construct
  misc_feature 1..43: note = Synthetic oligonucleotide
  source 1..43: mol_type = other DNA
ccccttagggataacagggtaatctgcctgtgtccggagctgt

SEQ ID NO: 136 - DNA, 43 bp, synthetic construct
  misc_feature 1..43: note = Synthetic oligonucleotide
  source 1..43: mol_type = other DNA
ccccttagggataacagggtaatccctaccggggcaagacagc

SEQ ID NO: 137 - DNA, 50 bp, synthetic construct
  misc_feature 1..50: note = Synthetic oligonucleotide
  source 1..50: mol_type = other DNA
ccccttagggataacagggtaatctggtgacaactaccactctagaattt

SEQ ID NO: 138 - DNA, 49 bp, synthetic construct
  misc_feature 1..49: note = Synthetic oligonucleotide
  source 1..49: mol_type = other DNA
ccccttagggataacagggtaatctgtcctaaaacccctgcttggattt

SEQ ID NO: 139 - DNA, 46 bp, synthetic construct
  misc_feature 1..46: note = Synthetic oligonucleotide
  source 1..46: mol_type = other DNA
ccccttagggataacagggtaatctgcggaacacaggagctagtct

SEQ ID NO: 140 - DNA, 48 bp, synthetic construct
  misc_feature 1..48: note = Synthetic oligonucleotide
  source 1..48: mol_type = other DNA
ccccttagggataacagggtaatctcagtagctccccaacgttactgt

SEQ ID NO: 141 - DNA, 47 bp, synthetic construct
  misc_feature 1..47: note = Synthetic oligonucleotide
  source 1..47: mol_type = other DNA
ccccttagggataacagggtaatcctgtctcccaaaatcaggcctgt

SEQ ID NO: 142 - DNA, 42 bp, synthetic construct
  misc_feature 1..42: note = Synthetic oligonucleotide
  source 1..42: mol_type = other DNA
ccccttagggataacagggtaatctttgtgcgccctgccctt

SEQ ID NO: 143 - DNA, 44 bp, synthetic construct
  misc_feature 1..44: note = Synthetic oligonucleotide
  source 1..44: mol_type = other DNA
ccccttagggataacagggtaatcggggaggagggtgtttacgg

SEQ ID NO: 144 - DNA, 47 bp, synthetic construct
  misc_feature 1..47: note = Synthetic oligonucleotide
  source 1..47: mol_type = other DNA
ccccttagggataacagggtaatcaggcagtgcaggcgttataaact

SEQ ID NO: 145 - DNA, 46 bp, synthetic construct
  misc_feature 1..46: note = Synthetic oligonucleotide
  source 1..46: mol_type = other DNA
ccccttagggataacagggtaatccactttccctggggcttgctta

SEQ ID NO: 146 - DNA, 43 bp, synthetic construct
  misc_feature 1..43: note = Synthetic oligonucleotide
  source 1..43: mol_type = other DNA
ccccttagggataacagggtaatcggaaggggcgtgggaagcg

SEQ ID NO: 147 - DNA, 46 bp, synthetic construct
  misc_feature 1..46: note = Synthetic oligonucleotide
  source 1..46: mol_type = other DNA
ccccttagggataacagggtaatcccgcagacagagcaagcgcgtt

SEQ ID NO: 148 - DNA, 44 bp, synthetic construct
  misc_feature 1..44: note = Synthetic oligonucleotide
  source 1..44: mol_type = other DNA
ccccttagggataacagggtaatcgccccagctaccacctcctc

SEQ ID NO: 149 - DNA, 42 bp, synthetic construct
  misc_feature 1..42: note = Synthetic oligonucleotide
  source 1..42: mol_type = other DNA
ccccttagggataacagggtaatccgcggaccacggctcctc

SEQ ID NO: 150 - DNA, 56 bp, synthetic construct
  misc_feature 1..56: note = Synthetic oligonucleotide
  source 1..56: mol_type = other DNA
gtgactggagttcagacgtgtgctcttccgatctgccctgggtctctgaagaagct

SEQ ID NO: 151 - DNA, 60 bp, synthetic construct
  misc_feature 1..60: note = Synthetic oligonucleotide
  source 1..60: mol_type = other DNA
gtgactggagttcagacgtgtgctcttccgatctggcgggtgggtcacaaaataaaatgt

SEQ ID NO: 152 - DNA, 58 bp, synthetic construct
  misc_feature 1..58: note = Synthetic oligonucleotide
  source 1..58: mol_type = other DNA
gtgactggagttcagacgtgtgctcttccgatctacaggacagggtcagcgtttaaga

SEQ ID NO: 153 - DNA, 57 bp, synthetic construct
  misc_feature 1..57: note = Synthetic oligonucleotide
  source 1..57: mol_type = other DNA
gtgactggagttcagacgtgtgctcttccgatctaagtttgctggccctggtttaga

SEQ ID NO: 154 - DNA, 56 bp, synthetic construct
  misc_feature 1..56: note = Synthetic oligonucleotide
  source 1..56: mol_type = other DNA
gtgactggagttcagacgtgtgctcttccgatcttgcagtacagtgatgggaccgt

SEQ ID NO: 155 - DNA, 53 bp, synthetic construct
  misc_feature 1..53: note = Synthetic oligonucleotide
  source 1..53: mol_type = other DNA
gtgactggagttcagacgtgtgctcttccgatctccttagggggccagcagtg

SEQ ID NO: 156 - DNA, 54 bp, synthetic construct
  misc_feature 1..54: note = Synthetic oligonucleotide
  source 1..54: mol_type = other DNA
gtgactggagttcagacgtgtgctcttccgatctgcagacgcccccatcaagcc

SEQ ID NO: 157 - DNA, 59 bp, synthetic construct
  misc_feature 1..59: note = Synthetic oligonucleotide
  source 1..59: mol_type = other DNA
gtgactggagttcagacgtgtgctcttccgatcttggagttaagggtgtctccgaggtg

SEQ ID NO: 158 - DNA, 52 bp, synthetic construct
  misc_feature 1..52: note = Synthetic oligonucleotide
  source 1..52: mol_type = other DNA
gtgactggagttcagacgtgtgctcttccgatctgcgcgcacccctttccca

SEQ ID NO: 159 - DNA, 55 bp, synthetic construct
  misc_feature 1..55: note = Synthetic oligonucleotide
  source 1..55: mol_type = other DNA
gtgactggagttcagacgtgtgctcttccgatctggtgaggccgtgcagttggtc

SEQ ID NO: 160 - DNA, 60 bp, synthetic construct
  misc_feature 1..60: note = Synthetic oligonucleotide
  source 1..60: mol_type = other DNA
gtgactggagttcagacgtgtgctcttccgatctaagcctcttgaacatgccgaaatgta

SEQ ID NO: 161 - DNA, 57 bp, synthetic construct
  misc_feature 1..57: note = Synthetic oligonucleotide
  source 1..57: mol_type = other DNA
gtgactggagttcagacgtgtgctcttccgatcttcgtctcctggagctctggacac

SEQ ID NO: 162 - DNA, 59 bp, synthetic construct
  misc_feature 1..59: note = Synthetic oligonucleotide
  source 1..59: mol_type = other DNA
gtgactggagttcagacgtgtgctcttccgatctccacctgtggctgatagtgacgtct

SEQ ID NO: 163 - DNA, 54 bp, synthetic construct
  misc_feature 1..54: note = Synthetic oligonucleotide
  source 1..54: mol_type = other DNA
gtgactggagttcagacgtgtgctcttccgatctaggtctggggagcggagtcc

SEQ ID NO: 164 - DNA, 53 bp, synthetic construct
  misc_feature 1..53: note = Synthetic oligonucleotide
  source 1..53: mol_type = other DNA
gtgactggagttcagacgtgtgctcttccgatctaccgcctccacccccaagg

SEQ ID NO: 165 - DNA, 56 bp, synthetic construct
  misc_feature 1..56: note = Synthetic oligonucleotide
  source 1..56: mol_type = other DNA
gtgactggagttcagacgtgtgctcttccgatctggatctggggagagaggtgacc

SEQ ID NO: 166 - DNA, 59 bp, synthetic construct
  misc_feature 1..59: note = Synthetic oligonucleotide
  source 1..59: mol_type = other DNA
gtgactggagttcagacgtgtgctcttccgatcttgtatgtatgcaaagccccgtcacg

SEQ ID NO: 167 - DNA, 60 bp, synthetic construct
  misc_feature 1..60: note = Synthetic oligonucleotide
  source 1..60: mol_type = other DNA
gtgactggagttcagacgtgtgctcttccgatctgactggagttcccttcaccatttcaa

SEQ ID NO: 168 - DNA, 55 bp, synthetic construct
  misc_feature 1..55: note = Synthetic oligonucleotide
  source 1..55: mol_type = other DNA
gtgactggagttcagacgtgtgctcttccgatctcctggacagacgaaggcagca

SEQ ID NO: 169 - DNA, 57 bp, synthetic construct
  misc_feature 1..57: note = Synthetic oligonucleotide
  source 1..57: mol_type = other DNA
gtgactggagttcagacgtgtgctcttccgatctgcctgtagagatcaaggctgctc

SEQ ID NO: 170 - DNA, 60 bp, synthetic construct
  misc_feature 1..60: note = Synthetic oligonucleotide
  source 1..60: mol_type = other DNA
gtgactggagttcagacgtgtgctcttccgatcttcgggggtgattagttgctttttgtt

SEQ ID NO: 171 - DNA, 55 bp, synthetic construct
  misc_feature 1..55: note = Synthetic oligonucleotide
  source 1..55: mol_type = other DNA
gtgactggagttcagacgtgtgctcttccgatctcccgacacctaccggctctcc

SEQ ID NO: 172 - DNA, 60 bp, synthetic construct
  misc_feature 1..60: note = Synthetic oligonucleotide
  source 1..60: mol_type = other DNA
gtgactggagttcagacgtgtgctcttccgatctggtcctggtgctgttcagcccatctt

SEQ ID NO: 173 - DNA, 56 bp, synthetic construct
  misc_feature 1..56: note = Synthetic oligonucleotide
  source 1..56: mol_type = other DNA
gtgactggagttcagacgtgtgctcttccgatctggtgggtcaactttggcagggt

SEQ ID NO: 174 - DNA, 58 bp, synthetic construct
  misc_feature 1..58: note = Synthetic oligonucleotide
  source 1..58: mol_type = other DNA
gtgactggagttcagacgtgtgctcttccgatctggtgggcgtggctatgtaaacgga

SEQ ID NO: 175 - DNA, 56 bp, synthetic construct
  misc_feature 1..56: note = Synthetic oligonucleotide
  source 1..56: mol_type = other DNA
gtgactggagttcagacgtgtgctcttccgatctctggactgcaccatcgctcagg

SEQ ID NO: 176 - DNA, 56 bp, synthetic construct
  misc_feature 1..56: note = Synthetic oligonucleotide
  source 1..56: mol_type = other DNA
gtgactggagttcagacgtgtgctcttccgatctaagggagtctcaggcccgtgag

SEQ ID NO: 177 - DNA, 55 bp, synthetic construct
  misc_feature 1..55: note = Synthetic oligonucleotide
  source 1..55: mol_type = other DNA
gtgactggagttcagacgtgtgctcttccgatctgtcctgcgtcccttcccctga

SEQ ID NO: 178 - DNA, 52 bp, synthetic construct
  misc_feature 1..52: note = Synthetic oligonucleotide
  source 1..52: mol_type = other DNA
gtgactggagttcagacgtgtgctcttccgatctgcaccggcagccacctgt

SEQ ID NO: 179 - DNA, 58 bp, synthetic construct
  misc_feature 1..58: note = Synthetic oligonucleotide
  source 1..58: mol_type = other DNA
gtgactggagttcagacgtgtgctcttccgatctaactgccactactcccgtcctcag

SEQ ID NO: 180 - DNA, 53 bp, synthetic construct
  misc_feature 1..53: note = Synthetic oligonucleotide
  source 1..53: mol_type = other DNA
gtgactggagttcagacgtgtgctcttccgatcttgtccgcgtctgtgtcggt

SEQ ID NO: 181 - DNA, 52 bp, synthetic construct
  misc_feature 1..52: note = Synthetic oligonucleotide
  source 1..52: mol_type = other DNA
gtgactggagttcagacgtgtgctcttccgatctcccgggcctcgagcagac

SEQ ID NO: 182 - DNA, 57 bp, synthetic construct
  misc_feature 1..57: note = Synthetic oligonucleotide
  source 1..57: mol_type = other DNA
gtgactggagttcagacgtgtgctcttccgatctcttggacagctgcagtactccct

SEQ ID NO: 183 - DNA, 55 bp, synthetic construct
  misc_feature 1..55: note = Synthetic oligonucleotide
  source 1..55: mol_type = other DNA
gtgactggagttcagacgtgtgctcttccgatctagtgcggtggagaaaggcaag

SEQ ID NO: 184 - DNA, 58 bp, synthetic construct
  misc_feature 1..58: note = Synthetic oligonucleotide
  source 1..58: mol_type = other DNA
gtgactggagttcagacgtgtgctcttccgatctgcgtctctttggacccgtacttgc

SEQ ID NO: 185 - DNA, 60 bp, synthetic construct
  misc_feature 1..60: note = Synthetic oligonucleotide
  source 1..60: mol_type = other DNA
gtgactggagttcagacgtgtgctcttccgatcttcctggctttagcgctatacgtttga

SEQ ID NO: 186 - DNA, 57 bp, synthetic construct
  misc_feature 1..57: note = Synthetic oligonucleotide
  source 1..57: mol_type = other DNA
gtgactggagttcagacgtgtgctcttccgatctcatttgtccaggaacccctagcc

SEQ ID NO: 187 - DNA, 60 bp, synthetic construct
  misc_feature 1..60: note = Synthetic oligonucleotide
  source 1..60: mol_type = other DNA
gtgactggagttcagacgtgtgctcttccgatctaccatttgtgctgatctgtgggtatc

SEQ ID NO: 188 - DNA, 59 bp, synthetic construct
  misc_feature 1..59: note = Synthetic oligonucleotide
  source 1..59: mol_type = other DNA
gtgactggagttcagacgtgtgctcttccgatctcagggttgcatgggaacttcctctg

SEQ ID NO: 189 - DNA, 57 bp, synthetic construct
  misc_feature 1..57: note = Synthetic oligonucleotide
  source 1..57: mol_type = other DNA
gtgactggagttcagacgtgtgctcttccgatctcccaccctcgtcactctctgacc

SEQ ID NO: 190 - DNA, 53 bp, synthetic construct
  misc_feature 1..53: note = Synthetic oligonucleotide
  source 1..53: mol_type = other DNA
gtgactggagttcagacgtgtgctcttccgatctccggcgacactggggaatg

SEQ ID NO: 191 - DNA, 52 bp, synthetic construct
  misc_feature 1..52: note = Synthetic oligonucleotide
  source 1..52: mol_type = other DNA
gtgactggagttcagacgtgtgctcttccgatctcccgggctccgtgcaaac

SEQ ID NO: 192 - DNA, 52 bp, synthetic construct
  misc_feature 1..52: note = Synthetic oligonucleotide
  source 1..52: mol_type = other DNA
gtgactggagttcagacgtgtgctcttccgatctaggccccctggagctgca

SEQ ID NO: 193 - DNA, 54 bp, synthetic construct
  misc_feature 1..54: note = Synthetic oligonucleotide
  source 1..54: mol_type = other DNA
gtgactggagttcagacgtgtgctcttccgatctccaccctgctccacacacct

SEQ ID NO: 194 - DNA, 52 bp, synthetic construct
  misc_feature 1..52: note = Synthetic oligonucleotide
  source 1..52: mol_type = other DNA
gtgactggagttcagacgtgtgctcttccgatctacggccctggcaccacct

SEQ ID NO: 195 - DNA, 53 bp, synthetic construct
  misc_feature 1..53: note = Synthetic oligonucleotide
  source 1..53: mol_type = other DNA
gtgactggagttcagacgtgtgctcttccgatctctgccgcgctcctccttcc

SEQ ID NO: 196 - DNA, 60 bp, synthetic construct
  misc_feature 1..60: note = Synthetic oligonucleotide
  source 1..60: mol_type = other DNA
gtgactggagttcagacgtgtgctcttccgatctcctcattggggacgtctgttgtgaaa

SEQ ID NO: 197 - DNA, 59 bp, synthetic construct
  misc_feature 1..59: note = Synthetic oligonucleotide
  source 1..59: mol_type = other DNA
gtgactggagttcagacgtgtgctcttccgatctagtccccagttacggagacaaatct

SEQ ID NO: 198 - DNA, 60 bp, synthetic construct
  misc_feature 1..60: note = Synthetic oligonucleotide
  source 1..60: mol_type = other DNA
gtgactggagttcagacgtgtgctcttccgatctggccccagagaggtgaggtcactata

SEQ ID NO: 199 - DNA, 53 bp, synthetic construct
  misc_feature 1..53: note = Synthetic oligonucleotide
  source 1..53: mol_type = other DNA
gtgactggagttcagacgtgtgctcttccgatctggtaccccgtgcctcagct

SEQ ID NO: 200 - DNA, 57 bp, synthetic construct
  misc_feature 1..57: note = Synthetic oligonucleotide
  source 1..57: mol_type = other DNA
gtgactggagttcagacgtgtgctcttccgatctccaggttgtttatctgggcctct

SEQ ID NO: 201 - DNA, 58 bp, synthetic construct
  misc_feature 1..58: note = Synthetic oligonucleotide
  source 1..58: mol_type = other DNA
gtgactggagttcagacgtgtgctcttccgatctgcaggacacagcacaggtaaggga

SEQ ID NO: 202 - DNA, 58 bp, synthetic construct
  misc_feature 1..58: note = Synthetic oligonucleotide
  source 1..58: mol_type = other DNA
gtgactggagttcagacgtgtgctcttccgatctctggggtgactccaaggcttttcg

SEQ ID NO: 203 - DNA, 54 bp, synthetic construct
  misc_feature 1..54: note = Synthetic oligonucleotide
  source 1..54: mol_type = other DNA
gtgactggagttcagacgtgtgctcttccgatctccccgagccctcctctcttg

SEQ ID NO: 204 - DNA, 53 bp, synthetic construct
  misc_feature 1..53: note = Synthetic oligonucleotide
  source 1..53: mol_type = other DNA
gtgactggagttcagacgtgtgctcttccgatctagctccgactccgctcgct

SEQ ID NO: 205 - DNA, 55 bp, synthetic construct
  misc_feature 1..55: note = Synthetic oligonucleotide
  source 1..55: mol_type = other DNA
gtgactggagttcagacgtgtgctcttccgatctttgcccggggtaggaaagtga

SEQ ID NO: 206 - DNA, 60 bp, synthetic construct
  misc_feature 1..60: note = Synthetic oligonucleotide
  source 1..60: mol_type = other DNA
gtgactggagttcagacgtgtgctcttccgatctgtatcagctgccttctcatcacaaga

SEQ ID NO: 207 - DNA, 60 bp, synthetic construct
  misc_feature 1..60: note = Synthetic oligonucleotide
  source 1..60: mol_type = other DNA
gtgactggagttcagacgtgtgctcttccgatctgtgccattctcagcctaaaaggtaga

SEQ ID NO: 208 - DNA, 53 bp, synthetic construct
  misc_feature 1..53: note = Synthetic oligonucleotide
  source 1..53: mol_type = other DNA
gtgactggagttcagacgtgtgctcttccgatctacgcagtcggaggccatcc

SEQ ID NO: 209 - DNA, 56 bp, synthetic construct
  misc_feature 1..56: note = Synthetic oligonucleotide
  source 1..56: mol_type = other DNA
gtgactggagttcagacgtgtgctcttccgatctagcacacccaccaccactccta

SEQ ID NO: 210 - DNA, 59 bp, synthetic construct
  misc_feature 1..59: note = Synthetic oligonucleotide
  source 1..59: mol_type = other DNA
gtgactggagttcagacgtgtgctcttccgatctcttgtcccacatcacagcaaactct

SEQ ID NO: 211 - DNA, 53 bp, synthetic construct
  misc_feature 1..53: note = Synthetic oligonucleotide
  source 1..53: mol_type = other DNA
gtgactggagttcagacgtgtgctcttccgatctcggggctgcggaaggatcc

SEQ ID NO: 212 - DNA, 54 bp, synthetic construct
  misc_feature 1..54: note = Synthetic oligonucleotide
  source 1..54: mol_type = other DNA
gtgactggagttcagacgtgtgctcttccgatctcaacgtggcctcagctgctc

SEQ ID NO: 213 - DNA, 53 bp, synthetic construct
  misc_feature 1..53: note = Synthetic oligonucleotide
  source 1..53: mol_type = other DNA
gtgactggagttcagacgtgtgctcttccgatctacgaggctggctccccact

SEQ ID NO: 214 - DNA, 60 bp, synthetic construct
  misc_feature 1..60: note = Synthetic oligonucleotide
  source 1..60: mol_type = other DNA
gtgactggagttcagacgtgtgctcttccgatctctccttagaaagtcatgccccaggag

SEQ ID NO: 215 - DNA, 53 bp, synthetic construct
  misc_feature 1..53: note = Synthetic oligonucleotide
  source 1..53: mol_type = other DNA
gtgactggagttcagacgtgtgctcttccgatctgggctgggaggggattggc

SEQ ID NO: 216 - DNA, 56 bp, synthetic construct
  misc_feature 1..56: note = Synthetic oligonucleotide
  source 1..56: mol_type = other DNA
gtgactggagttcagacgtgtgctcttccgatctgcctccctgcaggatgattggc

SEQ ID NO: 217 - DNA, 60 bp, synthetic construct
  misc_feature 1..60: note = Synthetic oligonucleotide
  source 1..60: mol_type = other DNA
gtgactggagttcagacgtgtgctcttccgatctatgccacagatgactattgcacacct

SEQ ID NO: 218 - DNA, 52 bp, synthetic construct
  misc_feature 1..52: note = Synthetic oligonucleotide
  source 1..52: mol_type = other DNA
gtgactggagttcagacgtgtgctcttccgatctacctcaggggccgctgca

SEQ ID NO: 219 - DNA, 57 bp, synthetic construct
  misc_feature 1..57: note = Synthetic oligonucleotide
  source 1..57: mol_type = other DNA
gtgactggagttcagacgtgtgctcttccgatctgctggcactgatctgtggtccca

SEQ ID NO: 220 - DNA, 60 bp, synthetic construct
  misc_feature 1..60: note = Synthetic oligonucleotide
  source 1..60: mol_type = other DNA
gtgactggagttcagacgtgtgctcttccgatctaaccagttcacccagttacagtagac

SEQ ID NO: 221 - DNA, 60 bp, synthetic construct
  misc_feature 1..60: note = Synthetic oligonucleotide
  source 1..60: mol_type = other DNA
gtgactggagttcagacgtgtgctcttccgatctaccgcttaaattaaccctgagtgacg

SEQ ID NO: 222 - DNA, 52 bp, synthetic construct
  misc_feature 1..52: note = Synthetic oligonucleotide
  source 1..52: mol_type = other DNA
gtgactggagttcagacgtgtgctcttccgatcttagcgtgcggcccgagct

SEQ ID NO: 223 - DNA, 54 bp, synthetic construct
  misc_feature 1..54: note = Synthetic oligonucleotide
  source 1..54: mol_type = other DNA
gtgactggagttcagacgtgtgctcttccgatctgggggtgagctccaactctg

SEQ ID NO: 224 - DNA, 60 bp, synthetic construct
  misc_feature 1..60: note = Synthetic oligonucleotide
  source 1..60: mol_type = other DNA
gtgactggagttcagacgtgtgctcttccgatctatgtatctggcattacagctgagcag

SEQ ID NO: 225 - DNA, 54 bp, synthetic construct
  misc_feature 1..54: note = Synthetic oligonucleotide
  source 1..54: mol_type = other DNA
gtgactggagttcagacgtgtgctcttccgatctcagccactgcaccgacttca

SEQ ID NO: 226 - DNA, 60 bp, synthetic construct
  misc_feature 1..60: note = Synthetic oligonucleotide
  source 1..60: mol_type = other DNA
gtgactggagttcagacgtgtgctcttccgatctcggttccaagttatcggagtgagcca

SEQ ID NO: 227 - DNA, 60 bp, synthetic construct
  misc_feature 1..60: note = Synthetic oligonucleotide
  source 1..60: mol_type = other DNA
gtgactggagttcagacgtgtgctcttccgatctggactcatagcctgggggtaaatgtt

SEQ ID NO: 228 - DNA, 56 bp, synthetic construct
  misc_feature 1..56: note = Synthetic oligonucleotide
  source 1..56: mol_type = other DNA
gtgactggagttcagacgtgtgctcttccgatcttgcttgggctccgttgcaatcc

SEQ ID NO: 229 - DNA, 58 bp, synthetic construct
  misc_feature 1..58: note = Synthetic oligonucleotide
  source 1..58: mol_type = other DNA
gtgactggagttcagacgtgtgctcttccgatctaaaccaggggagagggccatagag

SEQ ID NO: 230 - DNA, 55 bp, synthetic construct
  misc_feature 1..55: note = Synthetic oligonucleotide
  source 1..55: mol_type = other DNA
gtgactggagttcagacgtgtgctcttccgatctgctgtgactcagagccatggc

SEQ ID NO: 231 - DNA, 57 bp, synthetic construct
  misc_feature 1..57: note = Synthetic oligonucleotide
  source 1..57: mol_type = other DNA
gtgactggagttcagacgtgtgctcttccgatctggcttcccatgggtgagtcctgt

SEQ ID NO: 232 - DNA, 60 bp, synthetic construct
  misc_feature 1..60: note = Synthetic oligonucleotide
  source 1..60: mol_type = other DNA
gtgactggagttcagacgtgtgctcttccgatctgaagagctgaacatagccaggcaatt

SEQ ID NO: 233 - DNA, 60 bp, synthetic construct
  misc_feature 1..60: note = Synthetic oligonucleotide
  source 1..60: mol_type = other DNA
gtgactggagttcagacgtgtgctcttccgatcttgactgaggtggatgaacccctaagc

SEQ ID NO: 234 - DNA, 56 bp, synthetic construct
  misc_feature 1..56: note = Synthetic oligonucleotide
  source 1..56: mol_type = other DNA
gtgactggagttcagacgtgtgctcttccgatctaaccccctgcagctgctcacaa

SEQ ID NO: 235 - DNA, 60 bp, synthetic construct
  misc_feature 1..60: note = Synthetic oligonucleotide
  source 1..60: mol_type = other DNA
gtgactggagttcagacgtgtgctcttccgatctaaatcccaagggcattgttcacataa

SEQ ID NO: 236 - DNA, 60 bp, synthetic construct
  misc_feature 1..60: note = Synthetic oligonucleotide
  source 1..60: mol_type = other DNA
gtgactggagttcagacgtgtgctcttccgatctaccctttttgaaaattagttgcccat

SEQ ID NO: 237 - DNA, 58 bp, synthetic construct
  misc_feature 1..58: note = Synthetic oligonucleotide
  source 1..58: mol_type = other DNA
gtgactggagttcagacgtgtgctcttccgatctgaggcagagggaactacaggtgca

SEQ ID NO: 238 - DNA, 59 bp, synthetic construct
  misc_feature 1..59: note = Synthetic oligonucleotide
  source 1..59: mol_type = other DNA
gtgactggagttcagacgtgtgctcttccgatctcctccctctcctaccaacttcatcc

SEQ ID NO: 239 - DNA, 60 bp, synthetic construct
  misc_feature 1..60: note = Synthetic oligonucleotide
  source 1..60: mol_type = other DNA
gtgactggagttcagacgtgtgctcttccgatctccaaacacagaaaggccatttattgt

SEQ ID NO: 240 - DNA, 56 bp, synthetic construct
  misc_feature 1..56: note = Synthetic oligonucleotide
  source 1..56: mol_type = other DNA
gtgactggagttcagacgtgtgctcttccgatctccatgatcgtgcactctagcct

SEQ ID NO: 241 - DNA, 60 bp, synthetic construct
  misc_feature 1..60: note = Synthetic oligonucleotide
  source 1..60: mol_type = other DNA
gtgactggagttcagacgtgtgctcttccgatctaggagtgtgtaccatttaaggatgtg

SEQ ID NO: 242 - DNA, 53 bp, synthetic construct
  misc_feature 1..53: note = Synthetic oligonucleotide
  source 1..53: mol_type = other DNA
gtgactggagttcagacgtgtgctcttccgatctcccacctggccctgcaaga

SEQ ID NO: 243 - DNA, 60 bp, synthetic construct
  misc_feature 1..60: note = Synthetic oligonucleotide
  source 1..60: mol_type = other DNA
gtgactggagttcagacgtgtgctcttccgatcttctctgcccctggacagattctatag

SEQ ID NO: 244 - DNA, 57 bp, synthetic construct
  misc_feature 1..57: note = Synthetic oligonucleotide
  source 1..57: mol_type = other DNA
gtgactggagttcagacgtgtgctcttccgatctgggccctgctgcactatgatcaa

SEQ ID NO: 245 - DNA, 58 bp, synthetic construct
  misc_feature 1..58: note = Synthetic oligonucleotide
  source 1..58: mol_type = other DNA
gtgactggagttcagacgtgtgctcttccgatctgggccagcttcatgacctgaaacc

SEQ ID NO: 246 - DNA, 52 bp, synthetic construct
  misc_feature 1..52: note = Synthetic oligonucleotide
  source 1..52: mol_type = other DNA
gtgactggagttcagacgtgtgctcttccgatcttgcacctggctgcaccac

SEQ ID NO: 247 - DNA, 60 bp, synthetic construct
  misc_feature 1..60: note = Synthetic oligonucleotide
  source 1..60: mol_type = other DNA
gtgactggagttcagacgtgtgctcttccgatctcatcctgaatgctcgttgagtggatg

SEQ ID NO: 248 - DNA, 58 bp, synthetic construct
  misc_feature 1..58: note = Synthetic oligonucleotide
  source 1..58: mol_type = other DNA
gtgactggagttcagacgtgtgctcttccgatctggccaagcccatgtagtactgcag

SEQ ID NO: 249 - DNA, 58 bp, synthetic construct
  misc_feature 1..58: note = Synthetic oligonucleotide
  source 1..58: mol_type = other DNA
gtgactggagttcagacgtgtgctcttccgatctccctcaactacttgcccaacatgc

SEQ ID NO: 250 - DNA, 60 bp, synthetic construct
  misc_feature 1..60: note = Synthetic oligonucleotide
  source 1..60: mol_type = other DNA
gtgactggagttcagacgtgtgctcttccgatctcggtgtcagcaaagctaggtaaggag

SEQ ID NO: 251 - DNA, 59 bp, synthetic construct
  misc_feature 1..59: note = Synthetic oligonucleotide
  source 1..59: mol_type = other DNA
gtgactggagttcagacgtgtgctcttccgatctccgatgaggcatgggttatgaagta

SEQ ID NO: 252 - DNA, 56 bp, synthetic construct
  misc_feature 1..56: note = Synthetic oligonucleotide
  source 1..56: mol_type = other DNA
gtgactggagttcagacgtgtgctcttccgatctcccctctggtatgccccctcat

SEQ ID NO: 253 - DNA, 58 bp, synthetic construct
  misc_feature 1..58: note = Synthetic oligonucleotide
  source 1..58: mol_type = other DNA
gtgactggagttcagacgtgtgctcttccgatcttgactggatgctgggttgtggaaa

SEQ ID NO: 254 - DNA, 55 bp, synthetic construct
  misc_feature 1..55: note = Synthetic oligonucleotide
  source 1..55: mol_type = other DNA
gtgactggagttcagacgtgtgctcttccgatctttctcccccgaggcctcagaa

SEQ ID NO: 255 - DNA, 60 bp, synthetic construct
  misc_feature 1..60: note = Synthetic oligonucleotide
  source 1..60: mol_type = other DNA
gtgactggagttcagacgtgtgctcttccgatctgacttgacataagcaccatacttcgg

SEQ ID NO: 256 - DNA, 54 bp, synthetic construct
  misc_feature 1..54: note = Synthetic oligonucleotide
  source 1..54: mol_type = other DNA
gtgactggagttcagacgtgtgctcttccgatctgggctgctgggtaggacctg

SEQ ID NO: 257 - DNA, 60 bp, synthetic construct
  misc_feature 1..60: note = Synthetic oligonucleotide
  source 1..60: mol_type = other DNA
gtgactggagttcagacgtgtgctcttccgatctcgtgattcgagttcctggcaatgcta

SEQ ID NO: 258 - DNA, 56 bp, synthetic construct
  misc_feature 1..56: note = Synthetic oligonucleotide
  source 1..56: mol_type = other DNA
gtgactggagttcagacgtgtgctcttccgatctggtcctgaaatgacccccaagg

SEQ ID NO: 259 - DNA, 55 bp, synthetic construct
  misc_feature 1..55: note = Synthetic oligonucleotide
  source 1..55: mol_type = other DNA
gtgactggagttcagacgtgtgctcttccgatctcgcagcctattgtctcctggt

SEQ ID NO: 260 - DNA, 52 bp, synthetic construct
  misc_feature 1..52: note = Synthetic oligonucleotide
  source 1..52: mol_type = other DNA
gtgactggagttcagacgtgtgctcttccgatctgcacgccgtggccgaaca

SEQ ID NO: 261 - DNA, 58 bp, synthetic construct
  misc_feature 1..58: note = Synthetic oligonucleotide
  source 1..58: mol_type = other DNA
gtgactggagttcagacgtgtgctcttccgatctaacttccataccagcagcagttcc

SEQ ID NO: 262 - DNA, 52 bp, synthetic construct
  misc_feature 1..52: note = Synthetic oligonucleotide
  source 1..52: mol_type = other DNA
gtgactggagttcagacgtgtgctcttccgatctccgctcgggccgctgact

SEQ ID NO: 263 - DNA, 59 bp, synthetic construct
  misc_feature 1..59: note = Synthetic oligonucleotide
  source 1..59: mol_type = other DNA
gtgactggagttcagacgtgtgctcttccgatctcgcccactctcgactcttcaggtag

SEQ ID NO: 264 - DNA, 57 bp, synthetic construct
  misc_feature 1..57: note = Synthetic oligonucleotide
  source 1..57: mol_type = other DNA
gtgactggagttcagacgtgtgctcttccgatctactccccaggtgctgaagagacg

SEQ ID NO: 265 - DNA, 52 bp, synthetic construct
  misc_feature 1..52: note = Synthetic oligonucleotide
  source 1..52: mol_type = other DNA
gtgactggagttcagacgtgtgctcttccgatctgcagggggtccgagtgca

SEQ ID NO: 266 - DNA, 53 bp, synthetic construct
  misc_feature 1..53: note = Synthetic oligonucleotide
  source 1..53: mol_type = other DNA
gtgactggagttcagacgtgtgctcttccgatcttgctgctcgttgcctggca

SEQ ID NO: 267 - DNA, 60 bp, synthetic construct
  misc_feature 1..60: note = Synthetic oligonucleotide
  source 1..60: mol_type = other DNA
gtgactggagttcagacgtgtgctcttccgatctcacttgaggtggacgtcagtttctgg

SEQ ID NO: 268 - DNA, 60 bp, synthetic construct
  misc_feature 1..60: note = Synthetic oligonucleotide
  source 1..60: mol_type = other DNA
gtgactggagttcagacgtgtgctcttccgatctgtgggttctggcagaagttcctatgt

SEQ ID NO: 269 - DNA, 52 bp, synthetic construct
  misc_feature 1..52: note = Synthetic oligonucleotide
  source 1..52: mol_type = other DNA
gtgactggagttcagacgtgtgctcttccgatctcccctctgccacccccat

SEQ ID NO: 270 - DNA, 57 bp, synthetic construct
  misc_feature 1..57: note = Synthetic oligonucleotide
  source 1..57: mol_type = other DNA
gtgactggagttcagacgtgtgctcttccgatctcctgtgtccggagctgtttctgc

SEQ ID NO: 271 - DNA, 54 bp, synthetic construct
  misc_feature 1..54: note = Synthetic oligonucleotide
  source 1..54: mol_type = other DNA
gtgactggagttcagacgtgtgctcttccgatctcctggctggaaaggcaaccc

SEQ ID NO: 272 - DNA, 59 bp, synthetic construct
  misc_feature 1..59: note = Synthetic oligonucleotide
  source 1..59: mol_type = other DNA
gtgactggagttcagacgtgtgctcttccgatctaccactctagaatttggcaagatgt

SEQ ID NO: 273 - DNA, 60 bp, synthetic construct
  misc_feature 1..60: note = Synthetic oligonucleotide
  source 1..60: mol_type = other DNA
gtgactggagttcagacgtgtgctcttccgatctatttctccacctaggtgtgctctctc

SEQ ID NO: 274 - DNA, 59 bp, synthetic construct
  misc_feature 1..59: note = Synthetic oligonucleotide
  source 1..59: mol_type = other DNA
gtgactggagttcagacgtgtgctcttccgatctgaacacaggagctagtctgggaaga

SEQ ID NO: 275 - DNA, 60 bp, synthetic construct
  misc_feature 1..60: note = Synthetic oligonucleotide
  source 1..60: mol_type = other DNA
gtgactggagttcagacgtgtgctcttccgatctcgttactgtgcattgaagtcacctga

SEQ ID NO: 276 - DNA, 60 bp, synthetic construct
  misc_feature 1..60: note = Synthetic oligonucleotide
  source 1..60: mol_type = other DNA
gtgactggagttcagacgtgtgctcttccgatctgcctgttcttgcacctggattcttac

SEQ ID NO: 277 - DNA, 54 bp, synthetic construct
  misc_feature 1..54: note = Synthetic oligonucleotide
  source 1..54: mol_type = other DNA
gtgactggagttcagacgtgtgctcttccgatctgcgccctgcccttcggataa

SEQ ID NO: 278 - DNA, 60 bp, synthetic construct
  misc_feature 1..60: note = Synthetic oligonucleotide
  source 1..60: mol_type = other DNA
gtgactggagttcagacgtgtgctcttccgatctacggttatctttgcgacttaggctca

SEQ ID NO: 279 - DNA, 60 bp, synthetic construct
  misc_feature 1..60: note = Synthetic oligonucleotide
  source 1..60: mol_type = other DNA
gtgactggagttcagacgtgtgctcttccgatctgcgttataaactccccgaatcttgga

SEQ ID NO: 280 - DNA, 60 bp, synthetic construct
  misc_feature 1..60: note = Synthetic oligonucleotide
  source 1..60: mol_type = other DNA
gtgactggagttcagacgtgtgctcttccgatcttccctggggcttgcttagtaaagtag

SEQ ID NO: 281 - DNA, 52 bp, synthetic construct
  misc_feature 1..52: note = Synthetic oligonucleotide
  source 1..52: mol_type = other DNA
gtgactggagttcagacgtgtgctcttccgatctaggtggccggcccagggt

SEQ ID NO: 282 - DNA, 52 bp, synthetic construct
  misc_feature 1..52: note = Synthetic oligonucleotide
  source 1..52: mol_type = other DNA
gtgactggagttcagacgtgtgctcttccgatcttcgggccggggcgtctga

SEQ ID NO: 283 - DNA, 52 bp, synthetic construct
  misc_feature 1..52: note = Synthetic oligonucleotide
  source 1..52: mol_type = other DNA
gtgactggagttcagacgtgtgctcttccgatctcggcggcggacagtggac

SEQ ID NO: 284 - DNA, 59 bp, synthetic construct
  misc_feature 1..59: note = Synthetic oligonucleotide
  source 1..59: mol_type = other DNA
gtgactggagttcagacgtgtgctcttccgatctccgaagcgagaacagcccagaagtt

SEQ ID NO: 285 - DNA, 46 bp, synthetic construct
  misc_feature 1..46: note = Synthetic oligonucleotide
  source 1..46: mol_type = other DNA
ccccttagggataacagggtaatcttggcatgacccaggtccatac

SEQ ID NO: 286 - DNA, 48 bp, synthetic construct
  misc_feature 1..48: note = Synthetic oligonucleotide
  source 1..48: mol_type = other DNA
ccccttagggataacagggtaatcaagagtctgggtgaatcagcagtc

SEQ ID NO: 287 - DNA, 42 bp, synthetic construct
  misc_feature 1..42: note = Synthetic oligonucleotide
  source 1..42: mol_type = other DNA
ccccttagggataacagggtaatcaggggccagcagcaaggt

SEQ ID NO: 288 - DNA, 47 bp, synthetic construct
  misc_feature 1..47: note = Synthetic oligonucleotide
  source 1..47: mol_type = other DNA
ccccttagggataacagggtaatctgagtcaggaggcagagatcctc

SEQ ID NO: 289 - DNA, 44 bp, synthetic construct
  misc_feature 1..44: note = Synthetic oligonucleotide
  source 1..44: mol_type = other DNA
ccccttagggataacagggtaatcaaatcccgttggccctcctg

SEQ ID NO: 290 - DNA, 42 bp, synthetic construct
  misc_feature 1..42: note = Synthetic oligonucleotide
  source 1..42: mol_type = other DNA
ccccttagggataacagggtaatcggggcgttgtgggtctga

SEQ ID NO: 291 - DNA, 42 bp, synthetic construct
  misc_feature 1..42: note = Synthetic oligonucleotide
  source 1..42: mol_type = other DNA
ccccttagggataacagggtaatccctaggcccctcccctct

SEQ ID NO: 292 - DNA, 44 bp, synthetic construct
  misc_feature 1..44: note = Synthetic oligonucleotide
  source 1..44: mol_type = other DNA
ccccttagggataacagggtaatcccaggtggtctcctccgact

SEQ ID NO: 293 - DNA, 45 bp, synthetic construct
  misc_feature 1..45: note = Synthetic oligonucleotide
  source 1..45: mol_type = other DNA
ccccttagggataacagggtaatcaggggagatgctcagtgtggt

SEQ ID NO: 294 - DNA, 46 bp, synthetic construct
  misc_feature 1..46: note = Synthetic oligonucleotide
  source 1..46: mol_type = other DNA
ccccttagggataacagggtaatctgagcacaaggtcgtctcctct

SEQ ID NO: 295 - DNA, 49 bp, synthetic construct
  misc_feature 1..49: note = Synthetic oligonucleotide
  source 1..49: mol_type = other DNA
ccccttagggataacagggtaatctggcagatgaataaggctcactcct

SEQ ID NO: 296 - DNA, 50 bp, synthetic construct
  misc_feature 1..50: note = Synthetic oligonucleotide
  source 1..50: mol_type = other DNA
ccccttagggataacagggtaatctccctacagagataaacagacgcaca

SEQ ID NO: 297 - DNA, 49 bp, synthetic construct
  misc_feature 1..49: note = Synthetic oligonucleotide
  source 1..49: mol_type = other DNA
ccccttagggataacagggtaatccagttctttgggtcctcatcacagt

SEQ ID NO: 298 - DNA, 47 bp, synthetic construct
  misc_feature 1..47: note = Synthetic oligonucleotide
  source 1..47: mol_type = other DNA
ccccttagggataacagggtaatcagcaacatacagatggggtggga

SEQ ID NO: 299 - DNA, 47 bp, synthetic construct
  misc_feature 1..47: note = Synthetic oligonucleotide
  source 1..47: mol_type = other DNA
ccccttagggataacagggtaatcggatgcttagcttccgttgggtt

SEQ ID NO: 300 - DNA, 42 bp, synthetic construct
  misc_feature 1..42: note = Synthetic oligonucleotide
  source 1..42: mol_type = other DNA
ccccttagggataacagggtaatcctgggcacggtggacagc

SEQ ID NO: 301 - DNA, 48 bp, synthetic construct
  misc_feature 1..48: note = Synthetic oligonucleotide
  source 1..48: mol_type = other DNA
ccccttagggataacagggtaatccctcttcaagtggtctgcatggaa

SEQ ID NO: 302 - DNA, 47 bp, synthetic construct
  misc_feature 1..47: note = Synthetic oligonucleotide
  source 1..47: mol_type = other DNA
ccccttagggataacagggtaatcggtggtctcctccgatttcaaca

SEQ ID NO: 303 - DNA, 44 bp, synthetic construct
  misc_feature 1..44: note = Synthetic oligonucleotide
  source 1..44: mol_type = other DNA
ccccttagggataacagggtaatcttgcggggaggggagattct

SEQ ID NO: 304 - DNA, 46 bp, synthetic construct
  misc_feature 1..46: note = Synthetic oligonucleotide
  source 1..46: mol_type = other DNA
ccccttagggataacagggtaatcccctacctcaccgccaatgttt

SEQ ID NO: 305 - DNA, 47 bp, synthetic construct
  misc_feature 1..47: note = Synthetic oligonucleotide
  source 1..47: mol_type = other DNA
ccccttagggataacagggtaatcgaggaggggagagtctcagtgtt

SEQ ID NO: 306 - DNA, 50 bp, synthetic construct
  misc_feature 1..50: note = Synthetic oligonucleotide
  source 1..50: mol_type = other DNA
ccccttagggataacagggtaatcactttaacagcatcacccactcttcc

SEQ ID NO: 307 - DNA, 50 bp, synthetic construct
  misc_feature 1..50: note = Synthetic oligonucleotide
  source 1..50: mol_type = other DNA
ccccttagggataacagggtaatctttcctgtattgcttttgccttgagc

SEQ ID NO: 308 - DNA, 46 bp, synthetic construct
  misc_feature 1..46: note = Synthetic oligonucleotide
  source 1..46: mol_type = other DNA
ccccttagggataacagggtaatcggagcctggaccactaagtcac

SEQ ID NO: 309 - DNA, 46 bp, synthetic construct
  misc_feature 1..46: note = Synthetic oligonucleotide
  source 1..46: mol_type = other DNA
ccccttagggataacagggtaatcgcgtggaggtgagctcatgtag

SEQ ID NO: 310 - DNA, 43 bp, synthetic construct
  misc_feature 1..43: note = Synthetic oligonucleotide
  source 1..43: mol_type = other DNA
ccccttagggataacagggtaatcgggcgctcagtaggtgtgc

SEQ ID NO: 311 - DNA, 50 bp, synthetic construct
  misc_feature 1..50: note = Synthetic oligonucleotide
  source 1..50: mol_type = other DNA
ccccttagggataacagggtaatcctgtgggccatcttcaagttcagtcc

SEQ ID NO: 312 - DNA, 49 bp, synthetic construct
  misc_feature 1..49: note = Synthetic oligonucleotide
  source 1..49: mol_type = other DNA
ccccttagggataacagggtaatcaaaaacctccacccttatgaagcct

SEQ ID NO: 313 - DNA, 45 bp, synthetic construct
  misc_feature 1..45: note = Synthetic oligonucleotide
  source 1..45: mol_type = other DNA
ccccttagggataacagggtaatctctctgctgtgtgctgtccac

SEQ ID NO: 314 - DNA, 43 bp, synthetic construct
  misc_feature 1..43: note = Synthetic oligonucleotide
  source 1..43: mol_type = other DNA
ccccttagggataacagggtaatcagcccctccctctccagga

SEQ ID NO: 315 - DNA, 42 bp, synthetic construct
  misc_feature 1..42: note = Synthetic oligonucleotide
  source 1..42: mol_type = other DNA
ccccttagggataacagggtaatcgatgctggggctggcact

SEQ ID NO: 316 - DNA, 47 bp, synthetic construct
  misc_feature 1..47: note = Synthetic oligonucleotide
  source 1..47: mol_type = other DNA
ccccttagggataacagggtaatcactgtgtccaggggagattctca

SEQ ID NO: 317 - DNA, 50 bp, synthetic construct
  misc_feature 1..50: note = Synthetic oligonucleotide
  source 1..50: mol_type = other DNA
ccccttagggataacagggtaatcacttacgcttaggtgtgatttgcgaa

SEQ ID NO: 318 - DNA, 46 bp, synthetic construct
  misc_feature 1..46: note = Synthetic oligonucleotide
  source 1..46: mol_type = other DNA
ccccttagggataacagggtaatccaggcaaggctgaatggaagcg

SEQ ID NO: 319 - DNA, 43 bp, synthetic construct
  misc_feature 1..43: note = Synthetic oligonucleotide
  source 1..43: mol_type = other DNA
ccccttagggataacagggtaatccctggggaagggccattca

SEQ ID NO: 320 - DNA, 43 bp, synthetic construct
  misc_feature 1..43: note = Synthetic oligonucleotide
  source 1..43: mol_type = other DNA
ccccttagggataacagggtaatcggagacggtgcaggagctc

SEQ ID NO: 321 - DNA, 45 bp, synthetic construct
  misc_feature 1..45: note = Synthetic oligonucleotide
  source 1..45: mol_type = other DNA
ccccttagggataacagggtaatcaggaccctcctcacgggatac

SEQ ID NO: 322 - DNA, 43 bp, synthetic construct
  misc_feature 1..43: note = Synthetic oligonucleotide
  source 1..43: mol_type = other DNA
ccccttagggataacagggtaatcgtgtggtgggggactgagc

SEQ ID NO: 323 - DNA, 43 bp, synthetic construct
  misc_feature 1..43: note = Synthetic oligonucleotide
  source 1..43: mol_type = other DNA
ccccttagggataacagggtaatcgatgctggggctgccattg

SEQ ID NO: 324 - DNA, 43 bp, synthetic construct
  misc_feature 1..43: note = Synthetic oligonucleotide
  source 1..43: mol_type = other DNA
ccccttagggataacagggtaatcctcctcaccacccccaagg

SEQ ID NO: 325 - DNA, 43 bp, synthetic construct
  misc_feature 1..43: note = Synthetic oligonucleotide
  source 1..43: mol_type = other DNA
ccccttagggataacagggtaatcggccaaagtccgccccaag

SEQ ID NO: 326 - DNA, 44 bp, synthetic construct
  misc_feature 1..44: note = Synthetic oligonucleotide
  source 1..44: mol_type = other DNA
ccccttagggataacagggtaatcggaggccccaggaactttca

SEQ ID NO: 327 - DNA, 44 bp, synthetic construct
  misc_feature 1..44: note = Synthetic oligonucleotide
  source 1..44: mol_type = other DNA
ccccttagggataacagggtaatccctcgggaggtgggtagtgt

SEQ ID NO: 328 - DNA, 47 bp, synthetic construct
  misc_feature 1..47: note = Synthetic oligonucleotide
  source 1..47: mol_type = other DNA
ccccttagggataacagggtaatcggaccagcttgttgaggacccta

SEQ ID NO: 329 - DNA, 46 bp, synthetic construct
  misc_feature 1..46: note = Synthetic oligonucleotide
  source 1..46: mol_type = other DNA
ccccttagggataacagggtaatcgagcctcatcagttgaccccaa

SEQ ID NO: 330 - DNA, 43 bp, synthetic construct
  misc_feature 1..43: note = Synthetic oligonucleotide
  source 1..43: mol_type = other DNA
ccccttagggataacagggtaatcggggtgcagcctggagaga

SEQ ID NO: 331 - DNA, 48 bp, synthetic construct
  misc_feature 1..48: note = Synthetic oligonucleotide
  source 1..48: mol_type = other DNA
ccccttagggataacagggtaatcagctttgctggggtaacaggacac

SEQ ID NO: 332 - DNA, 50 bp, synthetic construct
  misc_feature 1..50: note = Synthetic oligonucleotide
  source 1..50: mol_type = other DNA
ccccttagggataacagggtaatcgaaactatgaaactaccaggagaagt

SEQ ID NO: 333 - DNA, 49 bp, synthetic construct
  misc_feature 1..49: note = Synthetic oligonucleotide
  source 1..49: mol_type = other DNA
ccccttagggataacagggtaatcgttcaaagcatcatctgtgaatcaa

SEQ ID NO: 334 - DNA, 46 bp, synthetic construct
  misc_feature 1..46: note = Synthetic oligonucleotide
  source 1..46: mol_type = other DNA
ccccttagggataacagggtaatctctgaggccagcaaaaccttga

SEQ ID NO: 335 - DNA, 44 bp, synthetic construct
  misc_feature 1..44: note = Synthetic oligonucleotide
  source 1..44: mol_type = other DNA
ccccttagggataacagggtaatcactgacacctggaggcctga

SEQ ID NO: 336 - DNA, 44 bp, synthetic construct
  misc_feature 1..44: note = Synthetic oligonucleotide
  source 1..44: mol_type = other DNA
ccccttagggataacagggtaatcctggagggtgtatgcgtgct

SEQ ID NO: 337 - DNA, 42 bp, synthetic construct
  misc_feature 1..42: note = Synthetic oligonucleotide
  source 1..42: mol_type = other DNA
ccccttagggataacagggtaatcctggggttggcgtcacct

SEQ ID NO: 338 - DNA, 47 bp, synthetic construct
  misc_feature 1..47: note = Synthetic oligonucleotide
  source 1..47: mol_type = other DNA
ccccttagggataacagggtaatcattcttcagggggtctggcatga

SEQ ID NO: 339 - DNA, 50 bp, synthetic construct
  misc_feature 1..50: note = Synthetic oligonucleotide
  source 1..50: mol_type = other DNA
ccccttagggataacagggtaatccacccatatgcacacccacatatacc

SEQ ID NO: 340 - DNA, 47 bp, synthetic construct
  misc_feature 1..47: note = Synthetic oligonucleotide
  source 1..47: mol_type = other DNA
ccccttagggataacagggtaatcgaaaacgccctactgccctagat

SEQ ID NO: 341 - DNA, 50 bp, synthetic construct
  misc_feature 1..50: note = Synthetic oligonucleotide
  source 1..50: mol_type = other DNA
ccccttagggataacagggtaatcagtccgcccccttatcatcctctctg

SEQ ID NO: 342 - DNA, 47 bp, synthetic construct
  misc_feature 1..47: note = Synthetic oligonucleotide
  source 1..47: mol_type = other DNA
ccccttagggataacagggtaatcccaacgtggacatgaggatgcat

SEQ ID NO: 343 - DNA, 47 bp, synthetic construct
  misc_feature 1..47: note = Synthetic oligonucleotide
  source 1..47: mol_type = other DNA
ccccttagggataacagggtaatctggcttcccaacctgaggttttg

SEQ ID NO: 344 - DNA, 48 bp, synthetic construct
  misc_feature 1..48: note = Synthetic oligonucleotide
  source 1..48: mol_type = other DNA
ccccttagggataacagggtaatcgacacaggagaacccactgaacgc

SEQ ID NO: 345 - DNA, 48 bp, synthetic construct
  misc_feature 1..48: note = Synthetic oligonucleotide
  source 1..48: mol_type = other DNA
ccccttagggataacagggtaatctctccacagtacaatgaggccatg

SEQ ID NO: 346 - DNA, 44 bp, synthetic construct
  misc_feature 1..44: note = Synthetic oligonucleotide
  source 1..44: mol_type = other DNA
ccccttagggataacagggtaatccgtgcacaggggacagaagc

SEQ ID NO: 347 - DNA, 43 bp, synthetic construct
  misc_feature 1..43: note = Synthetic oligonucleotide
  source 1..43: mol_type = other DNA
ccccttagggataacagggtaatccccaggagctacgcctctg

SEQ ID NO: 348 - DNA, 45 bp, synthetic construct
  misc_feature 1..45: note = Synthetic oligonucleotide
  source 1..45: mol_type = other DNA
ccccttagggataacagggtaatcggctggcattgctctcaacga

SEQ ID NO: 349 - DNA, 49 bp, synthetic construct
  misc_feature 1..49: note = Synthetic oligonucleotide
  source 1..49: mol_type = other DNA
ccccttagggataacagggtaatccatgacgaggtcaggctccctaggc

SEQ ID NO: 350 - DNA, 44 bp, synthetic construct
  misc_feature 1..44: note = Synthetic oligonucleotide
  source 1..44: mol_type = other DNA
ccccttagggataacagggtaatcgtggtggacttcgcagacca

SEQ ID NO: 351 - DNA, 50 bp, synthetic construct
  misc_feature 1..50: note = Synthetic oligonucleotide
  source 1..50: mol_type = other DNA
ccccttagggataacagggtaatcgcccagcttaaaacatgagccattca

SEQ ID NO: 352 - DNA, 50 bp, synthetic construct
  misc_feature 1..50: note = Synthetic oligonucleotide
  source 1..50: mol_type = other DNA
ccccttagggataacagggtaatcgggagacaatggagatctacctcagt

SEQ ID NO: 353 - DNA, 43 bp, synthetic construct
  misc_feature 1..43: note = Synthetic oligonucleotide
  source 1..43: mol_type = other DNA
ccccttagggataacagggtaatccccactggcgtcttcagca

SEQ ID NO: 354 - DNA, 47 bp, synthetic construct
  misc_feature 1..47: note = Synthetic oligonucleotide
  source 1..47: mol_type = other DNA
ccccttagggataacagggtaatcgccaagggtgccaaacgttgata

SEQ ID NO: 355 - DNA, 46 bp, synthetic construct
  misc_feature 1..46: note = Synthetic oligonucleotide
  source 1..46: mol_type = other DNA
ccccttagggataacagggtaatccagcgtttcaggaagggagagg

SEQ ID NO: 356 - DNA, 42 bp, synthetic construct
  misc_feature 1..42: note = Synthetic oligonucleotide
  source 1..42: mol_type = other DNA
ccccttagggataacagggtaatctgtgcccccatgcatgcc

SEQ ID NO: 357 - DNA, 48 bp, synthetic construct
  misc_feature 1..48: note = Synthetic oligonucleotide
  source 1..48: mol_type = other DNA
ccccttagggataacagggtaatcgcattgccctcaacgaccactttt

SEQ ID NO: 358 - DNA, 50 bp, synthetic construct
  misc_feature 1..50: note = Synthetic oligonucleotide
  source 1..50: mol_type = other DNA
ccccttagggataacagggtaatccttaactctcacagggccatgtagtg

SEQ ID NO: 359 - DNA, 46 bp, synthetic construct
  misc_feature 1..46: note = Synthetic oligonucleotide
  source 1..46: mol_type = other DNA
ccccttagggataacagggtaatcaggggtctacatggcaactgtg

SEQ ID NO: 360 - DNA, 54 bp, synthetic construct
  misc_feature 1..54: note = Synthetic oligonucleotide
  source 1..54: mol_type = other DNA
gtgactggagttcagacgtgtgctcttccgatctggtccataccagggctgacc

SEQ ID NO: 361 - DNA, 54 bp, synthetic construct
  misc_feature 1..54: note = Synthetic oligonucleotide
  source 1..54: mol_type = other DNA
gtgactggagttcagacgtgtgctcttccgatctagtcaggcaggcgaggaaca

SEQ ID NO: 362 - DNA, 60 bp, synthetic construct
  misc_feature 1..60: note = Synthetic oligonucleotide
  source 1..60: mol_type = other DNA
gtgactggagttcagacgtgtgctcttccgatctaggtgaagaatttcatgctggcacat

SEQ ID NO: 363 - DNA, 55 bp, synthetic construct
  misc_feature 1..55: note = Synthetic oligonucleotide
  source 1..55: mol_type = other DNA
gtgactggagttcagacgtgtgctcttccgatctcaagacccagcgaccgactcc

SEQ ID NO: 364 - DNA, 57 bp, synthetic construct
  misc_feature 1..57: note = Synthetic oligonucleotide
  source 1..57: mol_type = other DNA
gtgactggagttcagacgtgtgctcttccgatctctcctgctcagctggctcatgtc

SEQ ID NO: 365 - DNA, 60 bp, synthetic construct
  misc_feature 1..60: note = Synthetic oligonucleotide
  source 1..60: mol_type = other DNA
gtgactggagttcagacgtgtgctcttccgatctcttaaagatcctccggccaccatgtg

SEQ ID NO: 366 - DNA, 53 bp, synthetic construct
  misc_feature 1..53: note = Synthetic oligonucleotide
  source 1..53: mol_type = other DNA
gtgactggagttcagacgtgtgctcttccgatctgcccctcccctcttcaagg

SEQ ID NO: 367 - DNA, 57 bp, synthetic construct
  misc_feature 1..57: note = Synthetic oligonucleotide
  source 1..57: mol_type = other DNA
gtgactggagttcagacgtgtgctcttccgatcttcaacagcaacacccactcttcc

SEQ ID NO: 368 - DNA, 52 bp, synthetic construct
  misc_feature 1..52: note = Synthetic oligonucleotide
  source 1..52: mol_type = other DNA
gtgactggagttcagacgtgtgctcttccgatctgtgtggtgggggctgagc

SEQ ID NO: 369 - DNA, 60 bp, synthetic construct
  misc_feature 1..60: note = Synthetic oligonucleotide
  source 1..60: mol_type = other DNA
gtgactggagttcagacgtgtgctcttccgatctctctgactttgacagtgacacccatt

SEQ ID NO: 370 - DNA, 60 bp, synthetic construct
  misc_feature 1..60: note = Synthetic oligonucleotide
  source 1..60: mol_type = other DNA
gtgactggagttcagacgtgtgctcttccgatctggctcactccttctcttgtaggtact

SEQ ID NO: 371 - DNA, 60 bp, synthetic construct
  misc_feature 1..60: note = Synthetic oligonucleotide
  source 1..60: mol_type = other DNA
gtgactggagttcagacgtgtgctcttccgatctgagagagagtaaggtcaggcatgtgg

SEQ ID NO: 372 - DNA, 60 bp, synthetic construct
  misc_feature 1..60: note = Synthetic oligonucleotide
  source 1..60: mol_type = other DNA
gtgactggagttcagacgtgtgctcttccgatcttcatcacagttaatgttgcagcggaa

SEQ ID NO: 373 - DNA, 53 bp, synthetic construct
  misc_feature 1..53: note = Synthetic oligonucleotide
  source 1..53: mol_type = other DNA
gtgactggagttcagacgtgtgctcttccgatctgctggagctgtgggggcaa

SEQ ID NO: 374 - DNA, 60 bp, synthetic construct
  misc_feature 1..60: note = Synthetic oligonucleotide
  source 1..60: mol_type = other DNA
gtgactggagttcagacgtgtgctcttccgatctgcttagcttccgttgggttgatgagg

SEQ ID NO: 375 - DNA, 53 bp, synthetic construct
  misc_feature 1..53: note = Synthetic oligonucleotide
  source 1..53: mol_type = other DNA
gtgactggagttcagacgtgtgctcttccgatctcacggtggacagcagtgca

SEQ ID NO: 376 - DNA, 59 bp, synthetic construct
  misc_feature 1..59: note = Synthetic oligonucleotide
  source 1..59: mol_type = other DNA
gtgactggagttcagacgtgtgctcttccgatctgcatggaaactgtgaggaggggagt

SEQ ID NO: 377 - DNA, 53 bp, synthetic construct
  misc_feature 1..53: note = Synthetic oligonucleotide
  source 1..53: mol_type = other DNA
gtgactggagttcagacgtgtgctcttccgatctagtgacaccccctcctcca

SEQ ID NO: 378 - DNA, 56 bp, synthetic construct
  misc_feature 1..56: note = Synthetic oligonucleotide
  source 1..56: mol_type = other DNA
gtgactggagttcagacgtgtgctcttccgatctagggaactggacacgtcaggga

SEQ ID NO: 379 - DNA, 59 bp, synthetic construct
  misc_feature 1..59: note = Synthetic oligonucleotide
  source 1..59: mol_type = other DNA
gtgactggagttcagacgtgtgctcttccgatctactttggtgggcgtataagcagttt

SEQ ID NO: 380 - DNA, 59 bp, synthetic construct
  misc_feature 1..59: note = Synthetic oligonucleotide
  source 1..59: mol_type = other DNA
gtgactggagttcagacgtgtgctcttccgatctaggggagagtctcagtgttgtggag

SEQ ID NO: 381 - DNA, 59 bp, synthetic construct
  misc_feature 1..59: note = Synthetic oligonucleotide
  source 1..59: mol_type = other DNA
gtgactggagttcagacgtgtgctcttccgatctggctacagcaacagggtagtagacc

SEQ ID NO: 382 - DNA, 60 bp, synthetic construct
  misc_feature 1..60: note = Synthetic oligonucleotide
  source 1..60: mol_type = other DNA
gtgactggagttcagacgtgtgctcttccgatcttgccttgagcttcttaccccagtgag

SEQ ID NO: 383 - DNA, 60 bp, synthetic construct
  misc_feature 1..60: note = Synthetic oligonucleotide
  source 1..60: mol_type = other DNA
gtgactggagttcagacgtgtgctcttccgatctttccaaccaaggtacctgtattggac

SEQ ID NO: 384 - DNA, 59 bp, synthetic construct
  misc_feature 1..59: note = Synthetic oligonucleotide
  source 1..59: mol_type = other DNA
gtgactggagttcagacgtgtgctcttccgatctccctgctcactggagaagttttccg

SEQ ID NO: 385 - DNA, 57 bp, synthetic construct
  misc_feature 1..57: note = Synthetic oligonucleotide
  source 1..57: mol_type = other DNA
gtgactggagttcagacgtgtgctcttccgatctgcgctcagtaggtgtgcaagcag

SEQ ID NO: 386 - DNA, 60 bp, synthetic construct
  misc_feature 1..60: note = Synthetic oligonucleotide
  source 1..60: mol_type = other DNA
gtgactggagttcagacgtgtgctcttccgatcttctcatttctggacctaggctgatgg

SEQ ID NO: 387 - DNA, 60 bp, synthetic construct
  misc_feature 1..60: note = Synthetic oligonucleotide
  source 1..60: mol_type = other DNA
gtgactggagttcagacgtgtgctcttccgatcttccacccttatgaagcctccttctag

SEQ ID NO: 388 - DNA, 60 bp, synthetic construct
  misc_feature 1..60: note = Synthetic oligonucleotide
  source 1..60: mol_type = other DNA
gtgactggagttcagacgtgtgctcttccgatctgtccactcacaggggtagaacatgtt

SEQ ID NO: 389 - DNA, 55 bp, synthetic construct
  misc_feature 1..55: note = Synthetic oligonucleotide
  source 1..55: mol_type = other DNA
gtgactggagttcagacgtgtgctcttccgatctaggtgggggactgagtgtgac

SEQ ID NO: 390 - DNA, 58 bp, synthetic construct
  misc_feature 1..58: note = Synthetic oligonucleotide
  source 1..58: mol_type = other DNA
gtgactggagttcagacgtgtgctcttccgatctgcaacagggtggtggaactcatgt

SEQ ID NO: 391 - DNA, 59 bp, synthetic construct
  misc_feature 1..59: note = Synthetic oligonucleotide
  source 1..59: mol_type = other DNA
gtgactggagttcagacgtgtgctcttccgatctcagtgtggtaagggactgagtgcgt

SEQ ID NO: 392 - DNA, 60 bp, synthetic construct
  misc_feature 1..60: note = Synthetic oligonucleotide
  source 1..60: mol_type = other DNA
gtgactggagttcagacgtgtgctcttccgatctacacattgctgccatgatctgtcgta

SEQ ID NO: 393 - DNA, 60 bp, synthetic construct
  misc_feature 1..60: note = Synthetic oligonucleotide
  source 1..60: mol_type = other DNA
gtgactggagttcagacgtgtgctcttccgatctgctgaatggaagcgagtgaagtgagc

SEQ ID NO: 394 - DNA, 59 bp, synthetic construct
  misc_feature 1..59: note = Synthetic oligonucleotide
  source 1..59: mol_type = other DNA
gtgactggagttcagacgtgtgctcttccgatctgggccattcacccttgatatcatca

SEQ ID NO: 395 - DNA, 52 bp, synthetic construct
  misc_feature 1..52: note = Synthetic oligonucleotide
  source 1..52: mol_type = other DNA
gtgactggagttcagacgtgtgctcttccgatctctgagcagcggggaggct

SEQ ID NO: 396 - DNA, 54 bp, synthetic construct
  misc_feature 1..54: note = Synthetic oligonucleotide
  source 1..54: mol_type = other DNA
gtgactggagttcagacgtgtgctcttccgatctacccagctttcagccagacc

SEQ ID NO: 397 - DNA, 54 bp, synthetic construct
  misc_feature 1..54: note = Synthetic oligonucleotide
  source 1..54: mol_type = other DNA
gtgactggagttcagacgtgtgctcttccgatctgtgggggactgagcatggca

SEQ ID NO: 398 - DNA, 53 bp, synthetic construct
  misc_feature 1..53: note = Synthetic oligonucleotide
  source 1..53: mol_type = other DNA
gtgactggagttcagacgtgtgctcttccgatctggctgccattgccctcagt

SEQ ID NO: 399 - DNA, 52 bp, synthetic construct
  misc_feature 1..52: note = Synthetic oligonucleotide
  source 1..52: mol_type = other DNA
gtgactggagttcagacgtgtgctcttccgatctggtgggggcacagtcctg

SEQ ID NO: 400 - DNA, 58 bp, synthetic construct
  misc_feature 1..58: note = Synthetic oligonucleotide
  source 1..58: mol_type = other DNA
gtgactggagttcagacgtgtgctcttccgatctccaaagtccgccccaaggtcaaaa

SEQ ID NO: 401 - DNA, 58 bp, synthetic construct
  misc_feature 1..58: note = Synthetic oligonucleotide
  source 1..58: mol_type = other DNA
gtgactggagttcagacgtgtgctcttccgatctggaggagaacgaggcatgtcttac

SEQ ID NO: 402 - DNA, 59 bp, synthetic construct
  misc_feature 1..59: note = Synthetic oligonucleotide
  source 1..59: mol_type = other DNA
gtgactggagttcagacgtgtgctcttccgatctctcgggaggtgggtagtgtatggtt

SEQ ID NO: 403 - DNA, 60 bp, synthetic construct
  misc_feature 1..60: note = Synthetic oligonucleotide
  source 1..60: mol_type = other DNA
gtgactggagttcagacgtgtgctcttccgatctccagcttgttgaggaccctaaaggct

SEQ ID NO: 404 - DNA, 60 bp, synthetic construct
  misc_feature 1..60: note = Synthetic oligonucleotide
  source 1..60: mol_type = other DNA
gtgactggagttcagacgtgtgctcttccgatctttgaccccaatgtcctgcatgtacta

SEQ ID NO: 405 - DNA, 58 bp, synthetic construct
  misc_feature 1..58: note = Synthetic oligonucleotide
  source 1..58: mol_type = other DNA
gtgactggagttcagacgtgtgctcttccgatctgagagagctgggttggctgacaga

SEQ ID NO: 406 - DNA, 59 bp, synthetic construct
  misc_feature 1..59: note = Synthetic oligonucleotide
  source 1..59: mol_type = other DNA
gtgactggagttcagacgtgtgctcttccgatctgggtaacaggacacattggctggga

SEQ ID NO: 407 - DNA, 56 bp, synthetic construct
  misc_feature 1..56: note = Synthetic oligonucleotide
  source 1..56: mol_type = other DNA
gtgactggagttcagacgtgtgctcttccgatctccaggagaagtttccagtggga

SEQ ID NO: 408 - DNA, 60 bp, synthetic construct
  misc_feature 1..60: note = Synthetic oligonucleotide
  source 1..60: mol_type = other DNA
gtgactggagttcagacgtgtgctcttccgatctagcatcatctgtgaatcaaaagtttt

SEQ ID NO: 409 - DNA, 60 bp, synthetic construct
  misc_feature 1..60: note = Synthetic oligonucleotide
  source 1..60: mol_type = other DNA
gtgactggagttcagacgtgtgctcttccgatctggccagcaaaaccttgacatgtaaac

SEQ ID NO: 410 - DNA, 55 bp, synthetic construct
  misc_feature 1..55: note = Synthetic oligonucleotide
  source 1..55: mol_type = other DNA
gtgactggagttcagacgtgtgctcttccgatctacctggaggcctgacttgcag

SEQ ID NO: 411 - DNA, 56 bp, synthetic construct
  misc_feature 1..56: note = Synthetic oligonucleotide
  source 1..56: mol_type = other DNA
gtgactggagttcagacgtgtgctcttccgatctagggtgtatgcgtgctctctga

SEQ ID NO: 412 - DNA, 59 bp, synthetic construct
  misc_feature 1..59: note = Synthetic oligonucleotide
  source 1..59: mol_type = other DNA
gtgactggagttcagacgtgtgctcttccgatctgcgtcaccttgaacgaccactttgt

SEQ ID NO: 413 - DNA, 60 bp, synthetic construct
  misc_feature 1..60: note = Synthetic oligonucleotide
  source 1..60: mol_type = other DNA
gtgactggagttcagacgtgtgctcttccgatctagggggtctggcatgaaaatgtgtta

SEQ ID NO: 414 - DNA, 60 bp, synthetic construct
  misc_feature 1..60: note = Synthetic oligonucleotide
  source 1..60: mol_type = other DNA
gtgactggagttcagacgtgtgctcttccgatctcacacccacatatacctgccaaaaga

SEQ ID NO: 415 - DNA, 60 bp, synthetic construct
  misc_feature 1..60: note = Synthetic oligonucleotide
  source 1..60: mol_type = other DNA
gtgactggagttcagacgtgtgctcttccgatctacgccctactgccctagattctaatt

SEQ ID NO: 416 - DNA, 53 bp, synthetic construct
  misc_feature 1..53: note = Synthetic oligonucleotide
  source 1..53: mol_type = other DNA
gtgactggagttcagacgtgtgctcttccgatcttgggggctctggggctact

SEQ ID NO: 417 - DNA, 60 bp, synthetic construct
  misc_feature 1..60: note = Synthetic oligonucleotide
  source 1..60: mol_type = other DNA
gtgactggagttcagacgtgtgctcttccgatctacgtggacatgaggatgcattaaagg

SEQ ID NO: 418 - DNA, 54 bp, synthetic construct
  misc_feature 1..54: note = Synthetic oligonucleotide
  source 1..54: mol_type = other DNA
gtgactggagttcagacgtgtgctcttccgatctatcccctcttccccaagcct

SEQ ID NO: 419 - DNA, 60 bp, synthetic construct
  misc_feature 1..60: note = Synthetic oligonucleotide
  source 1..60: mol_type = other DNA
gtgactggagttcagacgtgtgctcttccgatctgaacccactgaacgcttccacttcca

SEQ ID NO: 420 - DNA, 60 bp, synthetic construct
  misc_feature 1..60: note = Synthetic oligonucleotide
  source 1..60: mol_type = other DNA
gtgactggagttcagacgtgtgctcttccgatctagtacaatgaggccatgcagtttctt

SEQ ID NO: 421 - DNA, 55 bp, synthetic construct
  misc_feature 1..55: note = Synthetic oligonucleotide
  source 1..55: mol_type = other DNA
gtgactggagttcagacgtgtgctcttccgatctacaggggacagaagccatggg

SEQ ID NO: 422 - DNA, 56 bp, synthetic construct
  misc_feature 1..56: note = Synthetic oligonucleotide
  source 1..56: mol_type = other DNA
gtgactggagttcagacgtgtgctcttccgatctctacgcctctgccccatacacg

SEQ ID NO: 423 - DNA, 58 bp, synthetic construct
  misc_feature 1..58: note = Synthetic oligonucleotide
  source 1..58: mol_type = other DNA
gtgactggagttcagacgtgtgctcttccgatcttggcattgctctcaacgaccactt

SEQ ID NO: 424 - DNA, 56 bp, synthetic construct
  misc_feature 1..56: note = Synthetic oligonucleotide
  source 1..56: mol_type = other DNA
gtgactggagttcagacgtgtgctcttccgatctccctaggcccctccgtcttcag

SEQ ID NO: 425 - DNA, 55 bp, synthetic construct
  misc_feature 1..55: note = Synthetic oligonucleotide
  source 1..55: mol_type = other DNA
gtgactggagttcagacgtgtgctcttccgatctggacttcgcagaccacatggc

SEQ ID NO: 426 - DNA, 55 bp, synthetic construct
  misc_feature 1..55: note = Synthetic oligonucleotide
  source 1..55: mol_type = other DNA
gtgactggagttcagacgtgtgctcttccgatctgcctcggctggcctttacttg

SEQ ID NO: 427 - DNA, 60 bp, synthetic construct
  misc_feature 1..60: note = Synthetic oligonucleotide
  source 1..60: mol_type = other DNA
gtgactggagttcagacgtgtgctcttccgatctggcaaagtgagactaatctagctgct

SEQ ID NO: 428 - DNA, 59 bp, synthetic construct
  misc_feature 1..59: note = Synthetic oligonucleotide
  source 1..59: mol_type = other DNA
gtgactggagttcagacgtgtgctcttccgatcttcttcagcactacggagaagactgg

SEQ ID NO: 429 - DNA, 58 bp, synthetic construct
  misc_feature 1..58: note = Synthetic oligonucleotide
  source 1..58: mol_type = other DNA
gtgactggagttcagacgtgtgctcttccgatctgtgccaaacgttgatagtgcagga

SEQ ID NO: 430 - DNA, 55 bp, synthetic construct
  misc_feature 1..55: note = Synthetic oligonucleotide
  source 1..55: mol_type = other DNA
gtgactggagttcagacgtgtgctcttccgatcttgccctgtgctactggaaggc

SEQ ID NO: 431 - DNA, 56 bp, synthetic construct
  misc_feature 1..56: note = Synthetic oligonucleotide
  source 1..56: mol_type = other DNA
gtgactggagttcagacgtgtgctcttccgatctcccccatgcatgcctcactctc

SEQ ID NO: 432 - DNA, 55 bp, synthetic construct
  misc_feature 1..55: note = Synthetic oligonucleotide
  source 1..55: mol_type = other DNA
gtgactggagttcagacgtgtgctcttccgatctagcaacagggtgatggacctc

SEQ ID NO: 433 - DNA, 60 bp, synthetic construct
  misc_feature 1..60: note = Synthetic oligonucleotide
  source 1..60: mol_type = other DNA
gtgactggagttcagacgtgtgctcttccgatctcagggccatgtagtgtcttaaagctg

SEQ ID NO: 434 - DNA, 57 bp, synthetic construct
  misc_feature 1..57: note = Synthetic oligonucleotide
  source 1..57: mol_type = other DNA
gtgactggagttcagacgtgtgctcttccgatctgaggaggggagattcagtgtggt

SEQ ID NO: 435 - DNA, 48 bp, synthetic construct
  misc_feature 1..48: note = Synthetic oligonucleotide
  source 1..48: mol_type = other DNA
ccccttagggataacagggtaatcaggtgtgactcctttcccagatca

SEQ ID NO: 436 - DNA, 49 bp, synthetic construct
  misc_feature 1..49: note = Synthetic oligonucleotide
  source 1..49: mol_type = other DNA
ccccttagggataacagggtaatcagaagtcctgggtatggaggctttg

SEQ ID NO: 437 - DNA, 48 bp, synthetic construct
  misc_feature 1..48: note = Synthetic oligonucleotide
  source 1..48: mol_type = other DNA
ccccttagggataacagggtaatcccactaggctaagaggtacaccgt

SEQ ID NO: 438 - DNA, 46 bp, synthetic construct
  misc_feature 1..46: note = Synthetic oligonucleotide
  source 1..46: mol_type = other DNA
ccccttagggataacagggtaatcccagtggcatccccttttgtca

SEQ ID NO: 439 - DNA, 44 bp, synthetic construct
  misc_feature 1..44: note = Synthetic oligonucleotide
  source 1..44: mol_type = other DNA
ccccttagggataacagggtaatctttggcagcggtgatgaggt

SEQ ID NO: 440 - DNA, 47 bp, synthetic construct
  misc_feature 1..47: note = Synthetic oligonucleotide
  source 1..47: mol_type = other DNA
ccccttagggataacagggtaatcgcaagggtaacacctgagaaggt

SEQ ID NO: 441 - DNA, 48 bp, synthetic construct
  misc_feature 1..48: note = Synthetic oligonucleotide
  source 1..48: mol_type = other DNA
ccccttagggataacagggtaatctggcaggtgtagctttttctgtta

SEQ ID NO: 442 - DNA, 47 bp, synthetic construct
  misc_feature 1..47: note = Synthetic oligonucleotide
  source 1..47: mol_type = other DNA
ccccttagggataacagggtaatcgcggattaaagggaagggcttcg

SEQ ID NO: 443 - DNA, 47 bp, synthetic construct
  misc_feature 1..47: note = Synthetic oligonucleotide
  source 1..47: mol_type = other DNA
ccccttagggataacagggtaatcgccgttaccataagtcagcaggt

SEQ ID NO: 444 - DNA, 42 bp, synthetic construct
  misc_feature 1..42: note = Synthetic oligonucleotide
  source 1..42: mol_type = other DNA
ccccttagggataacagggtaatcacccaagcggcccttcct

SEQ ID NO: 445 - DNA, 48 bp, synthetic construct
  misc_feature 1..48: note = Synthetic oligonucleotide
  source 1..48: mol_type = other DNA
ccccttagggataacagggtaatcctgcacacacattgcccacttaca

SEQ ID NO: 446 - DNA, 49 bp, synthetic construct
  misc_feature 1..49: note = Synthetic oligonucleotide
  source 1..49: mol_type = other DNA
ccccttagggataacagggtaatcgtgaagttggaccagctgtcataca

SEQ ID NO: 447 - DNA, 50 bp, synthetic construct
  misc_feature 1..50: note = Synthetic oligonucleotide
  source 1..50: mol_type = other DNA
ccccttagggataacagggtaatctgtgtgtcacatcaattaatttgtgc

SEQ ID NO: 448 - DNA, 46 bp, synthetic construct
  misc_feature 1..46: note = Synthetic oligonucleotide
  source 1..46: mol_type = other DNA
ccccttagggataacagggtaatcgctctgcaagtactgactgcct

SEQ ID NO: 449 - DNA, 45 bp, synthetic construct
  misc_feature 1..45: note = Synthetic oligonucleotide
  source 1..45: mol_type = other DNA
ccccttagggataacagggtaatcatgaggggacaccagagggaa

SEQ ID NO: 450 - DNA, 46 bp, synthetic construct
  misc_feature 1..46: note = Synthetic oligonucleotide
  source 1..46: mol_type = other DNA
ccccttagggataacagggtaatcccctctggagtcccatcatcac

SEQ ID NO: 451 - DNA, 45 bp, synthetic construct
  misc_feature 1..45: note = Synthetic oligonucleotide
  source 1..45: mol_type = other DNA
ccccttagggataacagggtaatctgctgtgtctgctgtccatcc

SEQ ID NO: 452 - DNA, 43 bp, synthetic construct
  misc_feature 1..43: note = Synthetic oligonucleotide
  source 1..43: mol_type = other DNA
ccccttagggataacagggtaatcgctgctgctggagagccat

SEQ ID NO: 453 - DNA, 46 bp, synthetic construct
  misc_feature 1..46: note = Synthetic oligonucleotide
  source 1..46: mol_type = other DNA
ccccttagggataacagggtaatcgtcgaactgcatcccctggttt

SEQ ID NO: 454 - DNA, 45 bp, synthetic construct
  misc_feature 1..45: note = Synthetic oligonucleotide
  source 1..45: mol_type = other DNA
ccccttagggataacagggtaatcgttccgctacgtcagttgcca

SEQ ID NO: 455 - DNA, 43 bp, synthetic construct
  misc_feature 1..43: note = Synthetic oligonucleotide
  source 1..43: mol_type = other DNA
ccccttagggataacagggtaatcggaatggccacccttccct

SEQ ID NO: 456 - DNA, 49 bp, synthetic construct
  misc_feature 1..49: note = Synthetic oligonucleotide
  source 1..49: mol_type = other DNA
ccccttagggataacagggtaatccctcctggaggtctctctttaatgc

SEQ ID NO: 457 - DNA, 46 bp, synthetic construct
  misc_feature 1..46: note = Synthetic oligonucleotide
  source 1..46: mol_type = other DNA
ccccttagggataacagggtaatcgtcattctgctgggtgacaatg

SEQ ID NO: 458 - DNA, 49 bp, synthetic construct
  misc_feature 1..49: note = Synthetic oligonucleotide
  source 1..49: mol_type = other DNA
ccccttagggataacagggtaatctcacacagtggttaagaccctttgg

SEQ ID NO: 459 - DNA, 50 bp, synthetic construct
  misc_feature 1..50: note = Synthetic oligonucleotide
  source 1..50: mol_type = other DNA
ccccttagggataacagggtaatcgtgggctagaagctaagaagatcagc

SEQ ID NO: 460 - DNA, 49 bp, synthetic construct
  misc_feature 1..49: note = Synthetic oligonucleotide
  source 1..49: mol_type = other DNA
ccccttagggataacagggtaatcagtacgatgctgcttcacatggaac

SEQ ID NO: 461 - DNA, 47 bp, synthetic construct
  misc_feature 1..47: note = Synthetic oligonucleotide
  source 1..47: mol_type = other DNA
ccccttagggataacagggtaatcacgactgttctcactgaggggta

SEQ ID NO: 462 - DNA, 47 bp, synthetic construct
  misc_feature 1..47: note = Synthetic oligonucleotide
  source 1..47: mol_type = other DNA
ccccttagggataacagggtaatcgggagacttaccagcttcccgta

SEQ ID NO: 463 - DNA, 45 bp, synthetic construct
  misc_feature 1..45: note = Synthetic oligonucleotide
  source 1..45: mol_type = other DNA
ccccttagggataacagggtaatctaaggcagtgtgttgggtgct

SEQ ID NO: 464 - DNA, 49 bp, synthetic construct
  misc_feature 1..49: note = Synthetic oligonucleotide
  source 1..49: mol_type = other DNA
ccccttagggataacagggtaatcccttccttctccacccaagtagcta

SEQ ID NO: 465 - DNA, 48 bp, synthetic construct
  misc_feature 1..48: note = Synthetic oligonucleotide
  source 1..48: mol_type = other DNA
ccccttagggataacagggtaatcctcacactctacccttgtgctacg

SEQ ID NO: 466 - DNA, 46 bp, synthetic construct
  misc_feature 1..46: note = Synthetic oligonucleotide
  source 1..46: mol_type = other DNA
ccccttagggataacagggtaatccaactgggcatgctctcctagg

SEQ ID NO: 467 - DNA, 45 bp, synthetic construct
  misc_feature 1..45: note = Synthetic oligonucleotide
  source 1..45: mol_type = other DNA
ccccttagggataacagggtaatcctgtgtggccctcaggtgtaa

SEQ ID NO: 468 - DNA, 43 bp, synthetic construct
  misc_feature 1..43: note = Synthetic oligonucleotide
  source 1..43: mol_type = other DNA
ccccttagggataacagggtaatcaccacacccggctcactct

SEQ ID NO: 469 - DNA, 44 bp, synthetic construct
  misc_feature 1..44: note = Synthetic oligonucleotide
  source 1..44: mol_type = other DNA
ccccttagggataacagggtaatcggaggttgcaggttgctggt

SEQ ID NO: 470 - DNA, 43 bp, synthetic construct
  misc_feature 1..43: note = Synthetic oligonucleotide
  source 1..43: mol_type = other DNA
ccccttagggataacagggtaatcggctggagtcctggtcctg

SEQ ID NO: 471 - DNA, 44 bp, synthetic construct
  misc_feature 1..44: note = Synthetic oligonucleotide
  source 1..44: mol_type = other DNA
ccccttagggataacagggtaatcatggtcaccgccattcacgt

SEQ ID NO: 472 - DNA, 49 bp, synthetic construct
  misc_feature 1..49: note = Synthetic oligonucleotide
  source 1..49: mol_type = other DNA
ccccttagggataacagggtaatcctatcattacccacacccctgagac

SEQ ID NO: 473 - DNA, 50 bp, synthetic construct
  misc_feature 1..50: note = Synthetic oligonucleotide
  source 1..50: mol_type = other DNA
ccccttagggataacagggtaatcagctaccacggtgacagtaacatagc

SEQ ID NO: 474 - DNA, 43 bp, synthetic construct
  misc_feature 1..43: note = Synthetic oligonucleotide
  source 1..43: mol_type = other DNA
ccccttagggataacagggtaatcagctgccagcccacaagaa

SEQ ID NO: 475 - DNA, 45 bp, synthetic construct
  misc_feature 1..45: note = Synthetic oligonucleotide
  source 1..45: mol_type = other DNA
ccccttagggataacagggtaatcgggagacagggtatccaggct

SEQ ID NO: 476 - DNA, 46 bp, synthetic construct
  misc_feature 1..46: note = Synthetic oligonucleotide
  source 1..46: mol_type = other DNA
ccccttagggataacagggtaatcagttcagggtctggttctgtgc

SEQ ID NO: 477 - DNA, 45 bp, synthetic construct
  misc_feature 1..45: note = Synthetic oligonucleotide
  source 1..45: mol_type = other DNA
ccccttagggataacagggtaatccggcattcttcccggcaatga

SEQ ID NO: 478 - DNA, 48 bp, synthetic construct
  misc_feature 1..48: note = Synthetic oligonucleotide
  source 1..48: mol_type = other DNA
ccccttagggataacagggtaatctgactctcagcaccttgacactcc

SEQ ID NO: 479 - DNA, 50 bp, synthetic construct
  misc_feature 1..50: note = Synthetic oligonucleotide
  source 1..50: mol_type = other DNA
ccccttagggataacagggtaatcctttatatgtgggggatggaaaagac

SEQ ID NO: 480 - DNA, 49 bp, synthetic construct
  misc_feature 1..49: note = Synthetic oligonucleotide
  source 1..49: mol_type = other DNA
ccccttagggataacagggtaatccagtgccttttcctactacaccaca

SEQ ID NO: 481 - DNA, 50 bp, synthetic construct
  misc_feature 1..50: note = Synthetic oligonucleotide
  source 1..50: mol_type = other DNA
ccccttagggataacagggtaatccgaaggaaccaaacggaacttgtgta

SEQ ID NO: 482 - DNA, 45 bp, synthetic construct
  misc_feature 1..45: note = Synthetic oligonucleotide
  source 1..45: mol_type = other DNA
ccccttagggataacagggtaatcagctcatcgaggcaccaaaca

SEQ ID NO: 483 - DNA, 49 bp, synthetic construct
  misc_feature 1..49: note = Synthetic oligonucleotide
  source 1..49: mol_type = other DNA
ccccttagggataacagggtaatcatttgtcctggaacccatactgcat

SEQ ID NO: 484 - DNA, 49 bp, synthetic construct
  misc_feature 1..49: note = Synthetic oligonucleotide
  source 1..49: mol_type = other DNA
ccccttagggataacagggtaatctgaaagcatcaactctgggagcatg

SEQ ID NO: 485 - DNA, 45 bp, synthetic construct
  misc_feature 1..45: note = Synthetic oligonucleotide
  source 1..45: mol_type = other DNA
ccccttagggataacagggtaatcgccacagttccagtgcattcg

SEQ ID NO: 486 - DNA, 46 bp, synthetic construct
  misc_feature 1..46: note = Synthetic oligonucleotide
  source 1..46: mol_type = other DNA
ccccttagggataacagggtaatcggctccccagaagaagaagcct

SEQ ID NO: 487 - DNA, 50 bp, synthetic construct
  misc_feature 1..50: note = Synthetic oligonucleotide
  source 1..50: mol_type = other DNA
ccccttagggataacagggtaatcgcaagtggtaggcatgggttagaaga

SEQ ID NO: 488 - DNA, 46 bp, synthetic construct
  misc_feature 1..46: note = Synthetic oligonucleotide
  source 1..46: mol_type = other DNA
ccccttagggataacagggtaatcgcccggcaatcgttttctaggg

SEQ ID NO: 489 - DNA, 42 bp, synthetic construct
  misc_feature 1..42: note = Synthetic oligonucleotide
  source 1..42: mol_type = other DNA
ccccttagggataacagggtaatcacccccaggtcagcaagc

SEQ ID NO: 490 - DNA, 50 bp, synthetic construct
  misc_feature 1..50: note = Synthetic oligonucleotide
  source 1..50: mol_type = other DNA
ccccttagggataacagggtaatcctgattagggtggttcgttttgacgt

SEQ ID NO: 491 - DNA, 42 bp, synthetic construct
  misc_feature 1..42: note = Synthetic oligonucleotide
  source 1..42: mol_type = other DNA
ccccttagggataacagggtaatcgcacgaccgcggcagagt

SEQ ID NO: 492 - DNA, 43 bp, synthetic construct
  misc_feature 1..43: note = Synthetic oligonucleotide
  source 1..43: mol_type = other DNA
ccccttagggataacagggtaatcagctgcttcccaggccttg

SEQ ID NO: 493 - DNA, 44 bp, synthetic construct
  misc_feature 1..44: note = Synthetic oligonucleotide
  source 1..44: mol_type = other DNA
ccccttagggataacagggtaatcaatgcagaggccaggacacc

SEQ ID NO: 494 - DNA, 49 bp, synthetic construct
  misc_feature 1..49: note = Synthetic oligonucleotide
  source 1..49: mol_type = other DNA
ccccttagggataacagggtaatctcatgttgtggttggaagtgtggat

SEQ ID NO: 495 - DNA, 45 bp, synthetic construct
  misc_feature 1..45: note = Synthetic oligonucleotide
  source 1..45: mol_type = other DNA
ccccttagggataacagggtaatctggctggaagatggacggaga

SEQ ID NO: 496 - DNA, 45 bp, synthetic construct
  misc_feature 1..45: note = Synthetic oligonucleotide
  source 1..45: mol_type = other DNA
ccccttagggataacagggtaatccaccaggccactcacccaatt

SEQ ID NO: 497 - DNA, 50 bp, synthetic construct
  misc_feature 1..50: note = Synthetic oligonucleotide
  source 1..50: mol_type = other DNA
ccccttagggataacagggtaatcgagaccagtgatttcagagtggctag

SEQ ID NO: 498 - DNA, 48 bp, synthetic construct
  misc_feature 1..48: note = Synthetic oligonucleotide
  source 1..48: mol_type = other DNA
ccccttagggataacagggtaatcaccccgaacttggtgatgcagtac

SEQ ID NO: 499 - DNA, 45 bp, synthetic construct
  misc_feature 1..45: note = Synthetic oligonucleotide
  source 1..45: mol_type = other DNA
ccccttagggataacagggtaatcgggtggctcagaagtggttcc

SEQ ID NO: 500 - DNA, 48 bp, synthetic construct
  misc_feature 1..48: note = Synthetic oligonucleotide
  source 1..48: mol_type = other DNA
ccccttagggataacagggtaatcgtaggtgatagggaaacgccgaaa

SEQ ID NO: 501 - DNA, 45 bp, synthetic construct
  misc_feature 1..45: note = Synthetic oligonucleotide
  source 1..45: mol_type = other DNA
ccccttagggataacagggtaatctctgcagagcatggaggcaac

SEQ ID NO: 502 - DNA, 49 bp, synthetic construct
  misc_feature 1..49: note = Synthetic oligonucleotide
  source 1..49: mol_type = other DNA
ccccttagggataacagggtaatcaagtctgaaacgctgctctgctatt

SEQ ID NO: 503 - DNA, 46 bp, synthetic construct
  misc_feature 1..46: note = Synthetic oligonucleotide
  source 1..46: mol_type = other DNA
ccccttagggataacagggtaatcgcaccatttccacccagctttg

SEQ ID NO: 504 - DNA, 50 bp, synthetic construct
  misc_feature 1..50: note = Synthetic oligonucleotide
  source 1..50: mol_type = other DNA
ccccttagggataacagggtaatccaagtagctaggactcaaggcacatg

SEQ ID NO: 505 - DNA, 45 bp, synthetic construct
  misc_feature 1..45: note = Synthetic oligonucleotide
  source 1..45: mol_type = other DNA
ccccttagggataacagggtaatcgggggctgatatgggtcaacc

SEQ ID NO: 506 - DNA, 45 bp, synthetic construct
  misc_feature 1..45: note = Synthetic oligonucleotide
  source 1..45: mol_type = other DNA
ccccttagggataacagggtaatctgcatcgaagctggtggagac

SEQ ID NO: 507 - DNA, 49 bp, synthetic construct
  misc_feature 1..49: note = Synthetic oligonucleotide
  source 1..49: mol_type = other DNA
ccccttagggataacagggtaatcccagaccctgactcatggacacacc

SEQ ID NO: 508 - DNA, 44 bp, synthetic construct
  misc_feature 1..44: note = Synthetic oligonucleotide
  source 1..44: mol_type = other DNA
ccccttagggataacagggtaatcacgttcccgtctgctcagtg

SEQ ID NO: 509 - DNA, 47 bp, synthetic construct
  misc_feature 1..47: note = Synthetic oligonucleotide
  source 1..47: mol_type = other DNA
ccccttagggataacagggtaatcgaggttggaccagctgtcatacc

SEQ ID NO: 510 - DNA, 46 bp, synthetic construct
  misc_feature 1..46: note = Synthetic oligonucleotide
  source 1..46: mol_type = other DNA
ccccttagggataacagggtaatcgctagtctttccaggccaccct

SEQ ID NO: 511 - DNA, 46 bp, synthetic construct
  misc_feature 1..46: note = Synthetic oligonucleotide
  source 1..46: mol_type = other DNA
ccccttagggataacagggtaatcttggcaagcactcctcaatggc

SEQ ID NO: 512 - DNA, 46 bp, synthetic construct
  misc_feature 1..46: note = Synthetic oligonucleotide
  source 1..46: mol_type = other DNA
ccccttagggataacagggtaatcgcagagaggaggggctaaaggg

SEQ ID NO: 513 - DNA, 45 bp, synthetic construct
  misc_feature 1..45: note = Synthetic oligonucleotide
  source 1..45: mol_type = other DNA
ccccttagggataacagggtaatcctcccatccatacccccacct

SEQ ID NO: 514 - DNA, 47 bp, synthetic construct
  misc_feature 1..47: note = Synthetic oligonucleotide
  source 1..47: mol_type = other DNA
ccccttagggataacagggtaatcgccccaacccaagctagtctttc

SEQ ID NO: 515 - DNA, 45 bp, synthetic construct
  misc_feature 1..45: note = Synthetic oligonucleotide
  source 1..45: mol_type = other DNA
ccccttagggataacagggtaatccctttcccgttctccacccaa

SEQ ID NO: 516 - DNA, 49 bp, synthetic construct
  misc_feature 1..49: note = Synthetic oligonucleotide
  source 1..49: mol_type = other DNA
ccccttagggataacagggtaatcagcagtatgtccaactcccaaattg

SEQ ID NO: 517 - DNA, 49 bp, synthetic construct
  misc_feature 1..49: note = Synthetic oligonucleotide
  source 1..49: mol_type = other DNA
ccccttagggataacagggtaatcacacaggttttctcctctcagccta

SEQ ID NO: 518 - DNA, 44 bp, synthetic construct
  misc_feature 1..44: note = Synthetic oligonucleotide
  source 1..44: mol_type = other DNA
ccccttagggataacagggtaatcaacctggctccttcgcttcc

SEQ ID NO: 519 - DNA, 46 bp, synthetic construct
  misc_feature 1..46: note = Synthetic oligonucleotide
  source 1..46: mol_type = other DNA
ccccttagggataacagggtaatcctctgtctccacatgcccagtt

SEQ ID NO: 520 - DNA, 47 bp, synthetic construct
  misc_feature 1..47: note = Synthetic oligonucleotide
  source 1..47: mol_type = other DNA
ccccttagggataacagggtaatcccagggctgggcataaaagtcag

SEQ ID NO: 521 - DNA, 60 bp, synthetic construct
  misc_feature 1..60: note = Synthetic oligonucleotide
  source 1..60: mol_type = other DNA
gtgactggagttcagacgtgtgctcttccgatctgtgactcctttcccagatcagatagc

SEQ ID NO: 522 - DNA, 59 bp, synthetic construct
  misc_feature 1..59: note = Synthetic oligonucleotide
  source 1..59: mol_type = other DNA
gtgactggagttcagacgtgtgctcttccgatctcctgggtatggaggctttggcattc

SEQ ID NO: 523 - DNA, 60 bp, synthetic construct
  misc_feature 1..60: note = Synthetic oligonucleotide
  source 1..60: mol_type = other DNA
gtgactggagttcagacgtgtgctcttccgatctggctaagaggtacaccgtaacagaga

SEQ ID NO: 524 - DNA, 60 bp, synthetic construct
  misc_feature 1..60: note = Synthetic oligonucleotide
  source 1..60: mol_type = other DNA
gtgactggagttcagacgtgtgctcttccgatctagcatgtcatatggctaacaccggtt

SEQ ID NO: 525 - DNA, 60 bp, synthetic construct
  misc_feature 1..60: note = Synthetic oligonucleotide
  source 1..60: mol_type = other DNA
gtgactggagttcagacgtgtgctcttccgatctgaggtttctcatcctgcatgacgtat

SEQ ID NO: 526 - DNA, 54 bp, synthetic construct
  misc_feature 1..54: note = Synthetic oligonucleotide
  source 1..54: mol_type = other DNA
gtgactggagttcagacgtgtgctcttccgatctgtgtggggtaaggggagctg

SEQ ID NO: 527 - DNA, 59 bp, synthetic construct
  misc_feature 1..59: note = Synthetic oligonucleotide
  source 1..59: mol_type = other DNA
gtgactggagttcagacgtgtgctcttccgatctagaacattctgtcattccagtcaga

SEQ ID NO: 528 - DNA, 60 bp, synthetic construct
  misc_feature 1..60: note = Synthetic oligonucleotide
  source 1..60: mol_type = other DNA
gtgactggagttcagacgtgtgctcttccgatctagggaagggcttcgaatgagaatgct

SEQ ID NO: 529 - DNA, 60 bp, synthetic construct
  misc_feature 1..60: note = Synthetic oligonucleotide
  source 1..60: mol_type = other DNA
gtgactggagttcagacgtgtgctcttccgatctcagaaagtcacttccagcacttgtga

SEQ ID NO: 530 - DNA, 54 bp, synthetic construct
  misc_feature 1..54: note = Synthetic oligonucleotide
  source 1..54: mol_type = other DNA
gtgactggagttcagacgtgtgctcttccgatcttcctccaggcttgacttggc

SEQ ID NO: 531 - DNA, 55 bp, synthetic construct
  misc_feature 1..55: note = Synthetic oligonucleotide
  source 1..55: mol_type = other DNA
gtgactggagttcagacgtgtgctcttccgatctcaccccagaacacgagcaact

SEQ ID NO: 532 - DNA, 60 bp, synthetic construct
  misc_feature 1..60: note = Synthetic oligonucleotide
  source 1..60: mol_type = other DNA
gtgactggagttcagacgtgtgctcttccgatctgttggaccagctgtcatacacacaac

SEQ ID NO: 533 - DNA, 60 bp, synthetic construct
  misc_feature 1..60: note = Synthetic oligonucleotide
  source 1..60: mol_type = other DNA
gtgactggagttcagacgtgtgctcttccgatctttgtgcacaggtttaagaaacaaata

SEQ ID NO: 534 - DNA, 58 bp, synthetic construct
  misc_feature 1..58: note = Synthetic oligonucleotide
  source 1..58: mol_type = other DNA
gtgactggagttcagacgtgtgctcttccgatctgcaagtactgactgcctccccctt

SEQ ID NO: 535 - DNA, 56 bp, synthetic construct
  misc_feature 1..56: note = Synthetic oligonucleotide
  source 1..56: mol_type = other DNA
gtgactggagttcagacgtgtgctcttccgatctgggacaccagagggaagtgagg

SEQ ID NO: 536 - DNA, 57 bp, synthetic construct
  misc_feature 1..57: note = Synthetic oligonucleotide
  source 1..57: mol_type = other DNA
gtgactggagttcagacgtgtgctcttccgatctatcaccatctggcatcccttcac

SEQ ID NO: 537 - DNA, 58 bp, synthetic construct
  misc_feature 1..58: note = Synthetic oligonucleotide
  source 1..58: mol_type = other DNA
gtgactggagttcagacgtgtgctcttccgatctgtgtctgctgtccatccttcacat

SEQ ID NO: 538 - DNA, 60 bp, synthetic construct
  misc_feature 1..60: note = Synthetic oligonucleotide
  source 1..60: mol_type = other DNA
gtgactggagttcagacgtgtgctcttccgatcttgctggagagccatcttgaaactaag

SEQ ID NO: 539 - DNA, 52 bp, synthetic construct
  misc_feature 1..52: note = Synthetic oligonucleotide
  source 1..52: mol_type = other DNA
gtgactggagttcagacgtgtgctcttccgatctgccagggcagccttccag

SEQ ID NO: 540 - DNA, 58 bp, synthetic construct
  misc_feature 1..58: note = Synthetic oligonucleotide
  source 1..58: mol_type = other DNA
gtgactggagttcagacgtgtgctcttccgatctcgtcagttgccacttctgtatcca

SEQ ID NO: 541 - DNA, 60 bp, synthetic construct
  misc_feature 1..60: note = Synthetic oligonucleotide
  source 1..60: mol_type = other DNA
gtgactggagttcagacgtgtgctcttccgatctacccttccctccttatcagaaattgc

SEQ ID NO: 542 - DNA, 56 bp, synthetic construct
  misc_feature 1..56: note = Synthetic oligonucleotide
  source 1..56: mol_type = other DNA
gtgactggagttcagacgtgtgctcttccgatctgccccttttctcacagtgtgca

SEQ ID NO: 543 - DNA, 60 bp, synthetic construct
  misc_feature 1..60: note = Synthetic oligonucleotide
  source 1..60: mol_type = other DNA
gtgactggagttcagacgtgtgctcttccgatctcattctgctgggtgacaatgaaatat

SEQ ID NO: 544 - DNA, 60 bp, synthetic construct
  misc_feature 1..60: note = Synthetic oligonucleotide
  source 1..60: mol_type = other DNA
gtgactggagttcagacgtgtgctcttccgatctgtggttaagaccctttggcatgagag

SEQ ID NO: 545 - DNA, 59 bp, synthetic construct
  misc_feature 1..59: note = Synthetic oligonucleotide
  source 1..59: mol_type = other DNA
gtgactggagttcagacgtgtgctcttccgatctagaagctaagaagatcagccagcag

SEQ ID NO: 546 - DNA, 57 bp, synthetic construct
  misc_feature 1..57: note = Synthetic oligonucleotide
  source 1..57: mol_type = other DNA
gtgactggagttcagacgtgtgctcttccgatcttcacatggaacccagcaggaatc

SEQ ID NO: 547 - DNA, 55 bp, synthetic construct
  misc_feature 1..55: note = Synthetic oligonucleotide
  source 1..55: mol_type = other DNA
gtgactggagttcagacgtgtgctcttccgatctaggaggaaagggtggagctga

SEQ ID NO: 548 - DNA, 55 bp, synthetic construct
  misc_feature 1..55: note = Synthetic oligonucleotide
  source 1..55: mol_type = other DNA
gtgactggagttcagacgtgtgctcttccgatctaccagcttcccgtatctccct

SEQ ID NO: 549 - DNA, 59 bp, synthetic construct
  misc_feature 1..59: note = Synthetic oligonucleotide
  source 1..59: mol_type = other DNA
gtgactggagttcagacgtgtgctcttccgatctgctgttgcagaagggatagtcagag

SEQ ID NO: 550 - DNA, 54 bp, synthetic construct
  misc_feature 1..54: note = Synthetic oligonucleotide
  source 1..54: mol_type = other DNA
gtgactggagttcagacgtgtgctcttccgatctatgtgccctctgtgtgcctt

SEQ ID NO: 551 - DNA, 58 bp, synthetic construct
  misc_feature 1..58: note = Synthetic oligonucleotide
  source 1..58: mol_type = other DNA
gtgactggagttcagacgtgtgctcttccgatctctctacccttgtgctacgctgtct

SEQ ID NO: 552 - DNA, 53 bp, synthetic construct
  misc_feature 1..53: note = Synthetic oligonucleotide
  source 1..53: mol_type = other DNA
gtgactggagttcagacgtgtgctcttccgatctgcaaggggccagaaggtct

SEQ ID NO: 553 - DNA, 60 bp, synthetic construct
  misc_feature 1..60: note = Synthetic oligonucleotide
  source 1..60: mol_type = other DNA
gtgactggagttcagacgtgtgctcttccgatctggccctcaggtgtaacttaccctctc

SEQ ID NO: 554 - DNA, 58 bp, synthetic construct
  misc_feature 1..58: note = Synthetic oligonucleotide
  source 1..58: mol_type = other DNA
gtgactggagttcagacgtgtgctcttccgatctccacacccggctcactctccaatt

SEQ ID NO: 555 - DNA, 59 bp, synthetic construct
  misc_feature 1..59: note = Synthetic oligonucleotide
  source 1..59: mol_type = other DNA
gtgactggagttcagacgtgtgctcttccgatctgttgctggttgctgagatcatgcca

SEQ ID NO: 556 - DNA, 53 bp, synthetic construct
  misc_feature 1..53: note = Synthetic oligonucleotide
  source 1..53: mol_type = other DNA
gtgactggagttcagacgtgtgctcttccgatctccaatcacgggccctggga

SEQ ID NO: 557 - DNA, 58 bp, synthetic construct
  misc_feature 1..58: note = Synthetic oligonucleotide
  source 1..58: mol_type = other DNA
gtgactggagttcagacgtgtgctcttccgatctccgccattcacgtggtgcttactg

SEQ ID NO: 558 - DNA, 56 bp, synthetic construct
  misc_feature 1..56: note = Synthetic oligonucleotide
  source 1..56: mol_type = other DNA
gtgactggagttcagacgtgtgctcttccgatctcccacacccctgagactgcata

SEQ ID NO: 559 - DNA, 59 bp, synthetic construct
  misc_feature 1..59: note = Synthetic oligonucleotide
  source 1..59: mol_type = other DNA
gtgactggagttcagacgtgtgctcttccgatctcggtgacagtaacatagcccaggga

SEQ ID NO: 560 - DNA, 60 bp, synthetic construct
  misc_feature 1..60: note = Synthetic oligonucleotide
  source 1..60: mol_type = other DNA
gtgactggagttcagacgtgtgctcttccgatctaaaatggggcccttagtcctacaatg

SEQ ID NO: 561 - DNA, 60 bp, synthetic construct
  misc_feature 1..60: note = Synthetic oligonucleotide
  source 1..60: mol_type = other DNA
gtgactggagttcagacgtgtgctcttccgatctgagacagggtatccaggctgcataca

SEQ ID NO: 562 - DNA, 60 bp, synthetic construct
  misc_feature 1..60: note = Synthetic oligonucleotide
  source 1..60: mol_type = other DNA
gtgactggagttcagacgtgtgctcttccgatctttcagggtctggttctgtgcacataa

SEQ ID NO: 563 - DNA, 60 bp, synthetic construct
  misc_feature 1..60: note = Synthetic oligonucleotide
  source 1..60: mol_type = other DNA
gtgactggagttcagacgtgtgctcttccgatctggcattcttcccggcaatgaaatcct

SEQ ID NO: 564 - DNA, 60 bp, synthetic construct
  misc_feature 1..60: note = Synthetic oligonucleotide
  source 1..60: mol_type = other DNA
gtgactggagttcagacgtgtgctcttccgatctcagcaccttgacactccagatgaact

SEQ ID NO: 565 - DNA, 60 bp, synthetic construct
  misc_feature 1..60: note = Synthetic oligonucleotide
  source 1..60: mol_type = other DNA
gtgactggagttcagacgtgtgctcttccgatctatggaaaagacaacccatcatggtat

SEQ ID NO: 566 - DNA, 60 bp, synthetic construct
  misc_feature 1..60: note = Synthetic oligonucleotide
  source 1..60: mol_type = other DNA
gtgactggagttcagacgtgtgctcttccgatctcctactacaccacactgatgcctcca

SEQ ID NO: 567 - DNA, 58 bp, synthetic construct
  misc_feature 1..58: note = Synthetic oligonucleotide
  source 1..58: mol_type = other DNA
gtgactggagttcagacgtgtgctcttccgatcttctgggtgggagcagagtactctt

SEQ ID NO: 568 - DNA, 60 bp, synthetic construct
  misc_feature 1..60: note = Synthetic oligonucleotide
  source 1..60: mol_type = other DNA
gtgactggagttcagacgtgtgctcttccgatctgtggtgattacaaggccacatcctac

SEQ ID NO: 569 - DNA, 60 bp, synthetic construct
  misc_feature 1..60: note = Synthetic oligonucleotide
  source 1..60: mol_type = other DNA
gtgactggagttcagacgtgtgctcttccgatctcctggaacccatactgcattaggaag

SEQ ID NO: 570 - DNA, 60 bp, synthetic construct
  misc_feature 1..60: note = Synthetic oligonucleotide
  source 1..60: mol_type = other DNA
gtgactggagttcagacgtgtgctcttccgatctagcatgaaaaaggctgatgagtggga

SEQ ID NO: 571 - DNA, 60 bp, synthetic construct
  misc_feature 1..60: note = Synthetic oligonucleotide
  source 1..60: mol_type = other DNA
gtgactggagttcagacgtgtgctcttccgatctccacagttccagtgcattcggaagaa

SEQ ID NO: 572 - DNA, 55 bp, synthetic construct
  misc_feature 1..55: note = Synthetic oligonucleotide
  source 1..55: mol_type = other DNA
gtgactggagttcagacgtgtgctcttccgatctgcttgcagaaccacgagctga

SEQ ID NO: 573 - DNA, 60 bp, synthetic construct
  misc_feature 1..60: note = Synthetic oligonucleotide
  source 1..60: mol_type = other DNA
gtgactggagttcagacgtgtgctcttccgatcttcagctgtgcttctaatgtacaccct

SEQ ID NO: 574 - DNA, 60 bp, synthetic construct
  misc_feature 1..60: note = Synthetic oligonucleotide
  source 1..60: mol_type = other DNA
gtgactggagttcagacgtgtgctcttccgatctgcaatcgttttctagggcacgactta

SEQ ID NO: 575 - DNA, 60 bp, synthetic construct
  misc_feature 1..60: note = Synthetic oligonucleotide
  source 1..60: mol_type = other DNA
gtgactggagttcagacgtgtgctcttccgatctgtcagcaagcacttgatcagagcatt

SEQ ID NO: 576 - DNA, 60 bp, synthetic construct
  misc_feature 1..60: note = Synthetic oligonucleotide
  source 1..60: mol_type = other DNA
gtgactggagttcagacgtgtgctcttccgatctgtggttcgttttgacgtgtctgtttc

SEQ ID NO: 577 - DNA, 57 bp, synthetic construct
  misc_feature 1..57: note = Synthetic oligonucleotide
  source 1..57: mol_type = other DNA
gtgactggagttcagacgtgtgctcttccgatctcacgaccgcggcagagttatcag

SEQ ID NO: 578 - DNA, 59 bp, synthetic construct
  misc_feature 1..59: note = Synthetic oligonucleotide
  source 1..59: mol_type = other DNA
gtgactggagttcagacgtgtgctcttccgatctcccaggccttggcaatgagtttagg

SEQ ID NO: 579 - DNA, 54 bp, synthetic construct
  misc_feature 1..54: note = Synthetic oligonucleotide
  source 1..54: mol_type = other DNA
gtgactggagttcagacgtgtgctcttccgatctggccaggacaccaccatccc

SEQ ID NO: 580 - DNA, 60 bp, synthetic construct
  misc_feature 1..60: note = Synthetic oligonucleotide
  source 1..60: mol_type = other DNA
gtgactggagttcagacgtgtgctcttccgatcttgtggttggaagtgtggattactggt

SEQ ID NO: 581 - DNA, 60 bp, synthetic construct
  misc_feature 1..60: note = Synthetic oligonucleotide
  source 1..60: mol_type = other DNA
gtgactggagttcagacgtgtgctcttccgatcttggacggagagtggatcacagatgag

SEQ ID NO: 582 - DNA, 60 bp, synthetic construct
  misc_feature 1..60: note = Synthetic oligonucleotide
  source 1..60: mol_type = other DNA
gtgactggagttcagacgtgtgctcttccgatctccaggccactcacccaatttgacatg

SEQ ID NO: 583 - DNA, 60 bp, synthetic construct
  misc_feature 1..60: note = Synthetic oligonucleotide
  source 1..60: mol_type = other DNA
gtgactggagttcagacgtgtgctcttccgatctttcagagtggctaggtgttcactgat

SEQ ID NO: 584 - DNA, 54 bp, synthetic construct
  misc_feature 1..54: note = Synthetic oligonucleotide
  source 1..54: mol_type = other DNA
gtgactggagttcagacgtgtgctcttccgatcttacggggagcgggccgggtt

SEQ ID NO: 585 - DNA, 57 bp, synthetic construct
  misc_feature 1..57: note = Synthetic oligonucleotide
  source 1..57: mol_type = other DNA
gtgactggagttcagacgtgtgctcttccgatctgctcagaagtggttccagccaag

SEQ ID NO: 586 - DNA, 60 bp, synthetic construct
  misc_feature 1..60: note = Synthetic oligonucleotide
  source 1..60: mol_type = other DNA
gtgactggagttcagacgtgtgctcttccgatctagggaaacgccgaaagtattttaggt

SEQ ID NO: 587 - DNA, 55 bp, synthetic construct
  misc_feature 1..55: note = Synthetic oligonucleotide
  source 1..55: mol_type = other DNA
gtgactggagttcagacgtgtgctcttccgatctgcaactgctccctggtctctt

SEQ ID NO: 588 - DNA, 60 bp, synthetic construct
  misc_feature 1..60: note = Synthetic oligonucleotide
  source 1..60: mol_type = other DNA
gtgactggagttcagacgtgtgctcttccgatctcctgtgatcccttcgaagaatcttgt

SEQ ID NO: 589 - DNA, 55 bp, synthetic construct
  misc_feature 1..55: note = Synthetic oligonucleotide
  source 1..55: mol_type = other DNA
gtgactggagttcagacgtgtgctcttccgatcttccacccagctttgctcaagt

SEQ ID NO: 590 - DNA, 55 bp, synthetic construct
  misc_feature 1..55: note = Synthetic oligonucleotide
  source 1..55: mol_type = other DNA
gtgactggagttcagacgtgtgctcttccgatctccaccacggccagatcattga

SEQ ID NO: 591 - DNA, 58 bp, synthetic construct
  misc_feature 1..58: note = Synthetic oligonucleotide
  source 1..58: mol_type = other DNA
gtgactggagttcagacgtgtgctcttccgatctaactgggttgccatgaatctgctg

SEQ ID NO: 592 - DNA, 55 bp, synthetic construct
  misc_feature 1..55: note = Synthetic oligonucleotide
  source 1..55: mol_type = other DNA
gtgactggagttcagacgtgtgctcttccgatctgcagggctgaggtggaaagct

SEQ ID NO: 593 - DNA, 57 bp, synthetic construct
  misc_feature 1..57: note = Synthetic oligonucleotide
  source 1..57: mol_type = other DNA
gtgactggagttcagacgtgtgctcttccgatctgacacaccctcccccatctggca

SEQ ID NO: 594 - DNA, 56 bp, synthetic construct
  misc_feature 1..56: note = Synthetic oligonucleotide
  source 1..56: mol_type = other DNA
gtgactggagttcagacgtgtgctcttccgatcttggggtaaaggggactcactct

SEQ ID NO: 595 - DNA, 60 bp, synthetic construct
  misc_feature 1..60: note = Synthetic oligonucleotide
  source 1..60: mol_type = other DNA
gtgactggagttcagacgtgtgctcttccgatctagctgctttactgtcacacgtagcag

SEQ ID NO: 596 - DNA, 54 bp, synthetic construct
  misc_feature 1..54: note = Synthetic oligonucleotide
  source 1..54: mol_type = other DNA
gtgactggagttcagacgtgtgctcttccgatctccaccctctccgagccacct

SEQ ID NO: 597 - DNA, 55 bp, synthetic construct
  misc_feature 1..55: note = Synthetic oligonucleotide
  source 1..55: mol_type = other DNA
gtgactggagttcagacgtgtgctcttccgatctccagcttacaggcagggctgt

SEQ ID NO: 598 - DNA, 55 bp, synthetic construct
  misc_feature 1..55: note = Synthetic oligonucleotide
  source 1..55: mol_type = other DNA
gtgactggagttcagacgtgtgctcttccgatctggggcaggaagggagaagcac

SEQ ID NO: 599 - DNA, 56 bp, synthetic construct
  misc_feature 1..56: note = Synthetic oligonucleotide
  source 1..56: mol_type = other DNA
gtgactggagttcagacgtgtgctcttccgatcttccacccccaacctgagaagac

SEQ ID NO: 600 - DNA, 59 bp, synthetic construct
  misc_feature 1..59: note = Synthetic oligonucleotide
  source 1..59: mol_type = other DNA
gtgactggagttcagacgtgtgctcttccgatctcccaagctagtctttccaggccact

SEQ ID NO: 601 - DNA, 58 bp, synthetic construct
  misc_feature 1..58: note = Synthetic oligonucleotide
  source 1..58: mol_type = other DNA
gtgactggagttcagacgtgtgctcttccgatctccgttctccacccaatagctatgg

SEQ ID NO: 602 - DNA, 60 bp, synthetic construct
  misc_feature 1..60: note = Synthetic oligonucleotide
  source 1..60: mol_type = other DNA
gtgactggagttcagacgtgtgctcttccgatcttccaactcccaaattgaaagcacagc

SEQ ID NO: 603 - DNA, 55 bp, synthetic construct
  misc_feature 1..55: note = Synthetic oligonucleotide
  source 1..55: mol_type = other DNA
gtgactggagttcagacgtgtgctcttccgatctttcccttccctagacctgcct

SEQ ID NO: 604 - DNA, 60 bp, synthetic construct
  misc_feature 1..60: note = Synthetic oligonucleotide
  source 1..60: mol_type = other DNA
gtgactggagttcagacgtgtgctcttccgatctggctccttcgcttccatctgatcagg

SEQ ID NO: 605 - DNA, 60 bp, synthetic construct
  misc_feature 1..60: note = Synthetic oligonucleotide
  source 1..60: mol_type = other DNA
gtgactggagttcagacgtgtgctcttccgatctgtctccacatgcccagtttctattgg

SEQ ID NO: 606 - DNA, 60 bp, synthetic construct
  misc_feature 1..60: note = Synthetic oligonucleotide
  source 1..60: mol_type = other DNA
gtgactggagttcagacgtgtgctcttccgatctttcactagcaacctcaaacagacacc

SEQ ID NO: 607 - DNA, 49 bp, synthetic construct
  misc_feature 1..49: note = Synthetic oligonucleotide
  source 1..49: mol_type = other DNA
ccccttagggataacagggtaatctgggctgagagctagctttatgtga

SEQ ID NO: 608 - DNA, 44 bp, synthetic construct
  misc_feature 1..44: note = Synthetic oligonucleotide
  source 1..44: mol_type = other DNA
ccccttagggataacagggtaatcaggaggcagggacgtgaaac

SEQ ID NO: 609 - DNA, 44 bp, synthetic construct
  misc_feature 1..44: note = Synthetic oligonucleotide
  source 1..44: mol_type = other DNA
ccccttagggataacagggtaatcaggtgactccctggctttgc

SEQ ID NO: 610 - DNA, 45 bp, synthetic construct
  misc_feature 1..45: note = Synthetic oligonucleotide
  source 1..45: mol_type = other DNA
ccccttagggataacagggtaatctgatctgaggggcttggcaga

SEQ ID NO: 611 - DNA, 48 bp, synthetic construct
  misc_feature 1..48: note = Synthetic oligonucleotide
  source 1..48: mol_type = other DNA
ccccttagggataacagggtaatccacatgtggtacgtctggtccagt

SEQ ID NO: 612 - DNA, 43 bp, synthetic construct
  misc_feature 1..43: note = Synthetic oligonucleotide
  source 1..43: mol_type = other DNA
ccccttagggataacagggtaatcacgacgggtgtgtgggtga

SEQ ID NO: 613 - DNA, 44 bp, synthetic construct
  misc_feature 1..44: note = Synthetic oligonucleotide
  source 1..44: mol_type = other DNA
ccccttagggataacagggtaatccagctggggcgacatagtga

SEQ ID NO: 614 - DNA, 49 bp, synthetic construct
  misc_feature 1..49: note = Synthetic oligonucleotide
  source 1..49: mol_type = other DNA
ccccttagggataacagggtaatcggtaactgtaatatagagcccacca

SEQ ID NO: 615 - DNA, 44 bp, synthetic construct
  misc_feature 1..44: note = Synthetic oligonucleotide
  source 1..44: mol_type = other DNA
ccccttagggataacagggtaatcggggagggacaggttgtgag

SEQ ID NO: 616 - DNA, 49 bp, synthetic construct
  misc_feature 1..49: note = Synthetic oligonucleotide
  source 1..49: mol_type = other DNA
ccccttagggataacagggtaatctgaatcaccaactgccaaacacgtg

SEQ ID NO: 617 - DNA, 47 bp, synthetic construct
  misc_feature 1..47: note = Synthetic oligonucleotide
  source 1..47: mol_type = other DNA
ccccttagggataacagggtaatcggcccccagtgaatcaccaattg

SEQ ID NO: 618 - DNA, 46 bp, synthetic construct
  misc_feature 1..46: note = Synthetic oligonucleotide
  source 1..46: mol_type = other DNA
ccccttagggataacagggtaatcgcagaatcaagccagagcatgc

SEQ ID NO: 619 - DNA, 44 bp, synthetic construct
  misc_feature 1..44: note = Synthetic oligonucleotide
  source 1..44: mol_type = other DNA
ccccttagggataacagggtaatcagaggtgagggcgagctaga

SEQ ID NO: 620 - DNA, 50 bp, synthetic construct
  misc_feature 1..50: note = Synthetic oligonucleotide
  source 1..50: mol_type = other DNA
ccccttagggataacagggtaatctgccagtgatctttcctttccctctg

SEQ ID NO: 621 - DNA, 46 bp, synthetic construct
  misc_feature 1..46: note = Synthetic oligonucleotide
  source 1..46: mol_type = other DNA
ccccttagggataacagggtaatccaacagtcggtgtcctgatggt

SEQ ID NO: 622 - DNA, 46 bp, synthetic construct
  misc_feature 1..46: note = Synthetic oligonucleotide
  source 1..46: mol_type = other DNA
ccccttagggataacagggtaatctcctgtgccatgaccttcacac

SEQ ID NO: 623 - DNA, 42 bp, synthetic construct
  misc_feature 1..42: note = Synthetic oligonucleotide
  source 1..42: mol_type = other DNA
ccccttagggataacagggtaatcatggggaggcggcagtga

SEQ ID NO: 624 - DNA, 42 bp, synthetic construct
  misc_feature 1..42: note = Synthetic oligonucleotide
  source 1..42: mol_type = other DNA
ccccttagggataacagggtaatcggggctgggcagtcactc

SEQ ID NO: 625 - DNA, 45 bp, synthetic construct
  misc_feature 1..45: note = Synthetic oligonucleotide
  source 1..45: mol_type = other DNA
ccccttagggataacagggtaatcccagactgcgggtatgagagg

SEQ ID NO: 626 - DNA, 42 bp, synthetic construct
  misc_feature 1..42: note = Synthetic oligonucleotide
  source 1..42: mol_type = other DNA
ccccttagggataacagggtaatcggctccgacgctccacag

SEQ ID NO: 627 - DNA, 42 bp, synthetic construct
  misc_feature 1..42: note = Synthetic oligonucleotide
  source 1..42: mol_type = other DNA
ccccttagggataacagggtaatccccctagcggcccaggct

SEQ ID NO: 628 - DNA, 46 bp, synthetic construct
  misc_feature 1..46: note = Synthetic oligonucleotide
  source 1..46: mol_type = other DNA
ccccttagggataacagggtaatctcaggctctagcagtcccagta

SEQ ID NO: 629 - DNA, 48 bp, synthetic construct
  misc_feature 1..48: note = Synthetic oligonucleotide
  source 1..48: mol_type = other DNA
ccccttagggataacagggtaatcggcatggtgaagaaagaatgctac

SEQ ID NO: 630 - DNA, 50 bp, synthetic construct
  misc_feature 1..50: note = Synthetic oligonucleotide
  source 1..50: mol_type = other DNA
ccccttagggataacagggtaatcaggttcttgcttagaggcatgatgac

SEQ ID NO: 631 - DNA, 48 bp, synthetic construct
  misc_feature 1..48: note = Synthetic oligonucleotide
  source 1..48: mol_type = other DNA
ccccttagggataacagggtaatcgcccatgctgttcttatagcggta

SEQ ID NO: 632 - DNA, 50 bp, synthetic construct
  misc_feature 1..50: note = Synthetic oligonucleotide
  source 1..50: mol_type = other DNA
ccccttagggataacagggtaatctgtgcatactcagctactgtgctcta

SEQ ID NO: 633 - DNA, 47 bp, synthetic construct
  misc_feature 1..47: note = Synthetic oligonucleotide
  source 1..47: mol_type = other DNA
ccccttagggataacagggtaatctgcagatgatctggctgatggac

SEQ ID NO: 634 - DNA, 48 bp, synthetic construct
  misc_feature 1..48: note = Synthetic oligonucleotide
  source 1..48: mol_type = other DNA
ccccttagggataacagggtaatcccagattccctgctcagcaaagta

SEQ ID NO: 635 - DNA, 50 bp, synthetic construct
  misc_feature 1..50: note = Synthetic oligonucleotide
  source 1..50: mol_type = other DNA
ccccttagggataacagggtaatccaaccactgtgtaataagccgcttgt

SEQ ID NO: 636 - DNA, 48 bp, synthetic construct
  misc_feature 1..48: note = Synthetic oligonucleotide
  source 1..48: mol_type = other DNA
ccccttagggataacagggtaatcgctaaacttggcactggctttcac

SEQ ID NO: 637 - DNA, 46 bp, synthetic construct
  misc_feature 1..46: note = Synthetic oligonucleotide
  source 1..46: mol_type = other DNA
ccccttagggataacagggtaatcaaaccccacacaccacacgtat

SEQ ID NO: 638 - DNA, 42 bp, synthetic construct
  misc_feature 1..42: note = Synthetic oligonucleotide
  source 1..42: mol_type = other DNA
ccccttagggataacagggtaatcggggctcctgagggtgga

SEQ ID NO: 639 - DNA, 45 bp, synthetic construct
  misc_feature 1..45: note = Synthetic oligonucleotide
  source 1..45: mol_type = other DNA
ccccttagggataacagggtaatctgtctgcagtcacctgtccac

SEQ ID NO: 640 - DNA, 44 bp, synthetic construct
  misc_feature 1..44: note = Synthetic oligonucleotide
  source 1..44: mol_type = other DNA
ccccttagggataacagggtaatccactcccaggcgctcgagtt

SEQ ID NO: 641 - DNA, 44 bp, synthetic construct
  misc_feature 1..44: note = Synthetic oligonucleotide
  source 1..44: mol_type = other DNA
ccccttagggataacagggtaatcggacaaacacccacccaggt

SEQ ID NO: 642 - DNA, 50 bp, synthetic construct
  misc_feature 1..50: note = Synthetic oligonucleotide
  source 1..50: mol_type = other DNA
ccccttagggataacagggtaatctttaaccttcttagtagccagggaat

SEQ ID NO: 643 - DNA, 47 bp, synthetic construct
  misc_feature 1..47: note = Synthetic oligonucleotide
  source 1..47: mol_type = other DNA
ccccttagggataacagggtaatctgcacatattccacgtgggcata

SEQ ID NO: 644 - DNA, 47 bp, synthetic construct
  misc_feature 1..47: note = Synthetic oligonucleotide
  source 1..47: mol_type = other DNA
ccccttagggataacagggtaatcccacagacatcagagcagacaca

SEQ ID NO: 645 - DNA, 44 bp, synthetic construct
  misc_feature 1..44: note = Synthetic oligonucleotide
  source 1..44: mol_type = other DNA
ccccttagggataacagggtaatcacacctggtgagggcaactg

SEQ ID NO: 646 - DNA, 46 bp, synthetic construct
  misc_feature 1..46: note = Synthetic oligonucleotide
  source 1..46: mol_type = other DNA
ccccttagggataacagggtaatcgaggccaggtcctacattgagc

SEQ ID NO: 647 - DNA, 46 bp, synthetic construct
  misc_feature 1..46: note = Synthetic oligonucleotide
  source 1..46: mol_type = other DNA
ccccttagggataacagggtaatctctttctgtcagaggcaatggt

SEQ ID NO: 648 - DNA, 45 bp, synthetic construct
  misc_feature 1..45: note = Synthetic oligonucleotide
  source 1..45: mol_type = other DNA
ccccttagggataacagggtaatcccctgtctgccacctgttgtc

SEQ ID NO: 649 - DNA, 46 bp, synthetic construct
  misc_feature 1..46: note = Synthetic oligonucleotide
  source 1..46: mol_type = other DNA
ccccttagggataacagggtaatcggcctcttctcaatcccagtgc

SEQ ID NO: 650 - DNA, 49 bp, synthetic construct
  misc_feature 1..49: note = Synthetic oligonucleotide
  source 1..49: mol_type = other DNA
ccccttagggataacagggtaatccatccctgacagcaatgactcactc

SEQ ID NO: 651 - DNA, 48 bp, synthetic construct
  misc_feature 1..48: note = Synthetic oligonucleotide
  source 1..48: mol_type = other DNA
ccccttagggataacagggtaatctgtgagagtctggcctttactggt

SEQ ID NO: 652 - DNA, 43 bp, synthetic construct
  misc_feature 1..43: note = Synthetic oligonucleotide
  source 1..43: mol_type = other DNA
ccccttagggataacagggtaatcttggacctcccctgcgtga

SEQ ID NO: 653 - DNA, 50 bp, synthetic construct
  misc_feature 1..50: note = Synthetic oligonucleotide
  source 1..50: mol_type = other DNA
ccccttagggataacagggtaatcactatgtggactgtgggactctatga

SEQ ID NO: 654 - DNA, 48 bp, synthetic construct
  misc_feature 1..48: note = Synthetic oligonucleotide
  source 1..48: mol_type = other DNA
ccccttagggataacagggtaatctttcaaaggggaatgtactaccgt

SEQ ID NO: 655 - DNA, 42 bp, synthetic construct
  misc_feature 1..42: note = Synthetic oligonucleotide
  source 1..42: mol_type = other DNA
ccccttagggataacagggtaatcggcctgcaaccccgctac

SEQ ID NO: 656 - DNA, 45 bp, synthetic construct
  misc_feature 1..45: note = Synthetic oligonucleotide
  source 1..45: mol_type = other DNA
ccccttagggataacagggtaatcgctaggccctggagatgctac

SEQ ID NO: 657 - DNA, 50 bp, synthetic construct
  misc_feature 1..50: note = Synthetic oligonucleotide
  source 1..50: mol_type = other DNA
ccccttagggataacagggtaatcagtccagcgtttgaatcagatcatgg

SEQ ID NO: 658 - DNA, 44 bp, synthetic construct
  misc_feature 1..44: note = Synthetic oligonucleotide
  source 1..44: mol_type = other DNA
ccccttagggataacagggtaatccgtgggcactgagagcacca

SEQ ID NO: 659 - DNA, 50 bp, synthetic construct
  misc_feature 1..50: note = Synthetic oligonucleotide
  source 1..50: mol_type = other DNA
ccccttagggataacagggtaatcggattgcagggtatccacgtctaaat

SEQ ID NO: 660 - DNA, 44 bp, synthetic construct
  misc_feature 1..44: note = Synthetic oligonucleotide
  source 1..44: mol_type = other DNA
ccccttagggataacagggtaatcgtgtgtctcacgtggtgggt

SEQ ID NO: 661 - DNA, 49 bp, synthetic construct
  misc_feature 1..49: note = Synthetic oligonucleotide
  source 1..49: mol_type = other DNA
ccccttagggataacagggtaatcggctggaataccctttgtagttggg

SEQ ID NO: 662 - DNA, 42 bp, synthetic construct
  misc_feature 1..42: note = Synthetic oligonucleotide
  source 1..42: mol_type = other DNA
ccccttagggataacagggtaatcagcaaggcgtggctggtg

SEQ ID NO: 663 - DNA, 50 bp, synthetic construct
  misc_feature 1..50: note = Synthetic oligonucleotide
  source 1..50: mol_type = other DNA
ccccttagggataacagggtaatctccagtgccctatcagagtaattcct

SEQ ID NO: 664 - DNA, 50 bp, synthetic construct
  misc_feature 1..50: note = Synthetic oligonucleotide
  source 1..50: mol_type = other DNA
ccccttagggataacagggtaatcgaggatgtaagtagcgcttgtgaaca

SEQ ID NO: 665 - DNA, 46 bp, synthetic construct
  misc_feature 1..46: note = Synthetic oligonucleotide
  source 1..46: mol_type = other DNA
ccccttagggataacagggtaatctggagacagcgtaagtgtccct

SEQ ID NO: 666 - DNA, 49 bp, synthetic construct
  misc_feature 1..49: note = Synthetic oligonucleotide
  source 1..49: mol_type = other DNA
ccccttagggataacagggtaatcgcaataaacactgcctagagcctat

SEQ ID NO: 667 - DNA, 47 bp, synthetic construct
  misc_feature 1..47: note = Synthetic oligonucleotide
  source 1..47: mol_type = other DNA
ccccttagggataacagggtaatcggccttaaaaattgctgcgcagt

SEQ ID NO: 668 - DNA, 45 bp, synthetic construct
  misc_feature 1..45: note = Synthetic oligonucleotide
  source 1..45: mol_type = other DNA
ccccttagggataacagggtaatctgctcaagacaggccaaggac

SEQ ID NO: 669 - DNA, 47 bp, synthetic construct
  misc_feature 1..47: note = Synthetic oligonucleotide
  source 1..47: mol_type = other DNA
ccccttagggataacagggtaatctcttttctactgggcctccacct

SEQ ID NO: 670 - DNA, 50 bp, synthetic construct
  misc_feature 1..50: note = Synthetic oligonucleotide
  source 1..50: mol_type = other DNA
ccccttagggataacagggtaatcgcttccttagcctgaggtcactaaaa

SEQ ID NO: 671 - DNA, 50 bp, synthetic construct
  misc_feature 1..50: note = Synthetic oligonucleotide
  source 1..50: mol_type = other DNA
ccccttagggataacagggtaatcaatccaacctaataagcacaggcact

SEQ ID NO: 672 - DNA, 47 bp, synthetic construct
  misc_feature 1..47: note = Synthetic oligonucleotide
  source 1..47: mol_type = other DNA
ccccttagggataacagggtaatctcctaggcttctttcctctccca

SEQ ID NO: 673 - DNA, 44 bp, synthetic construct
  misc_feature 1..44: note = Synthetic oligonucleotide
  source 1..44: mol_type = other DNA
ccccttagggataacagggtaatcggggccactgagactcctct

SEQ ID NO: 674 - DNA, 49 bp, synthetic construct
  misc_feature 1..49: note = Synthetic oligonucleotide
  source 1..49: mol_type = other DNA
ccccttagggataacagggtaatcacctttggaacgatgggggtatttt

SEQ ID NO: 675 - DNA, 45 bp, synthetic construct
  misc_feature 1..45: note = Synthetic oligonucleotide
  source 1..45: mol_type = other DNA
ccccttagggataacagggtaatcctggagcatcgacgagggtga

SEQ ID NO: 676 - DNA, 44 bp, synthetic construct
  misc_feature 1..44: note = Synthetic oligonucleotide
  source 1..44: mol_type = other DNA
ccccttagggataacagggtaatcggagcatcgacgagggtgag

SEQ ID NO: 677 - DNA, 49 bp, synthetic construct
  misc_feature 1..49: note = Synthetic oligonucleotide
  source 1..49: mol_type = other DNA
ccccttagggataacagggtaatcgcctgcattcattcgtccacaatac

SEQ ID NO: 678 - DNA, 50 bp, synthetic construct
  misc_feature 1..50: note = Synthetic oligonucleotide
  source 1..50: mol_type = other DNA
ccccttagggataacagggtaatcagatgctgagagtttaccccctctac

SEQ ID NO: 679 - DNA, 48 bp, synthetic construct
  misc_feature 1..48: note = Synthetic oligonucleotide
  source 1..48: mol_type = other DNA
ccccttagggataacagggtaatctttttctccccaaacgtgagaaga

SEQ ID NO: 680 - DNA, 45 bp, synthetic construct
  misc_feature 1..45: note = Synthetic oligonucleotide
  source 1..45: mol_type = other DNA
ccccttagggataacagggtaatcactgttggggtgactaactgt

SEQ ID NO: 681 - DNA, 49 bp, synthetic construct
  misc_feature 1..49: note = Synthetic oligonucleotide
  source 1..49: mol_type = other DNA
ccccttagggataacagggtaatcttgctaacagtggtgagttgtaata

SEQ ID NO: 682 - DNA, 45 bp, synthetic construct
  misc_feature 1..45: note = Synthetic oligonucleotide
  source 1..45: mol_type = other DNA
ccccttagggataacagggtaatcagttcctgatccggctctgga

SEQ ID NO: 683 - DNA, 47 bp, synthetic construct
  misc_feature 1..47: note = Synthetic oligonucleotide
  source 1..47: mol_type = other DNA
ccccttagggataacagggtaatccgagaggctccaggaccatgact

SEQ ID NO: 684 - DNA, 42 bp, synthetic construct
  misc_feature 1..42: note = Synthetic oligonucleotide
  source 1..42: mol_type = other DNA
ccccttagggataacagggtaatcgggctggcggggtgggaa

SEQ ID NO: 685 - DNA, 49 bp, synthetic construct
  misc_feature 1..49: note = Synthetic oligonucleotide
  source 1..49: mol_type = other DNA
ccccttagggataacagggtaatcgtgctggctgaattaataggaggca

SEQ ID NO: 686 - DNA, 49 bp, synthetic construct
  misc_feature 1..49: note = Synthetic oligonucleotide
  source 1..49: mol_type = other DNA
ccccttagggataacagggtaatccaaggtctttcaacttgggccagat

SEQ ID NO: 687 - DNA, 44 bp, synthetic construct
  misc_feature 1..44: note = Synthetic oligonucleotide
  source 1..44: mol_type = other DNA
ccccttagggataacagggtaatcccttgggtcctgtcctggca

SEQ ID NO: 688 - DNA, 43 bp, synthetic construct
  misc_feature 1..43: note = Synthetic oligonucleotide
  source 1..43: mol_type = other DNA
ccccttagggataacagggtaatccggggttcactggcccaga

SEQ ID NO: 689 - DNA, 44 bp, synthetic construct
  misc_feature 1..44: note = Synthetic oligonucleotide
  source 1..44: mol_type = other DNA
ccccttagggataacagggtaatcaagggagcggggattatggc

SEQ ID NO: 690 - DNA, 45 bp, synthetic construct
  misc_feature 1..45: note = Synthetic oligonucleotide
  source 1..45: mol_type = other DNA
ccccttagggataacagggtaatcgatcatgcaccccgtcctgac

SEQ ID NO: 691 - DNA, 43 bp, synthetic construct
  misc_feature 1..43: note = Synthetic oligonucleotide
  source 1..43: mol_type = other DNA
ccccttagggataacagggtaatccagacctgccgtggacctt

SEQ ID NO: 692 - DNA, 42 bp, synthetic construct
  misc_feature 1..42: note = Synthetic oligonucleotide
  source 1..42: mol_type = other DNA
ccccttagggataacagggtaatcagccggcgctaagagcag

SEQ ID NO: 693 - DNA, 43 bp, synthetic construct
  misc_feature 1..43: note = Synthetic oligonucleotide
  source 1..43: mol_type = other DNA
ccccttagggataacagggtaatcgcctggatcccacccttgc

SEQ ID NO: 694 - DNA, 42 bp, synthetic construct
  misc_feature 1..42: note = Synthetic oligonucleotide
  source 1..42: mol_type = other DNA
ccccttagggataacagggtaatcctggtcccgccgcagcct

SEQ ID NO: 695 - DNA, 48 bp, synthetic construct
  misc_feature 1..48: note = Synthetic oligonucleotide
  source 1..48: mol_type = other DNA
ccccttagggataacagggtaatcgccctggctatttgcaaactgcat

SEQ ID NO: 696 - DNA, 50 bp, synthetic construct
  misc_feature 1..50: note = Synthetic oligonucleotide
  source 1..50: mol_type = other DNA
ccccttagggataacagggtaatcacagagatgcagatagccaggttaga

SEQ ID NO: 697 - DNA, 43 bp, synthetic construct
  misc_feature 1..43: note = Synthetic oligonucleotide
  source 1..43: mol_type = other DNA
ccccttagggataacagggtaatcgggtggaaggtccctccag

SEQ ID NO: 698 - DNA, 43 bp, synthetic construct
  misc_feature 1..43: note = Synthetic oligonucleotide
  source 1..43: mol_type = other DNA
ccccttagggataacagggtaatcagtggagaaggcggcactc

SEQ ID NO: 699 - DNA, 58 bp, synthetic construct
  misc_feature 1..58: note = Synthetic oligonucleotide
  source 1..58: mol_type = other DNA
gtgactggagttcagacgtgtgctcttccgatctcagtcaccacactgggtaactcct

SEQ ID NO: 700 - DNA, 57 bp, synthetic construct
  misc_feature 1..57: note = Synthetic oligonucleotide
  source 1..57: mol_type = other DNA
gtgactggagttcagacgtgtgctcttccgatctgtgaaacgctggggtgcaatttc

SEQ ID NO: 701 - DNA, 56 bp, synthetic construct
  misc_feature 1..56: note = Synthetic oligonucleotide
  source 1..56: mol_type = other DNA
gtgactggagttcagacgtgtgctcttccgatcttcctcttcccccaagctggctt

SEQ ID NO: 702 - DNA, 52 bp, synthetic construct
  misc_feature 1..52: note = Synthetic oligonucleotide
  source 1..52: mol_type = other DNA
gtgactggagttcagacgtgtgctcttccgatctggcagagaggcaccccaa

SEQ ID NO: 703 - DNA, 56 bp, synthetic construct
  misc_feature 1..56: note = Synthetic oligonucleotide
  source 1..56: mol_type = other DNA
gtgactggagttcagacgtgtgctcttccgatctcgtctggtccagtcagccttgc

SEQ ID NO: 704 - DNA, 55 bp, synthetic construct
  misc_feature 1..55: note = Synthetic oligonucleotide
  source 1..55: mol_type = other DNA
gtgactggagttcagacgtgtgctcttccgatctcgggtgtgtgggtgacaagcg

SEQ ID NO: 705 - DNA, 60 bp, synthetic construct
  misc_feature 1..60: note = Synthetic oligonucleotide
  source 1..60: mol_type = other DNA
gtgactggagttcagacgtgtgctcttccgatctggggagttaatgtaagggaggcaaca

SEQ ID NO: 706 - DNA, 53 bp, synthetic construct
  misc_feature 1..53: note = Synthetic oligonucleotide
  source 1..53: mol_type = other DNA
gtgactggagttcagacgtgtgctcttccgatctgagcccaccactcagcttt

SEQ ID NO: 707 - DNA, 57 bp, synthetic construct
  misc_feature 1..57: note = Synthetic oligonucleotide
  source 1..57: mol_type = other DNA
gtgactggagttcagacgtgtgctcttccgatcttgggcttggagttaaggggccta

SEQ ID NO: 708 - DNA, 60 bp, synthetic construct
  misc_feature 1..60: note = Synthetic oligonucleotide
  source 1..60: mol_type = other DNA
gtgactggagttcagacgtgtgctcttccgatctccaactgccaaacacgtgaatgaggt

SEQ ID NO: 709 - DNA, 57 bp, synthetic construct
  misc_feature 1..57: note = Synthetic oligonucleotide
  source 1..57: mol_type = other DNA
gtgactggagttcagacgtgtgctcttccgatctatgaggtcatctgaggccatccc

SEQ ID NO: 710 - DNA, 53 bp, synthetic construct
  misc_feature 1..53: note = Synthetic oligonucleotide
  source 1..53: mol_type = other DNA
gtgactggagttcagacgtgtgctcttccgatctagccagagcatgccaagca

SEQ ID NO: 711 - DNA, 59 bp, synthetic construct
  misc_feature 1..59: note = Synthetic oligonucleotide
  source 1..59: mol_type = other DNA
gtgactggagttcagacgtgtgctcttccgatctcgagctagagtagaaggtgccccat

SEQ ID NO: 712 - DNA, 59 bp, synthetic construct
  misc_feature 1..59: note = Synthetic oligonucleotide
  source 1..59: mol_type = other DNA
gtgactggagttcagacgtgtgctcttccgatctcctctgatgtgtcgatgccagcctt

SEQ ID NO: 713 - DNA, 59 bp, synthetic construct
  misc_feature 1..59: note = Synthetic oligonucleotide
  source 1..59: mol_type = other DNA
gtgactggagttcagacgtgtgctcttccgatctagtcggtgtcctgatggtagaaaac

SEQ ID NO: 714 - DNA, 58 bp, synthetic construct
  misc_feature 1..58: note = Synthetic oligonucleotide
  source 1..58: mol_type = other DNA
gtgactggagttcagacgtgtgctcttccgatctagccagtgatgaaaggtgcctcaa

SEQ ID NO: 715 - DNA, 54 bp, synthetic construct
  misc_feature 1..54: note = Synthetic oligonucleotide
  source 1..54: mol_type = other DNA
gtgactggagttcagacgtgtgctcttccgatctagcacaggagagggcctctg

SEQ ID NO: 716 - DNA, 58 bp, synthetic construct
  misc_feature 1..58: note = Synthetic oligonucleotide
  source 1..58: mol_type = other DNA
gtgactggagttcagacgtgtgctcttccgatcttcccccagctcccaaatcaatcaa

SEQ ID NO: 717 - DNA, 58 bp, synthetic construct
  misc_feature 1..58: note = Synthetic oligonucleotide
  source 1..58: mol_type = other DNA
gtgactggagttcagacgtgtgctcttccgatctggcagcctttccttttcacagatg

SEQ ID NO: 718 - DNA, 54 bp, synthetic construct
  misc_feature 1..54: note = Synthetic oligonucleotide
  source 1..54: mol_type = other DNA
gtgactggagttcagacgtgtgctcttccgatctccgacgctccacagcctgtc

SEQ ID NO: 719 - DNA, 52 bp, synthetic construct
  misc_feature 1..52: note = Synthetic oligonucleotide
  source 1..52: mol_type = other DNA
gtgactggagttcagacgtgtgctcttccgatctcggcccaggctcggactg

SEQ ID NO: 720 - DNA, 60 bp, synthetic construct
  misc_feature 1..60: note = Synthetic oligonucleotide
  source 1..60: mol_type = other DNA
gtgactggagttcagacgtgtgctcttccgatctaggctctagcagtcccagtaataagt

SEQ ID NO: 721 - DNA, 60 bp, synthetic construct
  misc_feature 1..60: note = Synthetic oligonucleotide
  source 1..60: mol_type = other DNA
gtgactggagttcagacgtgtgctcttccgatctatgctacacatacttcaccttaaggg

SEQ ID NO: 722 - DNA, 56 bp, synthetic construct
  misc_feature 1..56: note = Synthetic oligonucleotide
  source 1..56: mol_type = other DNA
gtgactggagttcagacgtgtgctcttccgatctcaactgtggagactgactggct

SEQ ID NO: 723 - DNA, 57 bp, synthetic construct
  misc_feature 1..57: note = Synthetic oligonucleotide
  source 1..57: mol_type = other DNA
gtgactggagttcagacgtgtgctcttccgatctgggagccatacctgagaaggaga

SEQ ID NO: 724 - DNA, 58 bp, synthetic construct
  misc_feature 1..58: note = Synthetic oligonucleotide
  source 1..58: mol_type = other DNA
gtgactggagttcagacgtgtgctcttccgatcttgagcttgaggatctgtcaggcaa

SEQ ID NO: 725 - DNA, 60 bp, synthetic construct
  misc_feature 1..60: note = Synthetic oligonucleotide
  source 1..60: mol_type = other DNA
gtgactggagttcagacgtgtgctcttccgatctatgatctggctgatggaccaaacatc

SEQ ID NO: 726 - DNA, 54 bp, synthetic construct
  misc_feature 1..54: note = Synthetic oligonucleotide
  source 1..54: mol_type = other DNA
gtgactggagttcagacgtgtgctcttccgatctacagcggctgttgctcttcc

SEQ ID NO: 727 - DNA, 60 bp, synthetic construct
  misc_feature 1..60: note = Synthetic oligonucleotide
  source 1..60: mol_type = other DNA
gtgactggagttcagacgtgtgctcttccgatctccgcttgtacaacggtctttcctcaa

SEQ ID NO: 728 - DNA, 60 bp, synthetic construct
  misc_feature 1..60: note = Synthetic oligonucleotide
  source 1..60: mol_type = other DNA
gtgactggagttcagacgtgtgctcttccgatctatttgcagcttcctctacacttcctg

SEQ ID NO: 729 - DNA, 58 bp, synthetic construct
  misc_feature 1..58: note = Synthetic oligonucleotide
  source 1..58: mol_type = other DNA
gtgactggagttcagacgtgtgctcttccgatctcacaccacacacgtcacagaaacc

SEQ ID NO: 730 - DNA, 53 bp, synthetic construct
  misc_feature 1..53: note = Synthetic oligonucleotide
  source 1..53: mol_type = other DNA
gtgactggagttcagacgtgtgctcttccgatctagaaggggtgggaggccaa

SEQ ID NO: 731 - DNA, 55 bp, synthetic construct
  misc_feature 1..55: note = Synthetic oligonucleotide
  source 1..55: mol_type = other DNA
gtgactggagttcagacgtgtgctcttccgatcttcacctgtccactcacagcac

SEQ ID NO: 732 - DNA, 56 bp, synthetic construct
  misc_feature 1..56: note = Synthetic oligonucleotide
  source 1..56: mol_type = other DNA
gtgactggagttcagacgtgtgctcttccgatctgcgctcgagttacagggccact

SEQ ID NO: 733 - DNA, 60 bp, synthetic construct
  misc_feature 1..60: note = Synthetic oligonucleotide
  source 1..60: mol_type = other DNA
gtgactggagttcagacgtgtgctcttccgatctaggtgatgtgatcttcctgcttgctc

SEQ ID NO: 734 - DNA, 59 bp, synthetic construct
  misc_feature 1..59: note = Synthetic oligonucleotide
  source 1..59: mol_type = other DNA
gtgactggagttcagacgtgtgctcttccgatctagcattacacaacccctagaaagtc

SEQ ID NO: 735 - DNA, 60 bp, synthetic construct
  misc_feature 1..60: note = Synthetic oligonucleotide
  source 1..60: mol_type = other DNA
gtgactggagttcagacgtgtgctcttccgatctcactgtgtcatattgcctgcatgtct

SEQ ID NO: 736 - DNA, 53 bp, synthetic construct
  misc_feature 1..53: note = Synthetic oligonucleotide
  source 1..53: mol_type = other DNA
gtgactggagttcagacgtgtgctcttccgatctcccccagccctagtccaca

SEQ ID NO: 737 - DNA, 59 bp, synthetic construct
  misc_feature 1..59: note = Synthetic oligonucleotide
  source 1..59: mol_type = other DNA
gtgactggagttcagacgtgtgctcttccgatctgtgagggcaactgacaaaagcaatt

SEQ ID NO: 738 - DNA, 60 bp, synthetic construct
  misc_feature 1..60: note = Synthetic oligonucleotide
  source 1..60: mol_type = other DNA
gtgactggagttcagacgtgtgctcttccgatctggccaggtcctacattgagcaatcat

SEQ ID NO: 739 - DNA, 55 bp, synthetic construct
  misc_feature 1..55: note = Synthetic oligonucleotide
  source 1..55: mol_type = other DNA
gtgactggagttcagacgtgtgctcttccgatctggcaatggtgtccactttgga

SEQ ID NO: 740 - DNA, 60 bp, synthetic construct
  misc_feature 1..60: note = Synthetic oligonucleotide
  source 1..60: mol_type = other DNA
gtgactggagttcagacgtgtgctcttccgatctcctgtctgccacctgttgtcattaac

SEQ ID NO: 741 - DNA, 60 bp, synthetic construct
  misc_feature 1..60: note = Synthetic oligonucleotide
  source 1..60: mol_type = other DNA
gtgactggagttcagacgtgtgctcttccgatctcctcttctcaatcccagtgcctactc

SEQ ID NO: 742 - DNA, 60 bp, synthetic construct
  misc_feature 1..60: note = Synthetic oligonucleotide
  source 1..60: mol_type = other DNA
gtgactggagttcagacgtgtgctcttccgatctctgacagcaatgactcactccccttg

SEQ ID NO: 743 - DNA, 60 bp, synthetic construct
  misc_feature 1..60: note = Synthetic oligonucleotide
  source 1..60: mol_type = other DNA
gtgactggagttcagacgtgtgctcttccgatcttgaatcaggaggggctatgtagttct

SEQ ID NO: 744 - DNA, 59 bp, synthetic construct
  misc_feature 1..59: note = Synthetic oligonucleotide
  source 1..59: mol_type = other DNA
gtgactggagttcagacgtgtgctcttccgatctacctcccctgcgtgaaactgttcta

SEQ ID NO: 745 - DNA, 60 bp, synthetic construct
  misc_feature 1..60: note = Synthetic oligonucleotide
  source 1..60: mol_type = other DNA
gtgactggagttcagacgtgtgctcttccgatcttggactgtgggactctatgaatgtgg

SEQ ID NO: 746 - DNA, 58 bp, synthetic construct
  misc_feature 1..58: note = Synthetic oligonucleotide
  source 1..58: mol_type = other DNA
gtgactggagttcagacgtgtgctcttccgatctaggggaatgtactaccgtcacttt

SEQ ID NO: 747 - DNA, 56 bp, synthetic construct
  misc_feature 1..56: note = Synthetic oligonucleotide
  source 1..56: mol_type = other DNA
gtgactggagttcagacgtgtgctcttccgatcttgcaaccccgctacttcctcct

SEQ ID NO: 748 - DNA, 57 bp, synthetic construct
  misc_feature 1..57: note = Synthetic oligonucleotide
  source 1..57: mol_type = other DNA
gtgactggagttcagacgtgtgctcttccgatctcagggatcaggccaggtaaaaca

SEQ ID NO: 749 - DNA, 60 bp, synthetic construct
  misc_feature 1..60: note = Synthetic oligonucleotide
  source 1..60: mol_type = other DNA
gtgactggagttcagacgtgtgctcttccgatctcatggaagatggctctagaggaagct

SEQ ID NO: 750 - DNA, 58 bp, synthetic construct
  misc_feature 1..58: note = Synthetic oligonucleotide
  source 1..58: mol_type = other DNA
gtgactggagttcagacgtgtgctcttccgatcttgggcactgagagcaccatcatgg

SEQ ID NO: 751 - DNA, 58 bp, synthetic construct
  misc_feature 1..58: note = Synthetic oligonucleotide
  source 1..58: mol_type = other DNA
gtgactggagttcagacgtgtgctcttccgatctatgcatgaaggccagcacaatggg

SEQ ID NO: 752 - DNA, 60 bp, synthetic construct
  misc_feature 1..60: note = Synthetic oligonucleotide
  source 1..60: mol_type = other DNA
gtgactggagttcagacgtgtgctcttccgatctcacgtggtgggtgatttttattccag

SEQ ID NO: 753 - DNA, 53 bp, synthetic construct
  misc_feature 1..53: note = Synthetic oligonucleotide
  source 1..53: mol_type = other DNA
gtgactggagttcagacgtgtgctcttccgatctgggggctgcctgtgtgtta

SEQ ID NO: 754 - DNA, 56 bp, synthetic construct
  misc_feature 1..56: note = Synthetic oligonucleotide
  source 1..56: mol_type = other DNA
gtgactggagttcagacgtgtgctcttccgatctcatgggcaagagcatgctggta

SEQ ID NO: 755 - DNA, 58 bp, synthetic construct
  misc_feature 1..58: note = Synthetic oligonucleotide
  source 1..58: mol_type = other DNA
gtgactggagttcagacgtgtgctcttccgatctcatagcttctttgctggccgacca

SEQ ID NO: 756 - DNA, 55 bp, synthetic construct
  misc_feature 1..55: note = Synthetic oligonucleotide
  source 1..55: mol_type = other DNA
gtgactggagttcagacgtgtgctcttccgatctcacagccccaggtcctttgcg

SEQ ID NO: 757 - DNA, 54 bp, synthetic construct
  misc_feature 1..54: note = Synthetic oligonucleotide
  source 1..54: mol_type = other DNA
gtgactggagttcagacgtgtgctcttccgatctagtgtccctgtcctcacgct

SEQ ID NO: 758 - DNA, 60 bp, synthetic construct
  misc_feature 1..60: note = Synthetic oligonucleotide
  source 1..60: mol_type = other DNA
gtgactggagttcagacgtgtgctcttccgatctcactgcctagagcctatattgcaaag

SEQ ID NO: 759 - DNA, 60 bp, synthetic construct
  misc_feature 1..60: note = Synthetic oligonucleotide
  source 1..60: mol_type = other DNA
gtgactggagttcagacgtgtgctcttccgatctcttaaaaattgctgcgcagtggctgt

SEQ ID NO: 760 - DNA, 60 bp, synthetic construct
  misc_feature 1..60: note = Synthetic oligonucleotide
  source 1..60: mol_type = other DNA
gtgactggagttcagacgtgtgctcttccgatctgctcaagacaggccaaggacttagaa

SEQ ID NO: 761 - DNA, 54 bp, synthetic construct
  misc_feature 1..54: note = Synthetic oligonucleotide
  source 1..54: mol_type = other DNA
gtgactggagttcagacgtgtgctcttccgatctgctgctcccttcccctccac

SEQ ID NO: 762 - DNA, 60 bp, synthetic construct
  misc_feature 1..60: note = Synthetic oligonucleotide
  source 1..60: mol_type = other DNA
gtgactggagttcagacgtgtgctcttccgatctgaggtcactaaaaatggccagtctgc

SEQ ID NO: 763 - DNA, 57 bp, synthetic construct
  misc_feature 1..57: note = Synthetic oligonucleotide
  source 1..57: mol_type = other DNA
gtgactggagttcagacgtgtgctcttccgatctactgagtgctggcatcaggattc

SEQ ID NO: 764 - DNA, 60 bp, synthetic construct
  misc_feature 1..60: note = Synthetic oligonucleotide
  source 1..60: mol_type = other DNA
gtgactggagttcagacgtgtgctcttccgatctccagtagcctgtagtcagaaagagtg

SEQ ID NO: 765 - DNA, 60 bp, synthetic construct
  misc_feature 1..60: note = Synthetic oligonucleotide
  source 1..60: mol_type = other DNA
gtgactggagttcagacgtgtgctcttccgatctctcctcttaggacaaccgaccatcct

SEQ ID NO: 766 - DNA, 60 bp, synthetic construct
  misc_feature 1..60: note = Synthetic oligonucleotide
  source 1..60: mol_type = other DNA
gtgactggagttcagacgtgtgctcttccgatctacctcttgtttctcaaaacgctgtcg

SEQ ID NO: 767 - DNA, 56 bp, synthetic construct
  misc_feature 1..56: note = Synthetic oligonucleotide
  source 1..56: mol_type = other DNA
gtgactggagttcagacgtgtgctcttccgatctcatcgacgagggtgagcgcatg

SEQ ID NO: 768 - DNA, 54 bp, synthetic construct
  misc_feature 1..54: note = Synthetic oligonucleotide
  source 1..54: mol_type = other DNA
gtgactggagttcagacgtgtgctcttccgatcttcgacgagggtgagcgcatg

SEQ ID NO: 769 - DNA, 53 bp, synthetic construct
  misc_feature 1..53: note = Synthetic oligonucleotide
  source 1..53: mol_type = other DNA
gtgactggagttcagacgtgtgctcttccgatctgccctgggcttggcatgaa

SEQ ID NO: 770 - DNA, 54 bp, synthetic construct
  misc_feature 1..54: note = Synthetic oligonucleotide
  source 1..54: mol_type = other DNA
gtgactggagttcagacgtgtgctcttccgatctccccctctacctcccacctt

SEQ ID NO: 771 - DNA, 59 bp, synthetic construct
  misc_feature 1..59: note = Synthetic oligonucleotide
  source 1..59: mol_type = other DNA
gtgactggagttcagacgtgtgctcttccgatcttccccaaacgtgagaagaaaagaga

SEQ ID NO: 772 - DNA, 59 bp, synthetic construct
  misc_feature 1..59: note = Synthetic oligonucleotide
  source 1..59: mol_type = other DNA
gtgactggagttcagacgtgtgctcttccgatctgactaactgtcatggttttcccacg

SEQ ID NO: 773 - DNA, 60 bp, synthetic construct
  misc_feature 1..60: note = Synthetic oligonucleotide
  source 1..60: mol_type = other DNA
gtgactggagttcagacgtgtgctcttccgatctaacagtggtgagttgtaatactagct

SEQ ID NO: 774 - DNA, 57 bp, synthetic construct
  misc_feature 1..57: note = Synthetic oligonucleotide
  source 1..57: mol_type = other DNA
gtgactggagttcagacgtgtgctcttccgatcttccggctctggatttgtgcacag

SEQ ID NO: 775 - DNA, 52 bp, synthetic construct
  misc_feature 1..52: note = Synthetic oligonucleotide
  source 1..52: mol_type = other DNA
gtgactggagttcagacgtgtgctcttccgatctgcgctgcacggcctccac

SEQ ID NO: 776 - DNA, 54 bp, synthetic construct
  misc_feature 1..54: note = Synthetic oligonucleotide
  source 1..54: mol_type = other DNA
gtgactggagttcagacgtgtgctcttccgatctgggtgggaagggagggtcag

SEQ ID NO: 777 - DNA, 60 bp, synthetic construct
  misc_feature 1..60: note = Synthetic oligonucleotide
  source 1..60: mol_type = other DNA
gtgactggagttcagacgtgtgctcttccgatctaataggaggcacatctcatccattgc

SEQ ID NO: 778 - DNA, 56 bp, synthetic construct
  misc_feature 1..56: note = Synthetic oligonucleotide
  source 1..56: mol_type = other DNA
gtgactggagttcagacgtgtgctcttccgatctgagcactgcaggacgttcagca

SEQ ID NO: 779 - DNA, 55 bp, synthetic construct
  misc_feature 1..55: note = Synthetic oligonucleotide
  source 1..55: mol_type = other DNA
gtgactggagttcagacgtgtgctcttccgatcttgctatgagctgcccctgggt

SEQ ID NO: 780 - DNA, 55 bp, synthetic construct
  misc_feature 1..55: note = Synthetic oligonucleotide
  source 1..55: mol_type = other DNA
gtgactggagttcagacgtgtgctcttccgatctttcactggcccagagctgtgc

SEQ ID NO: 781 - DNA, 59 bp, synthetic construct
  misc_feature 1..59: note = Synthetic oligonucleotide
  source 1..59: mol_type = other DNA
gtgactggagttcagacgtgtgctcttccgatctaggaccagggtcatgactagctaaa

SEQ ID NO: 782 - DNA, 55 bp, synthetic construct
  misc_feature 1..55: note = Synthetic oligonucleotide
  source 1..55: mol_type = other DNA
gtgactggagttcagacgtgtgctcttccgatctgtcctgaccctgacgctgcac

SEQ ID NO: 783 - DNA, 53 bp, synthetic construct
  misc_feature 1..53: note = Synthetic oligonucleotide
  source 1..53: mol_type = other DNA
gtgactggagttcagacgtgtgctcttccgatctgccgtggaccttggcttcc

SEQ ID NO: 784 - DNA, 54 bp, synthetic construct
  misc_feature 1..54: note = Synthetic oligonucleotide
  source 1..54: mol_type = other DNA
gtgactggagttcagacgtgtgctcttccgatctggcgctaagagcagctgacc

SEQ ID NO: 785 - DNA, 55 bp, synthetic construct
  misc_feature 1..55: note = Synthetic oligonucleotide
  source 1..55: mol_type = other DNA
gtgactggagttcagacgtgtgctcttccgatctgtgtggcacagtgaggggtgt

SEQ ID NO: 786 - DNA, 52 bp, synthetic construct
  misc_feature 1..52: note = Synthetic oligonucleotide
  source 1..52: mol_type = other DNA
gtgactggagttcagacgtgtgctcttccgatctccgccgcagcctcgcaga

SEQ ID NO: 787 - DNA, 59 bp, synthetic construct
  misc_feature 1..59: note = Synthetic oligonucleotide
  source 1..59: mol_type = other DNA
gtgactggagttcagacgtgtgctcttccgatctatgctgtcccagttctctcaccact

SEQ ID NO: 788 - DNA, 57 bp, synthetic construct
  misc_feature 1..57: note = Synthetic oligonucleotide
  source 1..57: mol_type = other DNA
gtgactggagttcagacgtgtgctcttccgatctggcagggataggtgagcttcaaa

SEQ ID NO: 789 - DNA, 53 bp, synthetic construct
  misc_feature 1..53: note = Synthetic oligonucleotide
  source 1..53: mol_type = other DNA
gtgactggagttcagacgtgtgctcttccgatctccctggctctgggacacct

SEQ ID NO: 790 - DNA, 54 bp, synthetic construct
  misc_feature 1..54: note = Synthetic oligonucleotide
  source 1..54: mol_type = other DNA
gtgactggagttcagacgtgtgctcttccgatctactctggtggggctgctcca

SEQ ID NO: 791 - DNA, 46 bp, synthetic construct
  misc_feature 1..46: note = Synthetic oligonucleotide
  source 1..46: mol_type = other DNA
ccccttagggataacagggtaatcaagtagggctcagggtcgaagg

SEQ ID NO: 792 - DNA, 45 bp, synthetic construct
  misc_feature 1..45: note = Synthetic oligonucleotide
  source 1..45: mol_type = other DNA
ccccttagggataacagggtaatcgcaatggccgctgggaaaaat

SEQ ID NO: 793 - DNA, 49 bp, synthetic construct
  misc_feature 1..49: note = Synthetic oligonucleotide
  source 1..49: mol_type = other DNA
ccccttagggataacagggtaatcctatcattgtagatggggccggaaa

SEQ ID NO: 794 - DNA, 43 bp, synthetic construct
  misc_feature 1..43: note = Synthetic oligonucleotide
  source 1..43: mol_type = other DNA
ccccttagggataacagggtaatcgccactgccactgtagcct

SEQ ID NO: 795 - DNA, 47 bp, synthetic construct
  misc_feature 1..47: note = Synthetic oligonucleotide
  source 1..47: mol_type = other DNA
ccccttagggataacagggtaatccaactccagggctcaagcaatcg

SEQ ID NO: 796 - DNA, 49 bp, synthetic construct
  misc_feature 1..49: note = Synthetic oligonucleotide
  source 1..49: mol_type = other DNA
ccccttagggataacagggtaatcgcagacatttgaccaccctataccc

SEQ ID NO: 797 - DNA, 44 bp, synthetic construct
  misc_feature 1..44: note = Synthetic oligonucleotide
  source 1..44: mol_type = other DNA
ccccttagggataacagggtaatcgcagtagggaaggggcaact

SEQ ID NO: 798 - DNA, 49 bp, synthetic construct
  misc_feature 1..49: note = Synthetic oligonucleotide
  source 1..49: mol_type = other DNA
ccccttagggataacagggtaatcgtctttctctggcaccaagcttttg

SEQ ID NO: 799 - DNA, 44 bp, synthetic construct
  misc_feature 1..44: note = Synthetic oligonucleotide
  source 1..44: mol_type = other DNA
ccccttagggataacagggtaatctggcacctgcaggaaacggt

SEQ ID NO: 800 - DNA, 42 bp, synthetic construct
  misc_feature 1..42: note = Synthetic oligonucleotide
  source 1..42: mol_type = other DNA
ccccttagggataacagggtaatcctgggccacctggtgtcg

SEQ ID NO: 801 - DNA, 43 bp, synthetic construct
  misc_feature 1..43: note = Synthetic oligonucleotide
  source 1..43: mol_type = other DNA
ccccttagggataacagggtaatcccttgggccagtcactgca

SEQ ID NO: 802 - DNA, 46 bp, synthetic construct
  misc_feature 1..46: note = Synthetic oligonucleotide
  source 1..46: mol_type = other DNA
ccccttagggataacagggtaatctgaccacatgtccaccgttcag

SEQ ID NO: 803 - DNA, 47 bp, synthetic construct
  misc_feature 1..47: note = Synthetic oligonucleotide
  source 1..47: mol_type = other DNA
ccccttagggataacagggtaatcagcttgggaggctggtactactg

SEQ ID NO: 804 - DNA, 46 bp, synthetic construct
  misc_feature 1..46: note = Synthetic oligonucleotide
  source 1..46: mol_type = other DNA
ccccttagggataacagggtaatccccagacactgcttccctggta

SEQ ID NO: 805 - DNA, 43 bp, synthetic construct
  misc_feature 1..43: note = Synthetic oligonucleotide
  source 1..43: mol_type = other DNA
ccccttagggataacagggtaatcttcctcctgccagggtgca

SEQ ID NO: 806 - DNA, 48 bp, synthetic construct
  misc_feature 1..48: note = Synthetic oligonucleotide
  source 1..48: mol_type = other DNA
ccccttagggataacagggtaatctgtaccatgaatgttgtggcgcat

SEQ ID NO: 807 - DNA, 45 bp, synthetic construct
  misc_feature 1..45: note = Synthetic oligonucleotide
  source 1..45: mol_type = other DNA
ccccttagggataacagggtaatcagtctgggtcaagtgctgtgg

SEQ ID NO: 808 - DNA, 48 bp, synthetic construct
  misc_feature 1..48: note = Synthetic oligonucleotide
  source 1..48: mol_type = other DNA
ccccttagggataacagggtaatcggaacaaaggacctacatgtggct

SEQ ID NO: 809 - DNA, 48 bp, synthetic construct
  misc_feature 1..48: note = Synthetic oligonucleotide
  source 1..48: mol_type = other DNA
ccccttagggataacagggtaatcacataagcgaaggatcaggagagt

SEQ ID NO: 810 - DNA, 47 bp, synthetic construct
  misc_feature 1..47: note = Synthetic oligonucleotide
  source 1..47: mol_type = other DNA
ccccttagggataacagggtaatctctagagaacatccggcaatgcc

SEQ ID NO: 811 - DNA, 50 bp, synthetic construct
  misc_feature 1..50: note = Synthetic oligonucleotide
  source 1..50: mol_type = other DNA
ccccttagggataacagggtaatcccttaggccaaacatccttgaccata

SEQ ID NO: 812 - DNA, 49 bp, synthetic construct
  misc_feature 1..49: note = Synthetic oligonucleotide
  source 1..49: mol_type = other DNA
ccccttagggataacagggtaatctccccaaagtctaaggaggctaaga

SEQ ID NO: 813 - DNA, 48 bp, synthetic construct
  misc_feature 1..48: note = Synthetic oligonucleotide
  source 1..48: mol_type = other DNA
ccccttagggataacagggtaatccctgaaaaacggatgagacttcag

SEQ ID NO: 814 - DNA, 48 bp, synthetic construct
  misc_feature 1..48: note = Synthetic oligonucleotide
  source 1..48: mol_type = other DNA
ccccttagggataacagggtaatcattgtgcttcagatcccgtgacat

SEQ ID NO: 815 - DNA, 43 bp, synthetic construct
  misc_feature 1..43: note = Synthetic oligonucleotide
  source 1..43: mol_type = other DNA
ccccttagggataacagggtaatcgtggggacttgctgctggt

SEQ ID NO: 816 - DNA, 48 bp, synthetic construct
  misc_feature 1..48: note = Synthetic oligonucleotide
  source 1..48: mol_type = other DNA
ccccttagggataacagggtaatcagctctgctacattcaggtaacat

SEQ ID NO: 817 - DNA, 48 bp, synthetic construct
  misc_feature 1..48: note = Synthetic oligonucleotide
  source 1..48: mol_type = other DNA
ccccttagggataacagggtaatctccctctttagcatcgccaaatcc

SEQ ID NO: 818 - DNA, 48 bp, synthetic construct
  misc_feature 1..48: note = Synthetic oligonucleotide
  source 1..48: mol_type = other DNA
ccccttagggataacagggtaatcttggtggccacaacttaggtgaga

SEQ ID NO: 819 - DNA, 50 bp, synthetic construct
  misc_feature 1..50: note = Synthetic oligonucleotide
  source 1..50: mol_type = other DNA
ccccttagggataacagggtaatccccaggtgttgctcatcagttcctct

SEQ ID NO: 820 - DNA, 49 bp, synthetic construct
  misc_feature 1..49: note = Synthetic oligonucleotide
  source 1..49: mol_type = other DNA
ccccttagggataacagggtaatcatcactttctcaagggacatgccat

SEQ ID NO: 821 - DNA, 50 bp, synthetic construct
  misc_feature 1..50: note = Synthetic oligonucleotide
  source 1..50: mol_type = other DNA
ccccttagggataacagggtaatcgtctcacaactcccagtcttgcttta

SEQ ID NO: 822 - DNA, 46 bp, synthetic construct
  misc_feature 1..46: note = Synthetic oligonucleotide
  source 1..46: mol_type = other DNA
ccccttagggataacagggtaatcaactgggctcgttggttaccct

SEQ ID NO: 823 - DNA, 50 bp, synthetic construct
  misc_feature 1..50: note = Synthetic oligonucleotide
  source 1..50: mol_type = other DNA
ccccttagggataacagggtaatctttggtttggttgctttgcagactac

SEQ ID NO: 824 - DNA, 49 bp, synthetic construct
  misc_feature 1..49: note = Synthetic oligonucleotide
  source 1..49: mol_type = other DNA
ccccttagggataacagggtaatccttttctgctgtcaccctcaaggat

SEQ ID NO: 825 - DNA, 50 bp, synthetic construct
  misc_feature 1..50: note = Synthetic oligonucleotide
  source 1..50: mol_type = other DNA
ccccttagggataacagggtaatcgagtgaatgcatgattgtgtgaccga

SEQ ID NO: 826 - DNA, 44 bp, synthetic construct
  misc_feature 1..44: note = Synthetic oligonucleotide
  source 1..44: mol_type = other DNA
ccccttagggataacagggtaatctgctagtgtcgaggtttgca

SEQ ID NO: 827 - DNA, 49 bp, synthetic construct
  misc_feature 1..49: note = Synthetic oligonucleotide
  source 1..49: mol_type = other DNA
ccccttagggataacagggtaatcgtggagaaagtgctaaacaagaaaa

SEQ ID NO: 828 - DNA, 48 bp, synthetic construct
  misc_feature 1..48: note = Synthetic oligonucleotide
  source 1..48: mol_type = other DNA
ccccttagggataacagggtaatcctccagagtctatgctcaactgaa

SEQ ID NO: 829 - DNA, 48 bp, synthetic construct
  misc_feature 1..48: note = Synthetic oligonucleotide
  source 1..48: mol_type = other DNA
ccccttagggataacagggtaatcatggccataagttgaaatttgcgt

SEQ ID NO: 830 - DNA, 43 bp, synthetic construct
  misc_feature 1..43: note = Synthetic oligonucleotide
  source 1..43: mol_type = other DNA
ccccttagggataacagggtaatcgggacctcaggtgctgctt

SEQ ID NO: 831 - DNA, 50 bp, synthetic construct
  misc_feature 1..50: note = Synthetic oligonucleotide
  source 1..50: mol_type = other DNA
ccccttagggataacagggtaatctgattcaatcttacatgcgacagcct

SEQ ID NO: 832 - DNA, 45 bp, synthetic construct
  misc_feature 1..45: note = Synthetic oligonucleotide
  source 1..45: mol_type = other DNA
ccccttagggataacagggtaatcagagaagcctgtcaggaccat

SEQ ID NO: 833 - DNA, 47 bp, synthetic construct
  misc_feature 1..47: note = Synthetic oligonucleotide
  source 1..47: mol_type = other DNA
ccccttagggataacagggtaatctcaccgtctacttctcttgtgtg

SEQ ID NO: 834 - DNA, 49 bp, synthetic construct
  misc_feature 1..49: note = Synthetic oligonucleotide
  source 1..49: mol_type = other DNA
ccccttagggataacagggtaatcccacatgcaaatgaacgacactgac

SEQ ID NO: 835 - DNA, 46 bp, synthetic construct
  misc_feature 1..46: note = Synthetic oligonucleotide
  source 1..46: mol_type = other DNA
ccccttagggataacagggtaatcgcagcaatttggtcccccatgg

SEQ ID NO: 836 - DNA, 47 bp, synthetic construct
  misc_feature 1..47: note = Synthetic oligonucleotide
  source 1..47: mol_type = other DNA
ccccttagggataacagggtaatctcagaccgtgactcagtatgttg

SEQ ID NO: 837 - DNA, 45 bp, synthetic construct
  misc_feature 1..45: note = Synthetic oligonucleotide
  source 1..45: mol_type = other DNA
ccccttagggataacagggtaatcaggcccctgtctctaccatcc

SEQ ID NO: 838 - DNA, 45 bp, synthetic construct
  misc_feature 1..45: note = Synthetic oligonucleotide
  source 1..45: mol_type = other DNA
ccccttagggataacagggtaatcgtggagaaggcagcctcccaa

SEQ ID NO: 839 - DNA, 44 bp, synthetic construct
  misc_feature 1..44: note = Synthetic oligonucleotide
  source 1..44: mol_type = other DNA
ccccttagggataacagggtaatctgcctggagtggtgtctggt

SEQ ID NO: 840 - DNA, 45 bp, synthetic construct
  misc_feature 1..45: note = Synthetic oligonucleotide
  source 1..45: mol_type = other DNA
ccccttagggataacagggtaatcacagacctcagagcccagtcc

SEQ ID NO: 841 - DNA, 48 bp, synthetic construct
  misc_feature 1..48: note = Synthetic oligonucleotide
  source 1..48: mol_type = other DNA
ccccttagggataacagggtaatcgcacacagccaacaagatgactca

SEQ ID NO: 842 - DNA, 50 bp, synthetic construct
  misc_feature 1..50: note = Synthetic oligonucleotide
  source 1..50: mol_type = other DNA
ccccttagggataacagggtaatccccctgtttttacctcaaccttaggg

SEQ ID NO: 843 - DNA, 43 bp, synthetic construct
  misc_feature 1..43: note = Synthetic oligonucleotide
  source 1..43: mol_type = other DNA
ccccttagggataacagggtaatctcactgctgcccccacaag

SEQ ID NO: 844 - DNA, 42 bp, synthetic construct
  misc_feature 1..42: note = Synthetic oligonucleotide
  source 1..42: mol_type = other DNA
ccccttagggataacagggtaatcggccaggccggagtcagg

SEQ ID NO: 845 - DNA, 49 bp, synthetic construct
  misc_feature 1..49: note = Synthetic oligonucleotide
  source 1..49: mol_type = other DNA
ccccttagggataacagggtaatcttggaatggcaatccgttggaaatg

SEQ ID NO: 846 - DNA, 50 bp, synthetic construct
  misc_feature 1..50: note = Synthetic oligonucleotide
  source 1..50: mol_type = other DNA
ccccttagggataacagggtaatctggaactgtgggcataagcatatgtc

SEQ ID NO: 847 - DNA, 45 bp, synthetic construct
  misc_feature 1..45: note = Synthetic oligonucleotide
  source 1..45: mol_type = other DNA
ccccttagggataacagggtaatcacaggtttcaggcggagtgga

SEQ ID NO: 848 - DNA, 50 bp, synthetic construct
  misc_feature 1..50: note = Synthetic oligonucleotide
  source 1..50: mol_type = other DNA
ccccttagggataacagggtaatcaggaggaattaaccctgtgaacatcg

SEQ ID NO: 849 - DNA, 49 bp, synthetic construct
  misc_feature 1..49: note = Synthetic oligonucleotide
  source 1..49: mol_type = other DNA
ccccttagggataacagggtaatctttcacaagaacggtactggccaat

SEQ ID NO: 850 - DNA, 50 bp, synthetic construct
  misc_feature 1..50: note = Synthetic oligonucleotide
  source 1..50: mol_type = other DNA
ccccttagggataacagggtaatcataagaggtgaactagcaagcagagc

SEQ ID NO: 851 - DNA, 49 bp, synthetic construct
  misc_feature 1..49: note = Synthetic oligonucleotide
  source 1..49: mol_type = other DNA
ccccttagggataacagggtaatcagagtgtaagctcaccctacagtct

SEQ ID NO: 852 - DNA, 47 bp, synthetic construct
  misc_feature 1..47: note = Synthetic oligonucleotide
  source 1..47: mol_type = other DNA
ccccttagggataacagggtaatcgacagcaagtccagactaaggca

SEQ ID NO: 853 - DNA, 47 bp, synthetic construct
  misc_feature 1..47: note = Synthetic oligonucleotide
  source 1..47: mol_type = other DNA
ccccttagggataacagggtaatcgaggtagggttcttcgtgttggc

SEQ ID NO: 854 - DNA, 50 bp, synthetic construct
  misc_feature 1..50: note = Synthetic oligonucleotide
  source 1..50: mol_type = other DNA
ccccttagggataacagggtaatccctaagtggagttgacctgtacaagg

SEQ ID NO: 855 - DNA, 49 bp, synthetic construct
  misc_feature 1..49: note = Synthetic oligonucleotide
  source 1..49: mol_type = other DNA
ccccttagggataacagggtaatccttcagccactcccttatgaggtag

SEQ ID NO: 856 - DNA, 47 bp, synthetic construct
  misc_feature 1..47: note = Synthetic oligonucleotide
  source 1..47: mol_type = other DNA
ccccttagggataacagggtaatcacaccaggctacaagtctcctga

SEQ ID NO: 857 - DNA, 47 bp, synthetic construct
  misc_feature 1..47: note = Synthetic oligonucleotide
  source 1..47: mol_type = other DNA
ccccttagggataacagggtaatcccctgctcctgtctgcctgatta

SEQ ID NO: 858 - DNA, 46 bp, synthetic construct
  misc_feature 1..46: note = Synthetic oligonucleotide
  source 1..46: mol_type = other DNA
ccccttagggataacagggtaatcaaggcttgttcaccctgaggag

SEQ ID NO: 859 - DNA, 50 bp, synthetic construct
  misc_feature 1..50: note = Synthetic oligonucleotide
  source 1..50: mol_type = other DNA
ccccttagggataacagggtaatcggaaagctaaaagatttgcgttgact

SEQ ID NO: 860 - DNA, 47 bp, synthetic construct
  misc_feature 1..47: note = Synthetic oligonucleotide
  source 1..47: mol_type = other DNA
ccccttagggataacagggtaatctccttccacggagttcactgagt

SEQ ID NO: 861 - DNA, 46 bp, synthetic construct
  misc_feature 1..46: note = Synthetic oligonucleotide
  source 1..46: mol_type = other DNA
ccccttagggataacagggtaatcagctttggcccctaggattctg

SEQ ID NO: 862 - DNA, 43 bp, synthetic construct
  misc_feature 1..43: note = Synthetic oligonucleotide
  source 1..43: mol_type = other DNA
ccccttagggataacagggtaatcctctgggtgcgggggaact

SEQ ID NO: 863 - DNA, 49 bp, synthetic construct
  misc_feature 1..49: note = Synthetic oligonucleotide
  source 1..49: mol_type = other DNA
ccccttagggataacagggtaatctccagtgatctagtaactccgtggt

SEQ ID NO: 864 - DNA, 48 bp, synthetic construct
  misc_feature 1..48: note = Synthetic oligonucleotide
  source 1..48: mol_type = other DNA
ccccttagggataacagggtaatcccttcagaaactagttagccctgt

SEQ ID NO: 865 - DNA, 49 bp, synthetic construct
  misc_feature 1..49: note = Synthetic oligonucleotide
  source 1..49: mol_type = other DNA
ccccttagggataacagggtaatcatggtccaaggtcagctggcggaca

SEQ ID NO: 866 - DNA, 44 bp, synthetic construct
  misc_feature 1..44: note = Synthetic oligonucleotide
  source 1..44: mol_type = other DNA
ccccttagggataacagggtaatcaggacccaccacggattcct

SEQ ID NO: 867 - DNA, 45 bp, synthetic construct
  misc_feature 1..45: note = Synthetic oligonucleotide
  source 1..45: mol_type = other DNA
ccccttagggataacagggtaatcactgcctcctccttagtcgat

SEQ ID NO: 868 - DNA, 48 bp, synthetic construct
  misc_feature 1..48: note = Synthetic oligonucleotide
  source 1..48: mol_type = other DNA
ccccttagggataacagggtaatcgctgtagacagattggcctcagtt

SEQ ID NO: 869 - DNA, 47 bp, synthetic construct
  misc_feature 1..47: note = Synthetic oligonucleotide
  source 1..47: mol_type = other DNA
ccccttagggataacagggtaatcccaaggtatgggggctaaccatt

SEQ ID NO: 870 - DNA, 50 bp, synthetic construct
  misc_feature 1..50: note = Synthetic oligonucleotide
  source 1..50: mol_type = other DNA
ccccttagggataacagggtaatcttctggaaattcgtcgaaggatggtc

SEQ ID NO: 871 - DNA, 47 bp, synthetic construct
  misc_feature 1..47: note = Synthetic oligonucleotide
  source 1..47: mol_type = other DNA
ccccttagggataacagggtaatcagctgtgctcttccgtttcagtg

SEQ ID NO: 872 - DNA, 47 bp, synthetic construct
  misc_feature 1..47: note = Synthetic oligonucleotide
  source 1..47: mol_type = other DNA
ccccttagggataacagggtaatcccacgaggcgtattcatctgcat

SEQ ID NO: 873 - DNA, 49 bp, synthetic construct
  misc_feature 1..49: note = Synthetic oligonucleotide
  source 1..49: mol_type = other DNA
ccccttagggataacagggtaatcaatggaaccacactacatcaagtta

SEQ ID NO: 874 - DNA, 45 bp, synthetic construct
  misc_feature 1..45: note = Synthetic oligonucleotide
  source 1..45: mol_type = other DNA
ccccttagggataacagggtaatccctgaccctgccgtgtaccag

SEQ ID NO: 875 - DNA, 43 bp, synthetic construct
  misc_feature 1..43: note = Synthetic oligonucleotide
  source 1..43: mol_type = other DNA
ccccttagggataacagggtaatccctgcgaaggcaccaaagc

SEQ ID NO: 876 - DNA, 56 bp, synthetic construct
  misc_feature 1..56: note = Synthetic oligonucleotide
  source 1..56: mol_type = other DNA
gtgactggagttcagacgtgtgctcttccgatctggctcagggtcgaaggctcact

SEQ ID NO: 877 - DNA, 60 bp, synthetic construct
  misc_feature 1..60: note = Synthetic oligonucleotide
  source 1..60: mol_type = other DNA
gtgactggagttcagacgtgtgctcttccgatcttcaaaccatcgggggaaaaatgacaa

SEQ ID NO: 878 - DNA, 60 bp, synthetic construct
  misc_feature 1..60: note = Synthetic oligonucleotide
  source 1..60: mol_type = other DNA
gtgactggagttcagacgtgtgctcttccgatctgtagatggggccggaaagtagaaaag

SEQ ID NO: 879 - DNA, 55 bp, synthetic construct
  misc_feature 1..55: note = Synthetic oligonucleotide
  source 1..55: mol_type = other DNA
gtgactggagttcagacgtgtgctcttccgatctcccagctccaagtccatctgg

SEQ ID NO: 880 - DNA, 52 bp, synthetic construct
  misc_feature 1..52: note = Synthetic oligonucleotide
  source 1..52: mol_type = other DNA
gtgactggagttcagacgtgtgctcttccgatctgctaccaagccccaccct

SEQ ID NO: 881 - DNA, 60 bp, synthetic construct
  misc_feature 1..60: note = Synthetic oligonucleotide
  source 1..60: mol_type = other DNA
gtgactggagttcagacgtgtgctcttccgatctcaccctatacccaccatactcacgtt

SEQ ID NO: 882 - DNA, 60 bp, synthetic construct
  misc_feature 1..60: note = Synthetic oligonucleotide
  source 1..60: mol_type = other DNA
gtgactggagttcagacgtgtgctcttccgatctagggaaggggcaacttttcaaaatct

SEQ ID NO: 883 - DNA, 60 bp, synthetic construct
  misc_feature 1..60: note = Synthetic oligonucleotide
  source 1..60: mol_type = other DNA
gtgactggagttcagacgtgtgctcttccgatctgcaccaagcttttgtgatgctccaac

SEQ ID NO: 884 - DNA, 58 bp, synthetic construct
  misc_feature 1..58: note = Synthetic oligonucleotide
  source 1..58: mol_type = other DNA
gtgactggagttcagacgtgtgctcttccgatctcacctgcaggaaacggttgcgttc

SEQ ID NO: 885 - DNA, 54 bp, synthetic construct
  misc_feature 1..54: note = Synthetic oligonucleotide
  source 1..54: mol_type = other DNA
gtgactggagttcagacgtgtgctcttccgatctgctgggccgcctgatctacc

SEQ ID NO: 886 - DNA, 54 bp, synthetic construct
  misc_feature 1..54: note = Synthetic oligonucleotide
  source 1..54: mol_type = other DNA
gtgactggagttcagacgtgtgctcttccgatctgccagtcactgcagctctct

SEQ ID NO: 887 - DNA, 58 bp, synthetic construct
  misc_feature 1..58: note = Synthetic oligonucleotide
  source 1..58: mol_type = other DNA
gtgactggagttcagacgtgtgctcttccgatctacatgtccaccgttcagacacagc

SEQ ID NO: 888 - DNA, 58 bp, synthetic construct
  misc_feature 1..58: note = Synthetic oligonucleotide
  source 1..58: mol_type = other DNA
gtgactggagttcagacgtgtgctcttccgatctgggaggctggtactactgggcatc

SEQ ID NO: 889 - DNA, 59 bp, synthetic construct
  misc_feature 1..59: note = Synthetic oligonucleotide
  source 1..59: mol_type = other DNA
gtgactggagttcagacgtgtgctcttccgatctagacactgcttccctggtaatggac

SEQ ID NO: 890 - DNA, 54 bp, synthetic construct
  misc_feature 1..54: note = Synthetic oligonucleotide
  source 1..54: mol_type = other DNA
gtgactggagttcagacgtgtgctcttccgatctcctgccagggtgcaagaact

SEQ ID NO: 891 - DNA, 60 bp, synthetic construct
  misc_feature 1..60: note = Synthetic oligonucleotide
  source 1..60: mol_type = other DNA
gtgactggagttcagacgtgtgctcttccgatctccatgaatgttgtggcgcattttcat

SEQ ID NO: 892 - DNA, 54 bp, synthetic construct
  misc_feature 1..54: note = Synthetic oligonucleotide
  source 1..54: mol_type = other DNA
gtgactggagttcagacgtgtgctcttccgatcttgctgtgggctcctttgctt

SEQ ID NO: 893 - DNA, 60 bp, synthetic construct
  misc_feature 1..60: note = Synthetic oligonucleotide
  source 1..60: mol_type = other DNA
gtgactggagttcagacgtgtgctcttccgatctcaaaggacctacatgtggctccaatt

SEQ ID NO: 894 - DNA, 60 bp, synthetic construct
  misc_feature 1..60: note = Synthetic oligonucleotide
  source 1..60: mol_type = other DNA
gtgactggagttcagacgtgtgctcttccgatctgcgaaggatcaggagagtactattag

SEQ ID NO: 895 - DNA, 54 bp, synthetic construct
  misc_feature 1..54: note = Synthetic oligonucleotide
  source 1..54: mol_type = other DNA
gtgactggagttcagacgtgtgctcttccgatctaggggtgggagagtgctact

SEQ ID NO: 896 - DNA, 60 bp, synthetic construct
  misc_feature 1..60: note = Synthetic oligonucleotide
  source 1..60: mol_type = other DNA
gtgactggagttcagacgtgtgctcttccgatcttgtatgttggttatgcgggaagagac

SEQ ID NO: 897 - DNA, 60 bp, synthetic construct
  misc_feature 1..60: note = Synthetic oligonucleotide
  source 1..60: mol_type = other DNA
gtgactggagttcagacgtgtgctcttccgatcttggatttccaaagagaagccctagtc

SEQ ID NO: 898 - DNA, 57 bp, synthetic construct
  misc_feature 1..57: note = Synthetic oligonucleotide
  source 1..57: mol_type = other DNA
gtgactggagttcagacgtgtgctcttccgatctacggatgagacttcagtgagtac

SEQ ID NO: 899 - DNA, 60 bp, synthetic construct
  misc_feature 1..60: note = Synthetic oligonucleotide
  source 1..60: mol_type = other DNA
gtgactggagttcagacgtgtgctcttccgatcttgcttcagatcccgtgacatcagtgt

SEQ ID NO: 900 - DNA, 60 bp, synthetic construct
  misc_feature 1..60: note = Synthetic oligonucleotide
  source 1..60: mol_type = other DNA
gtgactggagttcagacgtgtgctcttccgatctagtggggacttgctgctggtatctac

SEQ ID NO: 901 - DNA, 60 bp, synthetic construct
  misc_feature 1..60: note = Synthetic oligonucleotide
  source 1..60: mol_type = other DNA
gtgactggagttcagacgtgtgctcttccgatctgctacattcaggtaacatgtttctgc

SEQ ID NO: 902 - DNA, 54 bp, synthetic construct
  misc_feature 1..54: note = Synthetic oligonucleotide
  source 1..54: mol_type = other DNA
gtgactggagttcagacgtgtgctcttccgatctgccaaatcctcccaggtgca

SEQ ID NO: 903 - DNA, 60 bp, synthetic construct
  misc_feature 1..60: note = Synthetic oligonucleotide
  source 1..60: mol_type = other DNA
gtgactggagttcagacgtgtgctcttccgatctggccacaacttaggtgagagtgacga

SEQ ID NO: 904 - DNA, 59 bp, synthetic construct
  misc_feature 1..59: note = Synthetic oligonucleotide
  source 1..59: mol_type = other DNA
gtgactggagttcagacgtgtgctcttccgatctcctctgaactaagtgggagtttggc

SEQ ID NO: 905 - DNA, 60 bp, synthetic construct
  misc_feature 1..60: note = Synthetic oligonucleotide
  source 1..60: mol_type = other DNA
gtgactggagttcagacgtgtgctcttccgatcttgccatttctctaatcaaggggtgtg

SEQ ID NO: 906 - DNA, 60 bp, synthetic construct
  misc_feature 1..60: note = Synthetic oligonucleotide
  source 1..60: mol_type = other DNA
gtgactggagttcagacgtgtgctcttccgatcttcccagtcttgctttatactgtgcct

SEQ ID NO: 907 - DNA, 60 bp, synthetic construct
  misc_feature 1..60: note = Synthetic oligonucleotide
  source 1..60: mol_type = other DNA
gtgactggagttcagacgtgtgctcttccgatctctgggctcgttggttaccctattcct

SEQ ID NO: 908 - DNA, 54 bp, synthetic construct
  misc_feature 1..54: note = Synthetic oligonucleotide
  source 1..54: mol_type = other DNA
gtgactggagttcagacgtgtgctcttccgatctgaggagctaccagggcccta

SEQ ID NO: 909 - DNA, 59 bp, synthetic construct
  misc_feature 1..59: note = Synthetic oligonucleotide
  source 1..59: mol_type = other DNA
gtgactggagttcagacgtgtgctcttccgatctacctcatcatttctcaggcgaaagg

SEQ ID NO: 910 - DNA, 60 bp, synthetic construct
  misc_feature 1..60: note = Synthetic oligonucleotide
  source 1..60: mol_type = other DNA
gtgactggagttcagacgtgtgctcttccgatctgcatgattgtgtgaccgaatgcctca

SEQ ID NO: 911 - DNA, 57 bp, synthetic construct
  misc_feature 1..57: note = Synthetic oligonucleotide
  source 1..57: mol_type = other DNA
gtgactggagttcagacgtgtgctcttccgatctggtttgcaccatagaaagctgag

SEQ ID NO: 912 - DNA, 60 bp, synthetic construct
  misc_feature 1..60: note = Synthetic oligonucleotide
  source 1..60: mol_type = other DNA
gtgactggagttcagacgtgtgctcttccgatctggtaaaccagaactatctttctctcc

SEQ ID NO: 913 - DNA, 60 bp, synthetic construct
  misc_feature 1..60: note = Synthetic oligonucleotide
  source 1..60: mol_type = other DNA
gtgactggagttcagacgtgtgctcttccgatctgaacttgaaatgcttacagccagaat

SEQ ID NO: 914 - DNA, 60 bp, synthetic construct
  misc_feature 1..60: note = Synthetic oligonucleotide
  source 1..60: mol_type = other DNA
gtgactggagttcagacgtgtgctcttccgatctccataagttgaaatttgcgtttcggt

SEQ ID NO: 915 - DNA, 55 bp, synthetic construct
  misc_feature 1..55: note = Synthetic oligonucleotide
  source 1..55: mol_type = other DNA
gtgactggagttcagacgtgtgctcttccgatctcctcaggtgctgcttcctcaa

SEQ ID NO: 916 - DNA, 58 bp, synthetic construct
  misc_feature 1..58: note = Synthetic oligonucleotide
  source 1..58: mol_type = other DNA
gtgactggagttcagacgtgtgctcttccgatctcatgcgacagcctgatccgtttct

SEQ ID NO: 917 - DNA, 60 bp, synthetic construct
  misc_feature 1..60: note = Synthetic oligonucleotide
  source 1..60: mol_type = other DNA
gtgactggagttcagacgtgtgctcttccgatctgcctgtcaggaccatacaaatcttac

SEQ ID NO: 918 - DNA, 60 bp, synthetic construct
  misc_feature 1..60: note = Synthetic oligonucleotide
  source 1..60: mol_type = other DNA
gtgactggagttcagacgtgtgctcttccgatctttctcttgtgtgatccagagttgaca

SEQ ID NO: 919 - DNA, 60 bp, synthetic construct
  misc_feature 1..60: note = Synthetic oligonucleotide
  source 1..60: mol_type = other DNA
gtgactggagttcagacgtgtgctcttccgatctaacgacactgacagaaaacactcacg

SEQ ID NO: 920 - DNA, 60 bp, synthetic construct
  misc_feature 1..60: note = Synthetic oligonucleotide
  source 1..60: mol_type = other DNA
gtgactggagttcagacgtgtgctcttccgatctagcaatttggtcccccatggagagac

SEQ ID NO: 921 - DNA, 60 bp, synthetic construct
  misc_feature 1..60: note = Synthetic oligonucleotide
  source 1..60: mol_type = other DNA
gtgactggagttcagacgtgtgctcttccgatctaaaacttgactgttcattgggttcaa

SEQ ID NO: 922 - DNA, 60 bp, synthetic construct
  misc_feature 1..60: note = Synthetic oligonucleotide
  source 1..60: mol_type = other DNA
gtgactggagttcagacgtgtgctcttccgatctcccctgtctctaccatcctagacacc

SEQ ID NO: 923 - DNA, 57 bp, synthetic construct
  misc_feature 1..57: note = Synthetic oligonucleotide
  source 1..57: mol_type = other DNA
gtgactggagttcagacgtgtgctcttccgatctagaaggcagcctcccaaagcact

SEQ ID NO: 924 - DNA, 60 bp, synthetic construct
  misc_feature 1..60: note = Synthetic oligonucleotide
  source 1..60: mol_type = other DNA
gtgactggagttcagacgtgtgctcttccgatctgcctggagtggtgtctggtacaatga

SEQ ID NO: 925 - DNA, 60 bp, synthetic construct
  misc_feature 1..60: note = Synthetic oligonucleotide
  source 1..60: mol_type = other DNA
gtgactggagttcagacgtgtgctcttccgatctgtccctggccttaaagaaatgacaga

SEQ ID NO: 926 - DNA, 59 bp, synthetic construct
  misc_feature 1..59: note = Synthetic oligonucleotide
  source 1..59: mol_type = other DNA
gtgactggagttcagacgtgtgctcttccgatctagccttgattactgttcccactagc

SEQ ID NO: 927 - DNA, 58 bp, synthetic construct
  misc_feature 1..58: note = Synthetic oligonucleotide
  source 1..58: mol_type = other DNA
gtgactggagttcagacgtgtgctcttccgatctgggcttccttgctttggttactgt

SEQ ID NO: 928 - DNA, 58 bp, synthetic construct
  misc_feature 1..58: note = Synthetic oligonucleotide
  source 1..58: mol_type = other DNA
gtgactggagttcagacgtgtgctcttccgatctcactgctgcccccacaagcttaac

SEQ ID NO: 929 - DNA, 52 bp, synthetic construct
  misc_feature 1..52: note = Synthetic oligonucleotide
  source 1..52: mol_type = other DNA
gtgactggagttcagacgtgtgctcttccgatctgccggagtcaggggcatc

SEQ ID NO: 930 - DNA, 60 bp, synthetic construct
  misc_feature 1..60: note = Synthetic oligonucleotide
  source 1..60: mol_type = other DNA
gtgactggagttcagacgtgtgctcttccgatctatggcaatccgttggaaatgtcttct

SEQ ID NO: 931 - DNA, 56 bp, synthetic construct
  misc_feature 1..56: note = Synthetic oligonucleotide
  source 1..56: mol_type = other DNA
gtgactggagttcagacgtgtgctcttccgatctcccataccccactcccactact

SEQ ID NO: 932 - DNA, 60 bp, synthetic construct
  misc_feature 1..60: note = Synthetic oligonucleotide
  source 1..60: mol_type = other DNA
gtgactggagttcagacgtgtgctcttccgatctcaggtttcaggcggagtggaagaagt

SEQ ID NO: 933 - DNA, 59 bp, synthetic construct
  misc_feature 1..59: note = Synthetic oligonucleotide
  source 1..59: mol_type = other DNA
gtgactggagttcagacgtgtgctcttccgatctaaccctgtgaacatcgtgattccag

SEQ ID NO: 934 - DNA, 60 bp, synthetic construct
  misc_feature 1..60: note = Synthetic oligonucleotide
  source 1..60: mol_type = other DNA
gtgactggagttcagacgtgtgctcttccgatctcggtactggccaatgaaattttccca

SEQ ID NO: 935 - DNA, 60 bp, synthetic construct
  misc_feature 1..60: note = Synthetic oligonucleotide
  source 1..60: mol_type = other DNA
gtgactggagttcagacgtgtgctcttccgatctttggctctctggattgttcctctaaa

SEQ ID NO: 936 - DNA, 60 bp, synthetic construct
  misc_feature 1..60: note = Synthetic oligonucleotide
  source 1..60: mol_type = other DNA
gtgactggagttcagacgtgtgctcttccgatctcaccctacagtctatgttccaggtca

SEQ ID NO: 937 - DNA, 60 bp, synthetic construct
  misc_feature 1..60: note = Synthetic oligonucleotide
  source 1..60: mol_type = other DNA
gtgactggagttcagacgtgtgctcttccgatctccagactaaggcaagcaactgtaaca

SEQ ID NO: 938 - DNA, 55 bp, synthetic construct
  misc_feature 1..55: note = Synthetic oligonucleotide
  source 1..55: mol_type = other DNA
gtgactggagttcagacgtgtgctcttccgatctcttcgtgttggccaggtgggt

SEQ ID NO: 939 - DNA, 58 bp, synthetic construct
  misc_feature 1..58: note = Synthetic oligonucleotide
  source 1..58: mol_type = other DNA
gtgactggagttcagacgtgtgctcttccgatcttgaagctgagttacctgggagctc

SEQ ID NO: 940 - DNA, 60 bp, synthetic construct
  misc_feature 1..60: note = Synthetic oligonucleotide
  source 1..60: mol_type = other DNA
gtgactggagttcagacgtgtgctcttccgatctcttacgggaaagcaagttgactttgc

SEQ ID NO: 941 - DNA, 60 bp, synthetic construct
  misc_feature 1..60: note = Synthetic oligonucleotide
  source 1..60: mol_type = other DNA
gtgactggagttcagacgtgtgctcttccgatctacaaaacaaaaccctccggatggtct

SEQ ID NO: 942 - DNA, 60 bp, synthetic construct
  misc_feature 1..60: note = Synthetic oligonucleotide
  source 1..60: mol_type = other DNA
gtgactggagttcagacgtgtgctcttccgatcttgctcctgtctgcctgattacttact

SEQ ID NO: 943 - DNA, 54 bp, synthetic construct
  misc_feature 1..54: note = Synthetic oligonucleotide
  source 1..54: mol_type = other DNA
gtgactggagttcagacgtgtgctcttccgatctggtcatgcctccaacctgca

SEQ ID NO: 944 - DNA, 60 bp, synthetic construct
  misc_feature 1..60: note = Synthetic oligonucleotide
  source 1..60: mol_type = other DNA
gtgactggagttcagacgtgtgctcttccgatctttgcgttgacttaaatgaaagtgtcc

SEQ ID NO: 945 - DNA, 54 bp, synthetic construct
  misc_feature 1..54: note = Synthetic oligonucleotide
  source 1..54: mol_type = other DNA
gtgactggagttcagacgtgtgctcttccgatctactgtcgggagaaggcgtct

SEQ ID NO: 946 - DNA, 60 bp, synthetic construct
  misc_feature 1..60: note = Synthetic oligonucleotide
  source 1..60: mol_type = other DNA
gtgactggagttcagacgtgtgctcttccgatcttgatctgtttgtgaatggctcagaca

SEQ ID NO: 947 - DNA, 60 bp, synthetic construct
  misc_feature 1..60: note = Synthetic oligonucleotide
  source 1..60: mol_type = other DNA
gtgactggagttcagacgtgtgctcttccgatctactctgggtgcgggggaacttatttg

SEQ ID NO: 948 - DNA, 60 bp, synthetic construct
  misc_feature 1..60: note = Synthetic oligonucleotide
  source 1..60: mol_type = other DNA
gtgactggagttcagacgtgtgctcttccgatctccgtggtggatttaactcccctattg

SEQ ID NO: 949 - DNA, 54 bp, synthetic construct
  misc_feature 1..54: note = Synthetic oligonucleotide
  source 1..54: mol_type = other DNA
gtgactggagttcagacgtgtgctcttccgatctagcattctgcctctgacagg

SEQ ID NO: 950 - DNA, 55 bp, synthetic construct
  misc_feature 1..55: note = Synthetic oligonucleotide
  source 1..55: mol_type = other DNA
gtgactggagttcagacgtgtgctcttccgatcttccaaggtcagctggcggaca

SEQ ID NO: 951 - DNA, 60 bp, synthetic construct
  misc_feature 1..60: note = Synthetic oligonucleotide
  source 1..60: mol_type = other DNA
gtgactggagttcagacgtgtgctcttccgatctacggattcctgctgtactggctaaag

SEQ ID NO: 952 - DNA, 60 bp, synthetic construct
  misc_feature 1..60: note = Synthetic oligonucleotide
  source 1..60: mol_type = other DNA
gtgactggagttcagacgtgtgctcttccgatcttgcctcctccttagtcgattcttacc

SEQ ID NO: 953 - DNA, 58 bp, synthetic construct
  misc_feature 1..58: note = Synthetic oligonucleotide
  source 1..58: mol_type = other DNA
gtgactggagttcagacgtgtgctcttccgatctaacaagtgtccctggcaaatgtga

SEQ ID NO: 954 - DNA, 58 bp, synthetic construct
  misc_feature 1..58: note = Synthetic oligonucleotide
  source 1..58: mol_type = other DNA
gtgactggagttcagacgtgtgctcttccgatctgggggctaaccattggcaattgaa

SEQ ID NO: 955 - DNA, 60 bp, synthetic construct
  misc_feature 1..60: note = Synthetic oligonucleotide
  source 1..60: mol_type = other DNA
gtgactggagttcagacgtgtgctcttccgatctttcgtcgaaggatggtctctctgttg

SEQ ID NO: 956 - DNA, 60 bp, synthetic construct
  misc_feature 1..60: note = Synthetic oligonucleotide
  source 1..60: mol_type = other DNA
gtgactggagttcagacgtgtgctcttccgatcttgtgctcttccgtttcagtgtgaaaa

SEQ ID NO: 957 - DNA, 59 bp, synthetic construct
  misc_feature 1..59: note = Synthetic oligonucleotide
  source 1..59: mol_type = other DNA
gtgactggagttcagacgtgtgctcttccgatctatctgcatgcatgagtcctgacttc

SEQ ID NO: 958 - DNA, 60 bp, synthetic construct
  misc_feature 1..60: note = Synthetic oligonucleotide
  source 1..60: mol_type = other DNA
gtgactggagttcagacgtgtgctcttccgatctatcaagttacatagaaatggggaggt

SEQ ID NO: 959 - DNA, 58 bp, synthetic construct
  misc_feature 1..58: note = Synthetic oligonucleotide
  source 1..58: mol_type = other DNA
gtgactggagttcagacgtgtgctcttccgatctcctgccgtgtaccagctgagagac

SEQ ID NO: 960 - DNA, 56 bp, synthetic construct
  misc_feature 1..56: note = Synthetic oligonucleotide
  source 1..56: mol_type = other DNA
gtgactggagttcagacgtgtgctcttccgatctgctgttgttgaaggcgtttgca

SEQ ID NO: 961 - DNA, 23 bp, synthetic construct
  misc_feature 1..23: note = Synthetic oligonucleotide
  source 1..23: mol_type = other DNA
gaccccctccaccccgcctccgg

SEQ ID NO: 962 - DNA, 23 bp, synthetic construct
  misc_feature 1..23: note = Synthetic oligonucleotide
  source 1..23: mol_type = other DNA
ctacccctccaccccgcctccgg

SEQ ID NO: 963 - DNA, 23 bp, synthetic construct
  misc_feature 1..23: note = Synthetic oligonucleotide
  source 1..23: mol_type = other DNA
attcccccccaccccgcctcagg

SEQ ID NO: 964 - DNA, 23 bp, synthetic construct
  misc_feature 1..23: note = Synthetic oligonucleotide
  source 1..23: mol_type = other DNA
gggcccctccaccccgcctctgg

SEQ ID NO: 965 - DNA, 23 bp, synthetic construct
  misc_feature 1..23: note = Synthetic oligonucleotide
  source 1..23: mol_type = other DNA
gacccccttcaccccacctatgg

SEQ ID NO: 966 - DNA, 23 bp, synthetic construct
  misc_feature 1..23: note = Synthetic oligonucleotide
  source 1..23: mol_type = other DNA
taccccccacaccccgcctctgg

SEQ ID NO: 967 - DNA, 23 bp, synthetic construct
  misc_feature 1..23: note = Synthetic oligonucleotide
  source 1..23: mol_type = other DNA
gcccccacccaccccgcctctgg

SEQ ID NO: 968 - DNA, 23 bp, synthetic construct
  misc_feature 1..23: note = Synthetic oligonucleotide
  source 1..23: mol_type = other DNA
tgccccccccaccccacctctgg

SEQ ID NO: 969 - DNA, 23 bp, synthetic construct
  misc_feature 1..23: note = Synthetic oligonucleotide
  source 1..23: mol_type = other DNA
acacccccccaccccgcctcagg

SEQ ID NO: 970 - DNA, 23 bp, synthetic construct
  misc_feature 1..23: note = Synthetic oligonucleotide
  source 1..23: mol_type = other DNA
ctcccccccctccccgcctcggg

SEQ ID NO: 971 - DNA, 23 bp, synthetic construct
  misc_feature 1..23: note = Synthetic oligonucleotide
  source 1..23: mol_type = other DNA
tgcccctcccaccccgcctctgg

SEQ ID NO: 972 - DNA, 23 bp, synthetic construct
  misc_feature 1..23: note = Synthetic oligonucleotide
  source 1..23: mol_type = other DNA
cgccctccccaccccgcctccgg

SEQ ID NO: 973 - DNA, 23 bp, synthetic construct
  misc_feature 1..23: note = Synthetic oligonucleotide
  source 1..23: mol_type = other DNA
agccccccccaccccgactcagg

SEQ ID NO: 974 - DNA, 23 bp, synthetic construct
  misc_feature 1..23: note = Synthetic oligonucleotide
  source 1..23: mol_type = other DNA
gccccccaccaccccacctcggg

SEQ ID NO: 975 - DNA, 23 bp, synthetic construct
  misc_feature 1..23: note = Synthetic oligonucleotide
  source 1..23: mol_type = other DNA
gacacaccccaccccacctcagg

SEQ ID NO: 976 - DNA, 23 bp, synthetic construct
  misc_feature 1..23: note = Synthetic oligonucleotide
  source 1..23: mol_type = other DNA
ggccctctccactccacctcagg

SEQ ID NO: 977 - DNA, 23 bp, synthetic construct
  misc_feature 1..23: note = Synthetic oligonucleotide
  source 1..23: mol_type = other DNA
cccccccccccccccgcctccgg

SEQ ID NO: 978 - DNA, 23 bp, synthetic construct
  misc_feature 1..23: note = Synthetic oligonucleotide
  source 1..23: mol_type = other DNA
tcccccctcaaccccacctcagg

SEQ ID NO: 979 - DNA, 23 bp, synthetic construct
  misc_feature 1..23: note = Synthetic oligonucleotide
  source 1..23: mol_type = other DNA
ctgcccccccaccccgccactgg

SEQ ID NO: 980 - DNA, 23 bp, synthetic construct
  misc_feature 1..23: note = Synthetic oligonucleotide
  source 1..23: mol_type = other DNA
tgccccccccaccccgcccccgg

SEQ ID NO: 981 - DNA, 23 bp, synthetic construct
  misc_feature 1..23: note = Synthetic oligonucleotide
  source 1..23: mol_type = other DNA
gtcctccaccaccccgcctctgg

SEQ ID NO: 982 - DNA, 23 bp, synthetic construct
  misc_feature 1..23: note = Synthetic oligonucleotide
  source 1..23: mol_type = other DNA
gccacccaccaccccacctcagg

SEQ ID NO: 983 - DNA, 23 bp, synthetic construct
  misc_feature 1..23: note = Synthetic oligonucleotide
  source 1..23: mol_type = other DNA
taccccccccaccccgccacagg

SEQ ID NO: 984 - DNA, 23 bp, synthetic construct
  misc_feature 1..23: note = Synthetic oligonucleotide
  source 1..23: mol_type = other DNA
ctccccacccaccccgcctcagg

SEQ ID NO: 985 - DNA, 23 bp, synthetic construct
  misc_feature 1..23: note = Synthetic oligonucleotide
  source 1..23: mol_type = other DNA
caacccccccaccccgcttcagg

SEQ ID NO: 986 - DNA, 23 bp, synthetic construct
  misc_feature 1..23: note = Synthetic oligonucleotide
  source 1..23: mol_type = other DNA
gcttccctccaccccgcatccgg

SEQ ID NO: 987 - DNA, 23 bp, synthetic construct
  misc_feature 1..23: note = Synthetic oligonucleotide
  source 1..23: mol_type = other DNA
gtcactccccaccccgcctctgg

SEQ ID NO: 988 - DNA, 23 bp, synthetic construct
  misc_feature 1..23: note = Synthetic oligonucleotide
  source 1..23: mol_type = other DNA
atccccctccaccccacccctgg

SEQ ID NO: 989 - DNA, 23 bp, synthetic construct
  misc_feature 1..23: note = Synthetic oligonucleotide
  source 1..23: mol_type = other DNA
gaccccccccaccccgcccccgg

SEQ ID NO: 990 - DNA, 23 bp, synthetic construct
  misc_feature 1..23: note = Synthetic oligonucleotide
  source 1..23: mol_type = other DNA
gccaccttccaccccacctcagg

SEQ ID NO: 991 - DNA, 23 bp, synthetic construct
  misc_feature 1..23: note = Synthetic oligonucleotide
  source 1..23: mol_type = other DNA
cactccccccaccccgccccagg

SEQ ID NO: 992 - DNA, 23 bp, synthetic construct
  misc_feature 1..23: note = Synthetic oligonucleotide
  source 1..23: mol_type = other DNA
gacccctcccaccccgactccgg

SEQ ID NO: 993 - DNA, 23 bp, synthetic construct
  misc_feature 1..23: note = Synthetic oligonucleotide
  source 1..23: mol_type = other DNA
cccccccccccccccgcctcagg

SEQ ID NO: 994 - DNA, 23 bp, synthetic construct
  misc_feature 1..23: note = Synthetic oligonucleotide
  source 1..23: mol_type = other DNA
gcctctctgcaccccgcctcagg

SEQ ID NO: 995 - DNA, 23 bp, synthetic construct
  misc_feature 1..23: note = Synthetic oligonucleotide
  source 1..23: mol_type = other DNA
ccccccccccaccccgcccccgg

SEQ ID NO: 996 - DNA, 23 bp, synthetic construct
  misc_feature 1..23: note = Synthetic oligonucleotide
  source 1..23: mol_type = other DNA
ctctccccccaccccgcctctgg

SEQ ID NO: 997 - DNA, 23 bp, synthetic construct
  misc_feature 1..23: note = Synthetic oligonucleotide
  source 1..23: mol_type = other DNA
ccccacccccaccccgcctcagg

SEQ ID NO: 998 - DNA, 23 bp, synthetic construct
  misc_feature 1..23: note = Synthetic oligonucleotide
  source 1..23: mol_type = other DNA
gaccccccccaccccaccccagg

SEQ ID NO: 999 - DNA, 23 bp, synthetic construct
  misc_feature 1..23: note = Synthetic oligonucleotide
  source 1..23: mol_type = other DNA
ccacccccccaccccgccccagg

SEQ ID NO: 1000 - DNA, 23 bp, synthetic construct
  misc_feature 1..23: note = Synthetic oligonucleotide
  source 1..23: mol_type = other DNA
aggcccccccgccccgcctcagg

SEQ ID NO: 1001 - DNA, 23 bp, synthetic construct
  misc_feature 1..23: note = Synthetic oligonucleotide
  source 1..23: mol_type = other DNA
cccccccccccccccacccccag

SEQ ID NO: 1002 - DNA, 23 bp, synthetic construct
  misc_feature 1..23: note = Synthetic oligonucleotide
  source 1..23: mol_type = other DNA
gatcgactccaccccgcctctgg

SEQ ID NO: 1003 - DNA, 23 bp, synthetic construct
  misc_feature 1..23: note = Synthetic oligonucleotide
  source 1..23: mol_type = other DNA
agccaaccccaccccgcctctgg

SEQ ID NO: 1004 - DNA, 23 bp, synthetic construct
  misc_feature 1..23: note = Synthetic oligonucleotide
  source 1..23: mol_type = other DNA
tccaccccccaccccgccccggg

SEQ ID NO: 1005 - DNA, 23 bp, synthetic construct
  misc_feature 1..23: note = Synthetic oligonucleotide
  source 1..23: mol_type = other DNA
caccccccgcaccccgccccagg

SEQ ID NO: 1006 - DNA, 23 bp, synthetic construct
  misc_feature 1..23: note = Synthetic oligonucleotide
  source 1..23: mol_type = other DNA
cctcccccacaccccgcatccgg

SEQ ID NO: 1007 - DNA, 23 bp, synthetic construct
  misc_feature 1..23: note = Synthetic oligonucleotide
  source 1..23: mol_type = other DNA
ggcagcctccaccacgcctccgg

SEQ ID NO: 1008 - DNA, 23 bp, synthetic construct
  misc_feature 1..23: note = Synthetic oligonucleotide
  source 1..23: mol_type = other DNA
catcccccccaccccaccccggg

SEQ ID NO: 1009 - DNA, 23 bp, synthetic construct
  misc_feature 1..23: note = Synthetic oligonucleotide
  source 1..23: mol_type = other DNA
ccacccccccaccccgcccctgg

SEQ ID NO: 1010 - DNA, 23 bp, synthetic construct
  misc_feature 1..23: note = Synthetic oligonucleotide
  source 1..23: mol_type = other DNA
aggcccccacaccccgcctcagg

SEQ ID NO: 1011 - DNA, 23 bp, synthetic construct
  misc_feature 1..23: note = Synthetic oligonucleotide
  source 1..23: mol_type = other DNA
gtaccccaccaccccgccccagg

SEQ ID NO: 1012 - DNA, 23 bp, synthetic construct
  misc_feature 1..23: note = Synthetic oligonucleotide
  source 1..23: mol_type = other DNA
cataccccccaccccgccccggg

SEQ ID NO: 1013 - DNA, 23 bp, synthetic construct
  misc_feature 1..23: note = Synthetic oligonucleotide
  source 1..23: mol_type = other DNA
ccgcccctccaccccgccactgg

SEQ ID NO: 1014 - DNA, 23 bp, synthetic construct
  misc_feature 1..23: note = Synthetic oligonucleotide
  source 1..23: mol_type = other DNA
agtagcccccaccccgcctcggg

SEQ ID NO: 1015 - DNA, 23 bp, synthetic construct
  misc_feature 1..23: note = Synthetic oligonucleotide
  source 1..23: mol_type = other DNA
accccccccccccccgcccccgg

SEQ ID NO: 1016 - DNA, 23 bp, synthetic construct
  misc_feature 1..23: note = Synthetic oligonucleotide
  source 1..23: mol_type = other DNA
gccccgctcctccccgcctccgg

SEQ ID NO: 1017 - DNA, 23 bp, synthetic construct
  misc_feature 1..23: note = Synthetic oligonucleotide
  source 1..23: mol_type = other DNA
ccacccctccaccctgcttcggg

SEQ ID NO: 1018 - DNA, 23 bp, synthetic construct
  misc_feature 1..23: note = Synthetic oligonucleotide
  source 1..23: mol_type = other DNA
catttcccctaccccgcccctgg

SEQ ID NO: 1019 - DNA, 23 bp, synthetic construct
  misc_feature 1..23: note = Synthetic oligonucleotide
  source 1..23: mol_type = other DNA
aacacgccccaccccgccccagg

SEQ ID NO: 1020 - DNA, 23 bp, synthetic construct
  misc_feature 1..23: note = Synthetic oligonucleotide
  source 1..23: mol_type = other DNA
gagccactgtgcccagcctaggg

SEQ ID NO: 1021 - DNA, 23 bp, synthetic construct
  misc_feature 1..23: note = Synthetic oligonucleotide
  source 1..23: mol_type = other DNA
cactccccaccccccacccccag

SEQ ID NO: 1022 - DNA, 23 bp, synthetic construct
  misc_feature 1..23: note = Synthetic oligonucleotide
  source 1..23: mol_type = other DNA
ccctccccccaccccacaacagg

SEQ ID NO: 1023 - DNA, 23 bp, synthetic construct
  misc_feature 1..23: note = Synthetic oligonucleotide
  source 1..23: mol_type = other DNA
gtccctttccaccctgcctctgg

SEQ ID NO: 1024 - DNA, 23 bp, synthetic construct
  misc_feature 1..23: note = Synthetic oligonucleotide
  source 1..23: mol_type = other DNA
gagctcccccaccccgccccggg

SEQ ID NO: 1025 - DNA, 23 bp, synthetic construct
  misc_feature 1..23: note = Synthetic oligonucleotide
  source 1..23: mol_type = other DNA
aacacccgcccccccacccccgg

SEQ ID NO: 1026 - DNA, 23 bp, synthetic construct
  misc_feature 1..23: note = Synthetic oligonucleotide
  source 1..23: mol_type = other DNA
gattccctggaccacatctctgg

SEQ ID NO: 1027 - DNA, 23 bp, synthetic construct
  misc_feature 1..23: note = Synthetic oligonucleotide
  source 1..23: mol_type = other DNA
gagccaccaaacccagcctcagg

SEQ ID NO: 1028 - DNA, 23 bp, synthetic construct
  misc_feature 1..23: note = Synthetic oligonucleotide
  source 1..23: mol_type = other DNA
gaatcccaggagcccgcctcgag

SEQ ID NO: 1029 - DNA, 23 bp, synthetic construct
  misc_feature 1..23: note = Synthetic oligonucleotide
  source 1..23: mol_type = other DNA
ggccccctttcccacatctctgg

SEQ ID NO: 1030 - DNA, 23 bp, synthetic construct
  misc_feature 1..23: note = Synthetic oligonucleotide
  source 1..23: mol_type = other DNA
ctcccccagccccccacctcccg

SEQ ID NO: 1031 - DNA, 23 bp, synthetic construct
  misc_feature 1..23: note = Synthetic oligonucleotide
  source 1..23: mol_type = other DNA
cattctcgacaccccgcccccgg

SEQ ID NO: 1032 - DNA, 23 bp, synthetic construct
  misc_feature 1..23: note = Synthetic oligonucleotide
  source 1..23: mol_type = other DNA
tactccttcacccccaccccagg

SEQ ID NO: 1033 - DNA, 23 bp, synthetic construct
  misc_feature 1..23: note = Synthetic oligonucleotide
  source 1..23: mol_type = other DNA
cacactctcaacctcacttctag

SEQ ID NO: 1034 - DNA, 23 bp, synthetic construct
  misc_feature 1..23: note = Synthetic oligonucleotide
  source 1..23: mol_type = other DNA
tccatcctcagccccacctctcg

SEQ ID NO: 1035 - DNA, 23 bp, synthetic construct
  misc_feature 1..23: note = Synthetic oligonucleotide
  source 1..23: mol_type = other DNA
aacccattccaccctgcctcagg

SEQ ID NO: 1036 - DNA, 23 bp, synthetic construct
  misc_feature 1..23: note = Synthetic oligonucleotide
  source 1..23: mol_type = other DNA
gccaccccccaccctgcctccgg

SEQ ID NO: 1037 - DNA, 23 bp, synthetic construct
  misc_feature 1..23: note = Synthetic oligonucleotide
  source 1..23: mol_type = other DNA
caccaggtctgccccgcatcagg

SEQ ID NO: 1038 - DNA, 23 bp, synthetic construct
  misc_feature 1..23: note = Synthetic oligonucleotide
  source 1..23: mol_type = other DNA
aatcctctcacctcagcctccgg

SEQ ID NO: 1039 - DNA, 23 bp, synthetic construct
  misc_feature 1..23: note = Synthetic oligonucleotide
  source 1..23: mol_type = other DNA
gtgccactccaccccaccctggg

SEQ ID NO: 1040 - DNA, 23 bp, synthetic construct
  misc_feature 1..23: note = Synthetic oligonucleotide
  source 1..23: mol_type = other DNA
ccccccggcccccccaccccagg

SEQ ID NO: 1041 - DNA, 23 bp, synthetic construct
  misc_feature 1..23: note = Synthetic oligonucleotide
  source 1..23: mol_type = other DNA
caccccccgccccccgcccccgg

SEQ ID NO: 1042 - DNA, 23 bp, synthetic construct
  misc_feature 1..23: note = Synthetic oligonucleotide
  source 1..23: mol_type = other DNA
ctcaccataaactccgcctcccg

SEQ ID NO: 1043 - DNA, 23 bp, synthetic construct
  misc_feature 1..23: note = Synthetic oligonucleotide
  source 1..23: mol_type = other DNA
gagccactgcacccagcctcaag

SEQ ID NO: 1044 - DNA, 23 bp, synthetic construct
  misc_feature 1..23: note = Synthetic oligonucleotide
  source 1..23: mol_type = other DNA
gagccaccacaaccagcctcgag

SEQ ID NO: 1045 - DNA, 23 bp, synthetic construct
  misc_feature 1..23: note = Synthetic oligonucleotide
  source 1..23: mol_type = other DNA
gtttcccttcttcccgccccagg

SEQ ID NO: 1046 - DNA, 23 bp, synthetic construct
  misc_feature 1..23: note = Synthetic oligonucleotide
  source 1..23: mol_type = other DNA
cccccacccccccccacccccag

SEQ ID NO: 1047 - DNA, 23 bp, synthetic construct
  misc_feature 1..23: note = Synthetic oligonucleotide
  source 1..23: mol_type = other DNA
atcctcccacaccccacatcaga

SEQ ID NO: 1048 - DNA, 23 bp, synthetic construct
  misc_feature 1..23: note = Synthetic oligonucleotide
  source 1..23: mol_type = other DNA
caccgcgcccagccagcttctgg

SEQ ID NO: 1049 - DNA, 23 bp, synthetic construct
  misc_feature 1..23: note = Synthetic oligonucleotide
  source 1..23: mol_type = other DNA
gagccacctcacccagcctaaag

SEQ ID NO: 1050 - DNA, 23 bp, synthetic construct
  misc_feature 1..23: note = Synthetic oligonucleotide
  source 1..23: mol_type = other DNA
gagccaccacacccagcctaaag

SEQ ID NO: 1051 - DNA, 23 bp, synthetic construct
  misc_feature 1..23: note = Synthetic oligonucleotide
  source 1..23: mol_type = other DNA
gagccactgcgcccagccccagg

SEQ ID NO: 1052 - DNA, 23 bp, synthetic construct
  misc_feature 1..23: note = Synthetic oligonucleotide
  source 1..23: mol_type = other DNA
gaaccagacctccccatctccag

SEQ ID NO: 1053 - DNA, 23 bp, synthetic construct
  misc_feature 1..23: note = Synthetic oligonucleotide
  source 1..23: mol_type = other DNA
gagccactgcacctggcctcagg

SEQ ID NO: 1054 - DNA, 23 bp, synthetic construct
  misc_feature 1..23: note = Synthetic oligonucleotide
  source 1..23: mol_type = other DNA
gcacaccacccccccgccaccgg

SEQ ID NO: 1055 - DNA, 91 bp, synthetic construct
  misc_feature 1..91: note = Synthetic oligonucleotide
  source 1..91: mol_type = other DNA
tgtgaaaactaagagagagctccacccctctgtgccctcctcctgtcctgagtcggggtggggggggctggccttggagggggcgtcccct

SEQ ID NO: 1056 - DNA, 100 bp, synthetic construct
  misc_feature 1..100: note = Synthetic polynucleotide
  source 1..100: mol_type = other DNA
ggccacgtcgcccgtgtatgagatggcagcctccaccacgcctccggcacttcctgccgcctccatgcccagcagcatgttgggcaagtagttgagggag

SEQ ID NO: 1057 - protein, 33 aa, synthetic construct
  REGION 1..33: note = Synthetic polypeptide
AVDGTYSIAAEVVGGASGAAEMGLLMNPLYNLS

SEQ ID NO: 1058 - DNA, 88 bp, synthetic construct
  misc_feature 1..88: note = Synthetic oligonucleotide
  source 1..88: mol_type = other DNA
ccacccaccaccccacctcaggcaaatgcccagcccctgcctcgcctccagcctcctttccacaacccagcatccagtcactccagtc

SEQ ID NO: 1059 - DNA, 46 bp, synthetic construct
  misc_feature 1..46: note = Synthetic oligonucleotide
  source 1..46: mol_type = other DNA
gccccgggtttcaagtgattttcatacttcagcctcctgagtagct

SEQ ID NO: 1060 - DNA, 23 bp, synthetic construct
  misc_feature 1..23: note = Synthetic oligonucleotide
  source 1..23: mol_type = other DNA
agccccagcaagagcacaagagg

SEQ ID NO: 1061 - DNA, 23 bp, synthetic construct
  misc_feature 1..23: note = Synthetic oligonucleotide
  source 1..23: mol_type = other DNA
atcacccccaagagcacaagggg

SEQ ID NO: 1062 - DNA, 23 bp, synthetic construct
  misc_feature 1..23: note = Synthetic oligonucleotide
  source 1..23: mol_type = other DNA
agccccagtgagagcacaagagg

SEQ ID NO: 1063 - DNA, 23 bp, synthetic construct
  misc_feature 1..23: note = Synthetic oligonucleotide
  source 1..23: mol_type = other DNA
agttccagcaacagcacaaaagg

SEQ ID NO: 1064 - DNA, 23 bp, synthetic construct
  misc_feature 1..23: note = Synthetic oligonucleotide
  source 1..23: mol_type = other DNA
aactccagcgagagcacaagagg

SEQ ID NO: 1065 - DNA, 23 bp, synthetic construct
  misc_feature 1..23: note = Synthetic oligonucleotide
  source 1..23: mol_type = other DNA
agccccagtaagagcacaagagg

SEQ ID NO: 1066 - DNA, 23 bp, synthetic construct
  misc_feature 1..23: note = Synthetic oligonucleotide
  source 1..23: mol_type = other DNA
aacacaagcaagagcacgagagg

SEQ ID NO: 1067 - DNA, 23 bp, synthetic construct
  misc_feature 1..23: note = Synthetic oligonucleotide
  source 1..23: mol_type = other DNA
agccccagcaagagcacgagagg

SEQ ID NO: 1068 - DNA, 23 bp, synthetic construct
  misc_feature 1..23: note = Synthetic oligonucleotide
  source 1..23: mol_type = other DNA
agcctaagaaagaacacaagagg

SEQ ID NO: 1069 - DNA, 23 bp, synthetic construct
  misc_feature 1..23: note = Synthetic oligonucleotide
  source 1..23: mol_type = other DNA
agccccagctaaagcaaaagagg

SEQ ID NO: 1070 - DNA, 23 bp, synthetic construct
  misc_feature 1..23: note = Synthetic oligonucleotide
  source 1..23: mol_type = other DNA
tgccccagctaaaacacaagtgg

SEQ ID NO: 1071 - DNA, 23 bp, synthetic construct
  misc_feature 1..23: note = Synthetic oligonucleotide
  source 1..23: mol_type = other DNA
aaaccaaacaaggacacaagaga

SEQ ID NO: 1072 - DNA, 23 bp, synthetic construct
  misc_feature 1..23: note = Synthetic oligonucleotide
  source 1..23: mol_type = other DNA
aatcccagtgagagcacaagagg

SEQ ID NO: 1073 - DNA, 23 bp, synthetic construct
  misc_feature 1..23: note = Synthetic oligonucleotide
  source 1..23: mol_type = other DNA
acccctagctacagcacaagagg

SEQ ID NO: 1074 - DNA, 23 bp, synthetic construct
  misc_feature 1..23: note = Synthetic oligonucleotide
  source 1..23: mol_type = other DNA
tgcagcagcaagagcacaggcgg

SEQ ID NO: 1075 - DNA, 23 bp, synthetic construct
  misc_feature 1..23: note = Synthetic oligonucleotide
  source 1..23: mol_type = other DNA
cacaagagcaagagcacaagagg

SEQ ID NO: 1076 - DNA, 23 bp, synthetic construct
  misc_feature 1..23: note = Synthetic oligonucleotide
  source 1..23: mol_type = other DNA
gctctcagcaagaccacaagtgg

SEQ ID NO: 1077 - DNA, 23 bp, synthetic construct
  misc_feature 1..23: note = Synthetic oligonucleotide
  source 1..23: mol_type = other DNA
tgccccaagaacaacaaaaaaag

SEQ ID NO: 1078 - DNA, 23 bp, synthetic construct
  misc_feature 1..23: note = Synthetic oligonucleotide
  source 1..23: mol_type = other DNA
tgcctcagtcaaagcacagcagg

SEQ ID NO: 1079 - DNA, 23 bp, synthetic construct
  misc_feature 1..23: note = Synthetic oligonucleotide
  source 1..23: mol_type = other DNA
aaaaccaacaacagtacaaaagg

SEQ ID NO: 1080 - DNA, 23 bp, synthetic construct
  misc_feature 1..23: note = Synthetic oligonucleotide
  source 1..23: mol_type = other DNA
cactccagcctgggcaaaagagg

SEQ ID NO: 1081 - DNA, 23 bp, synthetic construct
  misc_feature 1..23: note = Synthetic oligonucleotide
  source 1..23: mol_type = other DNA
attctgaggaagaaaacaagggg

SEQ ID NO: 1082 - DNA, 23 bp, synthetic construct
  misc_feature 1..23: note = Synthetic oligonucleotide
  source 1..23: mol_type = other DNA
tccccctaccagagcacatacag

SEQ ID NO: 1083 - DNA, 23 bp, synthetic construct
  misc_feature 1..23: note = Synthetic oligonucleotide
  source 1..23: mol_type = other DNA
aggcaaatcaaaaccacaatgag

SEQ ID NO: 1084 - DNA, 23 bp, synthetic construct
  misc_feature 1..23: note = Synthetic oligonucleotide
  source 1..23: mol_type = other DNA
aaaacaagaaagaacaaaagaga

SEQ ID NO: 1085 - DNA, 23 bp, synthetic construct
  misc_feature 1..23: note = Synthetic oligonucleotide
  source 1..23: mol_type = other DNA
cttgccccacagggcagtaacgg

SEQ ID NO: 1086 - DNA, 23 bp, synthetic construct
  misc_feature 1..23: note = Synthetic oligonucleotide
  source 1..23: mol_type = other DNA
cttggcctgcagggcagttatgg

SEQ ID NO: 1087 - DNA, 23 bp, synthetic construct
  misc_feature 1..23: note = Synthetic oligonucleotide
  source 1..23: mol_type = other DNA
tctaccccacatggcagtaatgg

SEQ ID NO: 1088 - DNA, 23 bp, synthetic construct
  misc_feature 1..23: note = Synthetic oligonucleotide
  source 1..23: mol_type = other DNA
actgagcctcagggcagtaatgg

SEQ ID NO: 1089 - DNA, 23 bp, synthetic construct
  misc_feature 1..23: note = Synthetic oligonucleotide
  source 1..23: mol_type = other DNA
cctgccccacagggcaattatgg

SEQ ID NO: 1090 - DNA, 23 bp, synthetic construct
  misc_feature 1..23: note = Synthetic oligonucleotide
  source 1..23: mol_type = other DNA
cctctcccacagggcagtaaagg

SEQ ID NO: 1091 - DNA, 23 bp, synthetic construct
  misc_feature 1..23: note = Synthetic oligonucleotide
  source 1..23: mol_type = other DNA
gctgccccacagggcagcaaagg

SEQ ID NO: 1092 - DNA, 23 bp, synthetic construct
  misc_feature 1..23: note = Synthetic oligonucleotide
  source 1..23: mol_type = other DNA
cctccaatacagggcagtaaagg

SEQ ID NO: 1093 - DNA, 23 bp, synthetic construct
  misc_feature 1..23: note = Synthetic oligonucleotide
  source 1..23: mol_type = other DNA
cctgtcccacagggcaggaaggg

SEQ ID NO: 1094 - DNA, 23 bp, synthetic construct
  misc_feature 1..23: note = Synthetic oligonucleotide
  source 1..23: mol_type = other DNA
ctggcaccacagagcagaaaggg

SEQ ID NO: 1095 - DNA, 23 bp, synthetic construct
  misc_feature 1..23: note = Synthetic oligonucleotide
  source 1..23: mol_type = other DNA
catgctccacagagcagcaaagg

SEQ ID NO: 1096 - DNA, 23 bp, synthetic construct
  misc_feature 1..23: note = Synthetic oligonucleotide
  source 1..23: mol_type = other DNA
gggctgccccagggcagtaatgg

SEQ ID NO: 1097 - DNA, 23 bp, synthetic construct
  misc_feature 1..23: note = Synthetic oligonucleotide
  source 1..23: mol_type = other DNA
cttgctgcacaggacaataaagg

SEQ ID NO: 1098 - DNA, 23 bp, synthetic construct
  misc_feature 1..23: note = Synthetic oligonucleotide
  source 1..23: mol_type = other DNA
ctcgcccctcagggcagtagtgg

SEQ ID NO: 1099 - DNA, 23 bp, synthetic construct
  misc_feature 1..23: note = Synthetic oligonucleotide
  source 1..23: mol_type = other DNA
gttggccctcagggcagaaatgg

SEQ ID NO: 1100 - DNA, 23 bp, synthetic construct
  misc_feature 1..23: note = Synthetic oligonucleotide
  source 1..23: mol_type = other DNA
gaggcgccacagggcagtaatgg

SEQ ID NO: 1101 - DNA, 23 bp, synthetic construct
  misc_feature 1..23: note = Synthetic oligonucleotide
  source 1..23: mol_type = other DNA
gctgtgtcatagggcagtaacgg

SEQ ID NO: 1102 - DNA, 23 bp, synthetic construct
  misc_feature 1..23: note = Synthetic oligonucleotide
  source 1..23: mol_type = other DNA
ctttcttcacagggtagtaatgg

SEQ ID NO: 1103 - DNA, 23 bp, synthetic construct
  misc_feature 1..23: note = Synthetic oligonucleotide
  source 1..23: mol_type = other DNA
tgccccagacagggcagtaaggg

SEQ ID NO: 1104 - DNA, 23 bp, synthetic construct
  misc_feature 1..23: note = Synthetic oligonucleotide
  source 1..23: mol_type = other DNA
cttgcactacaagtcagtaatgg

SEQ ID NO: 1105 - DNA, 23 bp, synthetic construct
  misc_feature 1..23: note = Synthetic oligonucleotide
  source 1..23: mol_type = other DNA
atttcctcacagggcagaaaagg

SEQ ID NO: 1106 - DNA, 23 bp, synthetic construct
  misc_feature 1..23: note = Synthetic oligonucleotide
  source 1..23: mol_type = other DNA
tcacccccacaggccagtaaagg

SEQ ID NO: 1107 - DNA, 23 bp, synthetic construct
  misc_feature 1..23: note = Synthetic oligonucleotide
  source 1..23: mol_type = other DNA
gtcatgtcacagggcagtagtgg

SEQ ID NO: 1108 - DNA, 23 bp, synthetic construct
  misc_feature 1..23: note = Synthetic oligonucleotide
  source 1..23: mol_type = other DNA
ggccctgcccagggcagtaatgg

SEQ ID NO: 1109 - DNA, 23 bp, synthetic construct
  misc_feature 1..23: note = Synthetic oligonucleotide
  source 1..23: mol_type = other DNA
cttaatacacagggaaggaatgg

SEQ ID NO: 1110 - DNA, 23 bp, synthetic construct
  misc_feature 1..23: note = Synthetic oligonucleotide
  source 1..23: mol_type = other DNA
cttcaagagcaacagtgctgtgg

SEQ ID NO: 1111 - DNA, 23 bp, synthetic construct
  misc_feature 1..23: note = Synthetic oligonucleotide
  source 1..23: mol_type = other DNA
gagagacagcaacagtgctatgg

SEQ ID NO: 1112 - DNA, 23 bp, synthetic construct
  misc_feature 1..23: note = Synthetic oligonucleotide
  source 1..23: mol_type = other DNA
agcaaggagcaacagtgatgtgg

SEQ ID NO: 1113 - DNA, 23 bp, synthetic construct
  misc_feature 1..23: note = Synthetic oligonucleotide
  source 1..23: mol_type = other DNA
agcaaacatcaacagtgctgagg

SEQ ID NO: 1114 - DNA, 23 bp, synthetic construct
  misc_feature 1..23: note = Synthetic oligonucleotide
  source 1..23: mol_type = other DNA
taggaagagcaacagggctgtgg

SEQ ID NO: 1115 - DNA, 23 bp, synthetic construct
  misc_feature 1..23: note = Synthetic oligonucleotide
  source 1..23: mol_type = other DNA
catgaagggcaacagagctgagg

SEQ ID NO: 1116 - DNA, 23 bp, synthetic construct
  misc_feature 1..23: note = Synthetic oligonucleotide
  source 1..23: mol_type = other DNA
cactctaagcaacagtgctgggg

SEQ ID NO: 1117 - DNA, 23 bp, synthetic construct
  misc_feature 1..23: note = Synthetic oligonucleotide
  source 1..23: mol_type = other DNA
tgcgaggagcaacagtgcttggg

SEQ ID NO: 1118 - DNA, 23 bp, synthetic construct
  misc_feature 1..23: note = Synthetic oligonucleotide
  source 1..23: mol_type = other DNA
gtctctaggcaacagtgctgagg

SEQ ID NO: 1119 - DNA, 23 bp, synthetic construct
  misc_feature 1..23: note = Synthetic oligonucleotide
  source 1..23: mol_type = other DNA
gggcagcagctacagtgctgagg

SEQ ID NO: 1120 - DNA, 23 bp, synthetic construct
  misc_feature 1..23: note = Synthetic oligonucleotide
  source 1..23: mol_type = other DNA
gggcggtgctacaactgggctgg

SEQ ID NO: 1121 - DNA, 23 bp, synthetic construct
  misc_feature 1..23: note = Synthetic oligonucleotide
  source 1..23: mol_type = other DNA
gggtggttctacaaccaggctgg

SEQ ID NO: 1122 - DNA, 23 bp, synthetic construct
  misc_feature 1..23: note = Synthetic oligonucleotide
  source 1..23: mol_type = other DNA
gggcggtgctacaactgggctgg

SEQ ID NO: 1123 - DNA, 23 bp, synthetic construct
  misc_feature 1..23: note = Synthetic oligonucleotide
  source 1..23: mol_type = other DNA
gggaggtgccacatcagggccgg

SEQ ID NO: 1124 - DNA, 23 bp, synthetic construct
  misc_feature 1..23: note = Synthetic oligonucleotide
  source 1..23: mol_type = other DNA
gggcagtgatccaactgtgcagg

SEQ ID NO: 1125 - DNA, 23 bp, synthetic construct
  misc_feature 1..23: note = Synthetic oligonucleotide
  source 1..23: mol_type = other DNA
agctggggctacatctgggctgg

SEQ ID NO: 1126 - DNA, 23 bp, synthetic construct
  misc_feature 1..23: note = Synthetic oligonucleotide
  source 1..23: mol_type = other DNA
gctgggtgctacaacagggcagg

SEQ ID NO: 1127 - DNA, 23 bp, synthetic construct
  misc_feature 1..23: note = Synthetic oligonucleotide
  source 1..23: mol_type = other DNA
ctgtggtgcaacaactgggctgg

SEQ ID NO: 1128 - DNA, 23 bp, synthetic construct
  misc_feature 1..23: note = Synthetic oligonucleotide
  source 1..23: mol_type = other DNA
gggaggaggtacaactgggaggg

SEQ ID NO: 1129 - DNA, 23 bp, synthetic construct
  misc_feature 1..23: note = Synthetic oligonucleotide
  source 1..23: mol_type = other DNA
cagtgtggctacaactgcgcagg

SEQ ID NO: 1130 - DNA, 23 bp, synthetic construct
  misc_feature 1..23: note = Synthetic oligonucleotide
  source 1..23: mol_type = other DNA
ctggtcagctacaactggcctgg

SEQ ID NO: 1131 - DNA, 23 bp, synthetic construct
  misc_feature 1..23: note = Synthetic oligonucleotide
  source 1..23: mol_type = other DNA
ggtgtaaaatcaacaccctaagg

SEQ ID NO: 1132 - DNA, 23 bp, synthetic construct
  misc_feature 1..23: note = Synthetic oligonucleotide
  source 1..23: mol_type = other DNA
gctggaaaaaaaacaccctaggg

SEQ ID NO: 1133 - DNA, 23 bp, synthetic construct
  misc_feature 1..23: note = Synthetic oligonucleotide
  source 1..23: mol_type = other DNA
gaggtaaaaccaacaccttaagg

SEQ ID NO: 1134 - DNA, 23 bp, synthetic construct
  misc_feature 1..23: note = Synthetic oligonucleotide
  source 1..23: mol_type = other DNA
tggctgaaatcaacaccccaggg

SEQ ID NO: 1135 - DNA, 23 bp, synthetic construct
  misc_feature 1..23: note = Synthetic oligonucleotide
  source 1..23: mol_type = other DNA
tgacaccaatcaacaccttaagg

SEQ ID NO: 1136 - DNA, 23 bp, synthetic construct
  misc_feature 1..23: note = Synthetic oligonucleotide
  source 1..23: mol_type = other DNA
tctgatccatcaacaccctatgg

(19) United States
(12) Patent Application Publication
(10) Pub. No.: US 2026/0132463 A1
(43) Pub. Date: May 14, 2026

(54) METHODS OF ENRICHING TARGETED NUCLEIC ACID, IDENTIFYING OFF-TARGET AND EVALUATING GENE EDITING EFFICIENCY

(71) Applicant: GenEditBio Limited, Hong Kong (CN)

(72) Inventors: Wenjing ZHOU, Hong Kong (CN); Bang WANG, Hong Kong (CN); Zongli ZHENG, Hong Kong (CN)

(21) Appl. No.: 19/445,281

(22) Filed: Jan. 9, 2026

Related U.S. Application Data
(62) Division of application No. 18/510,106, filed on Nov. 15, 2023, now Pat. No. 12,545,958.
Application No. 18/510,106 is a continuation of application No. PCT/IB2022/000278, filed on May 16, 2022.
(60) Provisional application No. 63/201,861, filed on May 16, 2021.
(60) Provisional application No. 63/277,782, filed on Nov. 10, 2021.

(51) Int. Cl. C12Q 1/6876; C12Q 1/6811; C12Q 1/6855; C12Q 1/6874

(52) U.S. Cl. CPC C12Q 1/6876; C12Q 1/6855; C12Q 1/6874; C12Q 1/6811; C12Q 2600/16

(57) ABSTRACT

The present disclosure relates to enriching nucleic acid from a sample. In some embodiments, the present disclosure provides methods for enriching at least one targeted nucleic acid, identifying genome-wide gene editing off-targets, and evaluating gene editing efficiency from a sample comprising a plurality of single-strand nucleic acid fragments. Others example embodiments are also described herein.

### CROSS-REFERENCE

This application is a divisional of U.S. application Ser. No. 18/510,106, filed on Nov. 15, 2023, which is a continuation of International Application No. PCT/IB2022/000278, filed on May 16, 2022, which claims the benefit of U.S. Provisional Application No. 63/201,861, filed on May 16, 2021 and 63/277,782, filed on Nov. 10, 2021, each of which applications is incorporated herein by reference in its entirety for all purposes.

### DESCRIPTION OF THE TEXT FILE SUBMITTED ELECTRONICALLY

The contents of the electronic sequence listing (GEBL_001_03US_SeqList_ST26.xml; Size: 1,479,081 bytes; and Date of Creation: Jan. 9, 2026) are herein incorporated by reference in its entirety.

### FIELD

The present disclosure generally relates to enriching nucleic acid, identifying genome-wide gene editing off-targets, and evaluating gene editing efficiency.

### BACKGROUND

Genome-targeting, programmable nucleases such as ZFNs, TALENs and CRISPR are profoundly revolutionizing the community of genetic engineering and precise gene therapy. However, unwanted edits within genome (i.e., off-target effect) may cause unpredictable confounding results in research and severe side-effects in gene therapy. Detecting off-target, therefore, represents a necessary checkpoint for ensuring the precision of genome editing. Current off-target profiling methods have various disadvantages, such as being incompatible with in vivo editing, requiring high amounts of sample input, and being time-consuming if a validation is to be conducted. In addition, sensitivity and specificity of the current methods may fluctuate uncontrollably in outcome.

Some current methods employ a multiplex target enrichment using forward and reverse primers. The drawback of these methods is that unknown sequences contiguous to the target sequences cannot be enriched. The forward and reverse primer generated data has identical start and end positions, posing significant challenge in the data analysis of counting molecular complexing, controlling sequencing error, and calculating copy numbers and efficiency.

### SUMMARY

In one aspect, provided herein is a method of enriching at least one target nucleic acid from a sample comprising a plurality of single-strand nucleic acid fragments, the method comprising: (a) contacting a universal oligonucleotide adaptor with the sample to produce a ligation product, wherein the universal oligonucleotide adaptor is configured for ligating to a 5′ end of the single-strand nucleic acid fragments; (b) amplifying the ligation product by a first PCR with a first target-specific primer and optionally a first universal oligonucleotide adaptor primer to form a first PCR product; and (c) amplifying the first PCR product by a second PCR with a second target-specific primer and a second universal oligonucleotide adaptor primer to form a second PCR product, wherein the second target-specific primer is nested relative to the first target-specific primer.

In some embodiments, prior to (a), the method further comprises at least one of: blocking a 3′ end of the single-strand nucleic acid fragments; phosphorylating a 5′ end of the single-strand nucleic acid fragments; or adenylating the nucleic acid to produce a 3′-adenosine overhang on the single-strand nucleic acid fragments.

In some embodiments, the first PCR is a linear amplification of the ligation product with the first target-specific primer to obtain a nascent primer extension duplex. In some embodiments, the first PCR is an exponential amplification of the targeted nucleic acid with the first target-specific primer and the first universal oligonucleotide adaptor primer. In some embodiments, the first universal oligonucleotide adaptor primer and the second universal oligonucleotide adaptor primer are the same. In other embodiments, the first universal oligonucleotide adaptor primer and the second universal oligonucleotide adaptor primer are different.

In some embodiments, the universal oligonucleotide adaptor comprises: a 3′ recessive end, the 3′ recessive end configured for ligating to the 5′ end of the single-strand nucleic acid fragments; and/or a 5′ protrude end comprising three to twenty bases of random or degenerate nucleotides. In some embodiments, a duplex portion of the universal oligonucleotide adaptor is of sufficient length to remain in duplex form in (a). In some embodiments, the universal oligonucleotide adaptor comprises a hairpin loop connecting a portion of the duplex form. In some embodiments, the universal oligonucleotide adaptor comprises a single strand segment comprising three to twenty random nucleotides as a unique molecular index (UMI) for tracing individual original molecules.

In some embodiments, (c) further comprises forming a sequencing library with a sequencing specific adaptor pair. In some specific embodiments, after (c), further comprises sequencing the sequencing library using a sequencing primer pair, wherein the sequencing primer pair is at least partially complementary to opposite strands of the second PCR product, respectively.

In some embodiments, the sample comprises a plurality of DNA fragments prepared from high molecular weight DNA. In some embodiments, the plurality of single-strand nucleic acid fragments are prepared from denaturation of double-strand DNA fragments. In some embodiments, the sample comprises single-strand cDNA fragments prepared from reverse transcription of RNA fragments. In some embodiments, the method further comprises of analyzing the plurality of nucleic acids fragments. In some embodiments, the first PCR and/or second PCR are multiplexing PCR.

In some embodiments, the sample is from a mammal, and wherein optionally the sample is from human. In some specific embodiments, the human is an individual known to have or suspected of having a disease, and wherein optionally the disease is a cancer or a genetic disorder. In some embodiments, one or more of the target nucleic acids comprise one or more markers for the cancer. In some embodiments, the human is a fetus. In some embodiments, the sample is from a blood sample. In some embodiments, the sample comprises cell-free nucleic acids extracted from a blood sample. In some embodiments, the sample comprises nucleic acids extracted from circulating tumor cells. In some embodiments, the sample comprises nucleic acids extracted from lymphocytes in a blood sample for T-cell and B-cell receptor profiling. In some embodiments, the sample is a CRISPR gene edited sample. In some specific embodiments, the sample is meganucleases edited, zinc finger nucleases (ZFNs) edited, or transcription activator-like effector nucleases (TALENs) edited. In some embodiments, the sample is from CAR-T, CAR-NK, TCR-T, immortalized cell lines (e.g., engineered neural stem cell line CTX) or hematopoietic stem cells for therapeutics. In some embodiments, the sample is from genetically engineered cells (ex-vivo or in vivo), wherein the cells include but are not limited to fibroblasts, chondrocytes, keratinocytes, hepatocytes, pancreatic islet cells, stem cells (e.g., haematopoietic stem cells, mesenchymal stem cells, or skin stem cells), and immune cells (e.g., tumor infiltrating lymphocytes, viral reconstitution T cells, dendritic cells, γδ T cells, regulatory T cells (Treg) and macrophages).

In another aspect, provided herein is a method of enriching at least one target nucleic acid from a sample comprising a plurality of single-strand nucleic acid fragments, the method comprising: (a) ligating a universal oligonucleotide adaptor to a 5′ end of the single-strand nucleic acid fragments; (b) annealing a first target-specific primer to the single-strand nucleic acid fragments in the vicinity of a target sequence; (c) extending the first target-specific primer over the single-strand nucleic acid fragments using a DNA polymerase; (d) obtaining a nascent primer extension duplex; (e) dissociating the nascent primer extension duplex into single strands; and (f) amplifying a portion of the single stands of the nascent primer extension duplex with a second target-specific primer and a universal oligonucleotide adaptor primer.

In some embodiments, prior to (a), the method further comprises at least one of: blocking a 3′ end of the single-strand nucleic acid fragments; phosphorylating a 5′ end of the single-strand nucleic acid fragments; or adenylating the nucleic acid to produce a 3-adenosine overhang on the single-strand nucleic acid fragments.

In some embodiments, the universal oligonucleotide adaptor comprises: a 3′ recessive end, the 3′ recessive end configured for ligating to the 5′ end of the single-strand nucleic acid fragments; and/or a 5′ protrude end comprising three to twenty bases of random or degenerate nucleotides; wherein a duplex portion of the universal oligonucleotide adaptor is of sufficient length to remain in duplex form in (a). In some embodiments, the universal oligonucleotide adaptor comprises a hairpin loop connecting a portion of the duplex form. In some embodiments, (f) further comprises forming a sequencing library with a sequencing specific adaptor pair. In some embodiments, the method, after (f), further comprises sequencing the sequencing library using a sequencing primer pair, wherein the sequencing primer pair is at least partially complementary to opposite strands of the second PCR product, respectively. In some embodiments, the method further comprises repeating (b)-(f) for one or more cycles.

In some embodiments, the sample comprises a plurality of DNA fragments prepared from high molecular weight DNA. In some embodiments, the plurality of single-strand nucleic acid fragments are prepared from denaturation of double-strand DNA fragments. In some embodiments, the sample comprises single-strand cDNA fragments prepared from reverse transcription of RNA fragments. In some embodiments, the universal oligonucleotide adaptor comprises a single strand segment comprising three to twenty random nucleotides as a unique molecular index (UMI) for tracing individual original molecules. In some embodiments, the method further comprises analyzing the plurality of nucleic acids fragments.

In some embodiments, the sample is from a mammal, and wherein optionally the mammal is a human. In some embodiments, the human is an individual known to have or suspected of having a disease, and wherein optionally the disease is a cancer or a genetic disorder. In some embodiments, the human is a fetus.

In some embodiments, the sample is from a blood sample. In other embodiments, the sample comprises cell-free nucleic acids extracted from a blood sample. In other embodiments, the sample comprises nucleic acids extracted from circulating tumor cells. In other embodiments, the sample comprises nucleic acids extracted from lymphocytes in a blood sample for T-cell and B-cell receptor profiling. In other embodiments, the sample is a CRISPR gene edited sample. In some specific embodiments, the sample is meganucleases edited, zinc finger nucleases (ZFNs) edited, or transcription activator-like effector nucleases (TALENs) edited. In some embodiments, the sample is from CAR-T, CAR-NK, TCR-T, immortalized cell lines (e.g., engineered neural stem cell line CTX) or hematopoietic stem cells for therapeutics. In some embodiments, the sample is from genetically engineered cells (ex-vivo or in vivo), wherein the cells include but are not limited to fibroblasts, chondrocytes, keratinocytes, hepatocytes, pancreatic islet cells, stem cells (e.g., haematopoietic stem cells, mesenchymal stem cells, or skin stem cells), and immune cells (e.g., tumor infiltrating lymphocytes, viral reconstitution T cells, dendritic cells, γδ T cells, regulatory T cells (Treg) and macrophages).

In another aspect, provided herein is a method of identifying genome-wide gene editing off-target sites from a sample comprising a plurality of single-strand nucleic acid fragments, comprising: (a) contacting a universal oligonucleotide adaptor with the sample to produce a ligation product, wherein the universal oligonucleotide adaptor is configured for ligating to a 5′ end of the single-strand nucleic acid fragments; (b) amplifying the ligation product by performing a first PCR with a first target-specific primer to form a first PCR product; (c) amplifying the first PCR product by a second PCR with a sequencing specific adaptor primer and a second target-specific primer nested relative to the first target-specific primer, to form a sequencing library; (d) quantifying and reading the sequencing library to obtain sequencing results; and (e) mapping the sequencing results to a reference genome.

In another aspect, provided herein is a method of evaluating gene editing efficiency from a sample comprising a plurality of single-strand nucleic acid fragments, comprising: (a) contacting a universal oligonucleotide adaptor with the sample to produce a ligation product, wherein the universal oligonucleotide adaptor is configured for ligating to a 5′ end of the single-strand nucleic acid fragments; (b) amplifying the ligation product by performing a first PCR with a first target-specific primer to form a first PCR product, wherein the first target-specific primer is configured for annealing to the single-strand nucleic acid fragments at an on-target site, a predicted off-target site, or a known off-target sites; (c) amplifying the first PCR product by a second PCR with a sequencing specific adaptor primer and a second target-specific primer nested relative to the first target-specific primer, to form a sequencing library; (d) quantifying and reading the sequencing library to form sequencing results; and (e) mapping the sequencing results to a reference genome and evaluating gene editing efficiency.

In some embodiments, the predicted off-target site is predicted in silico based on softwares comprising E-CRISP, Cas-OFFinder, and/or CRISPRscan. In some embodiments, the E-CRISP has a cutoff of mismatch <=10, 9, 8, 7, or 6; the Cas-OFFinder has a mismatch <=6, 5, 4, 3, or 2 and a bulge <=3, 2, or 1; and the CRISPRscan has no threshold. In some embodiments, (e) further comprises: detecting translocation by obtaining a split read and a discordant read or determining an insertion and deletion (indel) frequency. In some specific embodiments, the split read and the discordant read are obtained by: identifying potential candidate translocations and estimating protospacer similarity to an on-target spacer and a cutting frequency determinant (CFD). In some specific embodiments, the indel frequency is obtained by: (a) aligning the mapped results by GATK-realigner to form aligned results; (b) filtering the aligned results not spanning a corresponding spacer region; (c) predicting an insertion and deletion occurring around 5-bp upstream or downstream of a cleavage site; and (d) determining the reliable indel frequency by an indel value of the sample with an elimination by a corresponding value of a negative control.

In another aspect, provided herein is a method of identifying genome-wide gene editing off-target sites from a sample comprising a plurality of single-strand nucleic acid fragments, comprising: (a) contacting a universal oligonucleotide adaptor with the sample to produce a ligation product, wherein the universal oligonucleotide adaptor is configured for ligating to a 5′ end of the single-strand nucleic acid fragments; (b) amplifying the ligation product by a first PCR with a first set of target-specific primers, wherein the target-specific primers in the first set are configured for annealing to the single-strand nucleic acid fragments 5′ of an on-target site and one or more predicted and/or known off-target sites; (c) amplifying the first PCR product by a second PCR with a second set of target-specific primers and a universal oligonucleotide adaptor primer to form a sequencing library, wherein each member of the second set of target-specific primers is nested relative to a corresponding primer of the first set of target-specific primers; and (d) sequencing the sequencing library to identify off-target sites.

In some embodiments, the predicted off-target sites in (b) are computationally predicted off-target sites. In some embodiments, the computationally predicted off-target sites are top 100, 90, 80, 70, 60, 50, 40, 30, 20, or 10 off-target sites predicted based on software comprising E-CRISP, Cas-OFFinder, or CRISPRscan. In some specific embodiments, the E-CRISP has a cutoff of mismatch <=10, 9, 8, 7, or 6; the Cas-OFFinder has a mismatch <=6, 5, 4, 3, or 2 and a bulge <=3, 2, or 1; and the CRISPRscan has no threshold.

In some embodiments, method further comprises: detecting translocation by obtaining a split read and a discordant read or determining an insertion and deletion (indel) frequency. In some specific embodiments, the split read and the discordant read are obtained by: identifying potential candidate translocations and estimating protospacer similarity to an on-target spacer and a cutting frequency determinant (CFD). In some specific embodiments, the indel frequency is obtained by: aligning the mapped results by GATK-realigner to form aligned results; filtering the aligned results not spanning a corresponding spacer region; predicting an insertion and deletion occurring around 5-bp upstream or downstream of a cleavage site; and determining the reliable indel frequency by an indel value of the sample with an elimination by a corresponding value of a negative control.

In some embodiments, prior to (a), the method further comprises at least one of: blocking a 3′ end of the single-strand nucleic acid fragments; phosphorylating a 5′ end of the single-strand nucleic acid fragments; or adenylating the nucleic acid to produce a 3′-adenosine overhang on the single-strand nucleic acid fragments.

In some embodiments, the universal oligonucleotide adaptor comprises: a 3′ recessive end, the 3′ recessive end configured for ligating to the 5′ end of the single-strand nucleic acid fragments; and/or a 5′ protrude end comprising three to twenty bases of random or degenerate nucleotides. In some embodiments, a duplex portion of the universal oligonucleotide adaptor is of sufficient length to remain in duplex form in (a). In some specific embodiments, the universal oligonucleotide adaptor comprises a hairpin loop connecting a portion of the duplex form. In some embodiments, the universal oligonucleotide adaptor comprises a single strand segment comprising three to twenty random nucleotides as a unique molecular index (UMI) for tracing individual original molecules. In some embodiments, (c) further comprises forming the sequencing library with a sequencing specific adaptor pair. In some embodiments, after (c), further comprises: sequencing the sequencing library using a sequencing primer pair, wherein the sequencing primer pair is at least partially complementary to opposite strands of the second PCR product, respectively. In some embodiments, the sample comprises a plurality of DNA fragments prepared from high molecular weight DNA. In some embodiments, the plurality of single-strand nucleic acid fragments are prepared from denaturation of double-strand DNA fragments.

In some embodiments, the sample comprises single-strand cDNA fragments prepared from reverse transcription of RNA fragments.

In some embodiments, the method further comprises analyzing the plurality of nucleic acids fragments.

In some embodiments, the sample is from a mammal, and wherein optionally the mammal is a human. In some specific embodiments, the human is an individual known to have or suspected of having a disease, and wherein optionally the disease is a cancer or a genetic disorder. In some embodiments, one or more of the target nucleic acids comprise one or more markers for the cancer. In some specific embodiments, the human is a fetus. In some embodiments, the sample is from a blood sample. In some embodiments, the sample comprises cell-free nucleic acids extracted from a blood sample. In some embodiments, the sample comprises nucleic acids extracted from circulating tumor cells. In some embodiments, the sample comprises nucleic acids extracted from lymphocytes in a blood sample for T-cell and B-cell receptor profiling. In some embodiments, the sample is a CRISPR gene edited sample. In some specific embodiments, the sample is meganucleases edited, zinc finger nucleases (ZFNs) edited, or transcription activator-like effector nucleases (TALENs) edited. In some embodiments, the sample is from CAR-T, CAR-NK, TCR-T, immortalized cell lines (e.g., engineered neural stem cell line CTX) or hematopoietic stem cells for therapeutics. In some embodiments, the sample is from genetically engineered cells (ex-vivo or in vivo), wherein the cells include but are not limited to fibroblasts, chondrocytes, keratinocytes, hepatocytes, pancreatic islet cells, stem cells (e.g., haematopoietic stem cells, mesenchymal stem cells, or skin stem cells), and immune cells (e.g., tumor infiltrating lymphocytes, viral reconstitution T cells, dendritic cells, γδ T cells, regulatory T cells (Treg) and macrophages).

### BRIEF DESCRIPTION OF FIGURES

The patent or application file contains at least one drawing executed in color. Copies of this patent or patent application publication with color drawing(s) will be provided by the Office upon request and payment of the necessary fee.

FIG. 1A is a schematic diagram which illustrates an example embodiment of a workflow for amplifying targeted nucleic acid from a sample.

FIG. 1B is a schematic diagram which illustrates another example embodiment of a workflow for amplifying targeted nucleic acid from a sample.

FIG. 2A and FIG. 2B are charts which show the off-target identification and validation (Chart 210, SEQ ID NOs: 961-1022 and Chart 210′, SEQ ID NOs: 1023-1054) using an example technique described in the present disclosure, namely EDITED-Seq, at VEGFA_2 locus edited by CRISPR-Cas9, according to an example embodiment.

FIG. 2C is a diagram which shows the correlation between EDITED-Seq score (Escore) and Indel frequencies (%), according to the same example embodiment of FIG. 2A and FIG. 2B.

FIG. 2D is a diagram which shows the detection titration of input genomic DNA at VEGFA_2 locus, according to the same example embodiment of FIG. 2A and FIG. 2B.

FIG. 2E is a diagram which shows a translocation circus plot of VEGFA_2 within chromosome coordinate, according to the same example embodiment of FIG. 2A and FIG. 2B.

FIG. 3A is a Venn diagram which shows a comparison between EDITED-Seq off-target profile and GUIDE-Seq and DISCOVER-Seq in detection of off-targets at VEGFA_2 locus, according to the example embodiment of FIGS. 2A-2E.

FIG. 3B is a diagram which shows a rank comparison of the commonly identified 35 sites based on the corresponding scoring values, e.g. Escore, GUIDE-Seq count, DISCOVER score, according to the same example embodiment of FIG. 3A.

FIG. 3C is a diagram which shows Paranal distributions of identified (true) and missed (false) off-targets of EDITED-Seq, compared to GUIDE-Seq and DISCOVER-Seq, according to the same example embodiment of FIG. 3A.

FIG. 3D is an exemplary result of deep amplicon sequencing shown in Integrated Genome Viewer, indicating additional off-target insertions (shown as “I”) and deletions in chromosome 10 were detected by EDITED-Seq, but not by DISCOVER-Seq or GUIDE-Seq (reference nucleotide sequence SEQ ID NO: 1055).

FIG. 3E is an exemplary result of deep amplicon sequencing shown in Integrated Genome Viewer, indicating additional off-target insertions (shown as “I”) and deletions in chromosome 17 were detected by EDITED-Seq, but not by DISCOVER-Seq or GUIDE-Seq.

FIG. 3F is an exemplary result of deep amplicon sequencing shown in Integrated Genome Viewer, indicating additional off-target insertions (shown as “I”) and deletions in chromosome 22 were detected by EDITED-Seq, but not by DISCOVER-Seq or GUIDE-Seq.

FIG. 3G is an exemplary result of deep amplicon sequencing shown in Integrated Genome Viewer, indicating additional off-target insertions (shown as “I”) and deletions in chromosome 11 were detected by EDITED-Seq, but not by DISCOVER-Seq or GUIDE-Seq (reference nucleotide sequence SEQ ID NO: 1056; reference amino acid sequence SEQ ID no: 1057).

FIG. 3H is an exemplary result of deep amplicon sequencing shown in Integrated Genome Viewer, indicating additional off-target insertions (shown as “I”) and deletions in chromosome 12 were detected by EDITED-Seq, but not by DISCOVER-Seq or GUIDE-Seq (reference nucleotide sequence SEQ ID NO: 1058).

FIG. 3I is an exemplary result of deep amplicon sequencing shown in Integrated Genome Viewer, indicating additional translocation in chromosome 7 were detected by EDITED-Seq, but not by DISCOVER-Seq or GUIDE-Seq (reference nucleotide sequence SEQ ID NO: 1059).

FIG. 3J is a cricos plot illustrating the translocation events detected by one set of primers for the on-target site of VEGFA_2.

FIG. 3K is a cricos plot illustrating the translocation events detected by 1 off-target site predicted in-silicon in CRISPR-Cas9 targeting VEGFA_2 locus. FIG. 3L is a cricos plot illustrating the translocation events detected by 2 off-target sites predicted in-silicon in CRISPR-Cas9 targeting VEGFA_2 locus. FIG. 3M is a cricos plot illustrating the translocation events detected by 3 off-target sites predicted in-silicon in CRISPR-Cas9 targeting VEGFA_2 locus. FIG. 3N is a cricos plot illustrating the translocation events detected by 4 off-target sites predicted in-silicon in CRISPR-Cas9 targeting VEGFA_2 locus. FIG. 3O is a cricos plot illustrating the translocation events detected by 5 off-target sites predicted in-silicon in CRISPR-Cas9 targeting VEGFA_2 locus. FIG. 3P is a cricos plot illustrating the translocation events detected by 6 off-target sites predicted in-silicon in CRISPR-Cas9 targeting VEGFA_2 locus. FIG. 3Q is a cricos plot illustrating the translocation events detected by 7 off-target sites predicted in-silicon in CRISPR-Cas9 targeting VEGFA_2 locus. FIG. 3R is a cricos plot illustrating the translocation events detected by 8 off-target sites predicted in-silicon in CRISPR-Cas9 targeting VEGFA_2 locus. FIG. 3S is a cricos plot illustrating the translocation events detected by 9 off-target sites predicted in-silicon in CRISPR-Cas9 targeting VEGFA_2 locus. FIG. 3T is a cricos plot illustrating the translocation events detected by 10 off-target sites predicted in-silicon in CRISPR-Cas9 targeting VEGFA_2 locus. FIG. 3U is a cricos plot illustrating the translocation events detected by 11 off-target sites predicted in-silicon in CRISPR-Cas9 targeting VEGFA_2 locus. FIG. 3V is a cricos plot illustrating the translocation events detected by 12 off-target sites predicted in-silicon in CRISPR-Cas9 targeting VEGFA_2 locus. FIG. 3W is a cricos plot illustrating the translocation events detected by 13 off-target sites predicted in-silicon in CRISPR-Cas9 targeting VEGFA_2 locus. FIG. 3X is a cricos plot illustrating the translocation events detected by 14 off-target sites predicted in-silicon in CRISPR-Cas9 targeting VEGFA_2 locus. FIG. 3Y is a cricos plot illustrating the translocation events detected by 15 off-target sites predicted in-silicon in CRISPR-Cas9 targeting VEGFA_2 locus. FIG. 3Z is a cricos plot illustrating the translocation events detected by 16 off-target sites predicted in-silicon in CRISPR-Cas9 targeting VEGFA_2 locus. FIG. 3AA is a cricos plot illustrating the translocation events detected by 17 off-target sites predicted in-silicon in CRISPR-Cas9 targeting VEGFA_2 locus. FIG. 3AB is a cricos plot illustrating the translocation events detected by 18 off-target sites predicted in-silicon in CRISPR-Cas9 targeting VEGFA_2 locus. FIG. 3AC is a cricos plot illustrating the translocation events detected by 19 off-target sites predicted in-silicon in CRISPR-Cas9 targeting VEGFA_2 locus. FIG. 3AD is a cricos plot illustrating the translocation events detected by 20 off-target sites predicted in-silicon in CRISPR-Cas9 targeting VEGFA_2 locus.

FIG. 4A is a schematic diagram which shows a workflow of iPSC editing by CRISPR-Cas9, according to an example embodiment.

FIG. 4B is a schematic diagram which shows a workflow of primary T-cell editing by CRISPR-Cas9, according to an example embodiment.

FIG. 4C is a chart which show off-target sites (Chart 411, SEQ ID NOs: 1060-1084; Chart 412, SEQ ID NOs: 1085-1109) in the iPSC at GAPDH and HBB sites, according to the same example embodiment of FIG. 4A.

FIG. 4D is a chart which shows off-targets (Chart 421, SEQ ID NOs: 1110-1119; Chart 422, SEQ ID NOs: 1120-1130) in the T-cell at TRAC and PD-1 sites, according to the same example embodiment of FIG. 4B.

FIG. 5A is a schematic diagram which illustrates a workflow of EDITED-Seq conducted in a mouse, according to an example embodiment.

FIG. 5B and FIG. 5C are charts which show off-targets (Chart 520, SEQ ID NOs: 1131-1136; Chart 530, SEQ ID NOs: 1131-1134) in a mouse at ALB site after 15 or 60 days, respectively, according to the same example embodiment of FIG. 5A.

FIG. 6 is a schematic diagram which illustrates the topology of a lentiCRISPR vector.

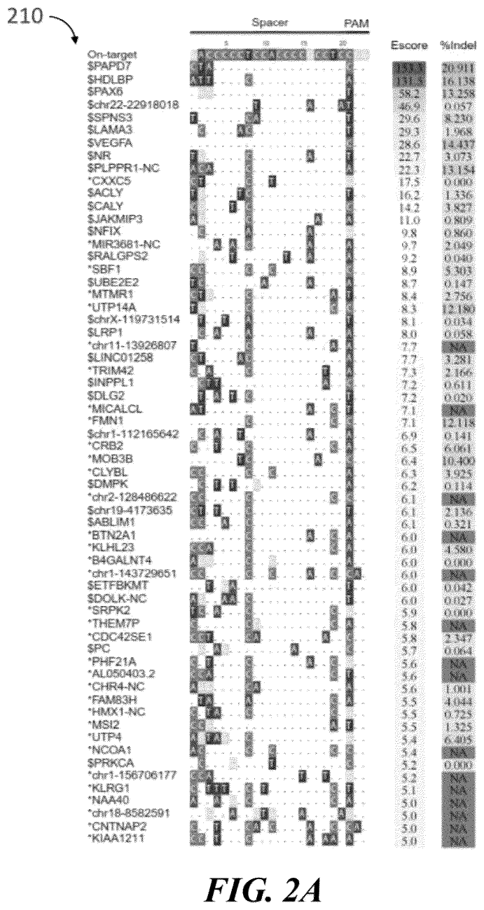

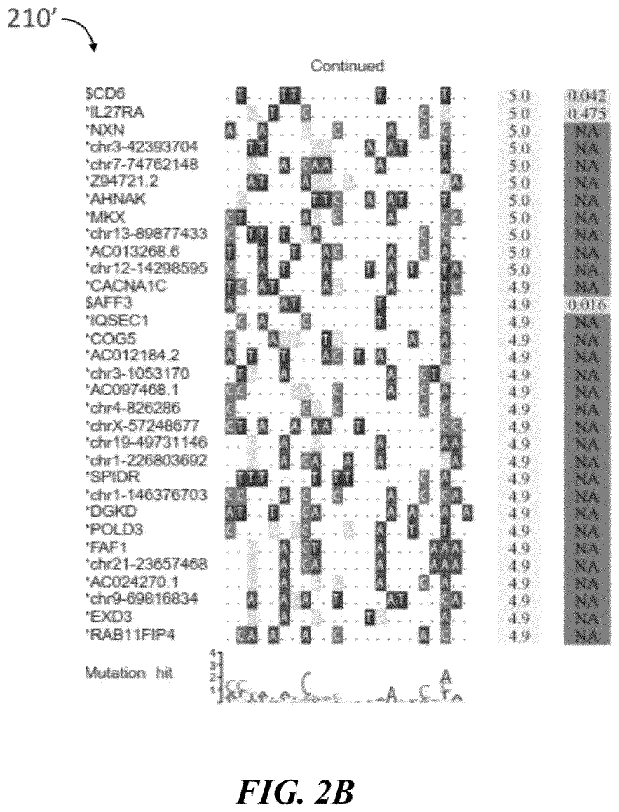

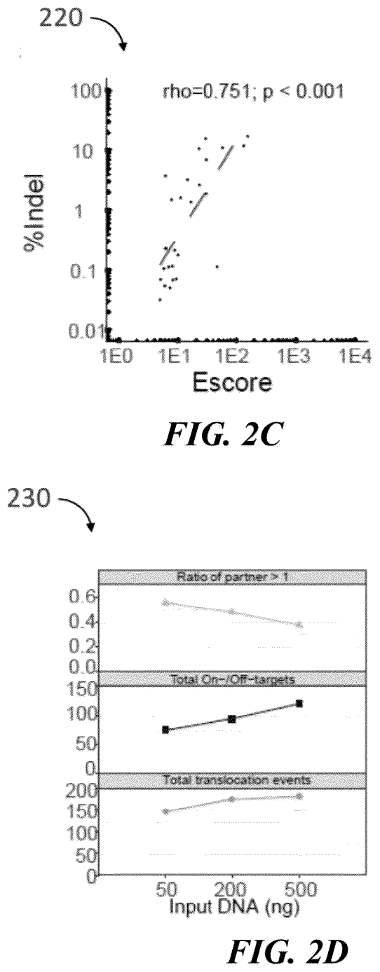

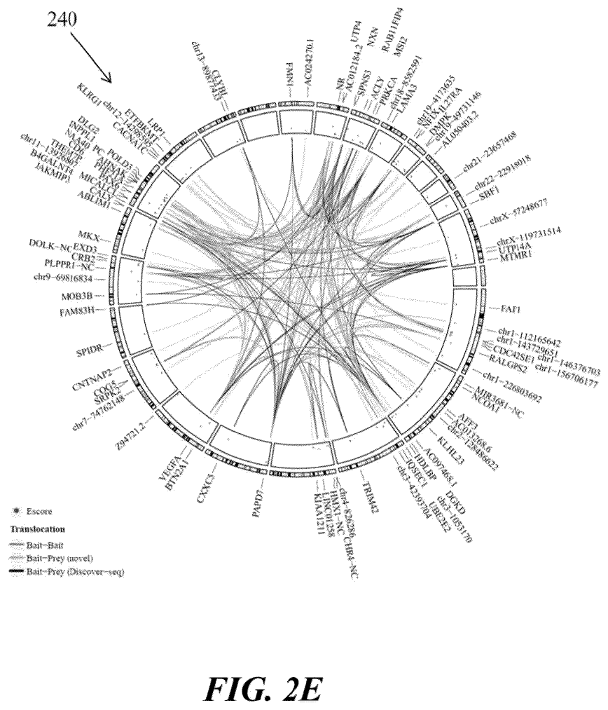

The sequences in FIGS. 2A, 2B, 4C, 4D, 5B, and 5C are shown in Table 1 below.

TABLE 1
Sequences in FIGS. 2A, 2B, 4C, 4D, 5B, and 5C

| SEQ ID | |
|---|---|
| NO: | Sequence |
| 961 | GACCCCCTCCACCCCGCCTCCGG |
| 962 | CTACCCCTCCACCCCGCCTCCGG |
| 963 | ATTCCCCCCCACCCCGCCTCAGG |
| 964 | GGGCCCCTCCACCCCGCCTCTGG |
| 965 | GACCCCCTTCACCCCACCTATGG |
| 966 | TACCCCCCACACCCCGCCTCTGG |
| 967 | GCCCCCACCCACCCCGCCTCTGG |
| 968 | TGCCCCCCCCACCCCACCTCTGG |
| 969 | ACACCCCCCCACCCCGCCTCAGG |
| 970 | CTCCCCCCCCTCCCCGCCTCGGG |
| 971 | TGCCCCTCCCACCCCGCCTCTGG |
| 972 | CGCCCTCCCCACCCCGCCTCCGG |
| 973 | AGCCCCCCCCACCCCGACTCAGG |
| 974 | GCCCCCCACCACCCCACCTCGGG |
| 975 | GACACACCCCACCCCACCTCAGG |
| 976 | GGCCCTCTCCACTCCACCTCAGG |
| 977 | CCCCCCCCCCCCCCCGCCTCCGG |
| 978 | TCCCCCCTCAACCCCACCTCAGG |
| 979 | CTGCCCCCCCACCCCGCCACTGG |
| 980 | TGCCCCCCCCACCCCGCCCCCGG |
| 981 | GTCCTCCACCACCCCGCCTCTGG |
| 982 | GCCACCCACCACCCCACCTCAGG |
| 983 | TACCCCCCCCACCCCGCCACAGG |
| 984 | CTCCCCACCCACCCCGCCTCAGG |
| 985 | CAACCCCCCCACCCCGCTTCAGG |
| 986 | GCTTCCCTCCACCCCGCATCCGG |
| 987 | GTCACTCCCCACCCCGCCTCTGG |
| 988 | ATCCCCCTCCACCCCACCCCTGG |
| 989 | GACCCCCCCCACCCCGCCCCCGG |
| 990 | GCCACCTTCCACCCCACCTCAGG |
| 991 | CACTCCCCCCACCCCGCCCCAGG |
| 992 | GACCCCTCCCACCCCGACTCCGG |
| 993 | CCCCCCCCCCCCCCCGCCTCAGG |
| 994 | GCCTCTCTGCACCCCGCCTCAGG |
| 995 | CCCCCCCCCCACCCCGCCCCCGG |
| 996 | CTCTCCCCCCACCCCGCCTCTGG |
| 997 | CCCCACCCCCACCCCGCCTCAGG |
| 998 | GACCCCCCCCACCCCACCCCAGG |
| 999 | CCACCCCCCCACCCCGCCCCAGG |
| 1000 | AGGCCCCCCCGCCCCGCCTCAGG |
| 1001 | CCCCCCCCCCCCCCCACCCCCAG |
| 1002 | GATCGACTCCACCCCGCCTCTGG |
| 1003 | AGCCAACCCCACCCCGCCTCTGG |
| 1004 | TCCACCCCCCACCCCGCCCCGGG |
| 1005 | CACCCCCCGCACCCCGCCCCAGG |
| 1006 | CCTCCCCCACACCCCGCATCCGG |
| 1007 | GGCAGCCTCCACCACGCCTCCGG |
| 1008 | CATCCCCCCCACCCCACCCCGGG |
| 1009 | CCACCCCCCCACCCCGCCCCTGG |
| 1010 | AGGCCCCCACACCCCGCCTCAGG |
| 1011 | GTACCCCACCACCCCGCCCCAGG |
| 1012 | CATACCCCCCACCCCGCCCCGGG |
| 1013 | CCGCCCCTCCACCCCGCCACTGG |
| 1014 | AGTAGCCCCCACCCCGCCTCGGG |
| 1015 | ACCCCCCCCCCCCCCGCCCCCGG |
| 1016 | GCCCCGCTCCTCCCCGCCTCCGG |
| 1017 | CCACCCCTCCACCCTGCTTCGGG |
| 1018 | CATTTCCCCTACCCCGCCCCTGG |
| 1019 | AACACGCCCCACCCCGCCCCAGG |
| 1020 | GAGCCACTGTGCCCAGCCTAGGG |
| 1021 | CACTCCCCACCCCCCACCCCCAG |
| 1022 | CCCTCCCCCCACCCCACAACAGG |
| 1023 | GTCCCTTTCCACCCTGCCTCTGG |
| 1024 | GAGCTCCCCCACCCCGCCCCGGG |
| 1025 | AACACCCGCCCCCCCACCCCCGG |
| 1026 | GATTCCCTGGACCACATCTCTGG |
| 1027 | GAGCCACCAAACCCAGCCTCAGG |
| 1028 | GAATCCCAGGAGCCCGCCTCGAG |
| 1029 | GGCCCCCTTTCCCACATCTCTGG |
| 1030 | CTCCCCCAGCCCCCCACCTCCCG |
| 1031 | CATTCTCGACACCCCGCCCCCGG |
| 1032 | TACTCCTTCACCCCCACCCCAGG |
| 1033 | CACACTCTCAACCTCACTTCTAG |
| 1034 | TCCATCCTCAGCCCCACCTCTCG |
| 1035 | AACCCATTCCACCCTGCCTCAGG |
| 1036 | GCCACCCCCCACCCTGCCTCCGG |
| 1037 | CACCAGGTCTGCCCCGCATCAGG |
| 1038 | AATCCTCTCACCTCAGCCTCCGG |
| 1039 | GTGCCACTCCACCCCACCCTGGG |
| 1040 | CCCCCCGGCCCCCCCACCCCAGG |
| 1041 | CACCCCCCGCCCCCCGCCCCCGG |
| 1042 | CTCACCATAAACTCCGCCTCCCG |
| 1043 | GAGCCACTGCACCCAGCCTCAAG |
| 1044 | GAGCCACCACAACCAGCCTCGAG |
| 1045 | GTTTCCCTTCTTCCCGCCCCAGG |
| 1046 | CCCCCACCCCCCCCCACCCCCAG |
| 1047 | ATCCTCCCACACCCCACATCAGA |
| 1048 | CACCGCGCCCAGCCAGCTTCTGG |
| 1049 | GAGCCACCTCACCCAGCCTAAAG |
| 1050 | GAGCCACCACACCCAGCCTAAAG |
| 1051 | GAGCCACTGCGCCCAGCCCCAGG |
| 1052 | GAACCAGACCTCCCCATCTCCAG |
| 1053 | GAGCCACTGCACCTGGCCTCAGG |
| 1054 | GCACACCACCCCCCCGCCACCGG |
| 1055 | TGTGAAAACTAAGAGAGAGCTCCACCCCTCTGTGCCCTC |
| | CTCCTGTCCTGAGTCGGGGTGGGGGGGGCTGGCCTTGGA |
| | GGGGGCGTCCCCT |
| 1056 | GGCCACGTCGCCCGTGTATGAGATGGCAGCCTCCACCAC |
| | GCCTCCGGCACTTCCTGCCGCCTCCATGCCCAGCAGCAT |
| | GTTGGGCAAGTAGTTGAGGGAG |
| 1057 | AVDGTYSIAAEVVGGASGAAEMGLLMNPLYNLS |
| 1058 | CCACCCACCACCCCACCTCAGGCAAATGCCCAGCCCCTG |
| | CCTCGCCTCCAGCCTCCTTTCCACAACCCAGCATCCAGT |
| | CACTCCAGTC |
| 1059 | GCCCCGGGTTTCAAGTGATTTTCATACTTCAGCCTCCTG |
| | AGTAGCT |
| 1060 | AGCCCCAGCAAGAGCACAAGAGG |
| 1061 | ATCACCCCCAAGAGCACAAGGGG |
| 1062 | AGCCCCAGTGAGAGCACAAGAGG |
| 1063 | AGTTCCAGCAACAGCACAAAAGG |
| 1064 | AACTCCAGCGAGAGCACAAGAGG |
| 1065 | AGCCCCAGTAAGAGCACAAGAGG |
| 1066 | AACACAAGCAAGAGCACGAGAGG |
| 1067 | AGCCCCAGCAAGAGCACGAGAGG |
| 1068 | AGCCTAAGAAAGAACACAAGAGG |
| 1069 | AGCCCCAGCTAAAGCAAAAGAGG |
| 1070 | TGCCCCAGCTAAAACACAAGTGG |
| 1071 | AAACCAAACAAGGACACAAGAGA |
| 1072 | AATCCCAGTGAGAGCACAAGAGG |
| 1073 | ACCCCTAGCTACAGCACAAGAGG |
| 1074 | TGCAGCAGCAAGAGCACAGGCGG |
| 1075 | CACAAGAGCAAGAGCACAAGAGG |
| 1076 | GCTCTCAGCAAGACCACAAGTGG |
| 1077 | TGCCCCAAGAACAACAAAAAAAG |
| 1078 | TGCCTCAGTCAAAGCACAGCAGG |
| 1079 | AAAACCAACAACAGTACAAAAGG |
| 1080 | CACTCCAGCCTGGGCAAAAGAGG |
| 1081 | ATTCTGAGGAAGAAAACAAGGGG |
| 1082 | TCCCCCTACCAGAGCACATACAG |
| 1083 | AGGCAAATCAAAACCACAATGAG |
| 1084 | AAAACAAGAAAGAACAAAAGAGA |
| 1085 | CTTGCCCCACAGGGCAGTAACGG |
| 1086 | CTTGGCCTGCAGGGCAGTTATGG |
| 1087 | TCTACCCCACATGGCAGTAATGG |
| 1088 | ACTGAGCCTCAGGGCAGTAATGG |
| 1089 | CCTGCCCCACAGGGCAATTATGG |
| 1090 | CCTCTCCCACAGGGCAGTAAAGG |
| 1091 | GCTGCCCCACAGGGCAGCAAAGG |
| 1092 | CCTCCAATACAGGGCAGTAAAGG |
| 1093 | CCTGTCCCACAGGGCAGGAAGGG |
| 1094 | CTGGCACCACAGAGCAGAAAGGG |
| 1095 | CATGCTCCACAGAGCAGCAAAGG |
| 1096 | GGGCTGCCCCAGGGCAGTAATGG |
| 1097 | CTTGCTGCACAGGACAATAAAGG |
| 1098 | CTCGCCCCTCAGGGCAGTAGTGG |
| 1099 | GTTGGCCCTCAGGGCAGAAATGG |
| 1100 | GAGGCGCCACAGGGCAGTAATGG |
| 1101 | GCTGTGTCATAGGGCAGTAACGG |
| 1102 | CTTTCTTCACAGGGTAGTAATGG |
| 1103 | TGCCCCAGACAGGGCAGTAAGGG |
| 1104 | CTTGCACTACAAGTCAGTAATGG |
| 1105 | ATTTCCTCACAGGGCAGAAAAGG |
| 1106 | TCACCCCCACAGGCCAGTAAAGG |
| 1107 | GTCATGTCACAGGGCAGTAGTGG |
| 1108 | GGCCCTGCCCAGGGCAGTAATGG |
| 1109 | CTTAATACACAGGGAAGGAATGG |
| 1110 | CTTCAAGAGCAACAGTGCTGTGG |
| 1111 | GAGAGACAGCAACAGTGCTATGG |
| 1112 | AGCAAGGAGCAACAGTGATGTGG |
| 1113 | AGCAAACATCAACAGTGCTGAGG |
| 1114 | TAGGAAGAGCAACAGGGCTGTGG |
| 1115 | CATGAAGGGCAACAGAGCTGAGG |
| 1116 | CACTCTAAGCAACAGTGCTGGGG |
| 1117 | TGCGAGGAGCAACAGTGCTTGGG |
| 1118 | GTCTCTAGGCAACAGTGCTGAGG |
| 1119 | GGGCAGCAGCTACAGTGCTGAGG |
| 1120 | GGGCGGTGCTACAACTGGGCTGG |
| 1121 | GGGTGGTTCTACAACCAGGCTGG |
| 1122 | GGGCGGTGCTACAACTGGGCTGG |
| 1123 | GGGAGGTGCCACATCAGGGCCGG |
| 1124 | GGGCAGTGATCCAACTGTGCAGG |
| 1125 | AGCTGGGGCTACATCTGGGCTGG |
| 1126 | GCTGGGTGCTACAACAGGGCAGG |
| 1127 | CTGTGGTGCAACAACTGGGCTGG |
| 1128 | GGGAGGAGGTACAACTGGGAGGG |
| 1129 | CAGTGTGGCTACAACTGCGCAGG |
| 1130 | CTGGTCAGCTACAACTGGCCTGG |
| 1131 | GGTGTAAAATCAACACCCTAAGG |
| 1132 | GCTGGAAAAAAAACACCCTAGGG |
| 1133 | GAGGTAAAACCAACACCTTAAGG |
| 1134 | TGGCTGAAATCAACACCCCAGGG |
| 1135 | TGACACCAATCAACACCTTAAGG |
| 1136 | TCTGATCCATCAACACCCTATGG |

### DETAILED DESCRIPTION

### Overview

Aspects described herein are methods for enriching or identifying at least one target nucleic acid. In some aspects, the method increases sensitivity of enriching or identifying the at least one target nucleic acid. In some aspects, the method increases specificity of enriching or identifying the at least one target nucleic acid. In some aspects, the method comprises ligating at least one adaptor to the at least one target nucleic acid. In some aspects, the method comprises performing at least one PCR to obtain at least one PCR product. In some aspects, the method comprises performing a first PCR to obtain a first PCR product followed by performing a second PCR to obtain a second PCR product, where the at least one adaptor is ligated to the at least one target nucleic acid or to the PCR product.

In some embodiments, the method comprises enriching at least one target nucleic acid from a sample comprising a plurality of single-strand nucleic acid fragments by contacting a universal oligonucleotide adaptor with the sample to produce a ligation product. In some embodiments, the universal oligonucleotide adaptor is configured for ligating to a 5′ end of the single-strand nucleic acid fragments. In some embodiments, the method comprises amplifying the ligation product by a first PCR to form a first PCR product. In some embodiments, the method comprises amplifying the first PCR product by a second PCR with a second target-specific primer and a universal oligonucleotide adaptor primer to form a second PCR product. In some embodiments, the second target-specific primer is nested relative to the first target-specific primer. In some embodiments, the method enriches at least one target nucleic acid from a sample comprising a plurality of single-strand nucleic acid fragments by ligating a universal oligonucleotide adaptor to a 5′ end of the single-strand nucleic acid fragments; annealing a first target-specific primer to the single-strand nucleic acid fragments in the vicinity of a target sequence; extending the first target-specific primer over the single-strand nucleic acid fragments using a DNA polymerase; obtaining a nascent primer extension duplex; dissociating the nascent primer extension duplex into single strands; and amplifying a portion of the single stands of the nascent primer extension duplex with a second target-specific primer and a universal oligonucleotide adaptor primer.

In some embodiments, the method described herein identifies genome-wide gene editing off-target sites from a sample comprising a plurality of single-strand nucleic acid fragments by contacting a universal oligonucleotide adaptor with the sample to produce a ligation product, wherein the universal oligonucleotide adaptor is configured for ligating to a 5′ end of the single-strand nucleic acid fragments; amplifying the ligation product by performing a first PCR with a first target-specific primer to form a first PCR product; amplifying the first PCR product by a second PCR with a sequencing specific adaptor primer and a second target-specific primer nested relative to the first target-specific primer, to form a sequencing library; quantifying and reading the sequencing library to obtain sequencing results; and mapping the sequencing results to a reference genome. In some embodiments, the method described herein can evaluate gene editing efficiency from a sample comprising a plurality of single-strand nucleic acid fragments by contacting a universal oligonucleotide adaptor with the sample to produce a ligation product, wherein the universal oligonucleotide adaptor is configured for ligating to a 5′ end of the single-strand nucleic acid fragments; amplifying the first ligation product by performing a first PCR with a first target-specific primer to form a first PCR product; amplifying the first PCR product by a second PCR with a sequencing specific adaptor primer and a second target-specific primer nested relative to the first target-specific primer, to form a sequencing library; quantifying and reading the sequencing library to form sequencing results; and mapping the sequencing results to a reference genome and evaluating gene editing efficiency. In some aspects, the evaluation of gene editing efficiency can be applied to evaluating translocation or indel frequency.

In some aspects, described herein is a method of identifying genome-wide gene editing off-target sites from a sample comprising at least one target nucleic acid by contacting a universal oligonucleotide adaptor with the sample to produce a ligation product, wherein the universal oligonucleotide adaptor is configured for ligating to a 5′ end of the single-strand nucleic acid fragments; amplifying the ligation product by a first PCR with a first set of target-specific primers, wherein the target-specific primers in the first set are configured for annealing to the single-strand nucleic acid fragments 5′ of an on-target site and one or more predicted and/or known off-target sites; amplifying the first PCR product by a second PCR with a second set of target-specific primers and a universal oligonucleotide adaptor primer to form a sequencing library, wherein each member of the second set of target-specific primers is nested relative to a corresponding primer of the first set of target-specific primers; and sequencing the sequencing library to identify off-target sites. In some embodiments, the method described herein can be combined with computation prediction for identifying off-target sites.

### Enrichment

In certain embodiments, provided is a method of enriching at least one targeted nucleic acid from a sample comprising a plurality of single-strand nucleic acid fragments, the method comprising: contacting a universal oligonucleotide adapter with the sample to produce a ligation product, where the universal oligonucleotide adaptor is configured for ligating to a 5′ end of the single-strand nucleic acid fragments. In some embodiments, the method comprises amplifying the ligation product by a first PCR with a first target-specific primer to form a first PCR product. In some embodiments, the method comprises amplifying the first PCR product by a second PCR with a second target-specific primer and a universal oligonucleotide adaptor primer to form a second PCR product, where the second target-specific primer is nested relative to the first target-specific primer. In some embodiments, the sample comprises a plurality of DNA fragments prepared from high molecular weight DNA (e.g., genomic DNA). In some embodiments, the plurality of DNA fragments are prepared by enzyme-based treatment. In other embodiments, the plurality of DNA fragments are prepared by being exposed to short-wavelength, high-frequency acoustic energy. In other embodiments, the plurality of DNA fragments are prepared by heating the DNA at 100° C. to 105° C. In other embodiments, the plurality of DNA fragments are prepared by centrifugal shearing. In other embodiments, the plurality of DNA fragments are prepared by hydrodynamic shear forces. In some embodiments, the plurality of DNA fragments are prepared by being exposed to ultrasound sonication. In some specific embodiments, the plurality of DNA fragments are prepared by Bioruptor® Pico or Diagenode One. In other embodiments, the plurality of DNA fragments are prepared by turbulent flow generated by formation of hydropores. In some specific embodiments, the plurality of DNA fragments are prepared by Megaruptor®, Nebulizer®, and/or Covaris®. In some embodiments, the preparation of the plurality of DNA fragments is analyzed and confirmed by agarose gel electrophoresis. In some embodiments, the preparation of the plurality of DNA fragments is analyzed and confirmed by Fragment Analyzer™. In some embodiments, the preparation of the plurality of DNA fragments is analyzed and confirmed by LabChip® GX Touch™ nucleic acid analyzer.

In some embodiments, the plurality of DNA fragments described herein are about 50 bp to about 5000 bp long. In some specific embodiments, the plurality of DNA fragments described herein are about 50 bp to about 200 bp long, about 50 bp to about 300 bp long, about 50 bp to about 400 bp long, about 50 bp to about 500 bp long, about 50 bp to about 600 bp long, about 50 bp to about 700 bp long, about 50 bp to about 800 bp long, about 50 bp to about 900 bp long, about 50 bp to about 500 bp long, about 50 bp to about 2000 bp long, about 50 bp to about 3000 bp long, about 50 bp to about 4000 bp long, or about 50 bp to about 5000 bp long. In some specific embodiments, the plurality of DNA fragments described herein are about 100 bp to about 200 bp long, about 100 bp to about 300 bp long, about 100 bp to about 400 bp long, about 100 bp to about 500 bp long, about 100 bp to about 600 bp long, about 100 bp to about 700 bp long, about 100 bp to about 800 bp long, about 100 bp to about 900 bp long, about 100 bp to about 1000 bp long, about 100 bp to about 2000 bp long, about 100 bp to about 3000 bp long, about 100 bp to about 4000 bp long, or about 100 bp to about 5000 bp long. In other specific embodiments, the plurality of DNA fragments described herein are about 300 bp to about 400 bp long, about 300 bp to about 500 bp long, about 300 bp to about 600 bp long, about 300 bp to about 700 bp long, about 300 bp to about 800 bp long, about 300 bp to about 900 bp long, about 300 bp to about 1000 bp long, about 300 bp to about 2000 bp long, about 300 bp to about 3000 bp long, about 300 bp to about 4000 bp long, or about 300 bp to about 5000 bp long. In other specific embodiments, the plurality of DNA fragments described herein are about 600 bp to about 700 bp long, about 600 bp to about 800 bp long, about 600 bp to about 900 bp long, about 600 bp to about 1000 bp long, about 600 bp to about 2000 bp long, about 600 bp to about 3000 bp long, about 600 bp to about 4000 bp long, or about 600 bp to about 5000 bp long. In other specific embodiments, the plurality of DNA fragments described herein are about 1000 bp to about 2000 bp long, about 1000 bp to about 3000 bp long, about 1000 bp to about 4000 bp long, or about 1000 bp to about 5000 bp long.

In some embodiments, the plurality of single-strand nucleic acid fragments are prepared from denaturation of double-strand DNA fragments. In some specific embodiments, the double-strand DNA fragments are heated at 95° C. for 1, 5, 10, 20, or 30 minutes. In other specific embodiments, the double-strand DNA fragments are heated at 95° C. for 1, 5, 10, 20, or 30 minutes, followed by being placed on ice for 1 minute. In other specific embodiments, the double-strand DNA fragments are disrupted with glass beads (Disruptor Beads™; Scientific Industries, Bohemia, NY, USA) for 1, 5, 10, 20, or 30 minutes at 2,500 rpm with a Disruptor Genie bead-beater (Scientific Industries); followed by centrifuging at 3,000 rpm for 30 seconds to precipitate out the beads. In other specific embodiments, the double-strand DNA fragments are subjected to direct sonication at 10 W for 30, 60, 90, 120, 150, 200, 250, or 300 seconds. In other specific embodiments, the double-strand DNA fragments are indirect sonication at 10 W, 22.4 kHz for 1, 5, 10, 20, or 30 minutes. In other specific embodiments, the double-strand DNA fragments are placed in tubes and immerged into the water of the ultrasonic bath at 40 kHz for 1, 5, 10, 20, or 30 minutes. In other specific embodiments, the double-strand DNA fragments are homogenized in 0.01, 0.1, or 1 mol/L NaOH with continuous pipetting and incubated at ambient temperature for 1, 2, 5, 10, 20, or 30 minutes. In other specific embodiments, the double-strand DNA fragments are homogenized gently with pipette in 25% and 50% formamide solution and incubated at room temperature. In other specific embodiments, the double-strand DNA fragments are homogenized gently with pipette in 25%, 50%, and 60% DMSO solution and incubated at room temperature. In some embodiments, the preparation of the plurality of single-strand nucleic acid fragments is confirmed by measuring the absorbance of DNA fragments at 260 nm.

In some embodiments, the method further comprises at least one of: blocking a 3′ end of the single-strand nucleic acid fragments; phosphorylating a 5′ end of the single-strand nucleic acid fragments; or adenylating the nucleic acid to produce a 3′-adenosine overhang on the single-strand nucleic acid fragments. In some embodiments, the universal oligonucleotide adaptor is single stranded. In some embodiments, the universal oligonucleotide adaptor is double stranded. In some embodiments, the universal oligonucleotide adaptor comprises: a 3′ recessive end, the 3′ recessive end configured for ligating to the 5′ end of the single-strand nucleic acid fragments; and a 5′ protrude end comprising three to twenty bases of random or degenerate nucleotides. A duplex portion of the universal oligonucleotide adaptor is of sufficient length to remain in duplex. In some embodiments, the universal oligonucleotide adaptor comprises a hairpin loop connecting a portion of the duplex form. In some embodiments, the universal oligonucleotide adaptor comprises a Y shape. In some embodiments, the universal oligonucleotide adaptor comprises a barcode. In some embodiments, the universal oligonucleotide adaptor comprises a single strand segment comprising three to twenty random nucleotides as a unique molecular index (UMI) for tracing individual original molecules.

In some embodiments, the universal oligonucleotide adaptor is ligated to the 5′ end of the single-stranded nucleic acid fragments. In some embodiments, the universal oligonucleotide adaptor is ligated to the 3′ end of the single-stranded nucleic acid fragments. In some embodiments, the universal oligonucleotide adaptor is ligated to the 5′ and 3′ end of the single-stranded nucleic acid fragments. In some embodiments, the universal oligonucleotide adaptor is ligated via a ligase. When the sample described herein is a targeted gene edited sample, the target of the first target-specific primer described herein is predetermined. In some embodiments, the target comprises an on-target site of the CRISPR gene editing. In other embodiments, the target comprises a predicted off-target site of the CRISPR gene editing. In other embodiments, the target comprises a spontaneous double-strand breakpoint.

The predicted off-target site described herein is computationally predicted. In some specific embodiments, the predicted off-target site described herein is predicted by E-CRISP. In other specific embodiments, the predicted off-target site described herein is predicted by Cas-OFFinder. In other specific embodiments, the predicted off-target site described herein is predicted by CRISPRscan. In other specific embodiments, the predicted off-target site described herein is predicted by CRISPRitz. In other specific embodiments, the predicted off-target site described herein is predicted by CRISPOR. In other specific embodiments, the predicted off-target site described herein is predicted by CRISPR Design website (http://crispr.mit.edu). In other specific embodiments, the predicted off-target site described herein is predicted by Ecrisp. In other specific embodiments, the predicted off-target site described herein is predicted by Crispr2vec. In other specific embodiments, the predicted off-target site described herein is predicted by Hsu-Zhang scores. In other specific embodiments, the predicted off-target site described herein is predicted by CHOPCHOP. In other specific embodiments, the predicted off-target site described herein is predicted by CFD. In other specific embodiments, the predicted off-target site described herein is predicted by CRISTA. In other specific embodiments, the predicted off-target site described herein is predicted by Elevation. In other specific embodiments, the predicted off-target site described herein is predicted by DeepCrispr. In other specific embodiments, the predicted off-target site described herein is predicted by DeepSpCas9. In other specific embodiments, the predicted off-target site described herein is predicted by CALITAS. In other specific embodiments, the predicted off-target site described herein is predicted by an algorithm with a deep convolutional neural network or a deep feedforward neural network. In some embodiments, the cutoff to set in one or more of the above-described prediction algorithms is mismatch(es) being less than or equal to 10, 9, 8, 7, 6, 5, 4, 3, 2, or 1 inside and/or outside of seed. In some embodiments, the cutoff to set in one or more of the above-described prediction algorithms is mismatch(es) being less than or equal to 10, 9, 8, 7, 6, 5, 4, 3, 2, or 1 inside and/or outside of protospacer adjacent motif (PAM). In other embodiments, the cutoff in one or more of the above-described prediction algorithms is set bulge(s) (insertion as DNA bulge or deletion as RNA bulge) being less than or equal to 4, 3, 2, or 1 respectively inside and/or outside of seed. In other embodiments, the cutoff in one or more of the above-described prediction algorithms is set bulge(s) (insertion as DNA bulge or deletion as RNA bulge) being less than or equal to 4, 3, 2, or 1 respectively inside and/or outside of PAM.

In some embodiments, the spontaneous double-strand breakpoints described herein are genome fragile sites. In some specific embodiments, the spontaneous double-strand breakpoints described herein comprise Chr 1: 89231183, Chr 1: 109838221.

The first target-specific primer described herein is designed to be in the vicinity of the target described herein. In some embodiments, the first target-specific primer described herein is reverse complementary to a DNA segment that is in the downstream of the target described herein on either strand. In some specific embodiments, the DNA segment described herein is about 5 bp to about 1000 bp downstream of one of the target described herein. In some specific embodiments, the DNA segment described herein is about 5 bp to about 500 bp downstream of one of the target described herein. In some specific embodiments, the DNA segment described herein is about 5 bp to about 10 bp, about 10 bp to about 30 bp, about 30 bp to about 50 bp, about 50 bp to about 70 bp, about 70 bp to about 90 bp, or about 90 bp to about 100 bp downstream of the target described herein. In other specific embodiments, the DNA segment described herein is about 100 bp to about 120 bp, about 120 bp to about 140 bp, about 140 bp to about 160 bp, about 160 bp to about 180 bp, about 180 bp to about 200 bp, downstream of the target described herein. In other specific embodiments, the DNA segment described herein is about 200 bp to about 220 bp, about 220 bp to about 240 bp, about 240 bp to about 260 bp, about 260 bp to about 280 bp, about 280 bp to about 300 bp downstream of the target described herein. In other specific embodiments, the DNA segment described herein is about 300 bp to about 400 bp, about 400 bp to about 500 bp, about 500 bp to about 600 bp, about 600 bp to about 700 bp, about 700 bp to about 800 bp, about 800 bp to about 900 bp, about 900 bp to about 100 bp downstream of the target described herein. In other specific embodiments, the DNA segment described herein is at least 10, 50, 100, 150, 200, 250, 300, 350, 400, 450, 500, 550, 600, 650, 700, 750, 800, 850, 900, 950, or 1000 bp downstream of the target described herein.

In some embodiments, the second target-specific primer described herein is designed to be in the vicinity of the target described herein. In some embodiments, the second target-specific primer described herein is reverse complementary to a DNA segment that is in the downstream of the target described herein on either strand. In some specific embodiments, the DNA segment described herein is about 3 bp to about 1000 bp downstream of one of the target described herein. In some specific embodiments, the DNA segment described herein is about 3 bp to about 300 bp downstream of one of the target described herein. In some specific embodiments, the DNA segment described herein is about 3 bp to about 10 bp, 10 bp to about 30 bp, about 30 bp to about 50 bp, about 50 bp to about 70 bp, about 70 bp to about 90 bp, or about 90 bp to about 100 bp downstream of the target described herein. In other specific embodiments, the DNA segment described herein is about 100 bp to about 120 bp, about 120 bp to about 140 bp, about 140 bp to about 160 bp, about 160 bp to about 180 bp, about 180 bp to about 200 bp, downstream of the target described herein. In other specific embodiments, the DNA segment described herein is about 200 bp to about 220 bp, about 220 bp to about 240 bp, about 240 bp to about 260 bp, about 260 bp to about 280 bp, about 280 bp to about 300 bp downstream of the target described herein. In other specific embodiments, the DNA segment described herein is about 300 bp to about 400 bp, about 400 bp to about 500 bp, about 500 bp to about 600 bp, about 600 bp to about 700 bp, about 700 bp to about 800 bp, about 800 bp to about 900 bp, about 900 bp to about 100 bp downstream of the target described herein. In other specific embodiments, the DNA segment described herein is at least 10, 50, 100, 150, 200, 250, 300, 350, 400, 450, 500, 550, 600, 650, 700, 750, 800, 850, 900, 950, or 1000 bp downstream of the target described herein.

The second target-specific primer described herein is designed to be in the vicinity of the first target-specific primer described herein. In some embodiments, the second target-specific primer described herein is reverse complementary to a DNA segment that is in the downstream of the first target-specific primer described herein on either strand. In some specific embodiments, the DNA segment described herein is about 3 bp to about 1000 bp downstream of one of the first target-specific primer described herein. In some specific embodiments, the DNA segment described herein is about 3 bp to about 300 bp downstream of the first target-specific primer described herein. In some specific embodiments, the DNA segment described herein is about 10 bp to about 30 bp, about 30 bp to about 50 bp, about 50 bp to about 70 bp, about 70 bp to about 90 bp, or about 90 bp to about 100 bp downstream of the first target-specific primer described herein. In other specific embodiments, the DNA segment described herein is about 100 bp to about 120 bp, about 120 bp to about 140 bp, about 140 bp to about 160 bp, about 160 bp to about 180 bp, about 180 bp to about 200 bp, downstream of the first target-specific primer described herein. In other specific embodiments, the DNA segment described herein is about 200 bp to about 220 bp, about 220 bp to about 240 bp, about 240 bp to about 260 bp, about 260 bp to about 280 bp, about 280 bp to about 300 bp downstream of the first target-specific primer described herein. In other specific embodiments, the DNA segment described herein is about 300 bp to about 400 bp, about 400 bp to about 500 bp, about 500 bp to about 600 bp, about 600 bp to about 700 bp, about 700 bp to about 800 bp, about 800 bp to about 900 bp, about 900 bp to about 100 bp downstream of the first target-specific primer described herein. In other specific embodiments, the DNA segment described herein is at least 10, 50, 100, 150, 200, 250, 300, 350, 400, 450, 500, 550, 600, 650, 700, 750, 800, 850, 900, 950, or 1000 bp downstream of the first target-specific primer described herein.

### Primer Design

The first target-specific primer is 16-32 bp in length. In some embodiments, the first target-specific primer is 16 bp in length. In other embodiments, the first target-specific primer is 17 bp in length. In other embodiments, the first target-specific primer is 18 bp in length. In other embodiments, the first target-specific primer is 19 bp in length. In other embodiments, the first target-specific primer is 20 bp in length. In other embodiments, the first target-specific primer is 21 bp in length. In other embodiments, the first target-specific primer is 22 bp in length. In other embodiments, the first target-specific primer is 23 bp in length. In other embodiments, the first target-specific primer is 24 bp in length. In other embodiments, the first target-specific primer is 25 bp in length. In other embodiments, the first target-specific primer is 26 bp in length. In other embodiments, the first target-specific primer is 27 bp in length. In other embodiments, the first target-specific primer is 28 bp in length. In other embodiments, the first target-specific primer is 29 bp in length. In other embodiments, the first target-specific primer is 30 bp in length. In other embodiments, the first target-specific primer is 31 bp in length. In other embodiments, the first target-specific primer is 32 bp in length.

The first target-specific primer has a GC content of about 40% to about 60%. In some embodiments, the first target-specific primer has a GC content of about 40%. In other embodiments, the first target-specific primer has a GC content of about 45%. In other embodiments, the first target-specific primer has a GC content of about 50%. In other embodiments, the first target-specific primer has a GC content of about 55%. In other embodiments, the first target-specific primer has a GC content of about 60%.

The first target-specific primer has a melting temperature of about 55° C. to about 72° C. In some embodiments, the first target-specific primer has a melting temperature of about 55° C. In some embodiments, the first target-specific primer has a melting temperature of about 56° C. In some embodiments, the first target-specific primer has a melting temperature of about 57° C. In some embodiments, the first target-specific primer has a melting temperature of about 58° C. In other embodiments, the first target-specific primer has a melting temperature of about 59° C. In other embodiments, the first target-specific primer has a melting temperature of about 60° C. In other embodiments, the first target-specific primer has a melting temperature of about 65° C. In other embodiments, the first target-specific primer has a melting temperature of about 70° C. In some embodiments, the first target-specific primer has a melting temperature of about 71° C. In some embodiments, the first target-specific primer has a melting temperature of about 72° C.

The sequence of the first target-specific primer is determined such that any secondary structures are minimized. In some embodiments, the first target-specific primer does not form hairpin structures. In other embodiments, the first target-specific primer does not form dimers between two molecules of the first target-specific primer.

The last five bases on the 3′ end of the first target-specific primer do not comprise too many G or C bases. In some embodiments, the last five bases on the 3′ end of the first target-specific primer comprise no G or C bases. In other embodiments, the last five bases on the 3′ end of the first target-specific primer comprise only one G or C base. In other embodiments, the last five bases on the 3′ end of the first target-specific primer comprise only two G or/and C bases. In other embodiments, the last five bases on the 3′ end of the first target-specific primer comprise only three G or/and C bases.

The sequence of the first target-specific primer comprises limited repeats of one base or dinucleotide repeats. In some embodiments, the sequence of the first target-specific primer comprises no repeats of one base or dinucleotide repeats. In other embodiments, the sequence of the first target-specific primer comprises one or more repeats of one base but no dinucleotide repeats, and wherein the one or more repeats of one base are repeats with the same base appearing only two times, only three times, or only four times. In other embodiments, the sequence of the first target-specific primer comprises no repeats of one base but one or more dinucleotide repeats, and wherein the one or more dinucleotide repeats are repeats with the same dinucleotide appearing only two times, only three times, or only four times. In other embodiments, the sequence of the first target-specific primer comprises one or more repeats of one base and one or more dinucleotide repeats, wherein the one or more repeats of one base are repeats with the same base appearing only two times, only three times, or only four times, and wherein the one or more dinucleotide repeats are repeats with the same dinucleotide appearing only two times, only three times, or only four times.

The sequence of the first target-specific primer is designed so that it is unlikely to generate additional (non-specific) PCR amplicons using Primer-BLAST, including SNP-containing genome databases. In some embodiments, the top non-specific PCR amplicons have at least four mismatches with the first target-specific primer. In other embodiments, the top non-specific PCR amplicons have at least five, at least six, at least seven, at least eight, at least nine, at least ten mismatches with the first target-specific primer

The first target-specific primer may be automatically design by available algorithms. In some embodiments, the first target-specific primer is designed by IDT. In other embodiments, the first target-specific primer is designed by Eurofins Genomics. In other embodiments, the first target-specific primer is designed by Primer-Blast. In other embodiments, the first target-specific primer is designed by Primer3. In other embodiments, the first target-specific primer is designed by NetPrimer. In other embodiments, the first target-specific primer is designed by PerlPrimer. In other embodiments, the first target-specific primer is designed by Primer Premier.

In some embodiments, the first PCR is a linear amplification of the ligation product to obtain a nascent primer extension duplex. In some embodiments, the method described herein further comprises performing a nested amplification of the nascent primer extension duplex. In another exemplary embodiments, the first PCR is an exponential amplification of the targeted nucleic acid with the first target-specific primer and a universal oligonucleotide adaptor primer. In some embodiments, the first PCR comprises annealing the first target-specific primer to single-stranded nucleic acid fragments. The annealing temperature is determined by the melting temperature of the first target-specific primer. In some embodiments, the annealing temperature is about 55° C. In other embodiments, the annealing temperature is about 58° C. In other embodiments, the annealing temperature is about 60° C. In other embodiments, the annealing temperature is about 58° C. In other embodiments, the annealing temperature is about 65° C. In other embodiments, the annealing temperature is about 70° C. In other embodiments, the annealing temperature is about 75° C. In other embodiments, the annealing temperature is about 78° C. In some embodiments, the annealing lasts for about 0.5 minute. In other embodiments, the annealing lasts for about 1 minute. In other embodiments, the annealing lasts for about 1.5 minutes. In other embodiments, the annealing lasts for about 2 minutes. In other embodiments, the annealing lasts for about 3 minutes. In other embodiments, the annealing lasts for about 4 minutes. In other embodiments, the annealing lasts for about 5 minutes. In other embodiments, the annealing lasts for about 6 minutes. In other embodiments, the annealing lasts for about 7 minutes. In other embodiments, the annealing lasts for about 8 minutes. In other embodiments, the annealing lasts for about 9 minutes. In other embodiments, the annealing lasts for about 10 minutes. In other embodiments, the annealing lasts for about 11 minutes. In other embodiments, the annealing lasts for about 12 minutes. In other embodiments, the annealing lasts for about 13 minutes. In other embodiments, the annealing lasts for about 14 minutes. In other embodiments, the annealing lasts for about 15 minutes.

In some embodiments, the first PCR comprises an extension. In some specific embodiments, the extension lasts for about 20 seconds. In some specific embodiments, the extension lasts for about 30 seconds. In some specific embodiments, the extension lasts for about 40 seconds. In some specific embodiments, the extension lasts for about 50 seconds. In some specific embodiments, the extension lasts for about 60 seconds. In some specific embodiments, the extension lasts for about 70 seconds. In some specific embodiments, the extension lasts for about 80 seconds. In some specific embodiments, the extension lasts for about 90 seconds. In some specific embodiments, the extension lasts for about 100 seconds. In some specific embodiments, the extension lasts for about 110 seconds. In some specific embodiments, the extension lasts for about 120 seconds. In some specific embodiments, the extension lasts for about 3 minutes. In some specific embodiments, the extension lasts for about 4 minutes. In some specific embodiments, the extension lasts for about 5 minutes. In some specific embodiments, the extension lasts for about 6 minutes. In some specific embodiments, the extension lasts for about 7 minutes. In some specific embodiments, the extension lasts for about 8 minutes. In some specific embodiments, the extension lasts for about 9 minutes. In some specific embodiments, the extension lasts for about 10 minutes. In some specific embodiments, the extension lasts for about 11 minutes. In some specific embodiments, the extension lasts for about 12 minutes. In some specific embodiments, the extension lasts for about 13 minutes. In some specific embodiments, the extension lasts for about 14 minutes. In some specific embodiments, the extension lasts for about 15 minutes.

The first PCR comprises multiple cycles of the above-described PCR steps (annealing, extension, and denature) so that targets can be searched among samples multiple times. In some embodiments, the cycle number is at least 3. In some embodiments, the cycle number is at least 4. In some embodiments, the cycle number is at least 5. In some embodiments, the cycle number is at least 10. In some embodiments, the cycle number is at least 15. In some embodiments, the cycle number is at least 20. In some embodiments, the cycle number is at least 25. In some embodiments, the cycle number is at least 30. In some embodiments, the cycle number is at least 35. In some embodiments, the cycle number is at least 40. In some embodiments, the cycle number is at least 45. In some embodiments, the cycle number is at least 50. In some embodiments, the cycle number is at least 55. In some embodiments, the cycle number is at least 65. In some embodiments, the cycle number is at least 70. In some embodiments, the cycle number is at least 75.

In some embodiments, the method comprises performing a second PCR (e.g., a nested PCR) with at least one second target-specific primer. The second target-specific primer is 16-32 bp in length. In some embodiments, the second target-specific primer is 16 bp in length. In other embodiments, the second target-specific primer is 17 bp in length. In other embodiments, the second target-specific primer is 18 bp in length. In other embodiments, the second target-specific primer is 19 bp in length. In other embodiments, the second target-specific primer is 20 bp in length. In other embodiments, the second target-specific primer is 21 bp in length. In other embodiments, the second target-specific primer is 22 bp in length. In other embodiments, the second target-specific primer is 23 bp in length. In other embodiments, the second target-specific primer is 24 bp in length. In other embodiments, the second target-specific primer is 25 bp in length. In other embodiments, the second target-specific primer is 26 bp in length. In other embodiments, the second target-specific primer is 27 bp in length. In other embodiments, the second target-specific primer is 28 bp in length. In other embodiments, the second target-specific primer is 29 bp in length. In other embodiments, the second target-specific primer is 30 bp in length. In other embodiments, the second target-specific primer is 31 bp in length. In other embodiments, the second target-specific primer is 32 bp in length.

The second target-specific primer has a GC content of about 40% to about 60%. In some embodiments, the second target-specific primer has a GC content of about 40%. In other embodiments, the second target-specific primer has a GC content of about 45%. In other embodiments, the second target-specific primer has a GC content of about 50%. In other embodiments, the second target-specific primer has a GC content of about 55%. In other embodiments, the second target-specific primer has a GC content of about 60%.

The second target-specific primer has a melting temperature of about 55° C. to about 80° C. In some embodiments, the second target-specific primer has a melting temperature of about 55° C. In some embodiments, the second target-specific primer has a melting temperature of about 56° C. In some embodiments, the second target-specific primer has a melting temperature of about 57° C. In some embodiments, the second target-specific primer has a melting temperature of about 58° C. In other embodiments, the second target-specific primer has a melting temperature of about 59° C. In other embodiments, the second target-specific primer has a melting temperature of about 60° C. In other embodiments, the second target-specific primer has a melting temperature of about 65° C. In other embodiments, the second target-specific primer has a melting temperature of about 70° C. In other embodiments, the second target-specific primer has a melting temperature of about 75° C. In other embodiments, the second target-specific primer has a melting temperature of about 76° C. In other embodiments, the second target-specific primer has a melting temperature of about 77° C. In other embodiments, the second target-specific primer has a melting temperature of about 78° C. In other embodiments, the second target-specific primer has a melting temperature of about 79° C. In other embodiments, the second target-specific primer has a melting temperature of about 80° C.

The sequence of the second target-specific primer is determined such that any secondary structures are minimized. In some embodiments, the second target-specific primer does not form hairpin structures. In other embodiments, the second target-specific primer does not form dimers between two molecules of the second target-specific primer.

The last five bases on the 3′ end of the second target-specific primer do not comprise too many G or C bases. In some embodiments, the last five bases on the 3′ end of the second target-specific primer comprise no G or C bases. In other embodiments, the last five bases on the 3′ end of the second target-specific primer comprise only one G or C base. In other embodiments, the last five bases on the 3′ end of the second target-specific primer comprise only two G or/and C bases. In other embodiments, the last five bases on the 3′ end of the second target-specific primer comprise only three G or/and C bases.

The sequence of the second target-specific primer comprises limited repeats of one base or dinucleotide repeats. In some embodiments, the sequence of the second target-specific primer comprises no repeats of one base or dinucleotide repeats. In other embodiments, the sequence of the second target-specific primer comprises one or more repeats of one base but no dinucleotide repeats, and wherein the one or more repeats of one base are repeats with the same base appearing only two times, only three times, or only four times. In other embodiments, the sequence of the second target-specific primer comprises no repeats of one base but one or more dinucleotide repeats, and wherein the one or more dinucleotide repeats are repeats with the same dinucleotide appearing only two times, only three times, or only four times. In other embodiments, the sequence of the second target-specific primer comprises one or more repeats of one base and one or more dinucleotide repeats, wherein the one or more repeats of one base are repeats with the same base appearing only two times, only three times, or only four times, and wherein the one or more dinucleotide repeats are repeats with the same dinucleotide appearing only two times, only three times, or only four times.

The sequence of the second target-specific primer is designed so that it is unlikely to generate additional (non-specific) PCR amplicons using Primer-BLAST, including SNP-containing genome databases. In some embodiments, the top non-specific PCR amplicons have at least four mismatches with the second target-specific primer. In other embodiments, the top non-specific PCR amplicons have at least five, at least six, at least seven, at least eight, at least nine, at least ten mismatches with the second target-specific primer

The second target-specific primer may be automatically design by available algorithms. In some embodiments, the second target-specific primer is designed by IDT. In other embodiments, the second target-specific primer is designed by Eurofins Genomics. In other embodiments, the second target-specific primer is designed by Primer-Blast. In other embodiments, the second target-specific primer is designed by Primer3. In other embodiments, the second target-specific primer is designed by NetPrimer. In other embodiments, the second target-specific primer is designed by PerlPrimer. In other embodiments, the second target-specific primer is designed by Primer Premier.

In some embodiments, the second PCR is a linear amplification of the ligation product to obtain a nascent primer extension duplex. In some embodiments, the method described herein further comprises performing a nested amplification of the nascent primer extension duplex. In another exemplary embodiments, the second PCR is an exponential amplification of the targeted nucleic acid with the second target-specific primer and a universal oligonucleotide adaptor primer. In some embodiments, the second PCR comprises annealing the second target-specific primer to single-stranded nucleic acid fragments. The annealing temperature is determined by the melting temperature of the second target-specific primer. In some embodiments, the annealing temperature is about 55° C. In other embodiments, the annealing temperature is about 58° C. In other embodiments, the annealing temperature is about 60° C. In other embodiments, the annealing temperature is about 58° C. In other embodiments, the annealing temperature is about 65° C. In other embodiments, the annealing temperature is about 70° C. In other embodiments, the annealing temperature is about 75° C. In other embodiments, the annealing temperature is about 78° C. In some embodiments, the annealing lasts for about 0.5 minute. In other embodiments, the annealing lasts for about 1 minute. In other embodiments, the annealing lasts for about 1.5 minutes. In other embodiments, the annealing lasts for about 2 minutes. In other embodiments, the annealing lasts for about 3 minutes. In other embodiments, the annealing lasts for about 4 minutes. In other embodiments, the annealing lasts for about 5 minutes. In other embodiments, the annealing lasts for about 6 minutes. In other embodiments, the annealing lasts for about 7 minutes. In other embodiments, the annealing lasts for about 8 minutes. In other embodiments, the annealing lasts for about 9 minutes. In other embodiments, the annealing lasts for about 10 minutes. In other embodiments, the annealing lasts for about 11 minutes. In other embodiments, the annealing lasts for about 12 minutes. In other embodiments, the annealing lasts for about 13 minutes. In other embodiments, the annealing lasts for about 14 minutes. In other embodiments, the annealing lasts for about 15 minutes.

In some embodiments, the second PCR comprises an extension. In some specific embodiments, the extension lasts for about 20 seconds. In some specific embodiments, the extension lasts for about 30 seconds. In some specific embodiments, the extension lasts for about 40 seconds. In some specific embodiments, the extension lasts for about 50 seconds. In some specific embodiments, the extension lasts for about 60 seconds. In some specific embodiments, the extension lasts for about 70 seconds. In some specific embodiments, the extension lasts for about 80 seconds. In some specific embodiments, the extension lasts for about 90 seconds. In some specific embodiments, the extension lasts for about 100 seconds. In some specific embodiments, the extension lasts for about 110 seconds. In some specific embodiments, the extension lasts for about 120 seconds. In some specific embodiments, the extension lasts for about 3 minutes. In some specific embodiments, the extension lasts for about 4 minutes. In some specific embodiments, the extension lasts for about 5 minutes. In some specific embodiments, the extension lasts for about 6 minutes. In some specific embodiments, the extension lasts for about 7 minutes. In some specific embodiments, the extension lasts for about 8 minutes. In some specific embodiments, the extension lasts for about 9 minutes. In some specific embodiments, the extension lasts for about 10 minutes. In some specific embodiments, the extension lasts for about 11 minutes. In some specific embodiments, the extension lasts for about 12 minutes. In some specific embodiments, the extension lasts for about 13 minutes. In some specific embodiments, the extension lasts for about 14 minutes. In some specific embodiments, the extension lasts for about 15 minutes.

The second PCR comprises multiple cycles of the above-described PCR steps (annealing, extension, and denature) so that targets can be searched among samples multiple times. In some embodiments, the cycle number is at least 3. In some embodiments, the cycle number is at least 4. In some embodiments, the cycle number is at least 5. In some embodiments, the cycle number is at least 10. In some embodiments, the cycle number is at least 15. In some embodiments, the cycle number is at least 20. In some embodiments, the cycle number is at least 25. In some embodiments, the cycle number is at least 30. In some embodiments, the cycle number is at least 35. In some embodiments, the cycle number is at least 40. In some embodiments, the cycle number is at least 45. In some embodiments, the cycle number is at least 50. In some embodiments, the cycle number is at least 55. In some embodiments, the cycle number is at least 65. In some embodiments, the cycle number is at least 70. In some embodiments, the cycle number is at least 75.

In some embodiments, the method comprises forming a sequencing library with the first or the second, or any other additional primer described herein. In some embodiments, the method comprises forming a sequencing library with a sequencing specific adaptor pair. In some embodiments, the method comprises sequencing the sequencing library using a sequencing primer pair, where the sequencing primer pair is at least partially complementary to opposite strands of the second PCR product, respectively. In some embodiments, the sample comprises single-strand cDNA fragments prepared from reverse transcription of RNA fragments. In some embodiments, the method further comprises analyzing the plurality of nucleic acids fragments. In some embodiments, the first PCR and/or second PCR are multiplexing PCR. In some embodiments, the sample is from a mammal, (e.g., a human). In some embodiments, the human is an individual known to have or suspected of having a disease, (e.g. a cancer or a genetic disorder). In some embodiments, one or more of the target sequences comprise one or more markers for the cancer. In another aspect, provided is a method of enriching at least one targeted nucleic acid from a sample comprising a plurality of single-strand nucleic acid fragments, the method comprising ligating a universal oligonucleotide adaptor to a 5′ end of the single-strand nucleic acid fragments. In some embodiments, the method comprises annealing a first target-specific primer to the single-strand nucleic acid fragments in the vicinity of a target sequence. In some embodiments, the method comprises extending the first target-specific primer over the single-strand nucleic acid fragments using a DNA polymerase. In some embodiments, the method comprises obtaining a nascent primer extension duplex. In some embodiments, the method comprises dissociating the nascent primer extension duplex into single strands. In some embodiments, the method comprises repeating for one or more cycles In some embodiments, the method comprises amplifying a portion of the single stands of the nascent primer extension duplex with a second target-specific primer and an adaptor primer.

In some embodiments, the method further comprises at least one of: blocking a 3′ end of the single-strand nucleic acid fragments; phosphorylating a 5′ end of the single-strand nucleic acid fragments; or adenylating the nucleic acid to produce a 3′-adenosine overhang on the single-strand nucleic acid fragments. In some embodiments, the universal oligonucleotide adaptor comprises: a 3′ recessive end, the 3′ recessive end configured for ligating to the 5′ end of the single-strand nucleic acid fragments; and a 5′ protrude end comprising three to twenty bases of random or degenerate nucleotides. A duplex portion of the universal oligonucleotide adaptor is of sufficient length to remain in duplex form. In some embodiments, the universal oligonucleotide adaptor comprises a hairpin loop connecting a portion of the duplex form. In some embodiments, the method comprises forming a sequencing library with a sequencing specific adaptor pair. In some embodiments, the method, further comprises sequencing the sequencing library using a sequencing primer pair, wherein the sequencing primer pair is at least partially complementary to opposite strands of the second PCR product, respectively. In some embodiments, the sample comprises a plurality of DNA fragments prepared from high molecular weight DNA (e.g., genomic DNA). In some embodiments, the plurality of single-strand nucleic acid fragments are prepared from denaturation of double-strand DNA fragments. In some embodiments, the sample comprises single-strand cDNA fragments prepared from reverse transcription of RNA fragments. In some embodiments, the universal oligonucleotide adaptor primer is added for exponential amplification of the target sequence. In some embodiments, the universal oligonucleotide adaptor comprises a single strand segment comprising three to twenty random nucleotides as a unique molecular index (UMI) for tracing individual original molecules. In some embodiments, the method further comprises analyzing the plurality of nucleic acids fragments. In some embodiments, the first PCR and/or second PCR are multiplexing PCR.

In some embodiments, the sample is from a mammal, (e.g., a human). In some embodiments, the human is an individual known to have or suspected of having a disease, (e.g. a cancer or a genetic disorder). In some embodiments, one or more of the target sequences comprise one or more markers for the cancer. In some embodiments, the human is a fetus. In some embodiments, the sample is from a blood sample. In some embodiments, the sample is cell-free nucleic acids extracted from a blood sample. In some embodiments, the sample is nucleic acids extracted from circulating tumor cells. In some embodiments, the sample is nucleic acids extracted from lymphocytes in a blood sample for T-cell and B-cell receptor profiling. In some embodiments, the sample is a CRISPR gene edited sample. In some specific embodiments, the sample is meganucleases edited, zinc finger nucleases (ZFNs) edited, or transcription activator-like effector nucleases (TALENs) edited. In some embodiments, the sample is from CAR-T, CAR-NK, TCR-T, immortalized cell lines (e.g., engineered neural stem cell line CTX) or hematopoietic stem cells for therapeutics. In some embodiments, the sample is from genetically engineered cells (ex-vivo or in vivo), wherein the cells include but are not limited to fibroblasts, chondrocytes, keratinocytes, hepatocytes, pancreatic islet cells, stem cells (e.g., haematopoietic stem cells, mesenchymal stem cells, or skin stem cells), and immune cells (e.g., tumor infiltrating lymphocytes, viral reconstitution T cells, dendritic cells, γδ T cells, regulatory T cells (Treg) and macrophages).

In another aspect, provided is a method of identifying genome-wide gene editing off-target sites from a sample comprising a plurality of single-strand nucleic acid fragments, comprising ligating a universal oligonucleotide adaptor to the sample to produce a ligation product, where the universal oligonucleotide adaptor is configured for ligating to a 5′ end of the single-strand nucleic acid fragments. In some embodiments, the method comprises amplifying the ligation product by performing a first PCR with a first target-specific primer to form a first PCR product. In some embodiments, the method comprises amplifying the first PCR product by a second PCR with a sequencing specific adaptor primer and a second target-specific primer nested relative to the first target-specific primer, to form a sequencing library. In some embodiments, the method comprises quantifying and reading the sequencing library to obtain sequencing results. In some embodiments, the method comprises mapping the sequencing results to a reference genome.

In another aspect, provided is a method of evaluating gene editing efficiency from a sample comprising a plurality of single-strand nucleic acid fragments, comprising ligating a universal oligonucleotide adaptor to the sample to produce a ligation product, wherein the universal oligonucleotide adaptor is configured for ligating to a 5′ end of the single-strand nucleic acid fragments. In some embodiments, the method comprises amplifying the first ligation product by performing a first PCR with a first target-specific primer to form a first PCR product. In some embodiments, the method comprises amplifying the first PCR product by a second PCR with a sequencing specific adaptor primer and a second target-specific primer nested relative to the first target-specific primer, to form a sequencing library. In some embodiments, the method comprises quantifying and reading the sequencing library to form sequencing results. In some embodiments, the method comprises mapping the sequencing results to a reference genome. In some embodiments, the method comprises validating computationally predicted off-target sites such that the gene editing efficiencies at the off-target sites are determined. In some embodiments, the predicted off-target sites are predicted in silico based on software (e.g., E-CRISP, Cas-OFFinder, and/or CRISPRscan). In some embodiments, the E-CRISP has a cutoff of mismatch <=10. In some embodiments, the E-CRISP has a cutoff of mismatch <=9. In some embodiments, the E-CRISP has a cutoff of mismatch <=8. In some embodiments, the E-CRISP has a cutoff of mismatch <=7. In some embodiments, the E-CRISP has a cutoff of mismatch <=6. In some embodiments, the E-CRISP has a cutoff of mismatch <=5. In some embodiments, the Cas-OFFinder has a mismatch <=6. In some embodiments, the Cas-OFFinder has a mismatch <=5. In some embodiments, the Cas-OFFinder has a mismatch <=4. In some embodiments, the Cas-OFFinder has a mismatch <=3. In some embodiments, the Cas-OFFinder has a mismatch <=2. In some embodiments, Cas-OFFinder has a bulge <=3. In some embodiments, Cas-OFFinder has a bulge <=2. In some embodiments, Cas-OFFinder has a bulge <=1. In some embodiments, the CRISPRscan has no threshold. In some embodiments, the E-CRISP has a cutoff of mismatch <=7; the Cas-OFFinder has a mismatch <=4 and a bulge <=2; and the CRISPRscan has no threshold. In some embodiments, the method comprises further: detecting translocation by obtaining a split read and a discordant read and/or determining an insertion and deletion (indel) frequency. In some embodiments, the split read and the discordant read are obtained by: identifying potential candidate translocations and estimating protospacer similarity to an on-target spacer and a cutting frequency determinant (CFD). In some embodiments, the indel frequency is obtained by: aligning the mapped results by GATK-realigner to form aligned results; filtering the aligned results not spanning a corresponding spacer region; predicting an insertion and deletion occurring around 5-bp upstream or downstream of a cleavage site; and determining the reliable indel frequency by an indel value of the sample with an elimination by a corresponding value of a negative control.

In some embodiments, the gene editing nucleases comprise the following types but not excluding others: CRISPR-Cas9, CRISPR-Cas12, CRISPR base editors, CRISPR prime editors, transposon-based gene editors and writers, transcription activator-like effector nucleases (TALEN), meganucleases, zinc finger nucleases (ZFN).

### Off-Target Identification

In another aspect, provided is a method of identifying genome-wide gene editing off-target sites from a sample comprising a plurality of single-strand nucleic acid fragments, comprising: contacting a universal oligonucleotide adaptor with the sample to produce a ligation product, wherein the universal oligonucleotide adaptor is configured for ligating to a 5′ end of the single-strand nucleic acid fragments. In some embodiments, the method comprises amplifying the ligation product by a first PCR with a first set of target-specific primers, wherein the target-specific primers in the first set are configured for annealing to the single-strand nucleic acid fragments 5′ of an on-target site and one or more predicted and/or known off-target sites. In some embodiments, the method comprises amplifying the first PCR product by a second PCR with a second set of target-specific primers and a universal oligonucleotide adaptor primer to form a sequencing library, wherein each member of the second set of target-specific primers is nested relative to a corresponding primer of the first set of target-specific primers. In some embodiments, the method comprises sequencing the sequencing library to identify off-target sites. In some embodiments the predicted off-target sites in (b) are computationally predicted off-target sites.

In some embodiments, the computationally predicted off-target sites are top 100, 90, 80, 70, 60, 50, 40, 30, 20, or 10 off-target sites predicted based on software comprising E-CRISP, Cas-OFFinder, or CRISPRscan. In some embodiments, the E-CRISP has a cutoff of mismatch <=10. In some embodiments, the E-CRISP has a cutoff of mismatch <=9. In some embodiments, the E-CRISP has a cutoff of mismatch <=8. In some embodiments, the E-CRISP has a cutoff of mismatch <=7. In some embodiments, the E-CRISP has a cutoff of mismatch <=6. In some embodiments, the E-CRISP has a cutoff of mismatch <=5. In some embodiments, the Cas-OFFinder has a mismatch <=6. In some embodiments, the Cas-OFFinder has a mismatch <=5. In some embodiments, the Cas-OFFinder has a mismatch <=4. In some embodiments, the Cas-OFFinder has a mismatch <=3. In some embodiments, the Cas-OFFinder has a mismatch <=2. In some embodiments, Cas-OFFinder has a bulge <=3. In some embodiments, Cas-OFFinder has a bulge <=2. In some embodiments, Cas-OFFinder has a bulge <=1. In some embodiments, the CRISPRscan has no threshold. In some embodiments the E-CRISP has a cutoff of mismatch <=7; the Cas-OFFinder has a mismatch <=4 and a bulge <=2; and the CRISPRscan has no threshold. In some embodiments, the method comprises detecting translocation by obtaining a split read and a discordant read or determining an insertion and deletion (indel) frequency. In some embodiments, the split read and the discordant read are obtained by: identifying potential candidate translocations and estimating protospacer similarity to an on-target spacer and a cutting frequency determinant (CFD). In some embodiments, the indel frequency is obtained by aligning the mapped results by GATK-realigner to form aligned results. In some embodiments, the indel frequency is obtained by filtering the aligned results not spanning a corresponding spacer region; predicting an insertion and deletion occurring around 5-bp upstream or downstream of a cleavage site. In some embodiments, the indel frequency is obtained by determining the reliable indel frequency by an indel value of the sample with an elimination by a corresponding value of a negative control. In some embodiments, the method comprises blocking a 3′ end of the single-strand nucleic acid fragments. In some embodiments, the method comprises phosphorylating a 5′ end of the single-strand nucleic acid fragments. In some embodiments, the method comprises adenylating the nucleic acid to produce a 3′-adenosine overhang on the single-strand nucleic acid fragments.

In some embodiments, the universal oligonucleotide adaptor comprises a 3′ recessive end, where the 3′ recessive end is configured for ligating to the 5′ end of the single-strand nucleic acid fragments. In some embodiments, the universal oligonucleotide adaptor comprises a 5′ protrude end comprising three to twenty bases of random or degenerate nucleotides, where a duplex portion of the universal oligonucleotide adaptor is of sufficient length to remain in duplex form. In some embodiments, the universal oligonucleotide adaptor comprises a hairpin loop connecting a portion of the duplex form. In some embodiments, the universal oligonucleotide adaptor comprises a single strand segment comprising three to twenty random nucleotides as a unique molecular index (UMI) for tracing individual original molecules. In some embodiments, the method comprises forming a sequencing library with a sequencing specific adaptor pair. In some embodiments, the method comprises sequencing the sequencing library using a sequencing primer pair, where the sequencing primer pair is at least partially complementary to opposite strands of the second PCR product, respectively.

### Nucleic Acid Fragment

In some embodiments, the sample comprises a plurality of DNA fragments prepared from high molecular weight DNA (e.g., genomic DNA). In some embodiments, the plurality of DNA fragments are prepared by enzyme-based treatment. In other embodiments, the plurality of DNA fragments are prepared by being exposed to short-wavelength, high-frequency acoustic energy. In other embodiments, the plurality of DNA fragments are prepared by centrifugal shearing. In other embodiments, the plurality of DNA fragments are prepared by heating the DNA at 100° C. to 105° C. In other embodiments, the plurality of DNA fragments are prepared by hydrodynamic shear forces. In some embodiments, the plurality of DNA fragments are prepared by being exposed to ultrasound sonication. In some specific embodiments, the plurality of DNA fragments are prepared by Bioruptor® Pico or Diagenode One. In other embodiments, the plurality of DNA fragments are prepared by turbulent flow generated by formation of hydropores. In some specific embodiments, the plurality of DNA fragments are prepared by Megaruptor®, Nebulizer®, and/or Covaris®. In some embodiments, the preparation of the plurality of DNA fragments is analyzed and confirmed by agarose gel electrophoresis. In some embodiments, the preparation of the plurality of DNA fragments is analyzed and confirmed by Fragment Analyzer™. In some embodiments, the preparation of the plurality of DNA fragments is analyzed and confirmed by LabChip® GX Touch™ nucleic acid analyzer.

In some embodiments, the plurality of DNA fragments described herein are about 50 bp to about 5000 bp long. In some specific embodiments, the plurality of DNA fragments described herein are about 50 bp to about 200 bp long, about 50 bp to about 300 bp long, about 50 bp to about 400 bp long, about 50 bp to about 500 bp long, about 50 bp to about 600 bp long, about 50 bp to about 700 bp long, about 50 bp to about 800 bp long, about 50 bp to about 900 bp long, about 50 bp to about 500 bp long, about 50 bp to about 2000 bp long, about 50 bp to about 3000 bp long, about 50 bp to about 4000 bp long, or about 50 bp to about 5000 bp long. In some specific embodiments, the plurality of DNA fragments described herein are about 100 bp to about 200 bp long, about 100 bp to about 300 bp long, about 100 bp to about 400 bp long, about 100 bp to about 500 bp long, about 100 bp to about 600 bp long, about 100 bp to about 700 bp long, about 100 bp to about 800 bp long, about 100 bp to about 900 bp long, about 100 bp to about 1000 bp long, about 100 bp to about 2000 bp long, about 100 bp to about 3000 bp long, about 100 bp to about 4000 bp long, or about 100 bp to about 5000 bp long. In other specific embodiments, the plurality of DNA fragments described herein are about 300 bp to about 400 bp long, about 300 bp to about 500 bp long, about 300 bp to about 600 bp long, about 300 bp to about 700 bp long, about 300 bp to about 800 bp long, about 300 bp to about 900 bp long, about 300 bp to about 1000 bp long, about 300 bp to about 2000 bp long, about 300 bp to about 3000 bp long, about 300 bp to about 4000 bp long, or about 300 bp to about 5000 bp long. In other specific embodiments, the plurality of DNA fragments described herein are about 600 bp to about 700 bp long, about 600 bp to about 800 bp long, about 600 bp to about 900 bp long, about 600 bp to about 1000 bp long, about 600 bp to about 2000 bp long, about 600 bp to about 3000 bp long, about 600 bp to about 4000 bp long, or about 600 bp to about 5000 bp long. In other specific embodiments, the plurality of DNA fragments described herein are about 1000 bp to about 2000 bp long, about 1000 bp to about 3000 bp long, about 1000 bp to about 4000 bp long, or about 1000 bp to about 5000 bp long.

In some embodiments, the plurality of single-strand nucleic acid fragments are prepared from denaturation of double-strand DNA fragments. In some specific embodiments, the double-strand DNA fragments are heated at 95° C. for 1, 5, 10, 20, or 30 minutes. In other specific embodiments, the double-strand DNA fragments are heated at 95° C. for 1, 5, 10, 20, or 30 minutes, followed by being placed on ice for 1 minute. In other specific embodiments, the double-strand DNA fragments are disrupted with glass beads (Disruptor Beads™; Scientific Industries, Bohemia, NY, USA) for 1, 5, 10, 20, or 30 minutes at 2,500 rpm with a Disruptor Genie bead-beater (Scientific Industries); followed by centrifuging at 3,000 rpm for 30 seconds to precipitate out the beads. In other specific embodiments, the double-strand DNA fragments are subjected to direct sonication at 10 W for 30, 60, 90, 120, 150, 200, 250, or 300 seconds. In other specific embodiments, the double-strand DNA fragments are indirect sonication at 10 W, 22.4 kHz for 1, 5, 10, 20, or 30 minutes. In other specific embodiments, the double-strand DNA fragments are placed in tubes and immerged into the water of the ultrasonic bath at 40 kHz for 1, 5, 10, 20, or 30 minutes. In other specific embodiments, the double-strand DNA fragments are homogenized in 0.01, 0.1, or 1 mol/L NaOH with continuous pipetting and incubated at ambient temperature for 1, 2, 5, 10, 20, or 30 minutes. In other specific embodiments, the double-strand DNA fragments are homogenized gently with pipette in 25% and 50% formamide solution and incubated at room temperature. In other specific embodiments, the double-strand DNA fragments are homogenized gently with pipette in 25%, 50%, and 60% DMSO solution and incubated at room temperature. In some embodiments, the preparation of the plurality of single-strand nucleic acid fragments is confirmed by measuring the absorbance of DNA fragments at 260 nm.

In some embodiments, prior to (a), the method further comprises at least one of: (i) blocking a 3′ end of the single-strand nucleic acid fragments; (ii) phosphorylating a 5′ end of the single-strand nucleic acid fragments; and (iii) adenylating the nucleic acid to produce a 3′-adenosine overhang on the single-strand nucleic acid fragments.

In some embodiments, the universal oligonucleotide adaptor is single stranded. In some embodiments, the universal oligonucleotide adaptor is double stranded. In some embodiments, the universal oligonucleotide adaptor comprises: a 3′ recessive end, the 3′ recessive end configured for ligating to the 5′ end of the single-strand nucleic acid fragments; and a 5′ protrude end comprising three to twenty bases of random or degenerate nucleotides. A duplex portion of the universal oligonucleotide adaptor is of sufficient length to remain in duplex form in (a).

In some embodiments, the universal oligonucleotide adaptor comprises a hairpin loop connecting a portion of the duplex form. In some embodiments, the universal oligonucleotide adaptor comprises a Y shape.

In some embodiments, the universal oligonucleotide adaptor comprises a barcode. In some embodiments, the universal oligonucleotide adaptor comprises a single strand segment comprising three to twenty random nucleotides as a unique molecular index (UMI) for tracing individual original molecules.

In some embodiments, the universal oligonucleotide adaptor is ligated to the 5′ end of the single-stranded nucleic acid fragments. In some embodiments, the universal oligonucleotide adaptor is ligated to the 3′ end of the single-stranded nucleic acid fragments. In some embodiments, the universal oligonucleotide adaptor is ligated to the 5′ and 3′ end of the single-stranded nucleic acid fragments. In some embodiments, the universal oligonucleotide adaptor is ligated via a ligase.

When the sample described herein is a targeted gene edited sample, the targets of the first set of target-specific primers described herein are predetermined. In some embodiments, the targets comprise an on-target site of the CRISPR gene editing. In other embodiments, the targets comprise one or more predicted off-target sites of the CRISPR gene editing. In other embodiments, the targets comprise one or more spontaneous double-strand breakpoints. In other embodiments, the targets comprise a combination of part or all of the sites described above.

### Computation Prediction

The predicted off-target sites described herein are computationally predicted. In some specific embodiments, the predicted off-target sites described herein are predicted by E-CRISP. In other specific embodiments, the predicted off-target sites described herein are predicted by Cas-OFFinder. In other specific embodiments, the predicted off-target sites described herein are predicted by CRISPRscan. In other specific embodiments, the predicted off-target sites described herein are predicted by CRISPRitz. In other specific embodiments, the predicted off-target sites described herein are predicted by CRISPOR. In other specific embodiments, the predicted off-target sites described herein are predicted by CRISPR Design website (http://crispr.mit.edu). In other specific embodiments, the predicted off-target sites described herein are predicted by Ecrisp. In other specific embodiments, the predicted off-target sites described herein are predicted by Crispr2vec. In other specific embodiments, the predicted off-target sites described herein are predicted by Hsu-Zhang scores. In other specific embodiments, the predicted off-target sites described herein are predicted by CHOPCHOP. In other specific embodiments, the predicted off-target sites described herein are predicted by CFD. In other specific embodiments, the predicted off-target sites described herein are predicted by CRISTA. In other specific embodiments, the predicted off-target sites described herein are predicted by Elevation. In other specific embodiments, the predicted off-target sites described herein are predicted by DeepCrispr. In other specific embodiments, the predicted off-target sites described herein are predicted by DeepSpCas9. In other specific embodiments, the predicted off-target sites described herein are predicted by CALITAS. In other specific embodiments, the predicted off-target sites described herein are predicted by an algorithm with a deep convolutional neural network or a deep feedforward neural network.

In some embodiments, the cutoff to set in one or more of the above-described prediction algorithms is mismatch(es) being less than or equal to 10, 9, 8, 7, 6, 5, 4, 3, 2, or 1 inside and/or outside of seed. In some embodiments, the cutoff to set in one or more of the above-described prediction algorithms is mismatch(es) being less than or equal to 10, 9, 8, 7, 6, 5, 4, 3, 2, or 1 inside and/or outside of protospacer adjacent motif (PAM). In other embodiments, the cutoff in one or more of the above-described prediction algorithms is set bulge(s) (insertion as DNA bulge or deletion as RNA bulge) being less than or equal to 4, 3, 2, or 1 respectively inside and/or outside of seed. In other embodiments, the cutoff in one or more of the above-described prediction algorithms is set bulge(s) (insertion as DNA bulge or deletion as RNA bulge) being less than or equal to 4, 3, 2, or 1 respectively inside and/or outside of PAM.

After proper cutoff setting in one or more chosen algorithms described herein, in some embodiments, about top 100 predicted off-target sites are selected for designing the first set of target-specific primers. In other embodiments, about top 90 predicted off-target sites are selected for designing the first set of target-specific primers. In other embodiments, about the top 80 predicted off-target sites are selected for designing the first set of target-specific primers. In other embodiments, about the top 70 predicted off-target sites are selected for designing the first set of target-specific primers. In other embodiments, about the top 60 predicted off-target sites are selected for designing the first set of target-specific primers. In other embodiments, about the top 50, 40, 30, 20, or 10 predicted off-target sites are selected for designing the first set of target-specific primers.

In some embodiments, the spontaneous double-strand breakpoints described herein are genome fragile sites. In some specific embodiments, the spontaneous double-strand breakpoints described herein comprise Chr 1: 89231183, Chr 1: 109838221.

The first set of target-specific primers described herein are designed to be in the vicinity of the targets described herein. In some embodiments, each of the first set of target-specific primers described herein is reverse complementary to a DNA segment that is in the downstream of the one of targets described herein on sense or antisense strand. In some specific embodiments, the DNA segment described herein is about 5 bp to about 1000 bp downstream of one of the targets described herein. In some specific embodiments, the DNA segment described herein is about 5 bp to about 500 bp downstream of one of the targets described herein. In some specific embodiments, the DNA segment described herein is about 5 bp to about 10 bp, about 10 bp to about 30 bp, about 30 bp to about 50 bp, about 50 bp to about 70 bp, about 70 bp to about 90 bp, or about 90 bp to about 100 bp downstream of one of the targets described herein. In other specific embodiments, the DNA segment described herein is about 100 bp to about 120 bp, about 120 bp to about 140 bp, about 140 bp to about 160 bp, about 160 bp to about 180 bp, about 180 bp to about 200 bp, downstream of one of the targets described herein. In other specific embodiments, the DNA segment described herein is about 200 bp to about 220 bp, about 220 bp to about 240 bp, about 240 bp to about 260 bp, about 260 bp to about 280 bp, about 280 bp to about 300 bp downstream of one of the targets described herein. In other specific embodiments, the DNA segment described herein is about 300 bp to about 400 bp, about 400 bp to about 500 bp, about 500 bp to about 600 bp, about 600 bp to about 700 bp, about 700 bp to about 800 bp, about 800 bp to about 900 bp, about 900 bp to about 100 bp downstream of one of the targets described herein. In other specific embodiments, the DNA segment described herein is at least 10, 50, 100, 150, 200, 250, 300, 350, 400, 450, 500, 550, 600, 650, 700, 750, 800, 850, 900, 950, or 1000 bp downstream of one of the targets described herein.

The first set of target-specific primers have relatively uniformed length. In some embodiments, each of the first set of target-specific primers is about 13-16 bp in length. In other embodiments, each of the first set of target-specific primers is about a 16-19 bp in length. In other embodiments, each of the first set of target-specific primers is about 19-22 bp in length. In other embodiments, each of the first set of target-specific primers is about 22-25 bp in length. In other embodiments, each of the first set of target-specific primers is about 25-28 bp in length. In other embodiments, each of the first set of target-specific primers is about 28-31 bp in length. In other embodiments, each of the first set of target-specific primers is about 31-34 bp in length.

The first set of target-specific primers have relatively uniformed GC contents of about 40% to about 60%. In some embodiments, the first set of target-specific primers have relatively uniformed GC contents of about 40%. In other embodiments, the first set of target-specific primers have relatively uniformed GC contents of about 45%. In other embodiments, the first set of target-specific primers have relatively uniformed GC contents of about 50%. In other embodiments, the first set of target-specific primers have relatively uniformed GC contents of about 55%. In other embodiments, the first set of target-specific primers have relatively uniformed GC contents of about 60%.

The first set of target-specific primers have relatively uniformed melting temperatures of about 55° C. to about 80° C. In some embodiments, the first set of target-specific primers have relatively uniformed melting temperatures of about 55° C. In some embodiments, the first set of target-specific primers have relatively uniformed melting temperatures of about 56° C. In some embodiments, the first set of target-specific primers have relatively uniformed melting temperatures of about 57° C. In some embodiments, the first set of target-specific primers have relatively uniformed melting temperatures of about 58° C. In other embodiments, the first set of target-specific primers have relatively uniformed melting temperatures of about 60° C. In other embodiments, the first set of target-specific primers have relatively uniformed melting temperatures of about 65° C. In other embodiments, the first set of target-specific primers have relatively uniformed melting temperatures of about 70° C. In other embodiments, the first set of target-specific primers have relatively uniformed melting temperatures of about 75° C. In other embodiments, the first set of target-specific primers have relatively uniformed melting temperatures of about 78° C. In other embodiments, the first set of target-specific primers have relatively uniformed melting temperatures of about 80° C.

The sequences of the first set of target-specific primers are determined such that secondary structures are minimized. In some embodiments, the first set of target-specific primers do not form hairpin structures. In other embodiments, the first set of target-specific primers do not form dimers between two molecules of the same target-specific primer. In other embodiments, the first set of target-specific primers do not form dimers between different target-specific primers.

The last five bases on the 3′ end of the first set of target-specific primers do not comprise too many G or C bases. In some embodiments, the last five bases on the 3′ end of the first set of target-specific primers comprise no G or C bases. In other embodiments, the last five bases on the 3′ end of the first set of target-specific primers comprise only one G or C base. In other embodiments, the last five bases on the 3′ end of the first set of target-specific primers comprise only two G or/and C bases. In other embodiments, the last five bases on the 3′ end of the first set of target-specific primers comprise only three G or/and C bases.

The sequences of the first set of target-specific primers comprise limited repeats of one base or dinucleotide repeats. In some embodiments, the sequences of the first set of target-specific primers comprise no repeats of one base or dinucleotide repeats. In other embodiments, the sequences of the first set of target-specific primers comprise one or more repeats of one base but no dinucleotide repeats, and wherein the one or more repeats of one base are repeats with the same base appearing only two times, only three times, or only four times. In other embodiments, the sequences of the first set of target-specific primers comprise no repeats of one base but one or more dinucleotide repeats, and wherein the one or more dinucleotide repeats are repeats with the same dinucleotide appearing only two times, only three times, or only four times. In other embodiments, the sequences of the first set of target-specific primers comprise one or more repeats of one base and one or more dinucleotide repeats, wherein the one or more repeats of one base are repeats with the same base appearing only two times, only three times, or only four times, and wherein the one or more dinucleotide repeats are repeats with the same dinucleotide appearing only two times, only three times, or only four times.

The sequences of the first set of target-specific primers are designed so that it is unlikely to generate additional (non-specific) PCR amplicons using Primer-BLAST, including SNP-containing genome databases. In some embodiments, the top non-specific PCR amplicons have at least four mismatches with the first set of target-specific primers. In other embodiments, the top non-specific PCR amplicons have at least five, at least six, at least seven, at least eight, at least nine, at least ten mismatches with the first set of target-specific primers

The first set of target-specific primers may be automatically design by available algorithms. In some embodiments, the first set of target-specific primers are designed by NGS-PrimerPlex. In other embodiments, the first set of target-specific primers are designed by PrimerPlex. In other embodiments, the first set of target-specific primers are designed by MPD. In other embodiments, the first set of target-specific primers are designed by MPprimer. In other embodiments, the first set of target-specific primers are designed by PRIMEval. In other embodiments, the first set of target-specific primers are designed by openPrimeR. In other embodiments, the first set of target-specific primers are designed by Visual OMP. In other embodiments, the first set of target-specific primers are designed by Oli2go.

In some embodiments, the first PCR comprises annealing the first set of target-specific primers to single-stranded nucleic acid fragments. The annealing temperature is determined by the lowest melting temperature among the first set of target-specific primers. In some embodiments, the annealing temperature is about 55° C. In some embodiments, the annealing temperature is about 56° C. In some embodiments, the annealing temperature is about 57° C. In other embodiments, the annealing temperature is about 58° C. In other embodiments, the annealing temperature is about 60° C. In other embodiments, the annealing temperature is about 65° C. In other embodiments, the annealing temperature is about 70° C. In other embodiments, the annealing temperature is about 75° C. In some embodiments, the annealing lasts for about 0.5 minute. In other embodiments, the annealing lasts for about 1 minute. In other embodiments, the annealing lasts for about 1.5 minutes. In other embodiments, the annealing lasts for about 2 minutes. In other embodiments, the annealing lasts for about 3 minutes. In other embodiments, the annealing lasts for about 4 minutes. In other embodiments, the annealing lasts for about 5 minutes. In other embodiments, the annealing lasts for about 6 minutes. In other embodiments, the annealing lasts for about 7 minutes. In other embodiments, the annealing lasts for about 8 minutes. In other embodiments, the annealing lasts for about 9 minutes. In other embodiments, the annealing lasts for about 10 minutes. In other embodiments, the annealing lasts for about 11 minutes. In other embodiments, the annealing lasts for about 12 minutes. In other embodiments, the annealing lasts for about 13 minutes. In other embodiments, the annealing lasts for about 14 minutes. In other embodiments, the annealing lasts for about 15 minutes.

In some embodiments, the first PCR comprises an extension. In some specific embodiments, the extension lasts for about 20 seconds. In some specific embodiments, the extension lasts for about 30 seconds. In some specific embodiments, the extension lasts for about 40 seconds. In some specific embodiments, the extension lasts for about 50 seconds. In some specific embodiments, the extension lasts for about 60 seconds. In some specific embodiments, the extension lasts for about 70 seconds. In some specific embodiments, the extension lasts for about 80 seconds. In some specific embodiments, the extension lasts for about 90 seconds. In some specific embodiments, the extension lasts for about 100 seconds. In some specific embodiments, the extension lasts for about 110 seconds. In some specific embodiments, the extension lasts for about 120 seconds. In some specific embodiments, the extension lasts for about 3 minutes. In some specific embodiments, the extension lasts for about 4 minutes. In some specific embodiments, the extension lasts for about 5 minutes. In some specific embodiments, the extension lasts for about 6 minutes. In some specific embodiments, the extension lasts for about 7 minutes. In some specific embodiments, the extension lasts for about 8 minutes. In some specific embodiments, the extension lasts for about 9 minutes. In some specific embodiments, the extension lasts for about 10 minutes. In some specific embodiments, the extension lasts for about 11 minutes. In some specific embodiments, the extension lasts for about 12 minutes. In some specific embodiments, the extension lasts for about 13 minutes. In some specific embodiments, the extension lasts for about 14 minutes. In some specific embodiments, the extension lasts for about 15 minutes.

The first PCR comprises multiple cycles of the above-described PCR (annealing, extension, and denature) so that targets can be searched among samples multiple times. In some embodiments, the cycle number is at least 3. In some embodiments, the cycle number is at least 4. In some embodiments, the cycle number is at least 5. In some embodiments, the cycle number is at least 10. In some embodiments, the cycle number is at least 15. In some embodiments, the cycle number is at least 20. In some embodiments, the cycle number is at least 25. In some embodiments, the cycle number is at least 30. In some embodiments, the cycle number is at least 35. In some embodiments, the cycle number is at least 40. In some embodiments, the cycle number is at least 45. In some embodiments, the cycle number is at least 50. In some embodiments, the cycle number is at least 55. In some embodiments, the cycle number is at least 65. In some embodiments, the cycle number is at least 70. In some embodiments, the cycle number is at least 75.

In some embodiments, the methods described herein can be used for identifying genome-wide gene editing off-target sites from a sample that is edited by CRISPR-Cas9. In some embodiments, the methods described herein can be used for identifying genome-wide gene editing off-target sites from a sample that is edited by CRISPR-Cas12. In some embodiments, the methods described herein can be used for identifying genome-wide gene editing off-target sites from a sample that is edited by a CRISPR-Cas system other than CRISPR-Cas9 or CRISPR-Cas12. In some embodiments, the methods described herein can be used for identifying genome-wide gene editing off-target sites from a sample that is edited by CRISPR base editors. In some embodiments, the methods described herein can be used for identifying genome-wide gene editing off-target sites from a sample that is edited by CRISPR prime editors. In some embodiments, the methods described herein can be used for identifying genome-wide gene editing off-target sites from a sample that is edited by transposon-based gene editors. In some embodiments, the methods described herein can be used for identifying genome-wide gene editing off-target sites from a sample that is edited by transcription activator-like effector nucleases (TALEN). In some embodiments, the methods described herein can be used for identifying genome-wide gene editing off-target sites from a sample that is edited by zinc finger nucleases (ZFN). In some embodiments, the methods described herein can be used for identifying genome-wide gene editing off-target sites from a sample that is edited by meganucleases.

In some embodiments, the methods described herein can be used to detect the random insertion site of a virus-vector delivery. In some embodiments, the methods described herein can be used to detect the random insertion site of a transposon. In some embodiments, the methods described herein can be used to detect insertion site of a donor DNA. In some embodiments, the methods described herein can be used to detect insertion site of virus, such as hepatitis B virus and human papillomavirus. In some embodiments, the methods described herein can be used to detect the neighboring sequences of any known sequences.

As used herein and in the claims, the terms “comprising” (or any related form such as “comprise” and “comprises”), “including” (or any related forms such as “include” or “includes”), “containing” (or any related forms such as “contain” or “contains”), means including the following elements but not excluding others. It shall be understood that for every embodiment in which the term “comprising” (or any related form such as “comprise” and “comprises”), “including” (or any related forms such as “include” or “includes”), or “containing” (or any related forms such as “contain” or “contains”) is used, this disclosure/application also includes alternate embodiments where the term “comprising”, “including,” or “containing,” is replaced with “consisting essentially of” or “consisting of”. These alternate embodiments that use “consisting of” or “consisting essentially of” are understood to be narrower embodiments of the “comprising”, “including,” or “containing,” embodiments.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular. Accordingly, terms such as “first” and “second” do not necessarily imply priority, order of importance, or order of acts.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to ±10% of a stated number or value.

The terms “increased”, “increasing”, or “increase” are used herein to generally mean an increase by a statically significant amount. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease by a statistically significant amount. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

For the sake of clarity, “characterized by” or “characterized in” (together with their related forms as described above), does not limit or change the nature of whether the list of terms following it are open or closed. For example, in a claim directed towards “a composition comprising A, B, C, and characterized in D, E, and F”, the elements D, E, and F are still open-ended terms and the claim is meant to include other elements due to the use of the word “comprising” earlier in the claim.

As used herein and in the claims, the singular forms “a”, “an” and “the” are intended to include the plural forms as well, unless the context clearly indicates otherwise. Where a range is referred in the specification, the range is understood to include each discrete point within the range. For example, 1-7 means 1, 2, 3, 4, 5, 6, and 7.

As used herein and in the claims, the term “about” or “around” is understood as within a range of normal tolerance in the art and not more than ±10% of a stated value. By way of example only, about 50 means from 45 to 55 including all values in between. As used herein, the phrase “about” a specific value also includes the specific value, for example, about 50 includes 50.

As used herein and in the claims, “enriching” means increasing the proportion of molecule target of interest among all molecules from a sample.

As used herein and in the claims, “nucleic acid fragments” means the nucleic acid has been fragmented into shorter pieces. In certain embodiments, the nucleic acid fragmented into typical sizes peaking at around 50 bp to 1000 bp long. In certain embodiments, the nucleic acid fragmented into typical sizes peaking at around 20 to 50 bp, 51 to 100 bp, 101 to 300 bp, 301 to 500, and 501 to 1000 bp.

As used herein and in the claims “high molecular weight DNA” refers to DNA that has not been fragmented into shorter pieces. In certain embodiments, a high molecular weight DNA can be around 300 bp or longer. In certain embodiments, a high molecular weight DNA can be around 500 bp or longer.

As used herein and in the claims, “indel” means an insertion or deletion of bases in the genome of an organism.

As used herein and in the claims, “off-target genome editing” refers to unintended genetic modifications that can arise through the use of engineered nuclease technologies, such as CRISPR-Cas9, CRISPR-Cas12 and other CRISPR-Cas systems, CRISPR base editors, CRISPR prime editors, transposon-based gene editors and writers, transcription activator-like effector nucleases (TALEN), meganucleases, and zinc finger nucleases (ZFN).

As used herein and in the claims, “off-target” or “off-targets” refer to one or more sites in a given genome or set of user-defined sequences that are subjected to genetic modifications by off-target genome editing.

As used herein and in the claims, “on-target genome editing” refers to intended or expected genetic modifications that can arise through the use of engineered nuclease technologies, such as CRISPR-Cas9, CRISPR-Cas12 and other CRISPR-Cas systems, CRISPR base editors, CRISPR prime editors, transposon-based gene editors and writers, transcription activator-like effector nucleases (TALEN), meganucleases, and zinc finger nucleases (ZFN).

As used herein and in the claims, “universal oligonucleotide adaptor” refers to a nucleic acid molecule comprised of two strands (a top strand and a bottom strand) and comprising a first ligatable 5′ protrude end and a second un-ligatable end. In some embodiments, the top strand of the universal oligonucleotide adaptor comprises a 5′ duplex portion, and the bottom strand comprises an unpaired 5′ portion, a 3′ duplex portion, and nucleic acid sequences identical to a first and second sequencing primers. The duplex portions of the adaptor may be substantially complementary and the duplex portion is of sufficient length to remain in duplex form at the ligation temperature. In certain embodiments, the top strand and the bottom strand are connected to each other and form a hairpin loop. The term “sufficient” means that the number of bases in the duplex portion is long enough so that the bonding therebetween can keep in duplex form at the ligation temperature.

As used herein and in the claims, “genome editing”, or “genome engineering”, or “gene editing”, is a type of genetic engineering in which DNA is inserted, deleted, modified or replaced in the genome of a living organism. As an example, genome editing targets the insertions to site specific locations.

As used herein and in the claims, “CRISPR (Clustered, Regularly Interspaced, Short Palindromic Repeats) gene editing” is a genetic engineering technique in molecular biology by which the genomes of living organisms may be modified by an engineered Cas (Clustered, Regularly Interspaced, Short Palindromic Repeats-associated protein) nuclease.

As used herein and in the claims, “GUIDE-Seq (Genome-wide, Unbiased Identification of DSBs Enabled by Sequencing)” is a molecular biology technique that allows for the unbiased in vitro and cell-based detection of off-target genome editing events in DNA caused by CRISPR/Cas nucleases as well as other RNA-guided nucleases in living cells.

As used herein and in the claims, “DISCOVER-Seq (Discovery of in situ Cas off-targets and verification by sequencing)” is a molecular biology technique that allows for unbiased CRISPR-Cas off-target identification in cells and tissues.

As used herein and in the claims, “EDITED-Seq (editing events detection by sequencing)” is a molecular biology technique as described in the present disclosure that allows for detection and/or evaluation of off-targets.

As used herein and in the claims, “anchored polymerase chain reaction” or “anchored PCR” refers to PCR performed with at least one anchored primer and extending from at least one end of the nucleic acid fragments. In certain embodiments, anchored PCR can be PCR performed with an anchored primer and extending from a single-end of the nucleic acid fragments. In certain embodiments, anchored PCR can be PCR performed with two anchored primers and extending from both ends of the nucleic acid fragments.

As used herein and in the claims, “a universal oligonucleotide adaptor primer” refers to a primer that can anneal to part of the sequence of the universal oligonucleotide adaptor. In some aspects, the universal oligonucleotide adaptor comprises at least one secondary structure such as a hairpin structure,

As used herein, “nested”, “nested amplification”, or “nested PCR” refers to a polymerase chain reaction for decreases non-specific binding in products due to the amplification of unexpected primer binding sites. Nested PCR comprises at least two sets of primers, used in at least two successive runs of PCR, where a second PCR amplifies a secondary target within the first PCR product. Such arrangement allows amplification for a low number of runs in the first PCR, limiting non-specific products. The second nested primer set can amplify the intended product from the first PCR. The at least one target nucleic acid undergoes the first PCR with a first set of primers. The PCR product from the first PCR can then be amplified with a second PCR with a second set of primers.

As used herein, “unique molecular index” refers to nucleic acid sequences added to the at least one target nucleic acid or any nucleic acid fragment described herein during nucleic acid library preparation for identifying the nucleic acid. The unique molecular index can be added before any round of the PCR described herein (e.g., first round of PCR, second round of PCR, etc) and can be used to decrease errors and quantitative bias introduced by the amplification.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### EXAMPLES

Provided herein are examples that describe in more detail certain embodiments of the present disclosure. The examples provided herein are merely for illustrative purposes and are not meant to limit the scope of the disclosure in any way.

### Example 1—Example Workflow

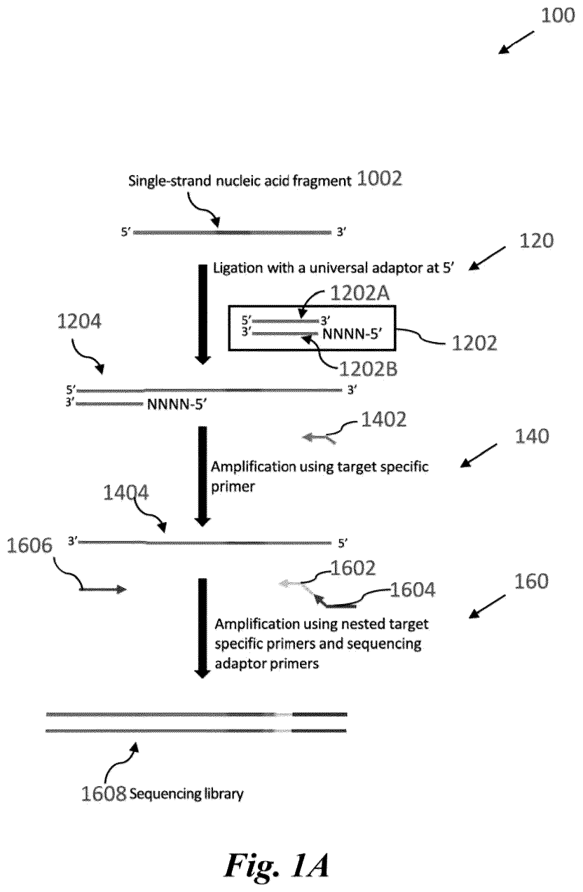

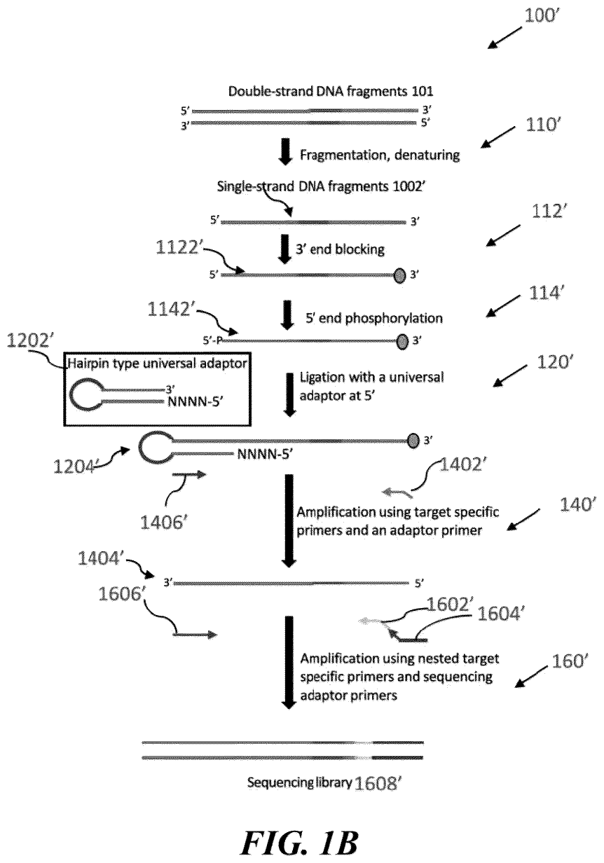

FIG. 1A shows a workflow of an example method 100 for amplifying targeted nucleic acid from a sample. In this example, the sample contains single-stranded nucleic acid fragment 1002, which contain a target nucleic acid sequence. By way of example, the sample is from a mammal, (e.g., a human). By way of example, the human is a fetus. By way of example, the human is an individual known to have or suspected of having a disease, (e.g. a cancer or a genetic disorder). By way of example, one or more of the target sequences comprise one or more markers for a disease, e.g., a cancer. By way of example, the sample is from a blood sample. By way of example, the sample is cell-free nucleic acids extracted from a blood sample. By way of example, the sample is nucleic acids extracted from circulating tumor cells. By way of example, the single-stranded nucleic acid 1002 in the sample is single-strand DNA fragments prepared from denaturation of double-strand DNA fragments. By way of example, the single-stranded nucleic acid 1002 in the sample is single-strand cDNA fragments prepared from reverse transcription of RNA fragments. By way of example, the sample is nucleic acids extracted from lymphocytes in a blood sample for T-cell and B-cell receptor profiling. By way of example, the sample is a CRISPR gene edited sample. By way of example, the sample is meganucleases edited, zinc finger nucleases (ZFNs) edited, or transcription activator-like effector nucleases (TALENs) edited. By way of example, the sample is from CAR-T, CAR-NK, TCR-T, immortalized cell lines (e.g., engineered neural stem cell line CTX) or hematopoietic stem cells for therapeutics. By way of example, the sample is from genetically engineered cells (ex-vivo or in vivo), wherein the cells include but are not limited to fibroblasts, chondrocytes, keratinocytes, hepatocytes, pancreatic islet cells, stem cells (e.g., haematopoietic stem cells, mesenchymal stem cells, or skin stem cells), and immune cells (e.g., tumor infiltrating lymphocytes, viral reconstitution T cells, dendritic cells, γδ T cells, regulatory T cells (Treg) and macrophages).

Still referring to FIG. 1A, in 120, a universal oligonucleotide adaptor (or universal adaptor) 1202 is ligated with the single-stranded nucleic acid fragment 1002 at the 5′ end to form a ligation product 1204. In this example, the universal oligonucleotide adaptor 1202 includes a top strand 1202A with a 3′ recessive end which is configured for ligating to the 5′ end of the single-stranded nucleic acid fragment 1002, and a bottom strand 1202B with a 5′ protrude end including multiple number bases of random or degenerate nucleotides, for example, three to twenty. In this example, the number of bases of random nucleotides is four. In some embodiments, the top strand 1202A of the universal oligonucleotide adaptor 1202 comprises a 5′ duplex portion, and the bottom strand 1202B comprises a 3′ duplex portion. The duplex portions of the adaptor may be substantially complementary and the duplex portion is of sufficient length to remain in duplex form at the ligation temperature. In some embodiments, the universal oligonucleotide adaptor 1202 may further comprise three to twenty random nucleotides incorporated in the duplex portion or in a 5′end of the top strand 1202A as a unique molecular index (UMI) for tracing individual original molecules. In 140, the ligation product 1204 is subsequently amplified by a first PCR with a first target-specific primer 1402 to form a first PCR product 1404. In this example, the first PCR is a linear amplification of the ligation product to obtain a nascent primer extension duplex. By way of example, the first PCR includes (1) annealing a first target-specific primer 1402 to the single-strand nucleic acid fragments 1002 in the vicinity of a target sequence, (2) extending the first target-specific primer 1402 over the single-strand nucleic acid fragments 1002 using a DNA polymerase, (3) obtaining a nascent primer extension duplex and (4) dissociating the nascent primer extension duplex into single strands. By way of example, the first PCR may further repeat the (1)-(4) in one or more cycles. In another example embodiment, the first PCR of the 140 is an exponential amplification of the targeted nucleic acid with the first target-specific primer 1402 and a universal oligonucleotide adaptor primer. By way of example, the first PCR product is optionally cleaned up to remove the first target-specific primer 1402 before the subsequent step(s). In 160, the first PCR product 1404 is amplified by a second PCR with a second target-specific primer 1602 nested relative to the first target-specific primer 1402 and a sequencing adaptor reverse primer 1606 (also referred to as a universal oligonucleotide adaptor primer in some embodiments). The second target-specific primer 1602 and the sequencing adaptor reverse primer 1606 are used in the amplification of the first PCR product 1404 to form a second PCR product 1608. By way of example, the first PCR is a linear PCR. By way of example, the first PCR is a gene-specific primer (GSP) PCR. By way of example, the first PCR and/or second PCR are multiplexing PCR. By way of example, the 160 may further include performing a nested amplification of the nascent primer extension duplex. Optionally, a sequencing adaptor forward primer 1604 is provided so that the second PCR product 1608 can be used as a sequencing library. By way of example, the sequencing adaptor primer 1604 is provided so that a plurality of 1602 can be bridged and sequenced using a same sequencing primer identical to 1604. By ways of example, the sequencing adaptor forward primer 1604 and the sequencing adaptor reverse primer 1606 are Illumina sequencing primers. By way of example, sequencing adaptor forward primer 1604 is not provided. By way of example, the sequencing library may be used for subsequent sequencing with a sequencing primer pair (not shown), which is at least partially complementary to opposite strands of the second PCR product 1608, respectively. In another example embodiment, the second target-specific primer 1602 includes the sequence of sequencing adaptor forward primer 1604.

Referring now to FIG. 1B, which shows workflow of alternative example method 100′ for amplifying targeted nucleic acid from a sample. For the sake of clarity, any one or more of the additional or alternate steps in this example can be added into or replaced with the corresponding steps in method 100 (FIG. 1A), respectively. In this example, the starting material of the nucleic acid is double-stranded DNA 101 which contains a targeted DNA sequence. By way of example, the sample includes a plurality of DNA fragments prepared from high molecular weight DNA, e.g., genomic DNA. In an additional 110′, the double-stranded DNA 101 is fragmented and denatured to form single-stranded DNA fragments 1002′. In an optional 112′, the 3′ end of the single-stranded DNA fragments 1002′ may be optionally blocked to form 3′ end blocked single-stranded DNA fragments 1122′. In an optional 114′, the 5′ end of the single-stranded DNA fragments 1002′ or 1122′ may be optionally phosphorylated to form 5′ end phosphorylated single-stranded DNA fragments 1142′. Then 5′ end phosphorylated single-stranded DNA fragments 1142′ is ready for the subsequent 120′ (or 120). Optionally, the single-stranded nucleic acid fragments as described may be further adenylated to produce a 3′-adenosine overhang on the single-strand nucleic acid fragments prior to ligation 120′. In alternative 120′, the universal oligonucleotide adaptor 1202′ which contain a hairpin loop connecting a portion of the duplex form (as shown in the box in FIG. 1B) is used to ligate to 5′ end phosphorylated single-stranded DNA fragments 1142′ at 5′ end to form a ligation product 1204′. By way of example, the single-stranded DNA fragments for ligation may be single-stranded DNA fragments 1002′ or 3′ end blocked single-stranded DNA fragments 1122′. In alternative 140′, the ligation product 1204′ is subsequently amplified by a first PCR with a first target-specific primer 1402′ and a first universal adaptor specific primer 1406′ to form a first PCR product 1404′. In 160′, the first PCR product 1404′ is amplified by a second PCR with a second target-specific primer 1602′ and a sequencing adaptor reverse primer 1606′ (also referred to as a universal oligonucleotide adaptor primer in some embodiments) to form a sequencing library 1608′, which is a double-stranded DNA product containing targeted DNA sequence with sequencing adaptor primer sequence. The second target-specific primer 1602′ is nested relative to the first target-specific primer 1402′. Optionally, a sequencing adaptor forward primer 1604′ is provided. In another example embodiment, the second target-specific primer 1602′ includes the sequence of sequencing adaptor forward primer 1604′.

### Example 2. Plasmid Construction

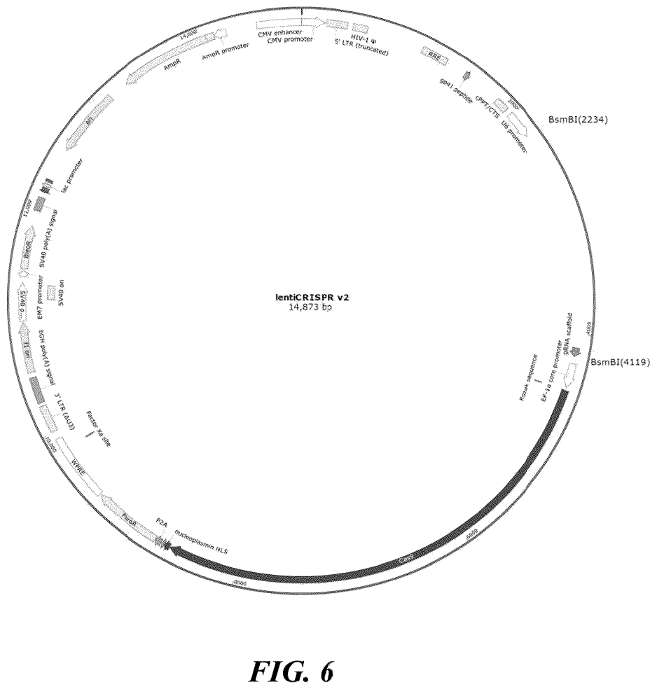

Paring protospacer oligos were annealed and inserted between two BsmI cleavage sites of the lentiCRISPR vector (Addgene #42230). The topology of the lentiCRISPR vector is shown in FIG. 6. Sequence authenticity of each vector was confirmed by Sanger sequencing. The sequences of paring protospacer oligos are shown in Table 2 below.

TABLE 2
Sequences of paring protospacer oligos

| Primer | | | SEQ ID |
|---|---|---|---|
| Name/ID | Sequence | Usage/Remarks | NO: |
| sgVEGFA4-F | caccgGACCCCCTCCACCCCGCCTC | sgRNA cloning | 1 |
| sgVEGFA4-R | aaacGAGGCGGGGTGGAGGGGGTCc | sgRNA cloning | 2 |
| sgHBB-F | caccgCTTGCCCCACAGGGCAGTAA | sgRNA cloning | 3 |
| sgHBB-R | aaacTTACTGCCCTGTGGGGCAAGc | sgRNA cloning | 4 |
| sgPD1-F | caccgGGGCGGTGCTACAACTGGGC | sgRNA cloning | 5 |
| sgPD1-R | aaacGCCCAGTTGTAGCACCGCCCc | sgRNA cloning | 6 |
| sgTRAC-F | caccgCTTCAAGAGCAACAGTGCTG | sgRNA cloning | 7 |
| sgTRAC-R | aaacCAGCACTGTTGCTCTTGAAGc | sgRNA cloning | 8 |
| sgALB-F | caccgGGTGTAAAATCAACACCCTA | sgRNA cloning | 9 |
| sgALB-R | aaacTAGGGTGTTGATTTTACACCc | sgRNA cloning | 10 |
| sgALB-F | caccgGGTGTAAAATCAACACCCTA | sgRNA cloning | 9 |
| sgALB-R | aaacTAGGGTGTTGATTTTACACCc | sgRNA cloning | 10 |
| sgGAPDH-F | caccgAGCCCCAGCAAGAGCACAAG | sgRNA cloning | 11 |
| sgGAPDH-R | aaacCTTGTGCTCTTGCTGGGGCTc | sgRNA cloning | 12 |
| Illumina.Y.adap- | AATGATACGGCGACCACCGAGATC | Illumina adaptor | 13 |
| tor.primer | TACACNNNNNNNNACACTCTTTC | | |
| | CCTACACGACGCTCTTCCGATCT | | |
| Illumina.i7.adap- | CAAGCAGAAGACGGCATACGAGA | Illumina adaptor | 14 |
| tor.primer | TNNNNNNNNGTGACTGGAGTTCA | | |
| | GACGTGTGCTCTTCCGATC | | |

### Example 3. Off-Targets Prediction and Anchored Multiplex Primers Design

Potential off-targets were initially predicted in silico based on three professional tools, E-CRISP, Cas-OFFinder, and CRISPRscan. The following cutoffs were used respectively, mismatch <=7 for E-CRISP, mismatch <=4 and bulge <=2 for Cas-OFFinder, and no threshold for CRISPRsan. To reduce false positive and computational bias, a combinatorial strategy was used that those sites found by at least two methods were applied to further primer design.

### Example 4. Cell Culture and Transfection

K562 cells were seeded in a flask containing 15 mL Roswell Park Memorial Institute 1640 medium (RPMI 1640; Thermo Fisher Scientific, Waltham, MA, USA), supplemented with 10% heat-inactivated fetal bovine serum (FBS, Thermo Fisher Scientific), grown at 37° C. within 5% carbon dioxide (CO₂). After grown for 20-24 hours to achieve a confluence of 70-90%, cells were harvested for Neon transfection. Neon transfection was conducted using a Neon transfection platform (Thermo Fisher Scientific) according to the manufacturer's instructions. Briefly, 2×10⁶ cells per test were suspended in the Electrolyte Buffer mixed with 5 μg of lentiCRSIPR-sgRNA plasmids to a final volume of 100 μL. Then cell/DNA mixture was pulsed by the Neon machine under the following parameters: voltage=1600 V; width=10 ms; number=3. Cells were continued typically for 72 hours followed by DNA and mRNA extraction. For GUIDE-Seq, 200 pmol of annealed double-stranded oligonucleotide (dsODN) was mixed with desired plasmid, followed by the same Neon transfection process described above.

HEK293 or NIH 3T3 cells were seeded at a density of 1.5×10⁵ cells/well in a 12-well plate, grown at 37° C. within 5% CO₂ in Dulbecco's modified Eagle's medium (DMEM; Life Technologies), supplemented with 10% FBS, 1% penicillin, and 1% streptomycin. After grown for 24 hours, transfection was carried out with Lipofectmin3000 (Thermo Fisher Scientific) according to the manufacturer's instruction. Briefly, 1 μg of lentiCRSIPR-sgRNA vectors, 2 μL of P3000, and 2.5 μL of Lipofectmin3000 were mixed gently with FBS-free DMEM to a final volume of 100 μL, incubated at room temperature for 15 min, and added to the medium. Cells were harvested after 72 hours post transfection for DNA extraction. For GUIDE-Seq experiment, 10 pmol of annealed dsODN was mixed and co-incubated with Lipofectmin3000, followed by the same protocol above.

### Example 5. DNA and Total RNA Extraction

Total DNA and RNA were extracted separately using the AllPrep DNA/RNA Kit (QIAGEN, Hilden, Germany) according to the manufacturer's instructions. Briefly, cells/tissues were lysed by Buffer RLT Plus (350 μL per test of <10⁷ cells or 30 mg tissues). The lysed mixture was filtered by AllPrep DNA column, followed by washing and elution of the column-bound genomic DNA. The flow-through from the column was used as RNA origin for mRNA extraction through AllPrep RNA column. Extracted DNA/RNA was quantified by the corresponding DNA/RNA Qubit Assay Kit (Thermo Fisher Scientific), and were stored at −80° C. until use.

### Example 6. Genome Editing in Primary Cells and iPSC

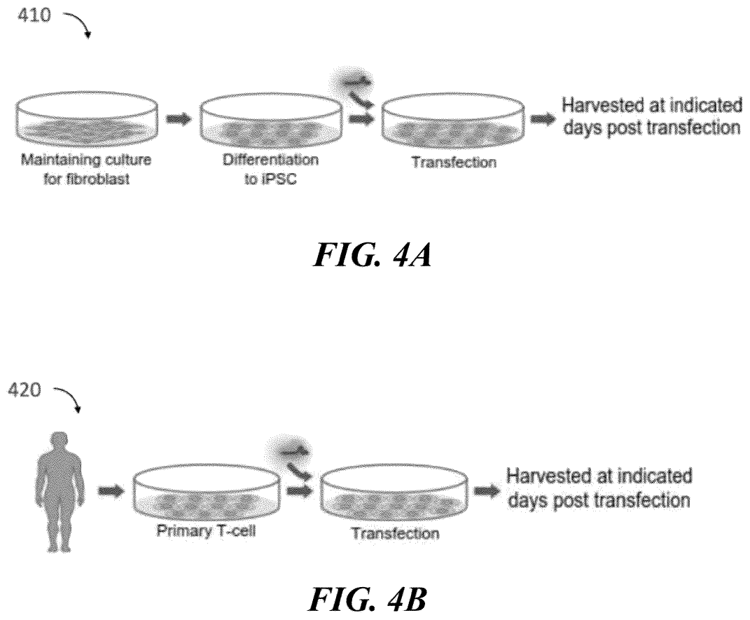

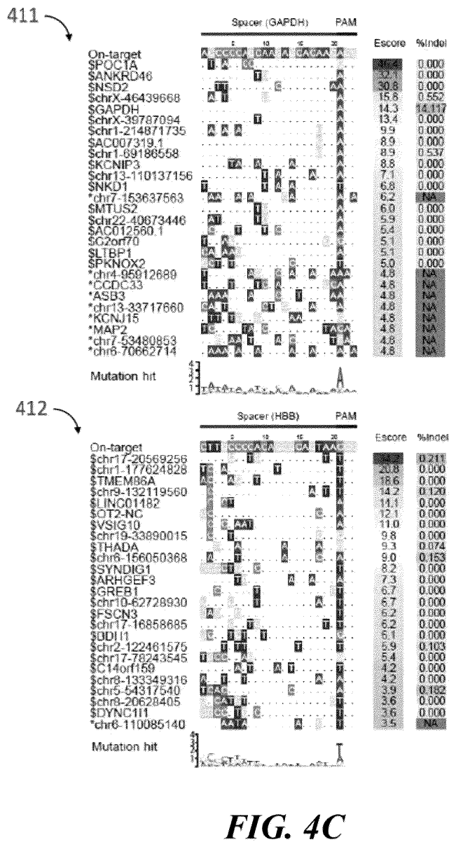

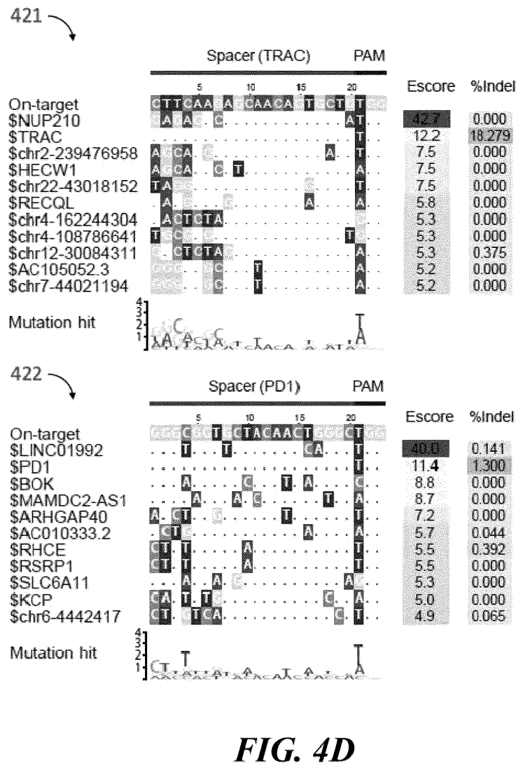

FIG. 4A shows a workflow of an example method 410 of iPSC editing by CRISPR-Cas9, according to an example embodiment. A culture for fibroblast was maintained and the culture was allowed to differentiate to iPSC. iPSCs were then transfected using Amaxa nucleofection (Lonza, Allendale, NJ, USA) according to the manufacturer's instructions. Briefly, cells were firstly dissociated into single cells using TrypLE. For each transfection, 5×10⁶ cells were mixed with 100 μL pre-warmed nucleofection reagents (82 μL solution-1 and 18 μL solution-B); then 10 μg DNA (6 μg Cas9+4 μg sgRNA) was added into the suspension and electroporated. Electroporated iPSCs were cultured on inactivated MEF feeders, with fresh medium changed daily for 4-5 days and then harvested for DNA isolation. The cells were harvested at indicated days post transfection.

FIG. 4B shows a workflow of an example method 420 of T-cell editing by CRISPR-Cas9, according to an example embodiment. In this example embodiment, the T-cells were transfected similarly as previously described for iPSC (FIG. 4A).

### Example 7. Genome Editing in Mouse

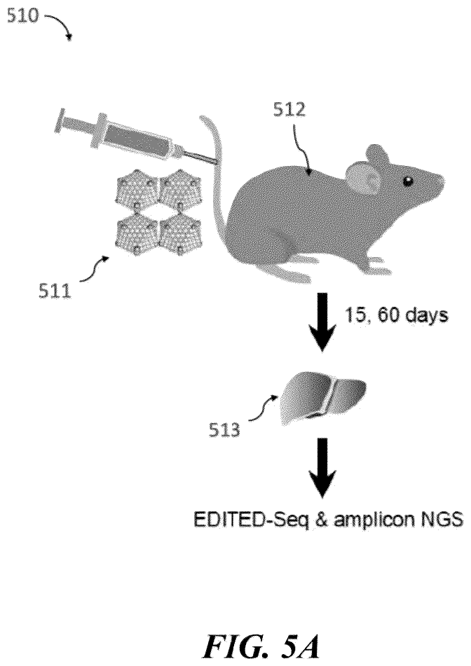

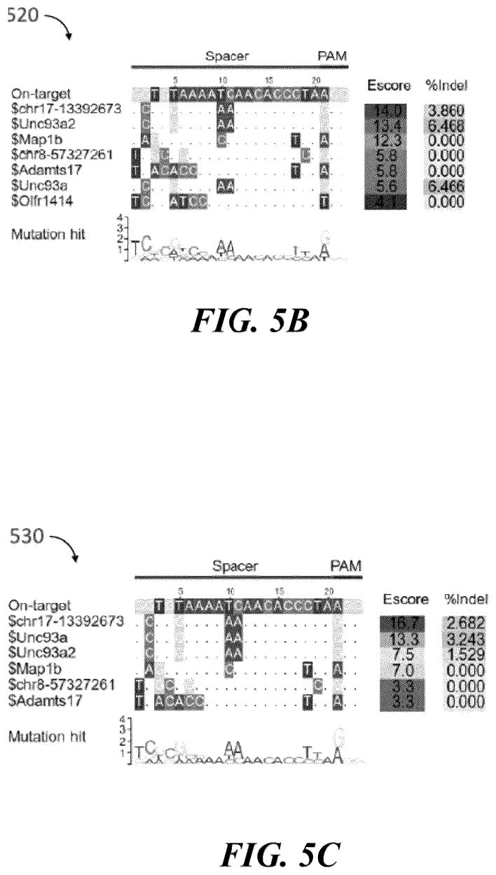

FIG. 5A shows a workflow of an example method 510 of EDITED-Seq conducted in a mouse, according to an example embodiment. A total of 10⁷-10⁸ TU AAV8 virus 511 were injected into nine- to eleven-week-old male C57BL/6 mice 512 (weighed before experiment) via tail vein within 5-7 s. Mouse (weighed before sacrifice) was euthanized by cardiac puncture after 15, 30, and 60 days. Blood was collected in EDTA-coated capillary tubes and kept on ice for up to 2 hours before extraction of centrifugation at 10,000 rpm for 20 min at 4° C. The liver organ 513 was dissected, snap-frozen in liquid nitrogen and stored at −80° C. until use. Ground tissues were lysed by Buffer RLT Plus (350 μL per 20 mg tissues) and extracted by AllPrep DNA/RNA Kit (Qiagen) according to manufacturer's instructions. DNA and RNA were stored at −80° C. until subjected to EDITED-Seq, amplicon-NGS and qRT-PCR.

### Example 8. EDITED-Seq Pipeline

Genomic DNA and anchored single-end multiplex primers were the inputs to generate EDITED-Seq library via two-round gene-specific primer (GSP) PCR, one anchored PCR and one nested anchored plus indexing PCR, according to the example methods 100 or 100′ as described in Example 1. In brief, indicated amount of DNA was fragmented to typical sizes peaking at 300-500 bp, then single-stranded adaptor was used to block the 3-termini of these DNA fragments. Indexed single-stranded adaptor was ligated to the 5-termini after phosphorylation by T4 polynucleotide kinase (T4 PNK; New England Biolabs, Ipswich, MA, USA) so as to improve the ligation efficiency, which was followed by first-round linear GSP PCR to capture all potential off-targets. The second-round nested GSP PCR was conducted after cleaning up the primers from the first round. Final sequencing library was checked by gel electrophoresis and quantified by quantitative PCR (qPCR) using the Illumina sequencing primers, followed by Next-Seq/MiSeq (Illumina, San Diego, CA, USA).

### Example 9. Detection of Gene Translocation and Edit of Potential Off-Targets

Qualified reads were mapped to human genome (GRCh38) using Burrows-Wheeler Alignment Tool (BWA mem) (version 0.7.17-r1188). Translocation can be observed when one read is split into different loci (split read) or the mate of one anchored read mapped to a new locus (discordant read). To identify split/discordant reads, Breakmer (version 0.0.7; with parameters: trl_sr_thresh 1, rearr_sr_thresh 1, and discread_only_thresh 1) were used to profile potential candidate translocations, followed by estimate of protospacer similarity to on-target spacer and cutting frequency determinant (CFD). The resulting off-target candidates with CFD above 0.01 were further filtered by the orientations of split/discordant reads at each corresponding locus and the negative control to minimize nonspecific fusion by false amplification and hotspot DSB sites.

For Indel frequency determination, mapped reads were re-aligned by GATK-realigner (version 3.8.0), then subjected to filtering those reads not spanning the corresponding spacer regions. The resulting reads were then estimated the insertion and deletion occurring around 5-bp up/downstream of cleavage site using custom script. Reliable Indel frequency was determined by the Indel value of treatment sample with an elimination by corresponding value of negative control.

### Example 10. EDITED-Seq Strategy

In this example embodiment, a method for editing events detection by sequencing (EDITED-Seq) was conducted according to procedures described in Examples 8 and 9 to simultaneously detect new and validate known or in-silico-predicted off-target sites.

In some embodiments, by using on-target as well as highly potential off-targets as seeds, novel CRISPR-edited off-target sites could be extensively hooked via linear amplification using targeted-primers because of fusions between double-strand breaks that are induced by CRISPR editing. Anchored polymerase chain reaction was implemented to capture and also validate all potential edited off-targets, without any preliminary experimental process before starting off-target profiling.

In this example embodiment, EDITED-Seq was initially performed according to Examples 8 and 9 on VEGFA_2 in K562 cells. The sequences of anchored primers for VEGFA_2 used in EDITED-Seq in this example embodiment is shown in Table 3 below.

TABLE 3
Sequences of anchored primers for VEGFA_2

| 1st PCR | | SEQ | 2nd PCR | | SEQ |
|---|---|---|---|---|---|
| primer | | ID | primer | | ID |
| name | Sequence | NO: | name | Sequence | NO: |
| ABLIM1_m1 | CCCCTTAGGGATA | 15 | ABLIM1_m2 | GTGACTGGAGTTCA | 150 |
| | ACAGGGTAATCCA | | | GACGTGTGCTCTTCC | |
| | ACTGCCATATGCC | | | GATCTGCCCTGGGTC | |
| | CTGGGT | | | TCTGAAGAAGCT | |
| ABLIM1_p1 | CCCCTTAGGGATA | 16 | ABLIM1_p2 | GTGACTGGAGTTCA | 151 |
| | ACAGGGTAATCCG | | | GACGTGTGCTCTTCC | |
| | GCGGGTGGGTCAC | | | GATCTGGCGGGTGG | |
| | AAA | | | GTCACAAAATAAAAT | |
| | | | | GT | |
| ACLY_m1 | CCCCTTAGGGATA | 17 | ACLY_m2 | GTGACTGGAGTTCA | 152 |
| | ACAGGGTAATCCA | | | GACGTGTGCTCTTCC | |
| | CAGGACAGGGTC | | | GATCTACAGGACAGG | |
| | AGCGT | | | GTCAGCGTTTAAGA | |
| ACLY_p1 | CCCCTTAGGGATA | 18 | ACLY_p2 | GTGACTGGAGTTCA | 153 |
| | ACAGGGTAATCGG | | | GACGTGTGCTCTTCC | |
| | CCCCTACAATACT | | | GATCTAAGTTTGCTG | |
| | ATCTTGACCCT | | | GCCCTGGTTTAGA | |
| ATL3-NC_m1 | CCCCTTAGGGATA | 19 | ATL3-NC_m2 | GTGACTGGAGTTCA | 154 |
| | ACAGGGTAATCTG | | | GACGTGTGCTCTTCC | |
| | AGAGACAGGGTCT | | | GATCTTGCAGTACAG | |
| | TGCTGTTG | | | TGATGGGACCGT | |
| B4GALNT4_m1 | CCCCTTAGGGATA | 20 | B4GALNT4_m2 | GTGACTGGAGTTCA | 155 |
| | ACAGGGTAATCCC | | | GACGTGTGCTCTTCC | |
| | AACTTGGTGGGGG | | | GATCTCCTTAGGGGG | |
| | TAGAGTG | | | CCAGCAGTG | |
| CALY_m1 | CCCCTTAGGGATA | 21 | CALY_m2 | GTGACTGGAGTTCA | 156 |
| | ACAGGGTAATCTC | | | GACGTGTGCTCTTCC | |
| | ACGCAGACGCCCC | | | GATCTGCAGACGCCC | |
| | CAT | | | CCATCAAGCC | |
| CALY_p1 | CCCCTTAGGGATA | 22 | CALY_p2 | GTGACTGGAGTTCA | 157 |
| | ACAGGGTAATCAG | | | GACGTGTGCTCTTCC | |
| | CCTGGAGTTAAGG | | | GATCTTGGAGTTAAG | |
| | GTGTCTCC | | | GGTGTCTCCGAGGTG | |
| CDC42SE1_m1 | CCCCTTAGGGATA | 23 | CDC42SE1_m2 | GTGACTGGAGTTCA | 158 |
| | ACAGGGTAATCCC | | | GACGTGTGCTCTTCC | |
| | CCAGGAGCGTGG | | | GATCTGCGCGCACCC | |
| | ATGACTAC | | | CTTTCCCA | |
| CDC42SE1_p1 | CCCCTTAGGGATA | 24 | CDC42SE1_p2 | GTGACTGGAGTTCA | 159 |
| | ACAGGGTAATCGC | | | GACGTGTGCTCTTCC | |
| | AGGTGAGGCCGTG | | | GATCTGGTGAGGCCG | |
| | CAG | | | TGCAGTTGGTC | |
| CDKN2C-NC_m1 | CCCCTTAGGGATA | 25 | CDKN2C-NC_m2 | GTGACTGGAGTTCA | 160 |
| | ACAGGGTAATCTG | | | GACGTGTGCTCTTCC | |
| | AGTTATGTGGTCC | | | GATCTAAGCCTCTTG | |
| | CCTCTAGGAA | | | AACATGCCGAAATGT | |
| | | | | A | |
| CDKN2C-NC_p1 | CCCCTTAGGGATA | 26 | CDKN2C-NC_p2 | GTGACTGGAGTTCA | 161 |
| | ACAGGGTAATCAG | | | GACGTGTGCTCTTCC | |
| | CGTCGTCTCCTGG | | | GATCTTCGTCTCCTG | |
| | AGCTC | | | GAGCTCTGGACAC | |
| Chr4-NC_m1 | CCCCTTAGGGATA | 27 | Chr4-NC_m2 | GTGACTGGAGTTCA | 162 |
| | ACAGGGTAATCTG | | | GACGTGTGCTCTTCC | |
| | ATGGCATCAAAAT | | | GATCTCCACCTGTGG | |
| | GTGTGTCCAGT | | | CTGATAGTGACGTCT | |
| Chr4-NC_p1 | CCCCTTAGGGATA | 28 | Chr4-NC_p2 | GTGACTGGAGTTCA | 163 |
| | ACAGGGTAATCGG | | | GACGTGTGCTCTTCC | |
| | AGGTGGCTTCACT | | | GATCTAGGTCTGGGG | |
| | TAGGAGGTC | | | AGCGGAGTCC | |
| Chr6-NC_m1 | CCCCTTAGGGATA | 29 | Chr6-NC_m2 | GTGACTGGAGTTCA | 164 |
| | ACAGGGTAATCAG | | | GACGTGTGCTCTTCC | |
| | CAAGGCTGACACC | | | GATCTACCGCCTCCA | |
| | AGGTG | | | CCCCCAAGG | |
| Chr6-NC_p1 | CCCCTTAGGGATA | 30 | Chr6-NC_p2 | GTGACTGGAGTTCA | 165 |
| | ACAGGGTAATCGG | | | GACGTGTGCTCTTCC | |
| | CTGGGATCTGGGG | | | GATCTGGATCTGGGG | |
| | AGAGAG | | | AGAGAGGTGACC | |
| CLYBL_m1 | CCCCTTAGGGATA | 31 | CLYBL_m2 | GTGACTGGAGTTCA | 166 |
| | ACAGGGTAATCAA | | | GACGTGTGCTCTTCC | |
| | TCATCAGGTGCAA | | | GATCTTGTATGTATGC | |
| | GGCAAGACTG | | | AAAGCCCCGTCACG | |
| CLYBL_p1 | CCCCTTAGGGATA | 32 | CLYBL_p2 | GTGACTGGAGTTCA | 167 |
| | ACAGGGTAATCTC | | | GACGTGTGCTCTTCC | |
| | TGACTGGAGTTCC | | | GATCTGACTGGAGTT | |
| | CTTCACCA | | | CCCTTCACCATTTCA | |
| | | | | A | |
| CRB2_m1 | CCCCTTAGGGATA | 33 | CRB2_m2 | GTGACTGGAGTTCA | 168 |
| | ACAGGGTAATCGA | | | GACGTGTGCTCTTCC | |
| | GGAGCCTGGACA | | | GATCTCCTGGACAGA | |
| | GACGAAG | | | CGAAGGCAGCA | |
| CRB2_p1 | CCCCTTAGGGATA | 34 | CRB2_p2 | GTGACTGGAGTTCA | 169 |
| | ACAGGGTAATCGC | | | GACGTGTGCTCTTCC | |
| | TGCCAGAAGCCTG | | | GATCTGCCTGTAGAG | |
| | TAGAGAT | | | ATCAAGGCTGCTC | |
| CXXC5_m1 | CCCCTTAGGGATA | 35 | CXXC5_m2 | GTGACTGGAGTTCA | 170 |
| | ACAGGGTAATCAG | | | GACGTGTGCTCTTCC | |
| | CTCGGGGGTGATT | | | GATCTTCGGGGGTGA | |
| | AGTTGC | | | TTAGTTGCTTTTTGTT | |
| CXXC5_p1 | CCCCTTAGGGATA | 36 | CXXC5_p2 | GTGACTGGAGTTCA | 171 |
| | ACAGGGTAATCGC | | | GACGTGTGCTCTTCC | |
| | CGTGGCCCGACAC | | | GATCTCCCGACACCT | |
| | CTA | | | ACCGGCTCTCC | |
| DOLK-NC_m1 | CCCCTTAGGGATA | 37 | DOLK-NC_m2 | GTGACTGGAGTTCA | 172 |
| | ACAGGGTAATCTA | | | GACGTGTGCTCTTCC | |
| | AGAAGGGCCCCTT | | | GATCTGGTCCTGGTG | |
| | GATGAGGTC | | | CTGTTCAGCCCATCT | |
| | | | | T | |
| DOLK-NC_p1 | CCCCTTAGGGATA | 38 | DOLK-NC_p2 | GTGACTGGAGTTCA | 173 |
| | ACAGGGTAATCGG | | | GACGTGTGCTCTTCC | |
| | GAGAGGTGGGTC | | | GATCTGGTGGGTCAA | |
| | AACTTTGG | | | CTTTGGCAGGGT | |
| ELL-NC_m1 | CCCCTTAGGGATA | 39 | ELL-NC_m2 | GTGACTGGAGTTCA | 174 |
| | ACAGGGTAATCGA | | | GACGTGTGCTCTTCC | |
| | GGGTGGGCGTGGC | | | GATCTGGTGGGCGTG | |
| | TATGTA | | | GCTATGTAAACGGA | |
| ELL-NC_p1 | CCCCTTAGGGATA | 40 | ELL-NC_p2 | GTGACTGGAGTTCA | 175 |
| | ACAGGGTAATCAT | | | GACGTGTGCTCTTCC | |
| | GAAGCTGGACTGC | | | GATCTCTGGACTGCA | |
| | ACCATCG | | | CCATCGCTCAGG | |
| EXD3_m1 | CCCCTTAGGGATA | 41 | EXD3_m2 | GTGACTGGAGTTCA | 176 |
| | ACAGGGTAATCTG | | | GACGTGTGCTCTTCC | |
| | GGGAGGGGCGAA | | | GATCTAAGGGAGTCT | |
| | GGTC | | | CAGGCCCGTGAG | |
| EXD3_p1 | CCCCTTAGGGATA | 42 | EXD3_p2 | GTGACTGGAGTTCA | 177 |
| | ACAGGGTAATCCC | | | GACGTGTGCTCTTCC | |
| | GGGTCCTGCGTCC | | | GATCTGTCCTGCGTC | |
| | CTT | | | CCTTCCCCTGA | |
| FAM83H_m1 | CCCCTTAGGGATA | 43 | FAM83H_m2 | GTGACTGGAGTTCA | 178 |
| | ACAGGGTAATCCC | | | GACGTGTGCTCTTCC | |
| | GCAGCCTCCAGAT | | | GATCTGCACCGGCAG | |
| | GCA | | | CCACCTGT | |
| FAM83H_p1 | CCCCTTAGGGATA | 44 | FAM83H_p2 | GTGACTGGAGTTCA | 179 |
| | ACAGGGTAATCCT | | | GACGTGTGCTCTTCC | |
| | GAGGCTCTTATCA | | | GATCTAACTGCCACT | |
| | AACAACTGCCA | | | ACTCCCGTCCTCAG | |
| FBXO2_m1 | CCCCTTAGGGATA | 45 | FBXO2_m2 | GTGACTGGAGTTCA | 180 |
| | ACAGGGTAATCCG | | | GACGTGTGCTCTTCC | |
| | AGTCCCGGCGCTG | | | GATCTTGTCCGCGTC | |
| | TCC | | | TGTGTCGGT | |
| FBXO2_p1 | CCCCTTAGGGATA | 46 | FBXO2_p2 | GTGACTGGAGTTCA | 181 |
| | ACAGGGTAATCCC | | | GACGTGTGCTCTTCC | |
| | TCCTCGGTCCGCT | | | GATCTCCCGGGCCTC | |
| | GAG | | | GAGCAGAC | |
| FMN1_m1 | CCCCTTAGGGATA | 47 | FMN1_m2 | GTGACTGGAGTTCA | 182 |
| | ACAGGGTAATCCA | | | GACGTGTGCTCTTCC | |
| | ATCTCTGACTTGG | | | GATCTCTTGGACAGC | |
| | ACAGCTGCA | | | TGCAGTACTCCCT | |
| FMN1_p1 | CCCCTTAGGGATA | 48 | FMN1_p2 | GTGACTGGAGTTCA | 183 |
| | ACAGGGTAATCTC | | | GACGTGTGCTCTTCC | |
| | GATGATGGCCTAT | | | GATCTAGTGCGGTGG | |
| | GGGTTGAAAA | | | AGAAAGGCAAG | |
| FSTL4_m1 | CCCCTTAGGGATA | 49 | FSTL4_m2 | GTGACTGGAGTTCA | 184 |
| | ACAGGGTAATCTG | | | GACGTGTGCTCTTCC | |
| | TGCTTCTTCCAAG | | | GATCTGCGTCTCTTT | |
| | CTGCGT | | | GGACCCGTACTTGC | |
| FSTL4_p1 | CCCCTTAGGGATA | 50 | FSTL4_p2 | GTGACTGGAGTTCA | 185 |
| | ACAGGGTAATCTG | | | GACGTGTGCTCTTCC | |
| | TGATTTTCCTGGCT | | | GATCTTCCTGGCTTT | |
| | TTAGCGCTA | | | AGCGCTATACGTTTG | |
| | | | | A | |
| HDLBP_m1 | CCCCTTAGGGATA | 51 | HDLBP_m2 | GTGACTGGAGTTCA | 186 |
| | ACAGGGTAATCTC | | | GACGTGTGCTCTTCC | |
| | TACAACCAAGCCC | | | GATCTCATTTGTCCA | |
| | ATTTGTCCA | | | GGAACCCCTAGCC | |
| HDLBP_p1 | CCCCTTAGGGATA | 52 | HDLBP_p2 | GTGACTGGAGTTCA | 187 |
| | ACAGGGTAATCAG | | | GACGTGTGCTCTTCC | |
| | CCTCTCTACCATTT | | | GATCTACCATTTGTG | |
| | GTGCTGA | | | CTGATCTGTGGGTAT | |
| | | | | C | |
| HMX1-NC_m1 | CCCCTTAGGGATA | 53 | HMX1-NC_m2 | GTGACTGGAGTTCA | 188 |
| | ACAGGGTAATCCC | | | GACGTGTGCTCTTCC | |
| | TGCCAGGGTTGCA | | | GATCTCAGGGTTGCA | |
| | TGGG | | | TGGGAACTTCCTCTG | |
| HMX1-NC_p1 | CCCCTTAGGGATA | 54 | HMX1-NC_p2 | GTGACTGGAGTTCA | 189 |
| | ACAGGGTAATCTT | | | GACGTGTGCTCTTCC | |
| | GTCCCCACCCTCG | | | GATCTCCCACCCTCG | |
| | TCACTC | | | TCACTCTCTGACC | |
| IL27RA_m1 | CCCCTTAGGGATA | 55 | IL27RA_m2 | GTGACTGGAGTTCA | 190 |
| | ACAGGGTAATCGG | | | GACGTGTGCTCTTCC | |
| | CAGGGACCCGGC | | | GATCTCCGGCGACAC | |
| | GACA | | | TGGGGAATG | |
| IL27RA_p1 | CCCCTTAGGGATA | 56 | IL27RA_p2 | GTGACTGGAGTTCA | 191 |
| | ACAGGGTAATCGG | | | GACGTGTGCTCTTCC | |
| | AAGGGAGGCGCTA | | | GATCTCCCGGGCTCC | |
| | GGCA | | | GTGCAAAC | |
| INPPL1_m1 | CCCCTTAGGGATA | 57 | INPPL1_m2 | GTGACTGGAGTTCA | 192 |
| | ACAGGGTAATCGC | | | GACGTGTGCTCTTCC | |
| | TGGGCCTGCACGC | | | GATCTAGGCCCCCTG | |
| | TCA | | | GAGCTGCA | |
| INPPL1_p1 | CCCCTTAGGGATA | 58 | INPPL1_p2 | GTGACTGGAGTTCA | 193 |
| | ACAGGGTAATCGA | | | GACGTGTGCTCTTCC | |
| | CAGCCACCCTGCT | | | GATCTCCACCCTGCT | |
| | CCAC | | | CCACACACCT | |
| IUQB-NC_m1 | CCCCTTAGGGATA | 59 | IUQB-NC_m2 | GTGACTGGAGTTCA | 194 |
| | ACAGGGTAATCCC | | | GACGTGTGCTCTTCC | |
| | TAGCAACGGCCCT | | | GATCTACGGCCCTGG | |
| | GGCA | | | CACCACCT | |
| IUQB-NC_p1 | CCCCTTAGGGATA | 60 | IUQB-NC_p2 | GTGACTGGAGTTCA | 195 |
| | ACAGGGTAATCCC | | | GACGTGTGCTCTTCC | |
| | CTACCCTGCCGCG | | | GATCTCTGCCGCGCT | |
| | CTCCT | | | CCTCCTTCC | |
| JAKMIP3_m1 | CCCCTTAGGGATA | 61 | JAKMIP3_m2 | GTGACTGGAGTTCA | 196 |
| | ACAGGGTAATCGG | | | GACGTGTGCTCTTCC | |
| | CACCTCATTGGGG | | | GATCTCCTCATTGGG | |
| | ACGT | | | GACGTCTGTTGTGAA | |
| | | | | A | |
| JAKMIP3_p1 | CCCCTTAGGGATA | 62 | JAKMIP3_p2 | GTGACTGGAGTTCA | 197 |
| | ACAGGGTAATCTG | | | GACGTGTGCTCTTCC | |
| | CTCTGAACCGAGG | | | GATCTAGTCCCCAGT | |
| | CCTTG | | | TACGGAGACAAATCT | |
| KCNQ1_m1 | CCCCTTAGGGATA | 63 | KCNQ1_m2 | GTGACTGGAGTTCA | 198 |
| | ACAGGGTAATCGC | | | GACGTGTGCTCTTCC | |
| | AGGGCCCCAGAG | | | GATCTGGCCCCAGAG | |
| | AGGT | | | AGGTGAGGTCACTAT | |
| | | | | A | |
| KCNQ1_p1 | CCCCTTAGGGATA | 64 | KCNQ1_p2 | GTGACTGGAGTTCA | 199 |
| | ACAGGGTAATCGC | | | GACGTGTGCTCTTCC | |
| | AGCGACGCCACTC | | | GATCTGGTACCCCGT | |
| | TTTATCT | | | GCCTCAGCT | |
| KLHL23_m1 | CCCCTTAGGGATA | 65 | KLHL23_m2 | GTGACTGGAGTTCA | 200 |
| | ACAGGGTAATCCG | | | GACGTGTGCTCTTCC | |
| | CGCTGACAGCTGT | | | GATCTCCAGGTTGTT | |
| | TGC | | | TATCTGGGCCTCT | |
| KLHL23_p1 | CCCCTTAGGGATA | 66 | KLHL23_p2 | GTGACTGGAGTTCA | 201 |
| | ACAGGGTAATCTG | | | GACGTGTGCTCTTCC | |
| | AGTTTCATGCTCA | | | GATCTGCAGGACAC | |
| | GTCCCTGCA | | | AGCACAGGTAAGGG | |
| | | | | A | |
| LAMA3_m1 | CCCCTTAGGGATA | 67 | LAMA3_m2 | GTGACTGGAGTTCA | 202 |
| | ACAGGGTAATCAG | | | GACGTGTGCTCTTCC | |
| | GGCTCTGGGGTGA | | | GATCTCTGGGGTGAC | |
| | CTCC | | | TCCAAGGCTTTTCG | |
| LAMA3_p1 | CCCCTTAGGGATA | 68 | LAMA3_p2 | GTGACTGGAGTTCA | 203 |
| | ACAGGGTAATCCT | | | GACGTGTGCTCTTCC | |
| | CCCTACTCAACCC | | | GATCTCCCCGAGCCC | |
| | CGAGCCCTCCT | | | TCCTCTCTTG | |
| LINC00415_m1 | CCCCTTAGGGATA | 69 | LINC00415_m2 | GTGACTGGAGTTCA | 204 |
| | ACAGGGTAATCGC | | | GACGTGTGCTCTTCC | |
| | GCCAGACCAGCTC | | | GATCTAGCTCCGACT | |
| | CGA | | | CCGCTCGCT | |
| LINC00415_p1 | CCCCTTAGGGATA | 70 | LINC00415_p2 | GTGACTGGAGTTCA | 205 |
| | ACAGGGTAATCCT | | | GACGTGTGCTCTTCC | |
| | CCTTGCCCGGGGT | | | GATCTTTGCCCGGGG | |
| | AGG | | | TAGGAAAGTGA | |
| LINC01258_m1 | CCCCTTAGGGATA | 71 | LINC01258_m2 | GTGACTGGAGTTCA | 206 |
| | ACAGGGTAATCCT | | | GACGTGTGCTCTTCC | |
| | TCTCATCCTTGTAT | | | GATCTGTATCAGCTG | |
| | CAGCTGCCTT | | | CCTTCTCATCACAAG | |
| | | | | A | |
| LINC01258_p1 | CCCCTTAGGGATA | 72 | LINC01258_p2 | GTGACTGGAGTTCA | 207 |
| | ACAGGGTAATCGG | | | GACGTGTGCTCTTCC | |
| | GAGAGTGCCATTC | | | GATCTGTGCCATTCT | |
| | TCAGCCTAA | | | CAGCCTAAAAGGTA | |
| | | | | GA | |
| LUC7L2_m1 | CCCCTTAGGGATA | 73 | LUC7L2_m2 | GTGACTGGAGTTCA | 208 |
| | ACAGGGTAATCGG | | | GACGTGTGCTCTTCC | |
| | TGGATCACGCAGT | | | GATCTACGCAGTCGG | |
| | CGGA | | | AGGCCATCC | |
| MIR3681-NC_m1 | CCCCTTAGGGATA | 74 | MIR3681-NC_m2 | GTGACTGGAGTTCA | 209 |
| | ACAGGGTAATCCA | | | GACGTGTGCTCTTCC | |
| | TGAGCACACCCAC | | | GATCTAGCACACCCA | |
| | CACCA | | | CCACCACTCCTA | |
| MIR3681-NC_p1 | CCCCTTAGGGATA | 75 | MIR3681-NC_p2 | GTGACTGGAGTTCA | 210 |
| | ACAGGGTAATCGC | | | GACGTGTGCTCTTCC | |
| | CTTGTCCCACATC | | | GATCTCTTGTCCCAC | |
| | ACAGCA | | | ATCACAGCAAACTCT | |
| MIR4647-NC_m1 | CCCCTTAGGGATA | 76 | MIR4647-NC_m2 | GTGACTGGAGTTCA | 211 |
| | ACAGGGTAATCCG | | | GACGTGTGCTCTTCC | |
| | CCTGGGACTACTT | | | GATCTCGGGGCTGCG | |
| | CTCGTTTGAAA | | | GAAGGATCC | |
| MIR4647-NC_p1 | CCCCTTAGGGATA | 77 | MIR4647-NC_p2 | GTGACTGGAGTTCA | 212 |
| | ACAGGGTAATCCC | | | GACGTGTGCTCTTCC | |
| | CCCAACGTGGCCT | | | GATCTCAACGTGGCC | |
| | CAG | | | TCAGCTGCTC | |
| MOB3B_m1 | CCCCTTAGGGATA | 78 | MOB3B_m2 | GTGACTGGAGTTCA | 213 |
| | ACAGGGTAATCCA | | | GACGTGTGCTCTTCC | |
| | CAGCTGTCCAAAC | | | GATCTACGAGGCTGG | |
| | GAGGCT | | | CTCCCCACT | |
| MOB3B_p1 | CCCCTTAGGGATA | 79 | MOB3B_p2 | GTGACTGGAGTTCA | 214 |
| | ACAGGGTAATCGG | | | GACGTGTGCTCTTCC | |
| | ATGCAACTGAGGG | | | GATCTCTCCTTAGAA | |
| | CTCCTTA | | | AGTCATGCCCCAGGA | |
| | | | | G | |
| MSI2_m1 | CCCCTTAGGGATA | 80 | MSI2_m2 | GTGACTGGAGTTCA | 215 |
| | ACAGGGTAATCGG | | | GACGTGTGCTCTTCC | |
| | AAGGTCGCTGGGA | | | GATCTGGGCTGGGA | |
| | AGCC | | | GGGGATTGGC | |
| MSI2_p1 | CCCCTTAGGGATA | 81 | MSI2_p2 | GTGACTGGAGTTCA | 216 |
| | ACAGGGTAATCTG | | | GACGTGTGCTCTTCC | |
| | CCCAGCCTCCCTG | | | GATCTGCCTCCCTGC | |
| | CAG | | | AGGATGATTGGC | |
| MTMR1_m1 | CCCCTTAGGGATA | 82 | MTMR1_m2 | GTGACTGGAGTTCA | 217 |
| | ACAGGGTAATCAG | | | GACGTGTGCTCTTCC | |
| | CTCCTCTGTGTGA | | | GATCTATGCCACAGA | |
| | CATGCC | | | TGACTATTGCACACC | |
| | | | | T | |
| MTMR1_p1 | CCCCTTAGGGATA | 83 | MTMR1_p2 | GTGACTGGAGTTCA | 218 |
| | ACAGGGTAATCAC | | | GACGTGTGCTCTTCC | |
| | CAACCAGCTAACA | | | GATCTACCTCAGGGG | |
| | CTGCTATGCA | | | CCGCTGCA | |
| NC-Chr12_m1 | CCCCTTAGGGATA | 84 | NC-Chr12_m2 | GTGACTGGAGTTCA | 219 |
| | ACAGGGTAATCAC | | | GACGTGTGCTCTTCC | |
| | TCAGGTGTGCTGG | | | GATCTGCTGGCACTG | |
| | CACTGAT | | | ATCTGTGGTCCCA | |
| NC-Chr12_p1 | CCCCTTAGGGATA | 85 | NC-Chr12_p2 | GTGACTGGAGTTCA | 220 |
| | ACAGGGTAATCAC | | | GACGTGTGCTCTTCC | |
| | ATACAACCAGTTC | | | GATCTAACCAGTTCA | |
| | ACCCAGTTAC | | | CCCAGTTACAGTAGA | |
| | | | | C | |
| NFIX_m1 | CCCCTTAGGGATA | 86 | NFIX_m2 | GTGACTGGAGTTCA | 221 |
| | ACAGGGTAATCGG | | | GACGTGTGCTCTTCC | |
| | TGTGTGTTTGCTG | | | GATCTACCGCTTAAA | |
| | TTACCGCTTA | | | TTAACCCTGAGTGAC | |
| | | | | G | |
| NFIX_p1 | CCCCTTAGGGATA | 87 | NFIX_p2 | GTGACTGGAGTTCA | 222 |
| | ACAGGGTAATCCC | | | GACGTGTGCTCTTCC | |
| | TGGAGCGAAGGC | | | GATCTTAGCGTGCGG | |
| | CTGGAG | | | CCCGAGCT | |
| NoName1_m1 | CCCCTTAGGGATA | 88 | NoName1_m2 | GTGACTGGAGTTCA | 223 |
| | ACAGGGTAATCTA | | | GACGTGTGCTCTTCC | |
| | CTGATGGGGGTGA | | | GATCTGGGGGTGAG | |
| | GCTCCA | | | CTCCAACTCTG | |
| NoName1_p1 | CCCCTTAGGGATA | 89 | NoName1_p2 | GTGACTGGAGTTCA | 224 |
| | ACAGGGTAATCTG | | | GACGTGTGCTCTTCC | |
| | TGTCTCTGCTTTCT | | | GATCTATGTATCTGGC | |
| | GTTGGCA | | | ATTACAGCTGAGCAG | |
| NoName10_m1 | CCCCTTAGGGATA | 90 | NoName10_m2 | GTGACTGGAGTTCA | 225 |
| | ACAGGGTAATCTC | | | GACGTGTGCTCTTCC | |
| | TTCAAGCAGCCCA | | | GATCTCAGCCACTGC | |
| | CCTTCTG | | | ACCGACTTCA | |
| NoName10_p1 | CCCCTTAGGGATA | 91 | NoName10_p2 | GTGACTGGAGTTCA | 226 |
| | ACAGGGTAATCAC | | | GACGTGTGCTCTTCC | |
| | TCCCGCCGGTTCC | | | GATCTCGGTTCCAAG | |
| | AAG | | | TTATCGGAGTGAGCC | |
| | | | | A | |
| NoName11_m1 | CCCCTTAGGGATA | 92 | NoName11_m2 | GTGACTGGAGTTCA | 227 |
| | ACAGGGTAATCCC | | | GACGTGTGCTCTTCC | |
| | AAAGCACAGGTG | | | GATCTGGACTCATAG | |
| | GGGACT | | | CCTGGGGGTAAATGT | |
| | | | | T | |
| NoName11_p1 | CCCCTTAGGGATA | 93 | NoName11_p2 | GTGACTGGAGTTCA | 228 |
| | ACAGGGTAATCCA | | | GACGTGTGCTCTTCC | |
| | GCTGCTTGGGCTC | | | GATCTTGCTTGGGCT | |
| | CGTTG | | | CCGTTGCAATCC | |
| NoName12_m1 | CCCCTTAGGGATA | 94 | NoName12_m2 | GTGACTGGAGTTCA | 229 |
| | ACAGGGTAATCCC | | | GACGTGTGCTCTTCC | |
| | CCAGGCCACAGG | | | GATCTAAACCAGGGG | |
| | AAACC | | | AGAGGGCCATAGAG | |
| NoName12_p1 | CCCCTTAGGGATA | 95 | NoName12_p2 | GTGACTGGAGTTCA | 230 |
| | ACAGGGTAATCGC | | | GACGTGTGCTCTTCC | |
| | TAGGGTGGCTGTG | | | GATCTGCTGTGACTC | |
| | ACTCAG | | | AGAGCCATGGC | |
| NoName13_m1 | CCCCTTAGGGATA | 96 | NoName13_m2 | GTGACTGGAGTTCA | 231 |
| | ACAGGGTAATCCC | | | GACGTGTGCTCTTCC | |
| | TCTGGCTTCCCAT | | | GATCTGGCTTCCCAT | |
| | GGGTGAG | | | GGGTGAGTCCTGT | |
| NoName13_p1 | CCCCTTAGGGATA | 97 | NoName13_p2 | GTGACTGGAGTTCA | 232 |
| | ACAGGGTAATCCT | | | GACGTGTGCTCTTCC | |
| | CCCTGAGAAGAGC | | | GATCTGAAGAGCTG | |
| | TGAACATAGC | | | AACATAGCCAGGCA | |
| | | | | ATT | |
| NoName14_m1 | CCCCTTAGGGATA | 98 | NoName14_m2 | GTGACTGGAGTTCA | 233 |
| | ACAGGGTAATCTC | | | GACGTGTGCTCTTCC | |
| | AACCCTTCCCATG | | | GATCTTGACTGAGGT | |
| | ACTGAGGTG | | | GGATGAACCCCTAAG | |
| | | | | C | |
| NoName14_p1 | CCCCTTAGGGATA | 99 | NoName14_p2 | GTGACTGGAGTTCA | 234 |
| | ACAGGGTAATCCC | | | GACGTGTGCTCTTCC | |
| | CAACCCCCTGCAG | | | GATCTAACCCCCTGC | |
| | CTG | | | AGCTGCTCACAA | |
| NoName15_m1 | CCCCTTAGGGATA | 100 | NoName15_m2 | GTGACTGGAGTTCA | 235 |
| | ACAGGGTAATCTC | | | GACGTGTGCTCTTCC | |
| | AAAATCCCAAGGG | | | GATCTAAATCCCAAG | |
| | CATTGTTC | | | GGCATTGTTCACATA | |
| | | | | A | |
| NoName15_p1 | CCCCTTAGGGATA | 101 | NoName15_p2 | GTGACTGGAGTTCA | 236 |
| | ACAGGGTAATCCA | | | GACGTGTGCTCTTCC | |
| | TTGTGTCTTCTTG | | | GATCTACCCTTTTTG | |
| | GTACCCTTTTT | | | AAAATTAGTTGCCCA | |
| | | | | T | |
| NoName16_m1 | CCCCTTAGGGATA | 102 | NoName16_m2 | GTGACTGGAGTTCA | 237 |
| | ACAGGGTAATCAG | | | GACGTGTGCTCTTCC | |
| | ATCACACGAGGCA | | | GATCTGAGGCAGAG | |
| | GAGGGAA | | | GGAACTACAGGTGC | |
| | | | | A | |
| NoName16_p1 | CCCCTTAGGGATA | 103 | NoName16_p2 | GTGACTGGAGTTCA | 238 |
| | ACAGGGTAATCGC | | | GACGTGTGCTCTTCC | |
| | AATCTCACCTCCT | | | GATCTCCTCCCTCTC | |
| | CCCTCTC | | | CTACCAACTTCATCC | |
| NoName2_m1 | CCCCTTAGGGATA | 104 | NoName2_m2 | GTGACTGGAGTTCA | 239 |
| | ACAGGGTAATCAG | | | GACGTGTGCTCTTCC | |
| | CCAAACACAGAA | | | GATCTCCAAACACAG | |
| | AGGCC | | | AAAGGCCATTTATTG | |
| | | | | T | |
| NoName2_p1 | CCCCTTAGGGATA | 105 | NoName2_p2 | GTGACTGGAGTTCA | 240 |
| | ACAGGGTAATCGT | | | GACGTGTGCTCTTCC | |
| | GAGCCATGATCGT | | | GATCTCCATGATCGT | |
| | GCACTC | | | GCACTCTAGCCT | |
| NoName3_p1 | CCCCTTAGGGATA | 106 | NoName3_p2 | GTGACTGGAGTTCA | 241 |
| | ACAGGGTAATCAC | | | GACGTGTGCTCTTCC | |
| | TACATTGGAGGAG | | | GATCTAGGAGTGTGT | |
| | TGTGTACC | | | ACCATTTAAGGATGT | |
| | | | | G | |
| NoName4_m1 | CCCCTTAGGGATA | 107 | NoName4_m2 | GTGACTGGAGTTCA | 242 |
| | ACAGGGTAATCCT | | | GACGTGTGCTCTTCC | |
| | CTGCTTTCCCCTC | | | GATCTCCCACCTGGC | |
| | CCACCT | | | CCTGCAAGA | |
| NoName4_p1 | CCCCTTAGGGATA | 108 | NoName4_p2 | GTGACTGGAGTTCA | 243 |
| | ACAGGGTAATCCT | | | GACGTGTGCTCTTCC | |
| | GCCCTGTTGGATA | | | GATCTTCTCTGCCCC | |
| | ACCCTTCT | | | TGGACAGATTCTATA | |
| | | | | G | |
| NoName5_m1 | CCCCTTAGGGATA | 109 | NoName5_m2 | GTGACTGGAGTTCA | 244 |
| | ACAGGGTAATCCT | | | GACGTGTGCTCTTCC | |
| | TGGAAAGGGATGC | | | GATCTGGGCCCTGCT | |
| | TCTGAATACCT | | | GCACTATGATCAA | |
| NoName5_p1 | CCCCTTAGGGATA | 110 | NoName5_p2 | GTGACTGGAGTTCA | 245 |
| | ACAGGGTAATCAG | | | GACGTGTGCTCTTCC | |
| | CTGCACTTTCTCC | | | GATCTGGGCCAGCTT | |
| | CGGACAA | | | CATGACCTGAAACC | |
| NoName6_m1 | CCCCTTAGGGATA | 111 | NoName6_m2 | GTGACTGGAGTTCA | 246 |
| | ACAGGGTAATCTG | | | GACGTGTGCTCTTCC | |
| | TTGTTAAGGCTGT | | | GATCTTGCACCTGGC | |
| | TGGCATCTGT | | | TGCACCAC | |
| NoName6_p1 | CCCCTTAGGGATA | 112 | NoName6_p2 | GTGACTGGAGTTCA | 247 |
| | ACAGGGTAATCAG | | | GACGTGTGCTCTTCC | |
| | GAAAACACGGTTG | | | GATCTCATCCTGAAT | |
| | CATCCTGA | | | GCTCGTTGAGTGGAT | |
| | | | | G | |
| NoName7_m1 | CCCCTTAGGGATA | 113 | NoName7_m2 | GTGACTGGAGTTCA | 248 |
| | ACAGGGTAATCGC | | | GACGTGTGCTCTTCC | |
| | ACCAGCTCTTCGG | | | GATCTGGCCAAGCCC | |
| | CCAAG | | | ATGTAGTACTGCAG | |
| NoName7_p1 | CCCCTTAGGGATA | 114 | NoName7_p2 | GTGACTGGAGTTCA | 249 |
| | ACAGGGTAATCTC | | | GACGTGTGCTCTTCC | |
| | CGTGTGTTTGACT | | | GATCTCCCTCAACTA | |
| | CCCTCAAC | | | CTTGCCCAACATGC | |
| NoName8_m1 | CCCCTTAGGGATA | 115 | NoName8_m2 | GTGACTGGAGTTCA | 250 |
| | ACAGGGTAATCGG | | | GACGTGTGCTCTTCC | |
| | CGGTGTCAGCAAA | | | GATCTCGGTGTCAGC | |
| | GCTAGG | | | AAAGCTAGGTAAGG | |
| | | | | AG | |
| NoName8_p1 | CCCCTTAGGGATA | 116 | NoName8_p2 | GTGACTGGAGTTCA | 251 |
| | ACAGGGTAATCAG | | | GACGTGTGCTCTTCC | |
| | CACCGATGAGGCA | | | GATCTCCGATGAGGC | |
| | TGGG | | | ATGGGTTATGAAGTA | |
| NoName9_m1 | CCCCTTAGGGATA | 117 | NoName9_m2 | GTGACTGGAGTTCA | 252 |
| | ACAGGGTAATCGT | | | GACGTGTGCTCTTCC | |
| | GCTGCCTCCCCCT | | | GATCTCCCCTCTGGT | |
| | CTGGTA | | | ATGCCCCCTCAT | |
| NoName9_p1 | CCCCTTAGGGATA | 118 | NoName9_p2 | GTGACTGGAGTTCA | 253 |
| | ACAGGGTAATCGG | | | GACGTGTGCTCTTCC | |
| | AGTGACTGGATGC | | | GATCTTGACTGGATG | |
| | TGGGTT | | | CTGGGTTGTGGAAA | |
| nr-HERPUD1_m1 | CCCCTTAGGGATA | 119 | nr-HERPUD1_m2 | GTGACTGGAGTTCA | 254 |
| | ACAGGGTAATCGG | | | GACGTGTGCTCTTCC | |
| | AGAGGGGCCTGG | | | GATCTTTCTCCCCCG | |
| | AAGATTCTC | | | AGGCCTCAGAA | |
| nr-HERPUD1_p1 | CCCCTTAGGGATA | 120 | nr-HERPUD1_p2 | GTGACTGGAGTTCA | 255 |
| | ACAGGGTAATCGG | | | GACGTGTGCTCTTCC | |
| | GTAGACTTGACAT | | | GATCTGACTTGACAT | |
| | AAGCACCA | | | AAGCACCATACTTCG | |
| | | | | G | |
| PAPD_m1 | CCCCTTAGGGATA | 121 | PAPD7_m2 | GTGACTGGAGTTCA | 256 |
| | ACAGGGTAATCAA | | | GACGTGTGCTCTTCC | |
| | GAAAAGGGGCTG | | | GATCTGGGCTGCTGG | |
| | CTGGGT | | | GTAGGACCTG | |
| PAPD7_p1 | CCCCTTAGGGATA | 122 | PAPD7_p2 | GTGACTGGAGTTCA | 257 |
| | ACAGGGTAATCGA | | | GACGTGTGCTCTTCC | |
| | CGTGATTCGAGTT | | | GATCTCGTGATTCGA | |
| | CCTGGCA | | | GTTCCTGGCAATGCT | |
| | | | | A | |
| PAX6_m1 | CCCCTTAGGGATA | 123 | PAX6_m2 | GTGACTGGAGTTCA | 258 |
| | ACAGGGTAATCGG | | | GACGTGTGCTCTTCC | |
| | GTCTGGGGTCCTG | | | GATCTGGTCCTGAAA | |
| | AAATGAC | | | TGACCCCCAAGG | |
| PAX6_p1 | CCCCTTAGGGATA | 124 | PAX6_p2 | GTGACTGGAGTTCA | 259 |
| | ACAGGGTAATCCC | | | GACGTGTGCTCTTCC | |
| | CACTAGATCCTGT | | | GATCTCGCAGCCTAT | |
| | CACAATTCCC | | | TGTCTCCTGGT | |
| PLPPR1-NC_m1 | CCCCTTAGGGATA | 125 | PLPPR1-NC_m2 | GTGACTGGAGTTCA | 260 |
| | ACAGGGTAATCTG | | | GACGTGTGCTCTTCC | |
| | TGCTCCCGCTCCC | | | GATCTGCACGCCGTG | |
| | ATGAG | | | GCCGAACA | |
| PLPPR1-NC_p1 | CCCCTTAGGGATA | 126 | PLPPR1-NC_p2 | GTGACTGGAGTTCA | 261 |
| | ACAGGGTAATCTG | | | GACGTGTGCTCTTCC | |
| | CACAAGAACCTGC | | | GATCTAACTTCCATA | |
| | TGTCTAAACTT | | | CCAGCAGCAGTTCC | |
| PRR19_m1 | CCCCTTAGGGATA | 127 | PRR19_m2 | GTGACTGGAGTTCA | 262 |
| | ACAGGGTAATCAC | | | GACGTGTGCTCTTCC | |
| | GACGGCCGCACA | | | GATCTCCGCTCGGGC | |
| | GTGG | | | CGCTGACT | |
| PRR19_p1 | CCCCTTAGGGATA | 128 | PRR19_p2 | GTGACTGGAGTTCA | 263 |
| | ACAGGGTAATCCC | | | GACGTGTGCTCTTCC | |
| | CGCCCACTCTCGA | | | GATCTCGCCCACTCT | |
| | CTCTT | | | CGACTCTTCAGGTAG | |
| SAMD11_m1 | CCCCTTAGGGATA | 129 | SAMD11_m2 | GTGACTGGAGTTCA | 264 |
| | ACAGGGTAATCCC | | | GACGTGTGCTCTTCC | |
| | AGGACTCCCCAGG | | | GATCTACTCCCCAGG | |
| | TGCT | | | TGCTGAAGAGACG | |
| SAMD11_p1 | CCCCTTAGGGATA | 130 | SAMD11_p2 | GTGACTGGAGTTCA | 265 |
| | ACAGGGTAATCCT | | | GACGTGTGCTCTTCC | |
| | CTAGCCCGAAAAG | | | GATCTGCAGGGGGTC | |
| | CCAAGCT | | | CGAGTGCA | |
| SBF1_m1 | CCCCTTAGGGATA | 131 | SBF1_m2 | GTGACTGGAGTTCA | 266 |
| | ACAGGGTAATCCT | | | GACGTGTGCTCTTCC | |
| | CTGCCAGATGCTG | | | GATCTTGCTGCTCGT | |
| | CTCGT | | | TGCCTGGCA | |
| SBF1_p1 | CCCCTTAGGGATA | 132 | SBF1_p2 | GTGACTGGAGTTCA | 267 |
| | ACAGGGTAATCGC | | | GACGTGTGCTCTTCC | |
| | TGTTGCAGGTCCA | | | GATCTCACTTGAGGT | |
| | GAGGACAC | | | GGACGTCAGTTTCTG | |
| | | | | G | |
| SLC22A1_m1 | CCCCTTAGGGATA | 133 | SLC22A1_m2 | GTGACTGGAGTTCA | 268 |
| | ACAGGGTAATCGA | | | GACGTGTGCTCTTCC | |
| | AGACGTGGGTTCT | | | GATCTGTGGGTTCTG | |
| | GGCAGA | | | GCAGAAGTTCCTATG | |
| | | | | T | |
| SLC22A1_p1 | CCCCTTAGGGATA | 134 | SLC22A1_p2 | GTGACTGGAGTTCA | 269 |
| | ACAGGGTAATCCC | | | GACGTGTGCTCTTCC | |
| | CCCGTCCCCTCTG | | | GATCTCCCCTCTGCC | |
| | CCA | | | ACCCCCAT | |
| SPNS3_m1 | CCCCTTAGGGATA | 135 | SPNS3_m2 | GTGACTGGAGTTCA | 270 |
| | ACAGGGTAATCTG | | | GACGTGTGCTCTTCC | |
| | CCTGTGTCCGGAG | | | GATCTCCTGTGTCCG | |
| | CTGT | | | GAGCTGTTTCTGC | |
| SPNS3_p1 | CCCCTTAGGGATA | 136 | SPNS3_p2 | GTGACTGGAGTTCA | 271 |
| | ACAGGGTAATCCC | | | GACGTGTGCTCTTCC | |
| | TACCGGGGCAAGA | | | GATCTCCTGGCTGGA | |
| | CAGC | | | AAGGCAACCC | |
| SRPK2_m1 | CCCCTTAGGGATA | 137 | SRPK2_m2 | GTGACTGGAGTTCA | 272 |
| | ACAGGGTAATCTG | | | GACGTGTGCTCTTCC | |
| | GTGACAACTACCA | | | GATCTACCACTCTAG | |
| | CTCTAGAATTT | | | AATTTGGCAAGATGT | |
| TBATA_m1 | CCCCTTAGGGATA | 138 | TBATA_m2 | GTGACTGGAGTTCA | 273 |
| | ACAGGGTAATCTG | | | GACGTGTGCTCTTCC | |
| | TCCTAAAACCCCT | | | GATCTATTTCTCCACC | |
| | GCTTGGATTT | | | TAGGTGTGCTCTCTC | |
| TBATA_p1 | CCCCTTAGGGATA | 139 | TBATA_p2 | GTGACTGGAGTTCA | 274 |
| | ACAGGGTAATCTG | | | GACGTGTGCTCTTCC | |
| | CGGAACACAGGA | | | GATCTGAACACAGG | |
| | GCTAGTCT | | | AGCTAGTCTGGGAA | |
| | | | | GA | |
| TRIM42_m1 | CCCCTTAGGGATA | 140 | TRIM42_m2 | GTGACTGGAGTTCA | 275 |
| | ACAGGGTAATCTC | | | GACGTGTGCTCTTCC | |
| | AGTAGCTCCCCAA | | | GATCTCGTTACTGTG | |
| | CGTTACTGT | | | CATTGAAGTCACCTG | |
| | | | | A | |
| TRIM42_p1 | CCCCTTAGGGATA | 141 | TRIM42_p2 | GTGACTGGAGTTCA | 276 |
| | ACAGGGTAATCCT | | | GACGTGTGCTCTTCC | |
| | GTCTCCCAAAATC | | | GATCTGCCTGTTCTT | |
| | AGGCCTGT | | | GCACCTGGATTCTTA | |
| | | | | C | |
| TSKU_m1 | CCCCTTAGGGATA | 142 | TSKU_m2 | GTGACTGGAGTTCA | 277 |
| | ACAGGGTAATCTT | | | GACGTGTGCTCTTCC | |
| | TGTGCGCCCTGCC | | | GATCTGCGCCCTGCC | |
| | CTT | | | CTTCGGATAA | |
| TSKU_p1 | CCCCTTAGGGATA | 143 | TSKU_p2 | GTGACTGGAGTTCA | 278 |
| | ACAGGGTAATCGG | | | GACGTGTGCTCTTCC | |
| | GGAGGAGGGTGTT | | | GATCTACGGTTATCTT | |
| | TACGG | | | TGCGACTTAGGCTCA | |
| UTP14A_m1 | CCCCTTAGGGATA | 144 | UTP14A_m2 | GTGACTGGAGTTCA | 279 |
| | ACAGGGTAATCAG | | | GACGTGTGCTCTTCC | |
| | GCAGTGCAGGCGT | | | GATCTGCGTTATAAA | |
| | TATAAACT | | | CTCCCCGAATCTTGG | |
| | | | | A | |
| UTP14A_p1 | CCCCTTAGGGATA | 145 | UTP14A_p2 | GTGACTGGAGTTCA | 280 |
| | ACAGGGTAATCCA | | | GACGTGTGCTCTTCC | |
| | CTTTCCCTGGGGC | | | GATCTTCCCTGGGGC | |
| | TTGCTTA | | | TTGCTTAGTAAAGTA | |
| | | | | G | |
| UTP4_m1 | CCCCTTAGGGATA | 146 | UTP4_m2 | GTGACTGGAGTTCA | 281 |
| | ACAGGGTAATCGG | | | GACGTGTGCTCTTCC | |
| | AAGGGGCGTGGG | | | GATCTAGGTGGCCGG | |
| | AAGCG | | | CCCAGGGT | |
| UTP4_p1 | CCCCTTAGGGATA | 147 | UTP4_p2 | GTGACTGGAGTTCA | 282 |
| | ACAGGGTAATCCC | | | GACGTGTGCTCTTCC | |
| | GCAGACAGAGCA | | | GATCTTCGGGCCGGG | |
| | AGCGCGTT | | | GCGTCTGA | |
| VEGFA_m1 | CCCCTTAGGGATA | 148 | VEGFA_m2 | GTGACTGGAGTTCA | 283 |
| | ACAGGGTAATCGC | | | GACGTGTGCTCTTCC | |
| | CCCAGCTACCACC | | | GATCTCGGCGGCGG | |
| | TCCTC | | | ACAGTGGAC | |
| VEGFA_p1 | CCCCTTAGGGATA | 149 | VEGFA_p2 | GTGACTGGAGTTCA | 284 |
| | ACAGGGTAATCCG | | | GACGTGTGCTCTTCC | |
| | CGGACCACGGCTC | | | GATCTCCGAAGCGA | |
| | CTC | | | GAACAGCCCAGAAG | |
| | | | | TT | |

Referring now to FIG. 2A and FIG. 2B, charts 210 and 210′ show the off-target identification and validation using EDITED-Seq at VEGFA_2 locus edited by CRISPR-Cas9, respectively. As shown in charts 210 and 210′, there were a portion of off-targets (64 out of 94) captured by the in silico-predicted off-targets as revealed by split-fusion detection. Furthermore, the vast majority (92%) of those sites found fusion events were also validated as there were Indels detected by EDITED-Seq.

Referring now to FIG. 2C, a diagram 220 shows the correlation between EDITED-Seq score (Escore) and Indel frequencies (%), according to the same example embodiment of FIG. 2A and FIG. 2B. EDITED-Seq score (Escore) showed strong correlation with Indel frequency simultaneously estimated from the same sequencing data. FIG. 2E shows a translocation circus plot 370 of VEGFA_2 within chromosome coordinate, showing that there were around 48% sites connecting to more than one fusion partner. Referring now to FIG. 2D, diagram 230 shows the detection titration of input genomic DNA at VEGFA_2 locus, according to the same example embodiment of FIG. 2A and FIG. 2B. EDITED-Seq required a total input cells of about 30,000-70,000 to saturation of detecting off-target number and total translocation partner. These results show that EDITED-Seq can easily and sensitively detect in situ post-edited off-targets through capturing translocations among Gas-induced DSBs in human genome.

### Example 11. Comparison of EDITED-Seq with DISCOVER-Seq and GUIDE-Seq

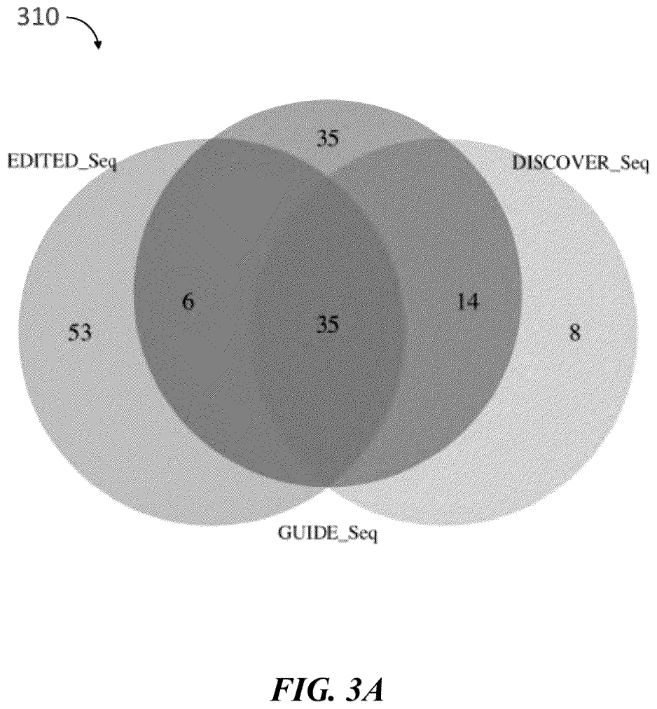

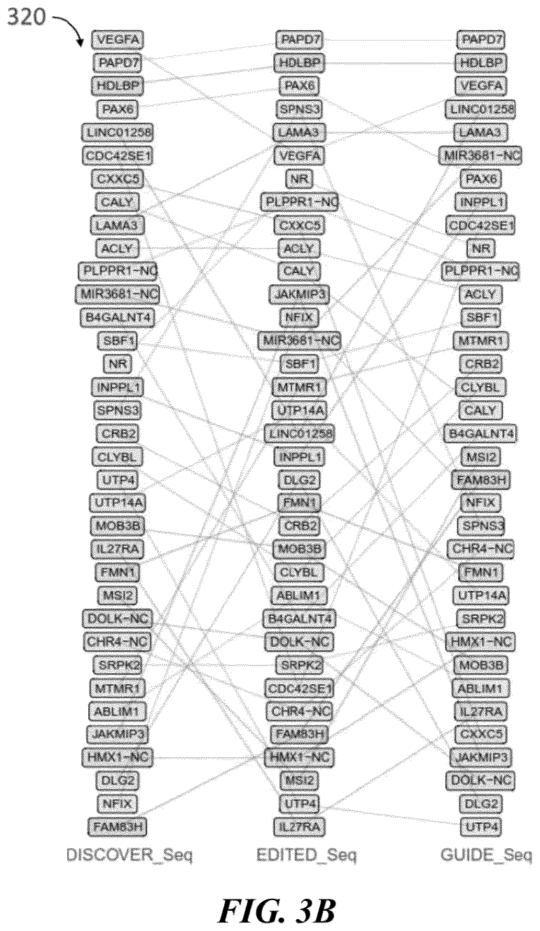

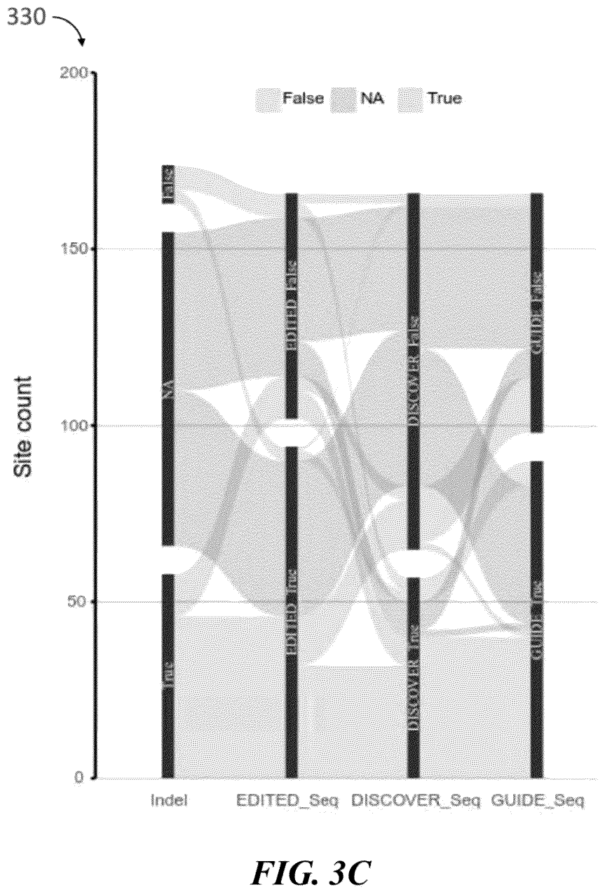

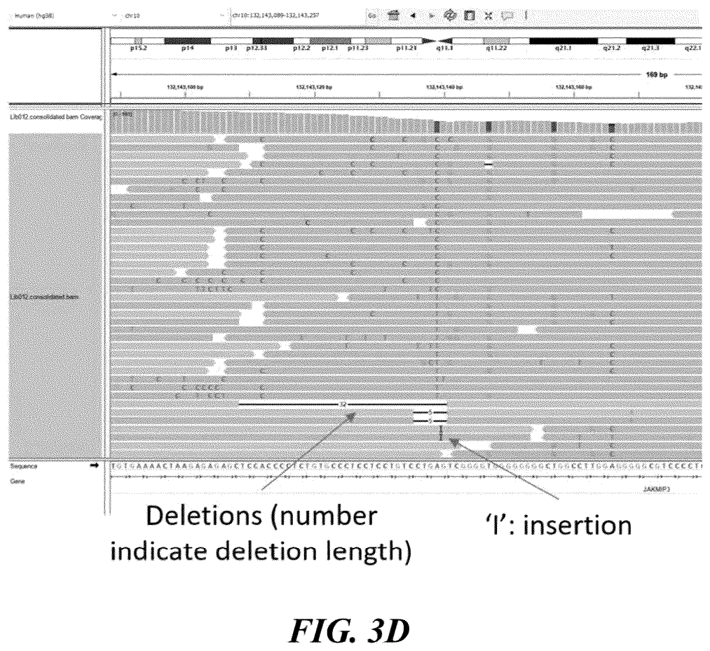

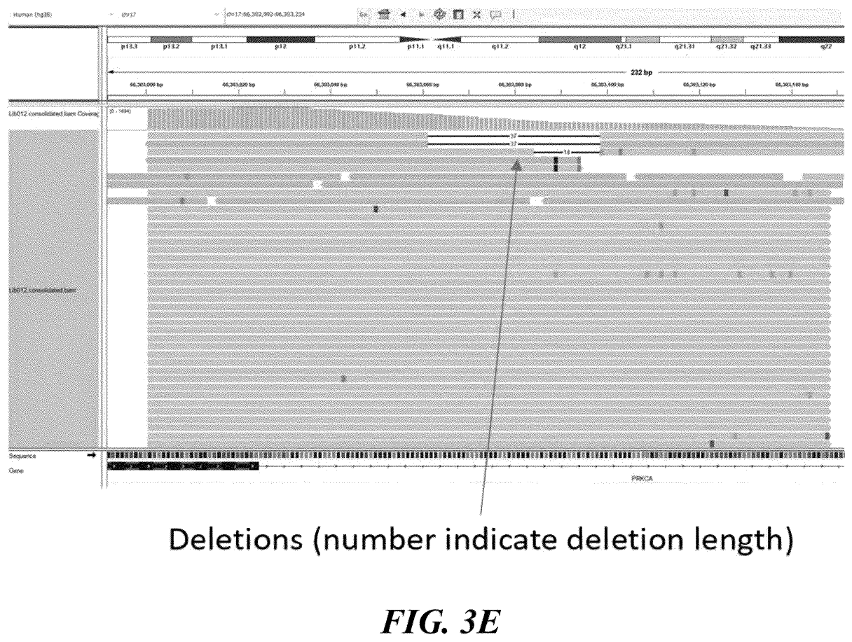

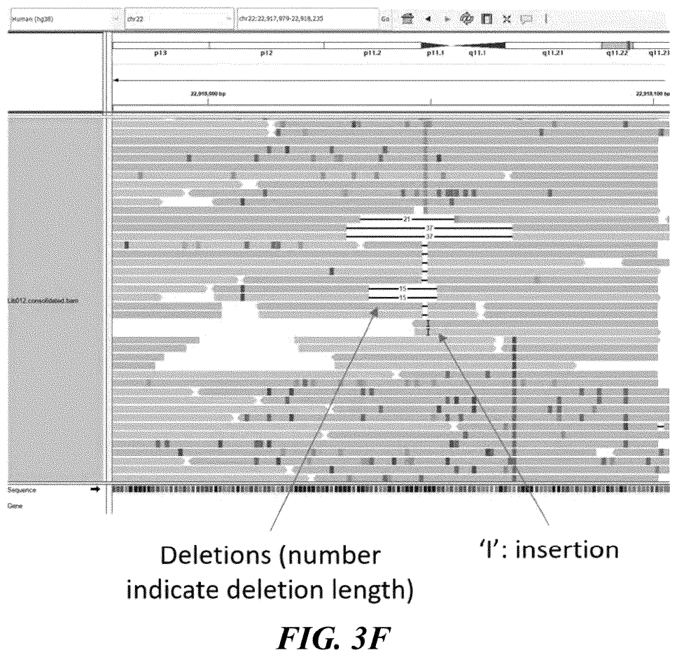

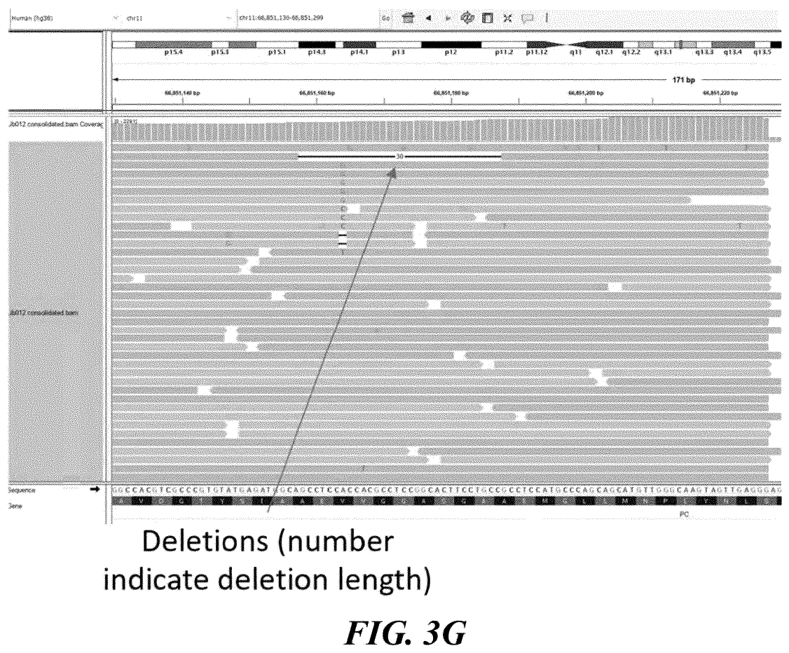

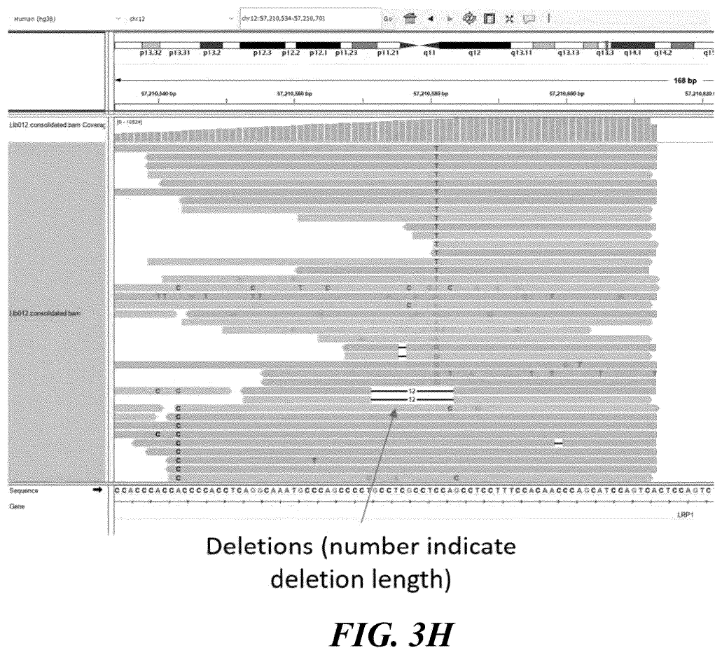

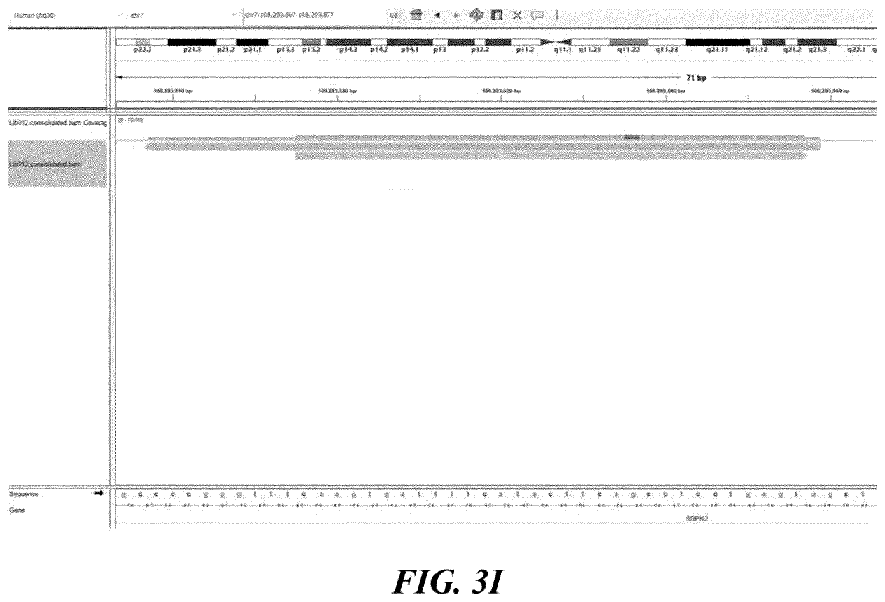

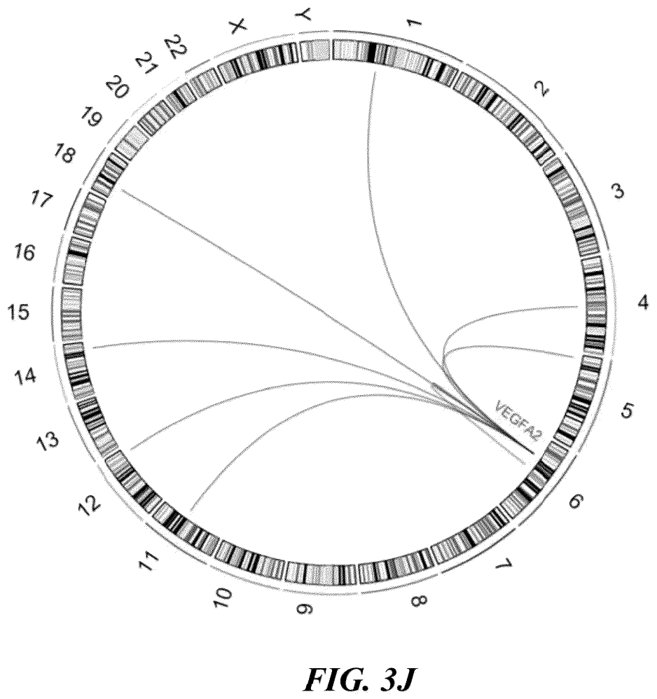

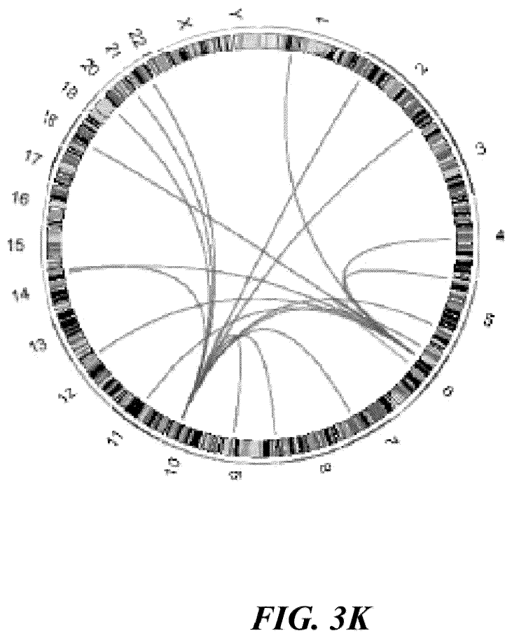

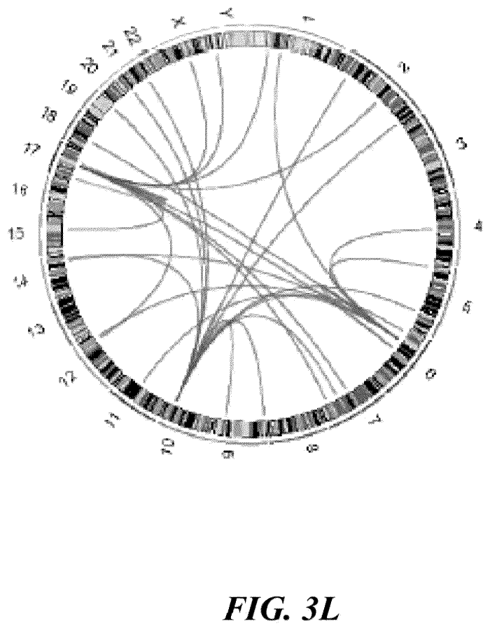

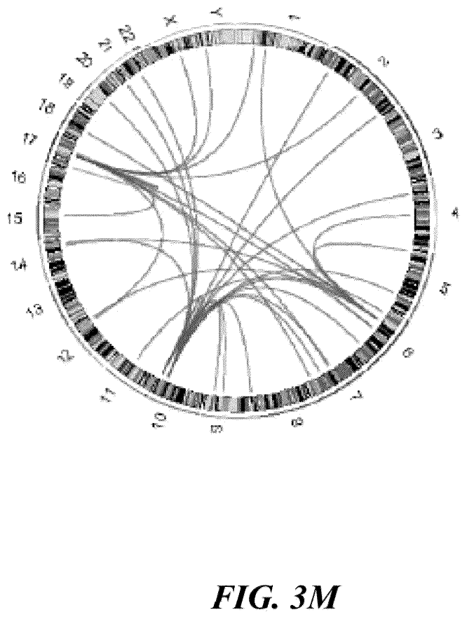

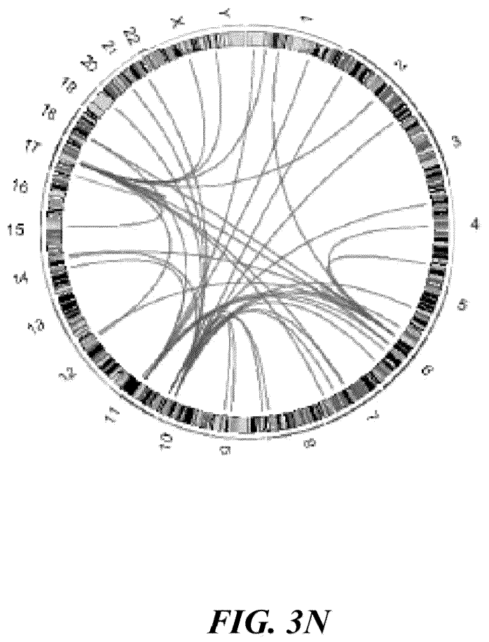

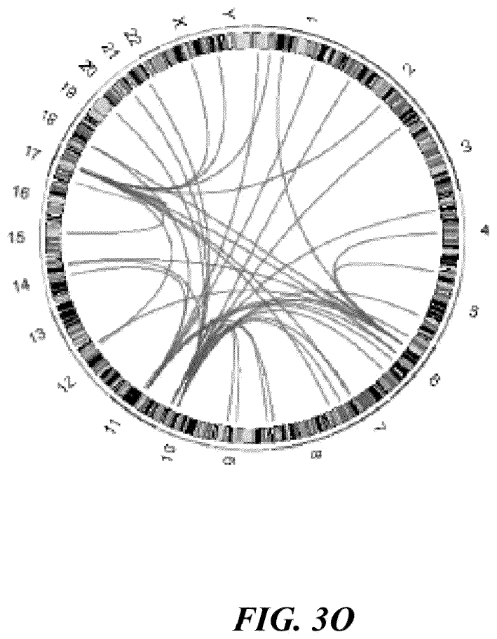

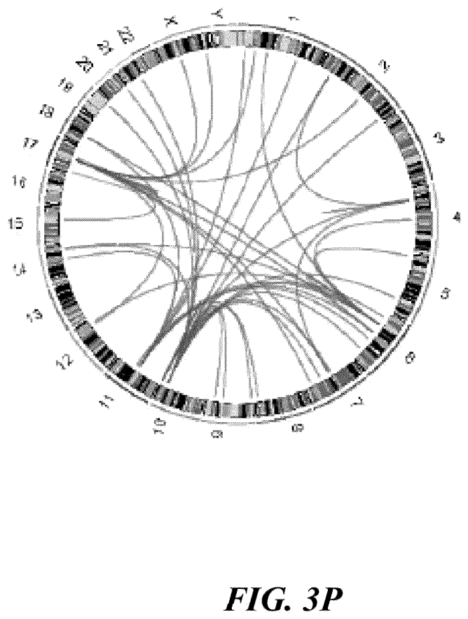

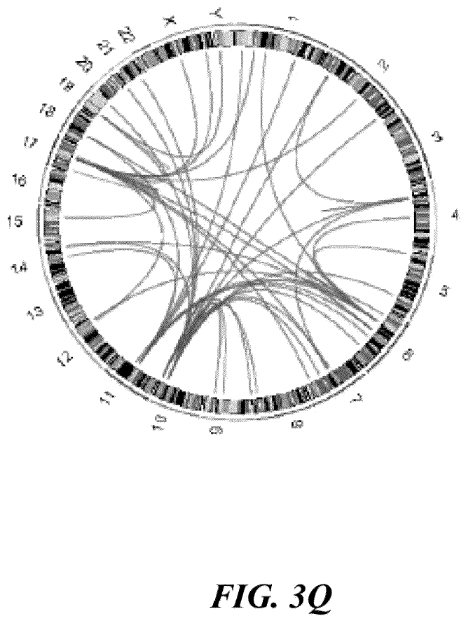

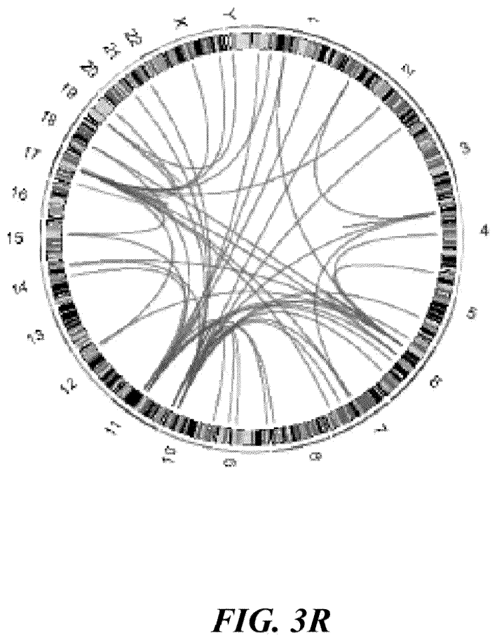

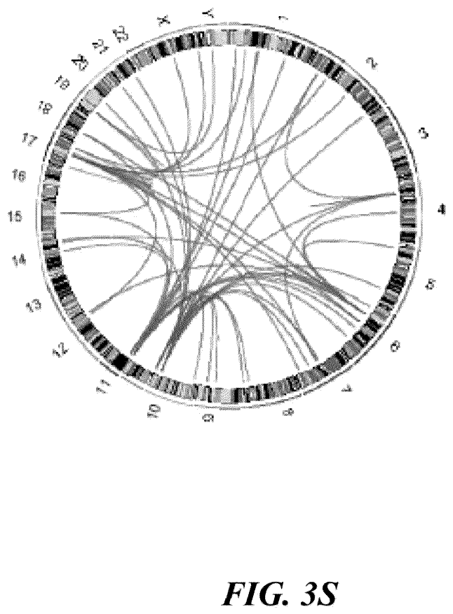

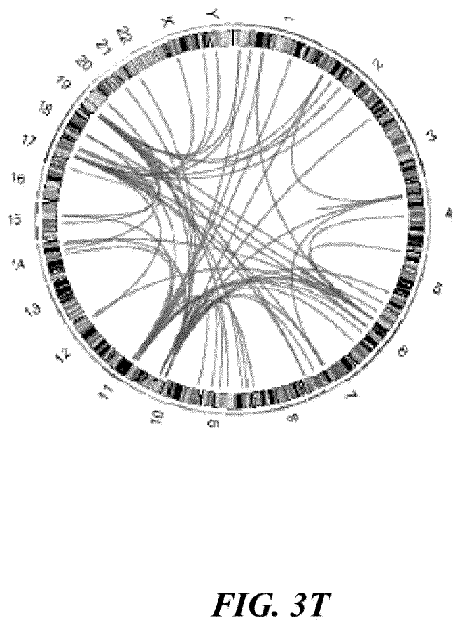

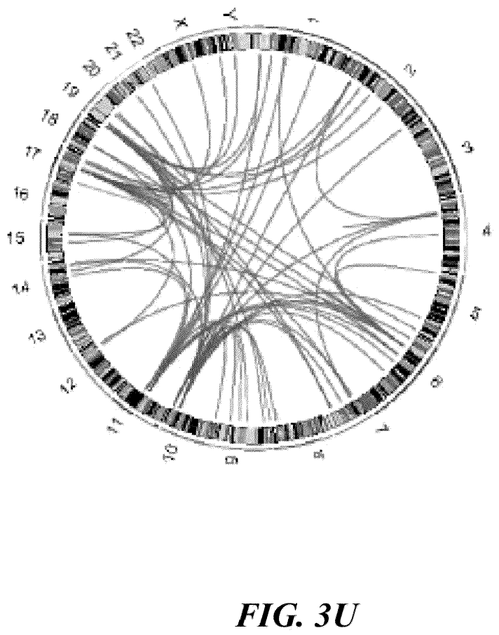

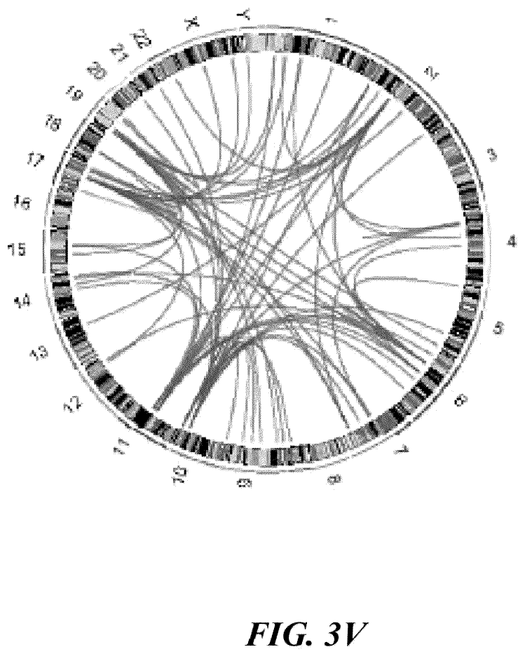

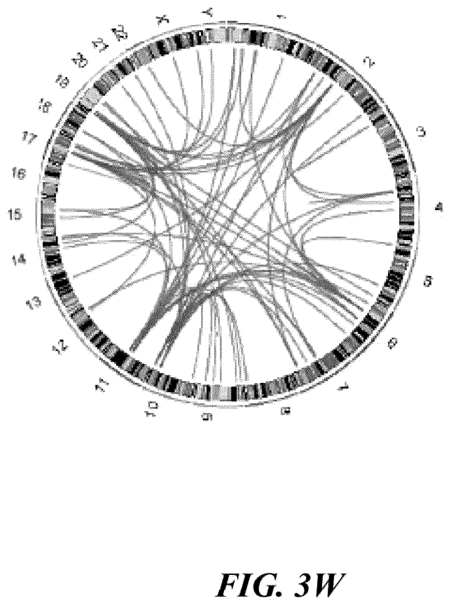

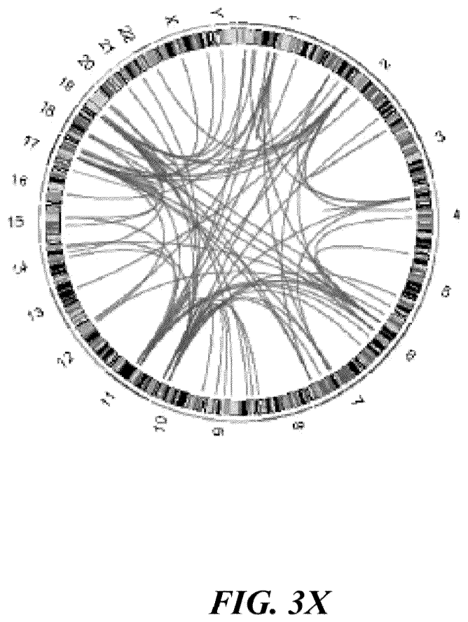

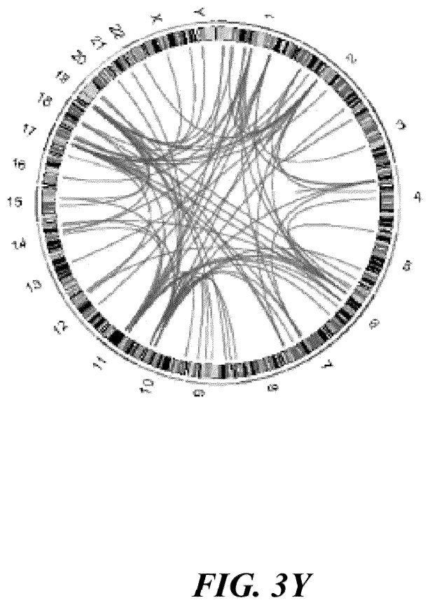

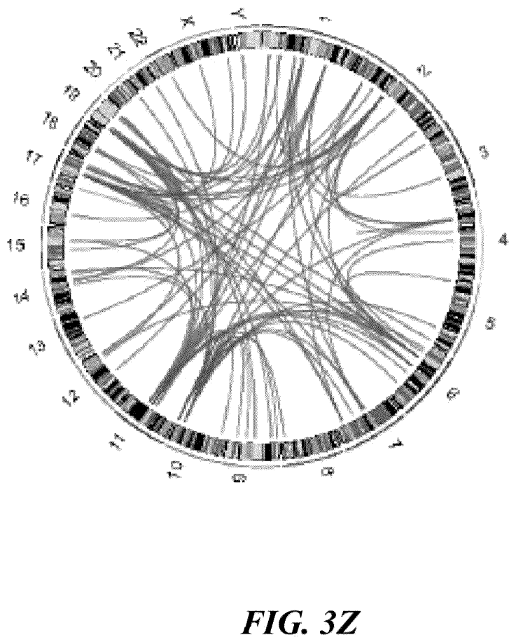

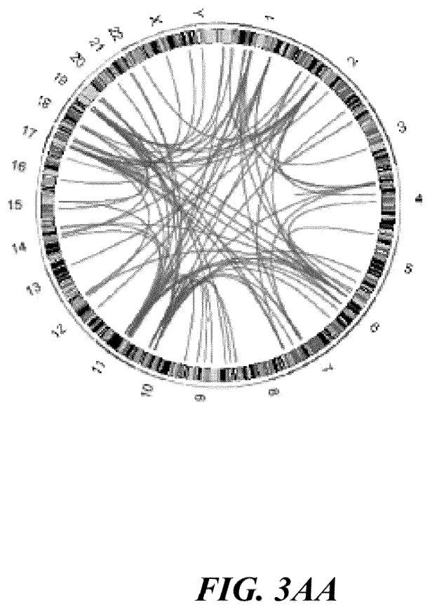

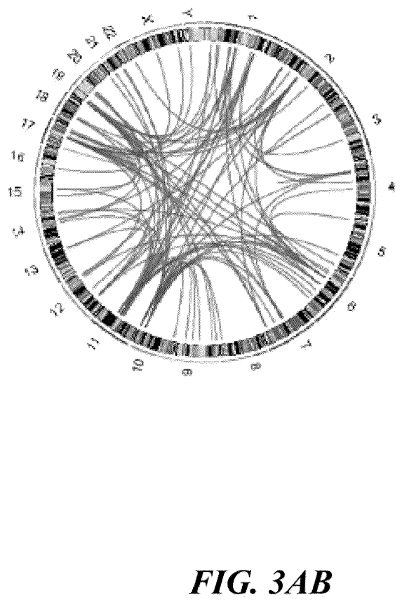

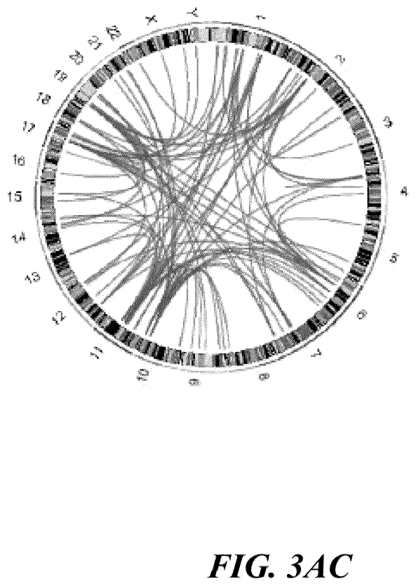

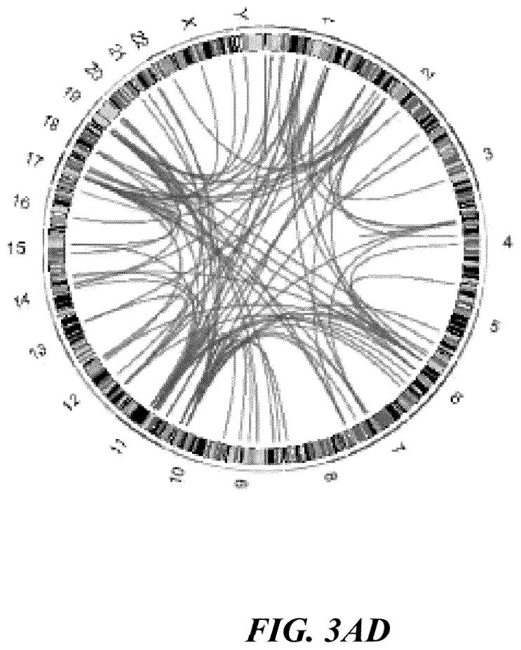

Referring now to FIG. 3A, the performance of EDITED-Seq with that of DISCOVER-Seq and GUIDE-Seq were compared in this example embodiment. As shown in a Venn diagram 310 comparing the three methods (EDITED-Seq, GUIDE-Seq and DISCOVER-Seq) in detection of off-targets at VEFGA_2 locus. It showed that 94, 90 and 57 off-targets were detected at VEFGA_2 locus by EDITED-Seq, DISCOVER-Seq and GUIDE-Seq respectively, indicating that EDITED-Seq can identify more off-targets. There were around 45.6% and 61.4% sites of GUIDE-Seq or DISCOVER-Seq that were identified by EDITED-Seq (FIG. 3A). On the other hand, there were more than a half (around 56.4%) sites of EDITED-Seq that were never identified by GUIDE-Seq nor DISCOVER-Seq, indicated that EDITED-Seq can surprisingly identify most unique off-targets that have never been identified. Therefore, EDITED-Seq showed the most unique off-targets, of which 92.3% were confirmed by NGS amplicon. Those unidentified by EDITED-Seq were most unlikely detected Indel or which Indel frequencies were below 0.001% (FIG. 2A and FIG. 2B).

Referring now to FIG. 3B, a diagram 320 showed a rank comparison of the commonly identified 35 sites based on the corresponding scoring values (e.g. Escore) of EDITED-Seq, GUIDE-Seq, and DISCOVER-Seq, according to the same example embodiment of FIG. 3A. Besides several top-scored sites showing consistent ranks across different methods, most of EDITED-Seq were not at the same level in the dataset of DISCOVER-Seq or GUIDE-Seq, respectively.

Referring now to FIG. 3C, a diagram 330 shows Paranal distributions of identified (i.e., true) and missed (i.e., false) off-targets of EDITED-Seq, compared to GUIDE-Seq and DISCOVER-Seq, according to the same example embodiment of FIG. 3A. There were few sites with Indel discovered by amplicon NGS that had not been detected in translocation. EDITED-Seq missed the least number of true sites that were validated by amplicon NGS (false negatives). Some highly ranked sites discovered by GUIDE-Seq showed few translocations. It is supposed that protospacer sequence context might trigger the recombination between two DSB ends. The results showed that the relative ratio of false off-targets of EDITED-Seq over the true off targets is significantly lower than the same ratio of DISCOVER-Seq or GUIDE-Seq. EDITED-Seq is a more accurate method compared to DISCOVER-Seq and GUIDE-Seq because it has a significantly lower ratio of false off-targets.

Furthermore, the targets that were missed by DISCOVER-seq and GUIDE-seq but were identified by EDITED-seq were confirmed by deep amplicon sequencing. Six exemplary views from Integrated Genome Viewer illustrate the low-level insertions and deletions (see FIG. 3E to FIG. 3H), or translocation (see FIG. 3I).

In addition, a detailed analysis on translocation was carried out. Using only one set of primers for the on-target site in CRISPR-Cas9 targeting VEGFA_2 locus, 8 off-target sites were identified (see FIG. 3J). Briefly, the on-target site VEGFA2, colored in red in FIG. 3J and located on chromosome 6, were shown to form translocations with 8 off-target sites.

Furthermore, using increasing numbers of primers derived from in-silico predicted off-target sites, increasing numbers of novel off-target sites were detected via translocations between on- and off-targets, and between off- and off-target sites. Specifically, a comprehensive identification of genome-wide off-target sites when targeting VEGFA2 and using EDITED-seq was illustrated in FIG. 3K to FIG. 3AD. Using increasing numbers for 1 to 20 off-target sites (from in-silicon prediction) in data analysis, the numbers of total targeting sites identified were 23, 36, 43, 52, 54, 58, 61, 66, 68, 79, 81, 91, 93, 101, 107, 110, 113, 119, 122, 125, and 132, respectively.

### Example 12. Off-Target Profiling in iPSC and Primary Cells Using EDITED-Seq

To test whether EDITED-Seq can act as a versatile implement in various types of cells, gene editing was conducted in iPSC (according to Example 6) and primary cells (according to Example 7), respectively, on four gene loci of functional importance, namely GAPDH, HBB, PD1 and TRAC. The sequences of anchored primers for GAPDH, HBB, PD1 and TRAC used in EDITED-Seq in this example embodiment is shown in Tables 4-7 respectively below.

TABLE 4
Sequences of anchored primers for GAPDH

| First PCR | | SEQ | Second PCR | | SEQ |
|---|---|---|---|---|---|
| primer | | ID | primer | | ID |
| name | Sequence | NO: | name | Sequence | NO: |
| NoName1_m1 | CCCCTTAGGGATAA | 285 | NoName1_m2 | GTGACTGGAGTTC | 360 |
| | CAGGGTAATCTTGG | | | AGACGTGTGCTCT | |
| | CATGACCCAGGTCC | | | TCCGATCTGGTCC | |
| | ATAC | | | ATACCAGGGCTGA | |
| | | | | CC | |
| NoName1_p1 | CCCCTTAGGGATAA | 286 | NoName1_p2 | GTGACTGGAGTTC | 361 |
| | CAGGGTAATCAAGA | | | AGACGTGTGCTCT | |
| | GTCTGGGTGAATCA | | | TCCGATCTAGTCA | |
| | GCAGTC | | | GGCAGGCGAGGA | |
| | | | | ACA | |
| NoName10_m1 | CCCCTTAGGGATAA | 287 | NoName10_m2 | GTGACTGGAGTTC | 362 |
| | CAGGGTAATCAGGG | | | AGACGTGTGCTCT | |
| | GCCAGCAGCAAGG | | | TCCGATCTAGGTG | |
| | T | | | AAGAATTTCATGC | |
| | | | | TGGCACAT | |
| NoName11_p1 | CCCCTTAGGGATAA | 288 | NoName11_p2 | GTGACTGGAGTTC | 363 |
| | CAGGGTAATCTGAG | | | AGACGTGTGCTCT | |
| | TCAGGAGGCAGAG | | | TCCGATCTCAAGA | |
| | ATCCTC | | | CCCAGCGACCGAC | |
| | | | | TCC | |
| NoName12_m1 | CCCCTTAGGGATAA | 289 | NoName12_m2 | GTGACTGGAGTTC | 364 |
| | CAGGGTAATCAAAT | | | AGACGTGTGCTCT | |
| | CCCGTTGGCCCTCC | | | TCCGATCTCTCCT | |
| | TG | | | GCTCAGCTGGCTC | |
| | | | | ATGTC | |
| NoName12_p1 | CCCCTTAGGGATAA | 290 | NoName12_p2 | GTGACTGGAGTTC | 365 |
| | CAGGGTAATCGGGG | | | AGACGTGTGCTCT | |
| | CGTTGTGGGTCTGA | | | TCCGATCTCTTAA | |
| | | | | AGATCCTCCGGCC | |
| | | | | ACCATGTG | |
| NoName13_m1 | CCCCTTAGGGATAA | 291 | NoName13_m2 | GTGACTGGAGTTC | 366 |
| | CAGGGTAATCCCTA | | | AGACGTGTGCTCT | |
| | GGCCCCTCCCCTCT | | | TCCGATCTGCCCC | |
| | | | | TCCCCTCTTCAAG | |
| | | | | G | |
| NoName13_p1 | CCCCTTAGGGATAA | 292 | NoName13_p2 | GTGACTGGAGTTC | 367 |
| | CAGGGTAATCCCAG | | | AGACGTGTGCTCT | |
| | GTGGTCTCCTCCGA | | | TCCGATCTTCAAC | |
| | CT | | | AGCAACACCCACT | |
| | | | | CTTCC | |
| NoName14_m1 | CCCCTTAGGGATAA | 293 | NoName14_m2 | GTGACTGGAGTTC | 368 |
| | CAGGGTAATCAGGG | | | AGACGTGTGCTCT | |
| | GAGATGCTCAGTGT | | | TCCGATCTGTGTG | |
| | GGT | | | GTGGGGGCTGAGC | |
| NoName14_p1 | CCCCTTAGGGATAA | 294 | NoName14_p2 | GTGACTGGAGTTC | 369 |
| | CAGGGTAATCTGAG | | | AGACGTGTGCTCT | |
| | CACAAGGTCGTCTC | | | TCCGATCTCTCTG | |
| | CTCT | | | ACTTTGACAGTGA | |
| | | | | CACCCATT | |
| NoName15_m1 | CCCCTTAGGGATAA | 295 | NoName15_m2 | GTGACTGGAGTTC | 370 |
| | CAGGGTAATCTGGC | | | AGACGTGTGCTCT | |
| | AGATGAATAAGGCT | | | TCCGATCTGGCTC | |
| | CACTCCT | | | ACTCCTTCTCTTGT | |
| | | | | AGGTACT | |
| NoName15_p1 | CCCCTTAGGGATAA | 296 | NoName15_p2 | GTGACTGGAGTTC | 371 |
| | CAGGGTAATCTCCC | | | AGACGTGTGCTCT | |
| | TACAGAGATAAACA | | | TCCGATCTGAGAG | |
| | GACGCACA | | | AGAGTAAGGTCAG | |
| | | | | GCATGTGG | |
| NoName16_m1 | CCCCTTAGGGATAA | 297 | NoName16_m2 | GTGACTGGAGTTC | 372 |
| | CAGGGTAATCCAGT | | | AGACGTGTGCTCT | |
| | TCTTTGGGTCCTCA | | | TCCGATCTTCATCA | |
| | TCACAGT | | | CAGTTAATGTTGC | |
| | | | | AGCGGAA | |
| NoName16_p1 | CCCCTTAGGGATAA | 298 | NoName16_p2 | GTGACTGGAGTTC | 373 |
| | CAGGGTAATCAGCA | | | AGACGTGTGCTCT | |
| | ACATACAGATGGGG | | | TCCGATCTGCTGG | |
| | TGGGA | | | AGCTGTGGGGGCA | |
| | | | | A | |
| NoName17_m1 | CCCCTTAGGGATAA | 299 | NoName17_m2 | GTGACTGGAGTTC | 374 |
| | CAGGGTAATCGGAT | | | AGACGTGTGCTCT | |
| | GCTTAGCTTCCGTT | | | TCCGATCTGCTTA | |
| | GGGTT | | | GCTTCCGTTGGGT | |
| | | | | TGATGAGG | |
| NoName17_p1 | CCCCTTAGGGATAA | 300 | NoName17_p2 | GTGACTGGAGTTC | 375 |
| | CAGGGTAATCCTGG | | | AGACGTGTGCTCT | |
| | GCACGGTGGACAG | | | TCCGATCTCACGG | |
| | C | | | TGGACAGCAGTGC | |
| | | | | A | |
| NoName18_m1 | CCCCTTAGGGATAA | 301 | NoName18_m2 | GTGACTGGAGTTC | 376 |
| | CAGGGTAATCCCTC | | | AGACGTGTGCTCT | |
| | TTCAAGTGGTCTGC | | | TCCGATCTGCATG | |
| | ATGGAA | | | GAAACTGTGAGG | |
| | | | | AGGGGAGT | |
| NoName18_p1 | CCCCTTAGGGATAA | 302 | NoName18_p2 | GTGACTGGAGTTC | 377 |
| | CAGGGTAATCGGTG | | | AGACGTGTGCTCT | |
| | GTCTCCTCCGATTT | | | TCCGATCTAGTGA | |
| | CAACA | | | CACCCCCTCCTCC | |
| | | | | A | |
| NoName19_m1 | CCCCTTAGGGATAA | 303 | NoName19_m2 | GTGACTGGAGTTC | 378 |
| | CAGGGTAATCTTGC | | | AGACGTGTGCTCT | |
| | GGGGAGGGGAGAT | | | TCCGATCTAGGGA | |
| | TCT | | | ACTGGACACGTCA | |
| | | | | GGGA | |
| NoName19_p1 | CCCCTTAGGGATAA | 304 | NoName19_p2 | GTGACTGGAGTTC | 379 |
| | CAGGGTAATCCCCT | | | AGACGTGTGCTCT | |
| | ACCTCACCGCCAAT | | | TCCGATCTACTTTG | |
| | GTTT | | | GTGGGCGTATAAG | |
| | | | | CAGTTT | |
| NoName2_m1 | CCCCTTAGGGATAA | 305 | NoName2_m2 | GTGACTGGAGTTC | 380 |
| | CAGGGTAATCGAGG | | | AGACGTGTGCTCT | |
| | AGGGGAGAGTCTC | | | TCCGATCTAGGGG | |
| | AGTGTT | | | AGAGTCTCAGTGT | |
| | | | | TGTGGAG | |
| NoName2_p1 | CCCCTTAGGGATAA | 306 | NoName2_p2 | GTGACTGGAGTTC | 381 |
| | CAGGGTAATCACTT | | | AGACGTGTGCTCT | |
| | TAACAGCATCACCC | | | TCCGATCTGGCTA | |
| | ACTCTTCC | | | CAGCAACAGGGTA | |
| | | | | GTAGACC | |
| NoName20_m1 | CCCCTTAGGGATAA | 307 | NoName20_m2 | GTGACTGGAGTTC | 382 |
| | CAGGGTAATCTTTC | | | AGACGTGTGCTCT | |
| | CTGTATTGCTTTTGC | | | TCCGATCTTGCCTT | |
| | CTTGAGC | | | GAGCTTCTTACCC | |
| | | | | CAGTGAG | |
| NoName20_p1 | CCCCTTAGGGATAA | 308 | NoName20_p2 | GTGACTGGAGTTC | 383 |
| | CAGGGTAATCGGAG | | | AGACGTGTGCTCT | |
| | CCTGGACCACTAAG | | | TCCGATCTTTCCA | |
| | TCAC | | | ACCAAGGTACCTG | |
| | | | | TATTGGAC | |
| NoName21_m1 | CCCCTTAGGGATAA | 309 | NoName21_m2 | GTGACTGGAGTTC | 384 |
| | CAGGGTAATCGCGT | | | AGACGTGTGCTCT | |
| | GGAGGTGAGCTCAT | | | TCCGATCTCCCTG | |
| | GTAG | | | CTCACTGGAGAAG | |
| | | | | TTTTCCG | |
| NoName21_p1 | CCCCTTAGGGATAA | 310 | NoName21_p2 | GTGACTGGAGTTC | 385 |
| | CAGGGTAATCGGGC | | | AGACGTGTGCTCT | |
| | GCTCAGTAGGTGTG | | | TCCGATCTGCGCT | |
| | C | | | CAGTAGGTGTGCA | |
| | | | | AGCAG | |
| NoName22_m1 | CCCCTTAGGGATAA | 311 | NoName22_m2 | GTGACTGGAGTTC | 386 |
| | CAGGGTAATCCTGT | | | AGACGTGTGCTCT | |
| | GGGCCATCTTCAAG | | | TCCGATCTTCTCAT | |
| | TTCAGTCC | | | TTCTGGACCTAGG | |
| | | | | CTGATGG | |
| NoName22_p1 | CCCCTTAGGGATAA | 312 | NoName22_p2 | GTGACTGGAGTTC | 387 |
| | CAGGGTAATCAAAA | | | AGACGTGTGCTCT | |
| | ACCTCCACCCTTAT | | | TCCGATCTTCCAC | |
| | GAAGCCT | | | CCTTATGAAGCCT | |
| | | | | CCTTCTAG | |
| NoName23_m1 | CCCCTTAGGGATAA | 313 | NoName23_m2 | GTGACTGGAGTTC | 388 |
| | CAGGGTAATCTCTC | | | AGACGTGTGCTCT | |
| | TGCTGTGTGCTGTC | | | TCCGATCTGTCCA | |
| | CAC | | | CTCACAGGGGTAG | |
| | | | | AACATGTT | |
| NoName23_p1 | CCCCTTAGGGATAA | 314 | NoName23_p2 | GTGACTGGAGTTC | 389 |
| | CAGGGTAATCAGCC | | | AGACGTGTGCTCT | |
| | CCTCCCTCTCCAGG | | | TCCGATCTAGGTG | |
| | A | | | GGGGACTGAGTGT | |
| | | | | GAC | |
| NoName24_m1 | CCCCTTAGGGATAA | 315 | NoName24_m2 | GTGACTGGAGTTC | 390 |
| | CAGGGTAATCGATG | | | AGACGTGTGCTCT | |
| | CTGGGGCTGGCACT | | | TCCGATCTGCAAC | |
| | | | | AGGGTGGTGGAA | |
| | | | | CTCATGT | |
| NoName24_p1 | CCCCTTAGGGATAA | 316 | NoName24_p2 | GTGACTGGAGTTC | 391 |
| | CAGGGTAATCACTG | | | AGACGTGTGCTCT | |
| | TGTCCAGGGGAGAT | | | TCCGATCTCAGTG | |
| | TCTCA | | | TGGTAAGGGACTG | |
| | | | | AGTGCGT | |
| NoName25_m1 | CCCCTTAGGGATAA | 317 | NoName25_m2 | GTGACTGGAGTTC | 392 |
| | CAGGGTAATCACTT | | | AGACGTGTGCTCT | |
| | ACGCTTAGGTGTGA | | | TCCGATCTACACA | |
| | TTTGCGAA | | | TTGCTGCCATGAT | |
| | | | | CTGTCGTA | |
| NoName26_m1 | CCCCTTAGGGATAA | 318 | NoName26_m2 | GTGACTGGAGTTC | 393 |
| | CAGGGTAATCCAGG | | | AGACGTGTGCTCT | |
| | CAAGGCTGAATGGA | | | TCCGATCTGCTGA | |
| | AGCG | | | ATGGAAGCGAGTG | |
| | | | | AAGTGAGC | |
| NoName26_p1 | CCCCTTAGGGATAA | 319 | NoName26_p2 | GTGACTGGAGTTC | 394 |
| | CAGGGTAATCCCTG | | | AGACGTGTGCTCT | |
| | GGGAAGGGCCATTC | | | TCCGATCTGGGCC | |
| | A | | | ATTCACCCTTGATA | |
| | | | | TCATCA | |
| NoName27_m1 | CCCCTTAGGGATAA | 320 | NoName27_m2 | GTGACTGGAGTTC | 395 |
| | CAGGGTAATCGGAG | | | AGACGTGTGCTCT | |
| | ACGGTGCAGGAGC | | | TCCGATCTCTGAG | |
| | TC | | | CAGCGGGGAGGC | |
| | | | | T | |
| NoName27_p1 | CCCCTTAGGGATAA | 321 | NoName27_p2 | GTGACTGGAGTTC | 396 |
| | CAGGGTAATCAGGA | | | AGACGTGTGCTCT | |
| | CCCTCCTCACGGGA | | | TCCGATCTACCCA | |
| | TAC | | | GCTTTCAGCCAGA | |
| | | | | CC | |
| NoName28_m1 | CCCCTTAGGGATAA | 322 | NoName28_m2 | GTGACTGGAGTTC | 397 |
| | CAGGGTAATCGTGT | | | AGACGTGTGCTCT | |
| | GGTGGGGGACTGA | | | TCCGATCTGTGGG | |
| | GC | | | GGACTGAGCATGG | |
| | | | | CA | |
| NoName28_p1 | CCCCTTAGGGATAA | 323 | NoName28_p2 | GTGACTGGAGTTC | 398 |
| | CAGGGTAATCGATG | | | AGACGTGTGCTCT | |
| | CTGGGGCTGCCATT | | | TCCGATCTGGCTG | |
| | G | | | CCATTGCCCTCAG | |
| | | | | T | |
| NoName29_m1 | CCCCTTAGGGATAA | 324 | NoName29_m2 | GTGACTGGAGTTC | 399 |
| | CAGGGTAATCCTCC | | | AGACGTGTGCTCT | |
| | TCACCACCCCCAAG | | | TCCGATCTGGTGG | |
| | G | | | GGGCACAGTCCTG | |
| NoName29_p1 | CCCCTTAGGGATAA | 325 | NoName29_p2 | GTGACTGGAGTTC | 400 |
| | CAGGGTAATCGGCC | | | AGACGTGTGCTCT | |
| | AAAGTCCGCCCCAA | | | TCCGATCTCCAAA | |
| | G | | | GTCCGCCCCAAGG | |
| | | | | TCAAAA | |
| NoName3_m1 | CCCCTTAGGGATAA | 326 | NoName3_m2 | GTGACTGGAGTTC | 401 |
| | CAGGGTAATCGGAG | | | AGACGTGTGCTCT | |
| | GCCCCAGGAACTTT | | | TCCGATCTGGAGG | |
| | CA | | | AGAACGAGGCATG | |
| | | | | TCTTAC | |
| NoName3_p1 | CCCCTTAGGGATAA | 327 | NoName3_p2 | GTGACTGGAGTTC | 402 |
| | CAGGGTAATCCCTC | | | AGACGTGTGCTCT | |
| | GGGAGGTGGGTAG | | | TCCGATCTCTCGG | |
| | TGT | | | GAGGTGGGTAGTG | |
| | | | | TATGGTT | |
| NoName30_m1 | CCCCTTAGGGATAA | 328 | NoName30_m2 | GTGACTGGAGTTC | 403 |
| | CAGGGTAATCGGAC | | | AGACGTGTGCTCT | |
| | CAGCTTGTTGAGGA | | | TCCGATCTCCAGC | |
| | CCCTA | | | TTGTTGAGGACCC | |
| | | | | TAAAGGCT | |
| NoName30_p1 | CCCCTTAGGGATAA | 329 | NoName30_p2 | GTGACTGGAGTTC | 404 |
| | CAGGGTAATCGAGC | | | AGACGTGTGCTCT | |
| | CTCATCAGTTGACC | | | TCCGATCTTTGAC | |
| | CCAA | | | CCCAATGTCCTGC | |
| | | | | ATGTACTA | |
| NoName31_m1 | CCCCTTAGGGATAA | 330 | NoName31_m2 | GTGACTGGAGTTC | 405 |
| | CAGGGTAATCGGGG | | | AGACGTGTGCTCT | |
| | TGCAGCCTGGAGA | | | TCCGATCTGAGAG | |
| | GA | | | AGCTGGGTTGGCT | |
| | | | | GACAGA | |
| NoName31_p1 | CCCCTTAGGGATAA | 331 | NoName31_p2 | GTGACTGGAGTTC | 406 |
| | CAGGGTAATCAGCT | | | AGACGTGTGCTCT | |
| | TTGCTGGGGTAACA | | | TCCGATCTGGGTA | |
| | GGACAC | | | ACAGGACACATTG | |
| | | | | GCTGGGA | |
| NoName32_p1 | CCCCTTAGGGATAA | 332 | NoName32_p2 | GTGACTGGAGTTC | 407 |
| | CAGGGTAATCGAAA | | | AGACGTGTGCTCT | |
| | CTATGAAACTACCA | | | TCCGATCTCCAGG | |
| | GGAGAAGT | | | AGAAGTTTCCAGT | |
| | | | | GGGA | |
| NoName33_m1 | CCCCTTAGGGATAA | 333 | NoName33_m2 | GTGACTGGAGTTC | 408 |
| | CAGGGTAATCGTTC | | | AGACGTGTGCTCT | |
| | AAAGCATCATCTGT | | | TCCGATCTAGCAT | |
| | GAATCAA | | | CATCTGTGAATCA | |
| | | | | AAAGTTTT | |
| NoName33_p1 | CCCCTTAGGGATAA | 334 | NoName33_p2 | GTGACTGGAGTTC | 409 |
| | CAGGGTAATCTCTG | | | AGACGTGTGCTCT | |
| | AGGCCAGCAAAAC | | | TCCGATCTGGCCA | |
| | CTTGA | | | GCAAAACCTTGAC | |
| | | | | ATGTAAAC | |
| NoName34_m1 | CCCCTTAGGGATAA | 335 | NoName34_m2 | GTGACTGGAGTTC | 410 |
| | CAGGGTAATCACTG | | | AGACGTGTGCTCT | |
| | ACACCTGGAGGCCT | | | TCCGATCTACCTG | |
| | GA | | | GAGGCCTGACTTG | |
| | | | | CAG | |
| NoName34_p1 | CCCCTTAGGGATAA | 336 | NoName34_p2 | GTGACTGGAGTTC | 411 |
| | CAGGGTAATCCTGG | | | AGACGTGTGCTCT | |
| | AGGGTGTATGCGTG | | | TCCGATCTAGGGT | |
| | CT | | | GTATGCGTGCTCT | |
| | | | | CTGA | |
| NoName35_m1 | CCCCTTAGGGATAA | 337 | NoName35_m2 | GTGACTGGAGTTC | 412 |
| | CAGGGTAATCCTGG | | | AGACGTGTGCTCT | |
| | GGTTGGCGTCACCT | | | TCCGATCTGCGTC | |
| | | | | ACCTTGAACGACC | |
| | | | | ACTTTGT | |
| NoName35_p1 | CCCCTTAGGGATAA | 338 | NoName35_p2 | GTGACTGGAGTTC | 413 |
| | CAGGGTAATCATTC | | | AGACGTGTGCTCT | |
| | TTCAGGGGGTCTGG | | | TCCGATCTAGGGG | |
| | CATGA | | | GTCTGGCATGAAA | |
| | | | | ATGTGTTA | |
| NoName36_m1 | CCCCTTAGGGATAA | 339 | NoName36_m2 | GTGACTGGAGTTC | 414 |
| | CAGGGTAATCCACC | | | AGACGTGTGCTCT | |
| | CATATGCACACCCA | | | TCCGATCTCACAC | |
| | CATATACC | | | CCACATATACCTGC | |
| | | | | CAAAAGA | |
| NoName37_m1 | CCCCTTAGGGATAA | 340 | NoName37_m2 | GTGACTGGAGTTC | 415 |
| | CAGGGTAATCGAAA | | | AGACGTGTGCTCT | |
| | ACGCCCTACTGCCC | | | TCCGATCTACGCC | |
| | TAGAT | | | CTACTGCCCTAGA | |
| | | | | TTCTAATT | |
| NoName37_p1 | CCCCTTAGGGATAA | 341 | NoName37_p2 | GTGACTGGAGTTC | 416 |
| | CAGGGTAATCAGTC | | | AGACGTGTGCTCT | |
| | CGCCCCCTTATCATC | | | TCCGATCTTGGGG | |
| | CTCTCTG | | | GCTCTGGGGCTAC | |
| | | | | T | |
| NoName38_m1 | CCCCTTAGGGATAA | 342 | NoName38_m2 | GTGACTGGAGTTC | 417 |
| | CAGGGTAATCCCAA | | | AGACGTGTGCTCT | |
| | CGTGGACATGAGGA | | | TCCGATCTACGTG | |
| | TGCAT | | | GACATGAGGATGC | |
| | | | | ATTAAAGG | |
| NoName38_p1 | CCCCTTAGGGATAA | 343 | NoName38_p2 | GTGACTGGAGTTC | 418 |
| | CAGGGTAATCTGGC | | | AGACGTGTGCTCT | |
| | TTCCCAACCTGAGG | | | TCCGATCTATCCCC | |
| | TTTTG | | | TCTTCCCCAAGCC | |
| | | | | T | |
| NoName39_m1 | CCCCTTAGGGATAA | 344 | NoName39_m2 | GTGACTGGAGTTC | 419 |
| | CAGGGTAATCGACA | | | AGACGTGTGCTCT | |
| | CAGGAGAACCCAC | | | TCCGATCTGAACC | |
| | TGAACGC | | | CACTGAACGCTTC | |
| | | | | CACTTCCA | |
| NoName39_p1 | CCCCTTAGGGATAA | 345 | NoName39_p2 | GTGACTGGAGTTC | 420 |
| | CAGGGTAATCTCTC | | | AGACGTGTGCTCT | |
| | CACAGTACAATGAG | | | TCCGATCTAGTAC | |
| | GCCATG | | | AATGAGGCCATGC | |
| | | | | AGTTTCTT | |
| NoName4_m1 | CCCCTTAGGGATAA | 346 | NoName4_m2 | GTGACTGGAGTTC | 421 |
| | CAGGGTAATCCGTG | | | AGACGTGTGCTCT | |
| | CACAGGGGACAGA | | | TCCGATCTACAGG | |
| | AGC | | | GGACAGAAGCCAT | |
| | | | | GGG | |
| NoName4_p1 | CCCCTTAGGGATAA | 347 | NoName4_p2 | GTGACTGGAGTTC | 422 |
| | CAGGGTAATCCCCA | | | AGACGTGTGCTCT | |
| | GGAGCTACGCCTCT | | | TCCGATCTCTACG | |
| | G | | | CCTCTGCCCCATA | |
| | | | | CACG | |
| NoName40_m1 | CCCCTTAGGGATAA | 348 | NoName40_m2 | GTGACTGGAGTTC | 423 |
| | CAGGGTAATCGGCT | | | AGACGTGTGCTCT | |
| | GGCATTGCTCTCAA | | | TCCGATCTTGGCA | |
| | CGA | | | TTGCTCTCAACGA | |
| | | | | CCACTT | |
| NoName40_p1 | CCCCTTAGGGATAA | 349 | NoName40_p2 | GTGACTGGAGTTC | 424 |
| | CAGGGTAATCCATG | | | AGACGTGTGCTCT | |
| | ACGAGGTCAGGCTC | | | TCCGATCTCCCTA | |
| | CCTAGGC | | | GGCCCCTCCGTCT | |
| | | | | TCAG | |
| NoName41_m1 | CCCCTTAGGGATAA | 350 | NoName41_m2 | GTGACTGGAGTTC | 425 |
| | CAGGGTAATCGTGG | | | AGACGTGTGCTCT | |
| | TGGACTTCGCAGAC | | | TCCGATCTGGACT | |
| | CA | | | TCGCAGACCACAT | |
| | | | | GGC | |
| NoName5_m1 | CCCCTTAGGGATAA | 351 | NoName5_m2 | GTGACTGGAGTTC | 426 |
| | CAGGGTAATCGCCC | | | AGACGTGTGCTCT | |
| | AGCTTAAAACATGA | | | TCCGATCTGCCTC | |
| | GCCATTCA | | | GGCTGGCCTTTAC | |
| | | | | TTG | |
| NoName5_p1 | CCCCTTAGGGATAA | 352 | NoName5_p2 | GTGACTGGAGTTC | 427 |
| | CAGGGTAATCGGGA | | | AGACGTGTGCTCT | |
| | GACAATGGAGATCT | | | TCCGATCTGGCAA | |
| | ACCTCAGT | | | AGTGAGACTAATC | |
| | | | | TAGCTGCT | |
| NoName6_m1 | CCCCTTAGGGATAA | 353 | NoName6_m2 | GTGACTGGAGTTC | 428 |
| | CAGGGTAATCCCCA | | | AGACGTGTGCTCT | |
| | CTGGCGTCTTCAGC | | | TCCGATCTTCTTCA | |
| | A | | | GCACTACGGAGAA | |
| | | | | GACTGG | |
| NoName6_p1 | CCCCTTAGGGATAA | 354 | NoName6_p2 | GTGACTGGAGTTC | 429 |
| | CAGGGTAATCGCCA | | | AGACGTGTGCTCT | |
| | AGGGTGCCAAACG | | | TCCGATCTGTGCC | |
| | TTGATA | | | AAACGTTGATAGT | |
| | | | | GCAGGA | |
| NoName7_m1 | CCCCTTAGGGATAA | 355 | NoName7_m2 | GTGACTGGAGTTC | 430 |
| | CAGGGTAATCCAGC | | | AGACGTGTGCTCT | |
| | GTTTCAGGAAGGG | | | TCCGATCTTGCCC | |
| | AGAGG | | | TGTGCTACTGGAA | |
| | | | | GGC | |
| NoName7_p1 | CCCCTTAGGGATAA | 356 | NoName7_p2 | GTGACTGGAGTTC | 431 |
| | CAGGGTAATCTGTG | | | AGACGTGTGCTCT | |
| | CCCCCATGCATGCC | | | TCCGATCTCCCCC | |
| | | | | ATGCATGCCTCAC | |
| | | | | TCTC | |
| NoName8_m1 | CCCCTTAGGGATAA | 357 | NoName8_m2 | GTGACTGGAGTTC | 432 |
| | CAGGGTAATCGCAT | | | AGACGTGTGCTCT | |
| | TGCCCTCAACGACC | | | TCCGATCTAGCAA | |
| | ACTTTT | | | CAGGGTGATGGAC | |
| | | | | CTC | |
| NoName9_m1 | CCCCTTAGGGATAA | 358 | NoName9_m2 | GTGACTGGAGTTC | 433 |
| | CAGGGTAATCCTTA | | | AGACGTGTGCTCT | |
| | ACTCTCACAGGGCC | | | TCCGATCTCAGGG | |
| | ATGTAGTG | | | CCATGTAGTGTCT | |
| | | | | TAAAGCTG | |
| GAPDH_p1 | CCCCTTAGGGATAA | 359 | GAPDH_p2 | GTGACTGGAGTTC | 434 |
| | CAGGGTAATCAGGG | | | AGACGTGTGCTCT | |
| | GTCTACATGGCAAC | | | TCCGATCTGAGGA | |
| | TGTG | | | GGGGAGATTCAGT | |
| | | | | GTGGT | |

TABLE 5
Sequences of anchored primers for HBB

| First PCR | | SEQ | Second PCR | | SEQ |
|---|---|---|---|---|---|
| primer | | ID | primer | | ID |
| name | Sequence | NO: | name | Sequence | NO: |
| NoName10_m1 | CCCCTTAGGGATAA | 435 | NoName10_m2 | GTGACTGGAGTT | 521 |
| | CAGGGTAATCAGGT | | | CAGACGTGTGCT | |
| | GTGACTCCTTTCCC | | | CTTCCGATCTGT | |
| | AGATCA | | | GACTCCTTTCCC | |
| | | | | AGATCAGATAGC | |
| NoName10_p1 | CCCCTTAGGGATAA | 436 | NoName10_p2 | GTGACTGGAGTT | 522 |
| | CAGGGTAATCAGAA | | | CAGACGTGTGCT | |
| | GTCCTGGGTATGGA | | | CTTCCGATCTCCT | |
| | GGCTTTG | | | GGGTATGGAGGC | |
| | | | | TTTGGCATTC | |
| NoName11_m1 | CCCCTTAGGGATAA | 437 | NoName11_m2 | GTGACTGGAGTT | 523 |
| | CAGGGTAATCCCAC | | | CAGACGTGTGCT | |
| | TAGGCTAAGAGGTA | | | CTTCCGATCTGG | |
| | CACCGT | | | CTAAGAGGTACA | |
| | | | | CCGTAACAGAGA | |
| NoName11_p1 | CCCCTTAGGGATAA | 438 | NoName11_p2 | GTGACTGGAGTT | 524 |
| | CAGGGTAATCCCAG | | | CAGACGTGTGCT | |
| | TGGCATCCCCTTTT | | | CTTCCGATCTAGC | |
| | GTCA | | | ATGTCATATGGCT | |
| | | | | AACACCGGTT | |
| NoName12_m1 | CCCCTTAGGGATAA | 439 | NoName12_m2 | GTGACTGGAGTT | 525 |
| | CAGGGTAATCTTTG | | | CAGACGTGTGCT | |
| | GCAGCGGTGATGAG | | | CTTCCGATCTGA | |
| | GT | | | GGTTTCTCATCCT | |
| | | | | GCATGACGTAT | |
| NoName12_p1 | CCCCTTAGGGATAA | 440 | NoName12_p2 | GTGACTGGAGTT | 526 |
| | CAGGGTAATCGCAA | | | CAGACGTGTGCT | |
| | GGGTAACACCTGAG | | | CTTCCGATCTGT | |
| | AAGGT | | | GTGGGGTAAGGG | |
| | | | | GAGCTG | |
| NoName13_m1 | CCCCTTAGGGATAA | 441 | NoName13_m2 | GTGACTGGAGTT | 527 |
| | CAGGGTAATCTGGC | | | CAGACGTGTGCT | |
| | AGGTGTAGCTTTTT | | | CTTCCGATCTAG | |
| | CTGTTA | | | AACATTCTGTCAT | |
| | | | | TCCAGTCAGA | |
| NoName14_m1 | CCCCTTAGGGATAA | 442 | NoName14_m2 | GTGACTGGAGTT | 528 |
| | CAGGGTAATCGCGG | | | CAGACGTGTGCT | |
| | ATTAAAGGGAAGG | | | CTTCCGATCTAG | |
| | GCTTCG | | | GGAAGGGCTTCG | |
| | | | | AATGAGAATGCT | |
| NoName14_p1 | CCCCTTAGGGATAA | 443 | NoName14_p2 | GTGACTGGAGTT | 529 |
| | CAGGGTAATCGCCG | | | CAGACGTGTGCT | |
| | TTACCATAAGTCAG | | | CTTCCGATCTCA | |
| | CAGGT | | | GAAAGTCACTTC | |
| | | | | CAGCACTTGTGA | |
| NoName15_m1 | CCCCTTAGGGATAA | 444 | NoName15_m2 | GTGACTGGAGTT | 530 |
| | CAGGGTAATCACCC | | | CAGACGTGTGCT | |
| | AAGCGGCCCTTCCT | | | CTTCCGATCTTCC | |
| | | | | TCCAGGCTTGAC | |
| | | | | TTGGC | |
| NoName15_p1 | CCCCTTAGGGATAA | 445 | NoName15_p2 | GTGACTGGAGTT | 531 |
| | CAGGGTAATCCTGC | | | CAGACGTGTGCT | |
| | ACACACATTGCCCA | | | CTTCCGATCTCA | |
| | CTTACA | | | CCCCAGAACACG | |
| | | | | AGCAACT | |
| NoName16_m1 | CCCCTTAGGGATAA | 446 | NoName16_m2 | GTGACTGGAGTT | 532 |
| | CAGGGTAATCGTGA | | | CAGACGTGTGCT | |
| | AGTTGGACCAGCTG | | | CTTCCGATCTGTT | |
| | TCATACA | | | GGACCAGCTGTC | |
| | | | | ATACACACAAC | |
| NoName16_p1 | CCCCTTAGGGATAA | 447 | NoName16_p2 | GTGACTGGAGTT | 533 |
| | CAGGGTAATCTGTG | | | CAGACGTGTGCT | |
| | TGTCACATCAATTA | | | CTTCCGATCTTTG | |
| | ATTTGTGC | | | TGCACAGGTTTA | |
| | | | | AGAAACAAATA | |
| NoName17_p1 | CCCCTTAGGGATAA | 448 | NoName17_p2 | GTGACTGGAGTT | 534 |
| | CAGGGTAATCGCTC | | | CAGACGTGTGCT | |
| | TGCAAGTACTGACT | | | CTTCCGATCTGC | |
| | GCCT | | | AAGTACTGACTG | |
| | | | | CCTCCCCCTT | |
| NoName18_m1 | CCCCTTAGGGATAA | 449 | NoName18_m2 | GTGACTGGAGTT | 535 |
| | CAGGGTAATCATGA | | | CAGACGTGTGCT | |
| | GGGGACACCAGAG | | | CTTCCGATCTGG | |
| | GGAA | | | GACACCAGAGG | |
| | | | | GAAGTGAGG | |
| NoName18_p1 | CCCCTTAGGGATAA | 450 | NoName18_p2 | GTGACTGGAGTT | 536 |
| | CAGGGTAATCCCCT | | | CAGACGTGTGCT | |
| | CTGGAGTCCCATCA | | | CTTCCGATCTATC | |
| | TCAC | | | ACCATCTGGCAT | |
| | | | | CCCTTCAC | |
| NoName19_m1 | CCCCTTAGGGATAA | 451 | NoName19_m2 | GTGACTGGAGTT | 537 |
| | CAGGGTAATCTGCT | | | CAGACGTGTGCT | |
| | GTGTCTGCTGTCCA | | | CTTCCGATCTGT | |
| | TCC | | | GTCTGCTGTCCA | |
| | | | | TCCTTCACAT | |
| NoName19_p1 | CCCCTTAGGGATAA | 452 | NoName19_p2 | GTGACTGGAGTT | 538 |
| | CAGGGTAATCGCTG | | | CAGACGTGTGCT | |
| | CTGCTGGAGAGCCA | | | CTTCCGATCTTGC | |
| | T | | | TGGAGAGCCATC | |
| | | | | TTGAAACTAAG | |
| NoName2_p1 | CCCCTTAGGGATAA | 453 | NoName2_p2 | GTGACTGGAGTT | 539 |
| | CAGGGTAATCGTCG | | | CAGACGTGTGCT | |
| | AACTGCATCCCCTG | | | CTTCCGATCTGC | |
| | GTTT | | | CAGGGCAGCCTT | |
| | | | | CCAG | |
| NoName20_p1 | CCCCTTAGGGATAA | 454 | NoName20_p2 | GTGACTGGAGTT | 540 |
| | CAGGGTAATCGTTC | | | CAGACGTGTGCT | |
| | CGCTACGTCAGTTG | | | CTTCCGATCTCGT | |
| | CCA | | | CAGTTGCCACTT | |
| | | | | CTGTATCCA | |
| NoName21_m1 | CCCCTTAGGGATAA | 455 | NoName21_m2 | GTGACTGGAGTT | 541 |
| | CAGGGTAATCGGAA | | | CAGACGTGTGCT | |
| | TGGCCACCCTTCCC | | | CTTCCGATCTACC | |
| | T | | | CTTCCCTCCTTAT | |
| | | | | CAGAAATTGC | |
| NoName21_p1 | CCCCTTAGGGATAA | 456 | NoName21_p2 | GTGACTGGAGTT | 542 |
| | CAGGGTAATCCCTC | | | CAGACGTGTGCT | |
| | CTGGAGGTCTCTCT | | | CTTCCGATCTGC | |
| | TTAATGC | | | CCCTTTTCTCAC | |
| | | | | AGTGTGCA | |
| NoName22_m1 | CCCCTTAGGGATAA | 457 | NoName22_m2 | GTGACTGGAGTT | 543 |
| | CAGGGTAATCGTCA | | | CAGACGTGTGCT | |
| | TTCTGCTGGGTGAC | | | CTTCCGATCTCAT | |
| | AATG | | | TCTGCTGGGTGA | |
| | | | | CAATGAAATAT | |
| NoName22_p1 | CCCCTTAGGGATAA | 458 | NoName22_p2 | GTGACTGGAGTT | 544 |
| | CAGGGTAATCTCAC | | | CAGACGTGTGCT | |
| | ACAGTGGTTAAGAC | | | CTTCCGATCTGT | |
| | CCTTTGG | | | GGTTAAGACCCT | |
| | | | | TTGGCATGAGAG | |
| NoName23_m1 | CCCCTTAGGGATAA | 459 | NoName23_m2 | GTGACTGGAGTT | 545 |
| | CAGGGTAATCGTGG | | | CAGACGTGTGCT | |
| | GCTAGAAGCTAAGA | | | CTTCCGATCTAG | |
| | AGATCAGC | | | AAGCTAAGAAGA | |
| | | | | TCAGCCAGCAG | |
| NoName23_p1 | CCCCTTAGGGATAA | 460 | NoName23_p2 | GTGACTGGAGTT | 546 |
| | CAGGGTAATCAGTA | | | CAGACGTGTGCT | |
| | CGATGCTGCTTCAC | | | CTTCCGATCTTCA | |
| | ATGGAAC | | | CATGGAACCCAG | |
| | | | | CAGGAATC | |
| NoName24_m1 | CCCCTTAGGGATAA | 461 | NoName24_m2 | GTGACTGGAGTT | 547 |
| | CAGGGTAATCACGA | | | CAGACGTGTGCT | |
| | CTGTTCTCACTGAG | | | CTTCCGATCTAG | |
| | GGGTA | | | GAGGAAAGGGT | |
| | | | | GGAGCTGA | |
| NoName24_p1 | CCCCTTAGGGATAA | 462 | NoName24_p2 | GTGACTGGAGTT | 548 |
| | CAGGGTAATCGGGA | | | CAGACGTGTGCT | |
| | GACTTACCAGCTTC | | | CTTCCGATCTACC | |
| | CCGTA | | | AGCTTCCCGTATC | |
| | | | | TCCCT | |
| NoName25_m1 | CCCCTTAGGGATAA | 463 | NoName25_m2 | GTGACTGGAGTT | 549 |
| | CAGGGTAATCTAAG | | | CAGACGTGTGCT | |
| | GCAGTGTGTTGGGT | | | CTTCCGATCTGCT | |
| | GCT | | | GTTGCAGAAGGG | |
| | | | | ATAGTCAGAG | |
| NoName25_p1 | CCCCTTAGGGATAA | 464 | NoName25_p2 | GTGACTGGAGTT | 550 |
| | CAGGGTAATCCCTT | | | CAGACGTGTGCT | |
| | CCTTCTCCACCCAA | | | CTTCCGATCTATG | |
| | GTAGCTA | | | TGCCCTCTGTGT | |
| | | | | GCCTT | |
| NoName26_m1 | CCCCTTAGGGATAA | 465 | NoName26_m2 | GTGACTGGAGTT | 551 |
| | CAGGGTAATCCTCA | | | CAGACGTGTGCT | |
| | CACTCTACCCTTGT | | | CTTCCGATCTCTC | |
| | GCTACG | | | TACCCTTGTGCTA | |
| | | | | CGCTGTCT | |
| NoName27_m1 | CCCCTTAGGGATAA | 466 | NoName27_m2 | GTGACTGGAGTT | 552 |
| | CAGGGTAATCCAAC | | | CAGACGTGTGCT | |
| | TGGGCATGCTCTCC | | | CTTCCGATCTGC | |
| | TAGG | | | AAGGGGCCAGA | |
| | | | | AGGTCT | |
| NoName27_p1 | CCCCTTAGGGATAA | 467 | NoName27_p2 | GTGACTGGAGTT | 553 |
| | CAGGGTAATCCTGT | | | CAGACGTGTGCT | |
| | GTGGCCCTCAGGTG | | | CTTCCGATCTGG | |
| | TAA | | | CCCTCAGGTGTA | |
| | | | | ACTTACCCTCTC | |
| NoName28_m1 | CCCCTTAGGGATAA | 468 | NoName28_m2 | GTGACTGGAGTT | 554 |
| | CAGGGTAATCACCA | | | CAGACGTGTGCT | |
| | CACCCGGCTCACTC | | | CTTCCGATCTCC | |
| | T | | | ACACCCGGCTCA | |
| | | | | CTCTCCAATT | |
| NoName29_p1 | CCCCTTAGGGATAA | 469 | NoName29_p2 | GTGACTGGAGTT | 555 |
| | CAGGGTAATCGGAG | | | CAGACGTGTGCT | |
| | GTTGCAGGTTGCTG | | | CTTCCGATCTGTT | |
| | GT | | | GCTGGTTGCTGA | |
| | | | | GATCATGCCA | |
| NoName3_m1 | CCCCTTAGGGATAA | 470 | NoName3_m2 | GTGACTGGAGTT | 556 |
| | CAGGGTAATCGGCT | | | CAGACGTGTGCT | |
| | GGAGTCCTGGTCCT | | | CTTCCGATCTCC | |
| | G | | | AATCACGGGCCC | |
| | | | | TGGGA | |
| NoName3_p1 | CCCCTTAGGGATAA | 471 | NoName3_p2 | GTGACTGGAGTT | 557 |
| | CAGGGTAATCATGG | | | CAGACGTGTGCT | |
| | TCACCGCCATTCAC | | | CTTCCGATCTCC | |
| | GT | | | GCCATTCACGTG | |
| | | | | GTGCTTACTG | |
| NoName30_m1 | CCCCTTAGGGATAA | 472 | NoName30_m2 | GTGACTGGAGTT | 558 |
| | CAGGGTAATCCTAT | | | CAGACGTGTGCT | |
| | CATTACCCACACCC | | | CTTCCGATCTCCC | |
| | CTGAGAC | | | ACACCCCTGAGA | |
| | | | | CTGCATA | |
| NoName30_p1 | CCCCTTAGGGATAA | 473 | NoName30_p2 | GTGACTGGAGTT | 559 |
| | CAGGGTAATCAGCT | | | CAGACGTGTGCT | |
| | ACCACGGTGACAGT | | | CTTCCGATCTCG | |
| | AACATAGC | | | GTGACAGTAACA | |
| | | | | TAGCCCAGGGA | |
| NoName31_m1 | CCCCTTAGGGATAA | 474 | NoName31_m2 | GTGACTGGAGTT | 560 |
| | CAGGGTAATCAGCT | | | CAGACGTGTGCT | |
| | GCCAGCCCACAAG | | | CTTCCGATCTAA | |
| | AA | | | AATGGGGCCCTT | |
| | | | | AGTCCTACAATG | |
| NoName31_p1 | CCCCTTAGGGATAA | 475 | NoName31_p2 | GTGACTGGAGTT | 561 |
| | CAGGGTAATCGGGA | | | CAGACGTGTGCT | |
| | GACAGGGTATCCAG | | | CTTCCGATCTGA | |
| | GCT | | | GACAGGGTATCC | |
| | | | | AGGCTGCATACA | |
| NoName32_m1 | CCCCTTAGGGATAA | 476 | NoName32_m2 | GTGACTGGAGTT | 562 |
| | CAGGGTAATCAGTT | | | CAGACGTGTGCT | |
| | CAGGGTCTGGTTCT | | | CTTCCGATCTTTC | |
| | GTGC | | | AGGGTCTGGTTC | |
| | | | | TGTGCACATAA | |
| NoName33_m1 | CCCCTTAGGGATAA | 477 | NoName33_m2 | GTGACTGGAGTT | 563 |
| | CAGGGTAATCCGGC | | | CAGACGTGTGCT | |
| | ATTCTTCCCGGCAA | | | CTTCCGATCTGG | |
| | TGA | | | CATTCTTCCCGG | |
| | | | | CAATGAAATCCT | |
| NoName33_p1 | CCCCTTAGGGATAA | 478 | NoName33_p2 | GTGACTGGAGTT | 564 |
| | CAGGGTAATCTGAC | | | CAGACGTGTGCT | |
| | TCTCAGCACCTTGA | | | CTTCCGATCTCA | |
| | CACTCC | | | GCACCTTGACAC | |
| | | | | TCCAGATGAACT | |
| NoName34_m1 | CCCCTTAGGGATAA | 479 | NoName34_m2 | GTGACTGGAGTT | 565 |
| | CAGGGTAATCCTTT | | | CAGACGTGTGCT | |
| | ATATGTGGGGGATG | | | CTTCCGATCTATG | |
| | GAAAAGAC | | | GAAAAGACAAC | |
| | | | | CCATCATGGTAT | |
| NoName35_m1 | CCCCTTAGGGATAA | 480 | NoName35_m2 | GTGACTGGAGTT | 566 |
| | CAGGGTAATCCAGT | | | CAGACGTGTGCT | |
| | GCCTTTTCCTACTAC | | | CTTCCGATCTCCT | |
| | ACCACA | | | ACTACACCACAC | |
| | | | | TGATGCCTCCA | |
| NoName35_p1 | CCCCTTAGGGATAA | 481 | NoName35_p2 | GTGACTGGAGTT | 567 |
| | CAGGGTAATCCGAA | | | CAGACGTGTGCT | |
| | GGAACCAAACGGA | | | CTTCCGATCTTCT | |
| | ACTTGTGTA | | | GGGTGGGAGCA | |
| | | | | GAGTACTCTT | |
| NoName36_m1 | CCCCTTAGGGATAA | 482 | NoName36_m2 | GTGACTGGAGTT | 568 |
| | CAGGGTAATCAGCT | | | CAGACGTGTGCT | |
| | CATCGAGGCACCAA | | | CTTCCGATCTGT | |
| | ACA | | | GGTGATTACAAG | |
| | | | | GCCACATCCTAC | |
| NoName36_p1 | CCCCTTAGGGATAA | 483 | NoName36_p2 | GTGACTGGAGTT | 569 |
| | CAGGGTAATCATTT | | | CAGACGTGTGCT | |
| | GTCCTGGAACCCAT | | | CTTCCGATCTCCT | |
| | ACTGCAT | | | GGAACCCATACT | |
| | | | | GCATTAGGAAG | |
| NoName37_m1 | CCCCTTAGGGATAA | 484 | NoName37_m2 | GTGACTGGAGTT | 570 |
| | CAGGGTAATCTGAA | | | CAGACGTGTGCT | |
| | AGCATCAACTCTGG | | | CTTCCGATCTAGC | |
| | GAGCATG | | | ATGAAAAAGGCT | |
| | | | | GATGAGTGGGA | |
| NoName37_p1 | CCCCTTAGGGATAA | 485 | NoName37_p2 | GTGACTGGAGTT | 571 |
| | CAGGGTAATCGCCA | | | CAGACGTGTGCT | |
| | CAGTTCCAGTGCAT | | | CTTCCGATCTCC | |
| | TCG | | | ACAGTTCCAGTG | |
| | | | | CATTCGGAAGAA | |
| NoName38_m1 | CCCCTTAGGGATAA | 486 | NoName38_m2 | GTGACTGGAGTT | 572 |
| | CAGGGTAATCGGCT | | | CAGACGTGTGCT | |
| | CCCCAGAAGAAGA | | | CTTCCGATCTGCT | |
| | AGCCT | | | TGCAGAACCACG | |
| | | | | AGCTGA | |
| NoName38_p1 | CCCCTTAGGGATAA | 487 | NoName38_p2 | GTGACTGGAGTT | 573 |
| | CAGGGTAATCGCAA | | | CAGACGTGTGCT | |
| | GTGGTAGGCATGGG | | | CTTCCGATCTTCA | |
| | TTAGAAGA | | | GCTGTGCTTCTA | |
| | | | | ATGTACACCCT | |
| NoName39_m1 | CCCCTTAGGGATAA | 488 | NoName39_m2 | GTGACTGGAGTT | 574 |
| | CAGGGTAATCGCCC | | | CAGACGTGTGCT | |
| | GGCAATCGTTTTCT | | | CTTCCGATCTGC | |
| | AGGG | | | AATCGTTTTCTAG | |
| | | | | GGCACGACTTA | |
| NoName39_p1 | CCCCTTAGGGATAA | 489 | NoName39_p2 | GTGACTGGAGTT | 575 |
| | CAGGGTAATCACCC | | | CAGACGTGTGCT | |
| | CCAGGTCAGCAAG | | | CTTCCGATCTGTC | |
| | C | | | AGCAAGCACTTG | |
| | | | | ATCAGAGCATT | |
| NoName4_m1 | CCCCTTAGGGATAA | 490 | NoName4_m2 | GTGACTGGAGTT | 576 |
| | CAGGGTAATCCTGA | | | CAGACGTGTGCT | |
| | TTAGGGTGGTTCGT | | | CTTCCGATCTGT | |
| | TTTGACGT | | | GGTTCGTTTTGA | |
| | | | | CGTGTCTGTTTC | |
| NoName4_p1 | CCCCTTAGGGATAA | 491 | NoName4_p2 | GTGACTGGAGTT | 577 |
| | CAGGGTAATCGCAC | | | CAGACGTGTGCT | |
| | GACCGCGGCAGAG | | | CTTCCGATCTCA | |
| | T | | | CGACCGCGGCAG | |
| | | | | AGTTATCAG | |
| NoName40_m1 | CCCCTTAGGGATAA | 492 | NoName40_m2 | GTGACTGGAGTT | 578 |
| | CAGGGTAATCAGCT | | | CAGACGTGTGCT | |
| | GCTTCCCAGGCCTT | | | CTTCCGATCTCCC | |
| | G | | | AGGCCTTGGCAA | |
| | | | | TGAGTTTAGG | |
| NoName40_p1 | CCCCTTAGGGATAA | 493 | NoName40_p2 | GTGACTGGAGTT | 579 |
| | CAGGGTAATCAATG | | | CAGACGTGTGCT | |
| | CAGAGGCCAGGAC | | | CTTCCGATCTGG | |
| | ACC | | | CCAGGACACCAC | |
| | | | | CATCCC | |
| NoName41_m1 | CCCCTTAGGGATAA | 494 | NoName41_m2 | GTGACTGGAGTT | 580 |
| | CAGGGTAATCTCAT | | | CAGACGTGTGCT | |
| | GTTGTGGTTGGAAG | | | CTTCCGATCTTGT | |
| | TGTGGAT | | | GGTTGGAAGTGT | |
| | | | | GGATTACTGGT | |
| NoName41_p1 | CCCCTTAGGGATAA | 495 | NoName41_p2 | GTGACTGGAGTT | 581 |
| | CAGGGTAATCTGGC | | | CAGACGTGTGCT | |
| | TGGAAGATGGACG | | | CTTCCGATCTTG | |
| | GAGA | | | GACGGAGAGTG | |
| | | | | GATCACAGATGA | |
| | | | | G | |
| NoName42_m1 | CCCCTTAGGGATAA | 496 | NoName42_m2 | GTGACTGGAGTT | 582 |
| | CAGGGTAATCCACC | | | CAGACGTGTGCT | |
| | AGGCCACTCACCCA | | | CTTCCGATCTCC | |
| | ATT | | | AGGCCACTCACC | |
| | | | | CAATTTGACATG | |
| NoName43_m1 | CCCCTTAGGGATAA | 497 | NoName43_m2 | GTGACTGGAGTT | 583 |
| | CAGGGTAATCGAGA | | | CAGACGTGTGCT | |
| | CCAGTGATTTCAGA | | | CTTCCGATCTTTC | |
| | GTGGCTAG | | | AGAGTGGCTAGG | |
| | | | | TGTTCACTGAT | |
| NoName44_m1 | CCCCTTAGGGATAA | 498 | NoName44_m2 | GTGACTGGAGTT | 584 |
| | CAGGGTAATCACCC | | | CAGACGTGTGCT | |
| | CGAACTTGGTGATG | | | CTTCCGATCTTAC | |
| | CAGTAC | | | GGGGAGCGGGC | |
| | | | | CGGGTT | |
| NoName44_p1 | CCCCTTAGGGATAA | 499 | NoName44_p2 | GTGACTGGAGTT | 585 |
| | CAGGGTAATCGGGT | | | CAGACGTGTGCT | |
| | GGCTCAGAAGTGGT | | | CTTCCGATCTGCT | |
| | TCC | | | CAGAAGTGGTTC | |
| | | | | CAGCCAAG | |
| NoName45_m1 | CCCCTTAGGGATAA | 500 | NoName45_m2 | GTGACTGGAGTT | 586 |
| | CAGGGTAATCGTAG | | | CAGACGTGTGCT | |
| | GTGATAGGGAAACG | | | CTTCCGATCTAG | |
| | CCGAAA | | | GGAAACGCCGA | |
| | | | | AAGTATTTTAGGT | |
| NoName45_p1 | CCCCTTAGGGATAA | 501 | NoName45_p2 | GTGACTGGAGTT | 587 |
| | CAGGGTAATCTCTG | | | CAGACGTGTGCT | |
| | CAGAGCATGGAGG | | | CTTCCGATCTGC | |
| | CAAC | | | AACTGCTCCCTG | |
| | | | | GTCTCTT | |
| NoName46_m1 | CCCCTTAGGGATAA | 502 | NoName46_m2 | GTGACTGGAGTT | 588 |
| | CAGGGTAATCAAGT | | | CAGACGTGTGCT | |
| | CTGAAACGCTGCTC | | | CTTCCGATCTCCT | |
| | TGCTATT | | | GTGATCCCTTCG | |
| | | | | AAGAATCTTGT | |
| NoName47_m1 | CCCCTTAGGGATAA | 503 | NoName47_m2 | GTGACTGGAGTT | 589 |
| | CAGGGTAATCGCAC | | | CAGACGTGTGCT | |
| | CATTTCCACCCAGC | | | CTTCCGATCTTCC | |
| | TTTG | | | ACCCAGCTTTGC | |
| | | | | TCAAGT | |
| NoName47_p1 | CCCCTTAGGGATAA | 504 | NoName47_p2 | GTGACTGGAGTT | 590 |
| | CAGGGTAATCCAAG | | | CAGACGTGTGCT | |
| | TAGCTAGGACTCAA | | | CTTCCGATCTCC | |
| | GGCACATG | | | ACCACGGCCAGA | |
| | | | | TCATTGA | |
| NoName48_m1 | CCCCTTAGGGATAA | 505 | NoName48_m2 | GTGACTGGAGTT | 591 |
| | CAGGGTAATCGGGG | | | CAGACGTGTGCT | |
| | GCTGATATGGGTCA | | | CTTCCGATCTAAC | |
| | ACC | | | TGGGTTGCCATG | |
| | | | | AATCTGCTG | |
| NoName5_m1 | CCCCTTAGGGATAA | 506 | NoName5_m2 | GTGACTGGAGTT | 592 |
| | CAGGGTAATCTGCA | | | CAGACGTGTGCT | |
| | TCGAAGCTGGTGGA | | | CTTCCGATCTGC | |
| | GAC | | | AGGGCTGAGGTG | |
| | | | | GAAAGCT | |
| NoName5_p1 | CCCCTTAGGGATAA | 507 | NoName5_p2 | GTGACTGGAGTT | 593 |
| | CAGGGTAATCCCAG | | | CAGACGTGTGCT | |
| | ACCCTGACTCATGG | | | CTTCCGATCTGA | |
| | ACACACC | | | CACACCCTCCCC | |
| | | | | CATCTGGCA | |
| NoName6_m1 | CCCCTTAGGGATAA | 508 | NoName6_m2 | GTGACTGGAGTT | 594 |
| | CAGGGTAATCACGT | | | CAGACGTGTGCT | |
| | TCCCGTCTGCTCAG | | | CTTCCGATCTTG | |
| | TG | | | GGGTAAAGGGGA | |
| | | | | CTCACTCT | |
| NoName6_p1 | CCCCTTAGGGATAA | 509 | NoName6_p2 | GTGACTGGAGTT | 595 |
| | CAGGGTAATCGAGG | | | CAGACGTGTGCT | |
| | TTGGACCAGCTGTC | | | CTTCCGATCTAGC | |
| | ATACC | | | TGCTTTACTGTCA | |
| | | | | CACGTAGCAG | |
| NoName7_m1 | CCCCTTAGGGATAA | 510 | NoName7_m2 | GTGACTGGAGTT | 596 |
| | CAGGGTAATCGCTA | | | CAGACGTGTGCT | |
| | GTCTTTCCAGGCCA | | | CTTCCGATCTCC | |
| | CCCT | | | ACCCTCTCCGAG | |
| | | | | CCACCT | |
| NoName7_p1 | CCCCTTAGGGATAA | 511 | NoName7_p2 | GTGACTGGAGTT | 597 |
| | CAGGGTAATCTTGG | | | CAGACGTGTGCT | |
| | CAAGCACTCCTCAA | | | CTTCCGATCTCC | |
| | TGGC | | | AGCTTACAGGCA | |
| | | | | GGGCTGT | |
| NoName8_m1 | CCCCTTAGGGATAA | 512 | NoName8_m2 | GTGACTGGAGTT | 598 |
| | CAGGGTAATCGCAG | | | CAGACGTGTGCT | |
| | AGAGGAGGGGCTA | | | CTTCCGATCTGG | |
| | AAGGG | | | GGCAGGAAGGG | |
| | | | | AGAAGCAC | |
| NoName8_p1 | CCCCTTAGGGATAA | 513 | NoName8_p2 | GTGACTGGAGTT | 599 |
| | CAGGGTAATCCTCC | | | CAGACGTGTGCT | |
| | CATCCATACCCCCA | | | CTTCCGATCTTCC | |
| | CCT | | | ACCCCCAACCTG | |
| | | | | AGAAGAC | |
| NoName9_m1 | CCCCTTAGGGATAA | 514 | NoName9_m2 | GTGACTGGAGTT | 600 |
| | CAGGGTAATCGCCC | | | CAGACGTGTGCT | |
| | CAACCCAAGCTAGT | | | CTTCCGATCTCCC | |
| | CTTTC | | | AAGCTAGTCTTT | |
| | | | | CCAGGCCACT | |
| OT1-NC_m1 | CCCCTTAGGGATAA | 515 | OT1-NC_m2 | GTGACTGGAGTT | 601 |
| | CAGGGTAATCCCTT | | | CAGACGTGTGCT | |
| | TCCCGTTCTCCACC | | | CTTCCGATCTCC | |
| | CAA | | | GTTCTCCACCCA | |
| | | | | ATAGCTATGG | |
| OT1-NC_p1 | CCCCTTAGGGATAA | 516 | OT1-NC_p2 | GTGACTGGAGTT | 602 |
| | CAGGGTAATCAGCA | | | CAGACGTGTGCT | |
| | GTATGTCCAACTCC | | | CTTCCGATCTTCC | |
| | CAAATTG | | | AACTCCCAAATT | |
| | | | | GAAAGCACAGC | |
| OT2-NC_m1 | CCCCTTAGGGATAA | 517 | OT2-NC_m2 | GTGACTGGAGTT | 603 |
| | CAGGGTAATCACAC | | | CAGACGTGTGCT | |
| | AGGTTTTCTCCTCT | | | CTTCCGATCTTTC | |
| | CAGCCTA | | | CCTTCCCTAGAC | |
| | | | | CTGCCT | |
| OT2-NC_p1 | CCCCTTAGGGATAA | 518 | OT2-NC_p2 | GTGACTGGAGTT | 604 |
| | CAGGGTAATCAACC | | | CAGACGTGTGCT | |
| | TGGCTCCTTCGCTT | | | CTTCCGATCTGG | |
| | CC | | | CTCCTTCGCTTCC | |
| | | | | ATCTGATCAGG | |
| HBB_m1 | CCCCTTAGGGATAA | 519 | HBB_m2 | GTGACTGGAGTT | 605 |
| | CAGGGTAATCCTCT | | | CAGACGTGTGCT | |
| | GTCTCCACATGCCC | | | CTTCCGATCTGTC | |
| | AGTT | | | TCCACATGCCCA | |
| | | | | GTTTCTATTGG | |
| HBB_p1 | CCCCTTAGGGATAA | 520 | HBB_p2 | GTGACTGGAGTT | 606 |
| | CAGGGTAATCCCAG | | | CAGACGTGTGCT | |
| | GGCTGGGCATAAAA | | | CTTCCGATCTTTC | |
| | GTCAG | | | ACTAGCAACCTC | |
| | | | | AAACAGACACC | |

TABLE_6
Sequences of anchored primers for PD1

| First | | | Second | | |
|---|---|---|---|---|---|
| PCR | | SEQ | PCR | | SEQ |
| primer | | ID | primer | | ID |
| name | Sequence | NO: | name | Sequence | NO: |
| NoName1_m1 | CCCCTTAGGGATAAC | 607 | NoName1_m2 | GTGACTGGAGTTCA | 699 |
| | AGGGTAATCTGGGCT | | | GACGTGTGCTCTTC | |
| | GAGAGCTAGCTTTAT | | | CGATCTCAGTCACC | |
| | GTGA | | | ACACTGGGTAACTC | |
| | | | | CT | |
| NoName1_p1 | CCCCTTAGGGATAAC | 608 | NoName1_p2 | GTGACTGGAGTTCA | 700 |
| | AGGGTAATCAGGAG | | | GACGTGTGCTCTTC | |
| | GCAGGGACGTGAAA | | | CGATCTGTGAAACG | |
| | C | | | CTGGGGTGCAATTT | |
| | | | | C | |
| NoName10_m1 | CCCCTTAGGGATAAC | 609 | NoName10_m2 | GTGACTGGAGTTCA | 701 |
| | AGGGTAATCAGGTGA | | | GACGTGTGCTCTTC | |
| | CTCCCTGGCTTTGC | | | CGATCTTCCTCTTCC | |
| | | | | CCCAAGCTGGCTT | |
| NoName10_p1 | CCCCTTAGGGATAAC | 610 | NoName10_p2 | GTGACTGGAGTTCA | 702 |
| | AGGGTAATCTGATCT | | | GACGTGTGCTCTTC | |
| | GAGGGGCTTGGCAG | | | CGATCTGGCAGAGA | |
| | A | | | GGCACCCCAA | |
| NoName11_m1 | CCCCTTAGGGATAAC | 611 | NoName11_m2 | GTGACTGGAGTTCA | 703 |
| | AGGGTAATCCACATG | | | GACGTGTGCTCTTC | |
| | TGGTACGTCTGGTCC | | | CGATCTCGTCTGGT | |
| | AGT | | | CCAGTCAGCCTTGC | |
| NoName11_p1 | CCCCTTAGGGATAAC | 612 | NoName11_p2 | GTGACTGGAGTTCA | 704 |
| | AGGGTAATCACGACG | | | GACGTGTGCTCTTC | |
| | GGTGTGTGGGTGA | | | CGATCTCGGGTGTG | |
| | | | | TGGGTGACAAGCG | |
| NoName12_m1 | CCCCTTAGGGATAAC | 613 | NoName12_m2 | GTGACTGGAGTTCA | 705 |
| | AGGGTAATCCAGCTG | | | GACGTGTGCTCTTC | |
| | GGGCGACATAGTGA | | | CGATCTGGGGAGTT | |
| | | | | AATGTAAGGGAGGC | |
| | | | | AACA | |
| NoName12_p1 | CCCCTTAGGGATAAC | 614 | NoName12_p2 | GTGACTGGAGTTCA | 706 |
| | AGGGTAATCGGTAAC | | | GACGTGTGCTCTTC | |
| | TGTAATATAGAGCCC | | | CGATCTGAGCCCAC | |
| | ACCA | | | CACTCAGCTTT | |
| NoName14_m1 | CCCCTTAGGGATAAC | 615 | NoName14_m2 | GTGACTGGAGTTCA | 707 |
| | AGGGTAATCGGGGA | | | GACGTGTGCTCTTC | |
| | GGGACAGGTTGTGA | | | CGATCTTGGGCTTG | |
| | G | | | GAGTTAAGGGGCCT | |
| | | | | A | |
| NoName14_p1 | CCCCTTAGGGATAAC | 616 | NoName14_p2 | GTGACTGGAGTTCA | 708 |
| | AGGGTAATCTGAATC | | | GACGTGTGCTCTTC | |
| | ACCAACTGCCAAAC | | | CGATCTCCAACTGC | |
| | ACGTG | | | CAAACACGTGAATG | |
| | | | | AGGT | |
| NoName15_m1 | CCCCTTAGGGATAAC | 617 | NoName15_m2 | GTGACTGGAGTTCA | 709 |
| | AGGGTAATCGGCCCC | | | GACGTGTGCTCTTC | |
| | CAGTGAATCACCAAT | | | CGATCTATGAGGTC | |
| | TG | | | ATCTGAGGCCATCC | |
| | | | | C | |
| NoName16_m1 | CCCCTTAGGGATAAC | 618 | NoName16_m2 | GTGACTGGAGTTCA | 710 |
| | AGGGTAATCGCAGA | | | GACGTGTGCTCTTC | |
| | ATCAAGCCAGAGCAT | | | CGATCTAGCCAGAG | |
| | GC | | | CATGCCAAGCA | |
| NoName16_p1 | CCCCTTAGGGATAAC | 619 | NoName16_p2 | GTGACTGGAGTTCA | 711 |
| | AGGGTAATCAGAGGT | | | GACGTGTGCTCTTC | |
| | GAGGGCGAGCTAGA | | | CGATCTCGAGCTAG | |
| | | | | AGTAGAAGGTGCCC | |
| | | | | CAT | |
| NoName17_m1 | CCCCTTAGGGATAAC | 620 | NoName17_m2 | GTGACTGGAGTTCA | 712 |
| | AGGGTAATCTGCCAG | | | GACGTGTGCTCTTC | |
| | TGATCTTTCCTTTCCC | | | CGATCTCCTCTGAT | |
| | TCTG | | | GTGTCGATGCCAGC | |
| | | | | CTT | |
| NoName17_p1 | CCCCTTAGGGATAAC | 621 | NoName17_p2 | GTGACTGGAGTTCA | 713 |
| | AGGGTAATCCAACAG | | | GACGTGTGCTCTTC | |
| | TCGGTGTCCTGATGG | | | CGATCTAGTCGGTG | |
| | T | | | TCCTGATGGTAGAA | |
| | | | | AAC | |
| NoName18_m1 | CCCCTTAGGGATAAC | 622 | NoName18_m2 | GTGACTGGAGTTCA | 714 |
| | AGGGTAATCTCCTGT | | | GACGTGTGCTCTTC | |
| | GCCATGACCTTCACA | | | CGATCTAGCCAGTG | |
| | C | | | ATGAAAGGTGCCTC | |
| | | | | AA | |
| NoName18_p1 | CCCCTTAGGGATAAC | 623 | NoName18_p2 | GTGACTGGAGTTCA | 715 |
| | AGGGTAATCATGGGG | | | GACGTGTGCTCTTC | |
| | AGGCGGCAGTGA | | | CGATCTAGCACAGG | |
| | | | | AGAGGGCCTCTG | |
| NoName19_m1 | CCCCTTAGGGATAAC | 624 | NoName19_m2 | GTGACTGGAGTTCA | 716 |
| | AGGGTAATCGGGGCT | | | GACGTGTGCTCTTC | |
| | GGGCAGTCACTC | | | CGATCTTCCCCCAG | |
| | | | | CTCCCAAATCAATC | |
| | | | | AA | |
| NoName19_p1 | CCCCTTAGGGATAAC | 625 | NoName19_p2 | GTGACTGGAGTTCA | 717 |
| | AGGGTAATCCCAGAC | | | GACGTGTGCTCTTC | |
| | TGCGGGTATGAGAGG | | | CGATCTGGCAGCCT | |
| | | | | TTCCTTTTCACAGA | |
| | | | | TG | |
| NoName2_m1 | CCCCTTAGGGATAAC | 626 | NoName2_m2 | GTGACTGGAGTTCA | 718 |
| | AGGGTAATCGGCTCC | | | GACGTGTGCTCTTC | |
| | GACGCTCCACAG | | | CGATCTCCGACGCT | |
| | | | | CCACAGCCTGTC | |
| NoName2_p1 | CCCCTTAGGGATAAC | 627 | NoName2_p2 | GTGACTGGAGTTCA | 719 |
| | AGGGTAATCCCCCTA | | | GACGTGTGCTCTTC | |
| | GCGGCCCAGGCT | | | CGATCTCGGCCCAG | |
| | | | | GCTCGGACTG | |
| NoName20_m1 | CCCCTTAGGGATAAC | 628 | NoName20_m2 | GTGACTGGAGTTCA | 720 |
| | AGGGTAATCTCAGGC | | | GACGTGTGCTCTTC | |
| | TCTAGCAGTCCCAGT | | | CGATCTAGGCTCTA | |
| | A | | | GCAGTCCCAGTAAT | |
| | | | | AAGT | |
| NoName20_p1 | CCCCTTAGGGATAAC | 629 | NoName20_p2 | GTGACTGGAGTTCA | 721 |
| | AGGGTAATCGGCATG | | | GACGTGTGCTCTTC | |
| | GTGAAGAAAGAATG | | | CGATCTATGCTACA | |
| | CTAC | | | CATACTTCACCTTA | |
| | | | | AGGG | |
| NoName21_m1 | CCCCTTAGGGATAAC | 630 | NoName21_m2 | GTGACTGGAGTTCA | 722 |
| | AGGGTAATCAGGTTC | | | GACGTGTGCTCTTC | |
| | TTGCTTAGAGGCATG | | | CGATCTCAACTGTG | |
| | ATGAC | | | GAGACTGACTGGCT | |
| NoName21_p1 | CCCCTTAGGGATAAC | 631 | NoName21_p2 | GTGACTGGAGTTCA | 723 |
| | AGGGTAATCGCCCAT | | | GACGTGTGCTCTTC | |
| | GCTGTTCTTATAGCG | | | CGATCTGGGAGCCA | |
| | GTA | | | TACCTGAGAAGGA | |
| | | | | GA | |
| NoName22_m1 | CCCCTTAGGGATAAC | 632 | NoName22_m2 | GTGACTGGAGTTCA | 724 |
| | AGGGTAATCTGTGCA | | | GACGTGTGCTCTTC | |
| | TACTCAGCTACTGTG | | | CGATCTTGAGCTTG | |
| | CTCTA | | | AGGATCTGTCAGGC | |
| | | | | AA | |
| NoName22_p1 | CCCCTTAGGGATAAC | 633 | NoName22_p2 | GTGACTGGAGTTCA | 725 |
| | AGGGTAATCTGCAGA | | | GACGTGTGCTCTTC | |
| | TGATCTGGCTGATGG | | | CGATCTATGATCTG | |
| | AC | | | GCTGATGGACCAAA | |
| | | | | CATC | |
| NoName23_m1 | CCCCTTAGGGATAAC | 634 | NoName23_m2 | GTGACTGGAGTTCA | 726 |
| | AGGGTAATCCCAGAT | | | GACGTGTGCTCTTC | |
| | TCCCTGCTCAGCAAA | | | CGATCTACAGCGGC | |
| | GTA | | | TGTTGCTCTTCC | |
| NoName23_p1 | CCCCTTAGGGATAAC | 635 | NoName23_p2 | GTGACTGGAGTTCA | 727 |
| | AGGGTAATCCAACCA | | | GACGTGTGCTCTTC | |
| | CTGTGTAATAAGCCG | | | CGATCTCCGCTTGT | |
| | CTTGT | | | ACAACGGTCTTTCC | |
| | | | | TCAA | |
| NoName24_p1 | CCCCTTAGGGATAAC | 636 | NoName24_p2 | GTGACTGGAGTTCA | 728 |
| | AGGGTAATCGCTAAA | | | GACGTGTGCTCTTC | |
| | CTTGGCACTGGCTTT | | | CGATCTATTTGCAG | |
| | CAC | | | CTTCCTCTACACTT | |
| | | | | CCTG | |
| NoName25_m1 | CCCCTTAGGGATAAC | 637 | NoName25_m2 | GTGACTGGAGTTCA | 729 |
| | AGGGTAATCAAACCC | | | GACGTGTGCTCTTC | |
| | CACACACCACACGTA | | | CGATCTCACACCAC | |
| | T | | | ACACGTCACAGAA | |
| | | | | ACC | |
| NoName25_p1 | CCCCTTAGGGATAAC | 638 | NoName25_p2 | GTGACTGGAGTTCA | 730 |
| | AGGGTAATCGGGGCT | | | GACGTGTGCTCTTC | |
| | CCTGAGGGTGGA | | | CGATCTAGAAGGGG | |
| | | | | TGGGAGGCCAA | |
| NoName26_m1 | CCCCTTAGGGATAAC | 639 | NoName26_m2 | GTGACTGGAGTTCA | 731 |
| | AGGGTAATCTGTCTG | | | GACGTGTGCTCTTC | |
| | CAGTCACCTGTCCAC | | | CGATCTTCACCTGT | |
| | | | | CCACTCACAGCAC | |
| NoName26_p1 | CCCCTTAGGGATAAC | 640 | NoName26_p2 | GTGACTGGAGTTCA | 732 |
| | AGGGTAATCCACTCC | | | GACGTGTGCTCTTC | |
| | CAGGCGCTCGAGTT | | | CGATCTGCGCTCGA | |
| | | | | GTTACAGGGCCACT | |
| NoName27_m1 | CCCCTTAGGGATAAC | 641 | NoName7_2m2 | GTGACTGGAGTTCA | 733 |
| | AGGGTAATCGGACA | | | GACGTGTGCTCTTC | |
| | AACACCCACCCAGG | | | CGATCTAGGTGATG | |
| | T | | | TGATCTTCCTGCTT | |
| | | | | GCTC | |
| NoName28_m1 | CCCCTTAGGGATAAC | 642 | NoName28_m2 | GTGACTGGAGTTCA | 734 |
| | AGGGTAATCTTTAAC | | | GACGTGTGCTCTTC | |
| | CTTCTTAGTAGCCAG | | | CGATCTAGCATTAC | |
| | GGAAT | | | ACAACCCCTAGAAA | |
| | | | | GTC | |
| NoName28_p1 | CCCCTTAGGGATAAC | 643 | NoName28_p2 | GTGACTGGAGTTCA | 735 |
| | AGGGTAATCTGCACA | | | GACGTGTGCTCTTC | |
| | TATTCCACGTGGGCA | | | CGATCTCACTGTGT | |
| | TA | | | CATATTGCCTGCATG | |
| | | | | TCT | |
| NoName29_m1 | CCCCTTAGGGATAAC | 644 | NoName29_m2 | GTGACTGGAGTTCA | 736 |
| | AGGGTAATCCCACAG | | | GACGTGTGCTCTTC | |
| | ACATCAGAGCAGAC | | | CGATCTCCCCCAGC | |
| | ACA | | | CCTAGTCCACA | |
| NoName29_p1 | CCCCTTAGGGATAAC | 645 | NoName29_p2 | GTGACTGGAGTTCA | 737 |
| | AGGGTAATCACACCT | | | GACGTGTGCTCTTC | |
| | GGTGAGGGCAACTG | | | CGATCTGTGAGGGC | |
| | | | | AACTGACAAAAGC | |
| | | | | AATT | |
| NoName3_m1 | CCCCTTAGGGATAAC | 646 | NoName3_m2 | GTGACTGGAGTTCA | 738 |
| | AGGGTAATCGAGGCC | | | GACGTGTGCTCTTC | |
| | AGGTCCTACATTGAG | | | CGATCTGGCCAGGT | |
| | C | | | CCTACATTGAGCAA | |
| | | | | TCAT | |
| NoName3_p1 | CCCCTTAGGGATAAC | 647 | NoName3_p2 | GTGACTGGAGTTCA | 739 |
| | AGGGTAATCTCTTTC | | | GACGTGTGCTCTTC | |
| | TGTCAGAGGCAATG | | | CGATCTGGCAATGG | |
| | GT | | | TGTCCACTTTGGA | |
| NoName30_m1 | CCCCTTAGGGATAAC | 648 | NoName30_m2 | GTGACTGGAGTTCA | 740 |
| | AGGGTAATCCCCTGT | | | GACGTGTGCTCTTC | |
| | CTGCCACCTGTTGTC | | | CGATCTCCTGTCTG | |
| | | | | CCACCTGTTGTCAT | |
| | | | | TAAC | |
| NoName30_p1 | CCCCTTAGGGATAAC | 649 | NoName30_p2 | GTGACTGGAGTTCA | 741 |
| | AGGGTAATCGGCCTC | | | GACGTGTGCTCTTC | |
| | TTCTCAATCCCAGTG | | | CGATCTCCTCTTCTC | |
| | C | | | AATCCCAGTGCCTA | |
| | | | | CTC | |
| NoName31_m1 | CCCCTTAGGGATAAC | 650 | NoName31_m2 | GTGACTGGAGTTCA | 742 |
| | AGGGTAATCCATCCC | | | GACGTGTGCTCTTC | |
| | TGACAGCAATGACTC | | | CGATCTCTGACAGC | |
| | ACTC | | | AATGACTCACTCCC | |
| | | | | CTTG | |
| NoName31_p1 | CCCCTTAGGGATAAC | 651 | NoName31_p2 | GTGACTGGAGTTCA | 743 |
| | AGGGTAATCTGTGAG | | | GACGTGTGCTCTTC | |
| | AGTCTGGCCTTTACT | | | CGATCTTGAATCAG | |
| | GGT | | | GAGGGGCTATGTAG | |
| | | | | TTCT | |
| NoName32_m1 | CCCCTTAGGGATAAC | 652 | NoName32_m2 | GTGACTGGAGTTCA | 744 |
| | AGGGTAATCTTGGAC | | | GACGTGTGCTCTTC | |
| | CTCCCCTGCGTGA | | | CGATCTACCTCCCC | |
| | | | | TGCGTGAAACTGTT | |
| | | | | CTA | |
| NoName32_p1 | CCCCTTAGGGATAAC | 653 | NoName32_p2 | GTGACTGGAGTTCA | 745 |
| | AGGGTAATCACTATG | | | GACGTGTGCTCTTC | |
| | TGGACTGTGGGACTC | | | CGATCTTGGACTGT | |
| | TATGA | | | GGGACTCTATGAAT | |
| | | | | GTGG | |
| NoName33_p1 | CCCCTTAGGGATAAC | 654 | NoName33_p2 | GTGACTGGAGTTCA | 746 |
| | AGGGTAATCTTTCAA | | | GACGTGTGCTCTTC | |
| | AGGGGAATGTACTAC | | | CGATCTAGGGGAAT | |
| | CGT | | | GTACTACCGTCACT | |
| | | | | TT | |
| NoName34_m1 | CCCCTTAGGGATAAC | 655 | NoName34_m2 | GTGACTGGAGTTCA | 747 |
| | AGGGTAATCGGCCTG | | | GACGTGTGCTCTTC | |
| | CAACCCCGCTAC | | | CGATCTTGCAACCC | |
| | | | | CGCTACTTCCTCCT | |
| NoName34_p1 | CCCCTTAGGGATAAC | 656 | NoName34_p2 | GTGACTGGAGTTCA | 748 |
| | AGGGTAATCGCTAGG | | | GACGTGTGCTCTTC | |
| | CCCTGGAGATGCTAC | | | CGATCTCAGGGATC | |
| | | | | AGGCCAGGTAAAA | |
| | | | | CA | |
| NoName35_m1 | CCCCTTAGGGATAAC | 657 | NoName35_m2 | GTGACTGGAGTTCA | 749 |
| | AGGGTAATCAGTCCA | | | GACGTGTGCTCTTC | |
| | GCGTTTGAATCAGAT | | | CGATCTCATGGAAG | |
| | CATGG | | | ATGGCTCTAGAGGA | |
| | | | | AGCT | |
| NoName35_p1 | CCCCTTAGGGATAAC | 658 | NoName35_p2 | GTGACTGGAGTTCA | 750 |
| | AGGGTAATCCGTGGG | | | GACGTGTGCTCTTC | |
| | CACTGAGAGCACCA | | | CGATCTTGGGCACT | |
| | | | | GAGAGCACCATCAT | |
| | | | | GG | |
| NoName36_p1 | CCCCTTAGGGATAAC | 659 | NoName36_p2 | GTGACTGGAGTTCA | 751 |
| | AGGGTAATCGGATTG | | | GACGTGTGCTCTTC | |
| | CAGGGTATCCACGTC | | | CGATCTATGCATGA | |
| | TAAAT | | | AGGCCAGCACAATG | |
| | | | | GG | |
| NoName37_m1 | CCCCTTAGGGATAAC | 660 | NoName37_m2 | GTGACTGGAGTTCA | 752 |
| | AGGGTAATCGTGTGT | | | GACGTGTGCTCTTC | |
| | CTCACGTGGTGGGT | | | CGATCTCACGTGGT | |
| | | | | GGGTGATTTTTATTC | |
| | | | | CAG | |
| NoName37_p1 | CCCCTTAGGGATAAC | 661 | NoName37_p2 | GTGACTGGAGTTCA | 753 |
| | AGGGTAATCGGCTGG | | | GACGTGTGCTCTTC | |
| | AATACCCTTTGTAGT | | | CGATCTGGGGGCTG | |
| | TGGG | | | CCTGTGTGTTA | |
| NoName38_m1 | CCCCTTAGGGATAAC | 662 | NoName38_m2 | GTGACTGGAGTTCA | 754 |
| | AGGGTAATCAGCAA | | | GACGTGTGCTCTTC | |
| | GGCGTGGCTGGTG | | | CGATCTCATGGGCA | |
| | | | | AGAGCATGCTGGTA | |
| NoName38_p1 | CCCCTTAGGGATAAC | 663 | NoName38_p2 | GTGACTGGAGTTCA | 755 |
| | AGGGTAATCTCCAGT | | | GACGTGTGCTCTTC | |
| | GCCCTATCAGAGTAA | | | CGATCTCATAGCTT | |
| | TTCCT | | | CTTTGCTGGCCGAC | |
| | | | | CA | |
| NoName39_m1 | CCCCTTAGGGATAAC | 664 | NoName39_m2 | GTGACTGGAGTTCA | 756 |
| | AGGGTAATCGAGGAT | | | GACGTGTGCTCTTC | |
| | GTAAGTAGCGCTTGT | | | CGATCTCACAGCCC | |
| | GAACA | | | CAGGTCCTTTGCG | |
| NoName39_p1 | CCCCTTAGGGATAAC | 665 | NoName39_p2 | GTGACTGGAGTTCA | 757 |
| | AGGGTAATCTGGAGA | | | GACGTGTGCTCTTC | |
| | CAGCGTAAGTGTCCC | | | CGATCTAGTGTCCC | |
| | T | | | TGTCCTCACGCT | |
| NoName4_m1 | CCCCTTAGGGATAAC | 666 | NoName4_m2 | GTGACTGGAGTTCA | 758 |
| | AGGGTAATCGCAATA | | | GACGTGTGCTCTTC | |
| | AACACTGCCTAGAGC | | | CGATCTCACTGCCT | |
| | CTAT | | | AGAGCCTATATTGC | |
| | | | | AAAG | |
| NoName40_p1 | CCCCTTAGGGATAAC | 667 | NoName40_p2 | GTGACTGGAGTTCA | 759 |
| | AGGGTAATCGGCCTT | | | GACGTGTGCTCTTC | |
| | AAAAATTGCTGCGCA | | | CGATCTCTTAAAAA | |
| | GT | | | TTGCTGCGCAGTGG | |
| | | | | CTGT | |
| NoName41_m1 | CCCCTTAGGGATAAC | 668 | NoName41_m2 | GTGACTGGAGTTCA | 760 |
| | AGGGTAATCTGCTCA | | | GACGTGTGCTCTTC | |
| | AGACAGGCCAAGGA | | | CGATCTGCTCAAGA | |
| | C | | | CAGGCCAAGGACTT | |
| | | | | AGAA | |
| NoName41_p1 | CCCCTTAGGGATAAC | 669 | NoName41_p2 | GTGACTGGAGTTCA | 761 |
| | AGGGTAATCTCTTTT | | | GACGTGTGCTCTTC | |
| | CTACTGGGCCTCCAC | | | CGATCTGCTGCTCC | |
| | CT | | | CTTCCCCTCCAC | |
| NoName42_m1 | CCCCTTAGGGATAAC | 670 | NoName42_m2 | GTGACTGGAGTTCA | 762 |
| | AGGGTAATCGCTTCC | | | GACGTGTGCTCTTC | |
| | TTAGCCTGAGGTCAC | | | CGATCTGAGGTCAC | |
| | TAAAA | | | TAAAAATGGCCAGT | |
| | | | | CTGC | |
| NoName42_p1 | CCCCTTAGGGATAAC | 671 | NoName42_p2 | GTGACTGGAGTTCA | 763 |
| | AGGGTAATCAATCCA | | | GACGTGTGCTCTTC | |
| | ACCTAATAAGCACAG | | | CGATCTACTGAGTG | |
| | GCACT | | | CTGGCATCAGGATT | |
| | | | | C | |
| NoName43_m1 | CCCCTTAGGGATAAC | 672 | NoName43_m2 | GTGACTGGAGTTCA | 764 |
| | AGGGTAATCTCCTAG | | | GACGTGTGCTCTTC | |
| | GCTTCTTTCCTCTCC | | | CGATCTCCAGTAGC | |
| | CA | | | CTGTAGTCAGAAAG | |
| | | | | AGTG | |
| NoName43_p1 | CCCCTTAGGGATAAC | 673 | NoName43_p2 | GTGACTGGAGTTCA | 765 |
| | AGGGTAATCGGGGCC | | | GACGTGTGCTCTTC | |
| | ACTGAGACTCCTCT | | | CGATCTCTCCTCTTA | |
| | | | | GGACAACCGACCAT | |
| | | | | CCT | |
| NoName44_m1 | CCCCTTAGGGATAAC | 674 | NoName44_m2 | GTGACTGGAGTTCA | 766 |
| | AGGGTAATCACCTTT | | | GACGTGTGCTCTTC | |
| | GGAACGATGGGGGT | | | CGATCTACCTCTTG | |
| | ATTTT | | | TTTCTCAAAACGCT | |
| | | | | GTCG | |
| NoName44_p1 | CCCCTTAGGGATAAC | 675 | NoName44_p2 | GTGACTGGAGTTCA | 767 |
| | AGGGTAATCCTGGAG | | | GACGTGTGCTCTTC | |
| | CATCGACGAGGGTG | | | CGATCTCATCGACG | |
| | A | | | AGGGTGAGCGCATG | |
| NoName45_p1 | CCCCTTAGGGATAAC | 676 | NoName45_p2 | GTGACTGGAGTTCA | 768 |
| | AGGGTAATCGGAGC | | | GACGTGTGCTCTTC | |
| | ATCGACGAGGGTGA | | | CGATCTTCGACGAG | |
| | G | | | GGTGAGCGCATG | |
| NoName46_m1 | CCCCTTAGGGATAAC | 677 | NoName46_m2 | GTGACTGGAGTTCA | 769 |
| | AGGGTAATCGCCTGC | | | GACGTGTGCTCTTC | |
| | ATTCATTCGTCCACA | | | CGATCTGCCCTGGG | |
| | ATAC | | | CTTGGCATGAA | |
| NoName46_p1 | CCCCTTAGGGATAAC | 678 | NoName46_p2 | GTGACTGGAGTTCA | 770 |
| | AGGGTAATCAGATGC | | | GACGTGTGCTCTTC | |
| | TGAGAGTTTACCCCC | | | CGATCTCCCCCTCT | |
| | TCTAC | | | ACCTCCCACCTT | |
| NoName47_m1 | CCCCTTAGGGATAAC | 679 | NoName47_m2 | GTGACTGGAGTTCA | 771 |
| | AGGGTAATCTTTTTC | | | GACGTGTGCTCTTC | |
| | TCCCCAAACGTGAG | | | CGATCTTCCCCAAA | |
| | AAGA | | | CGTGAGAAGAAAA | |
| | | | | GAGA | |
| NoName48_m1 | CCCCTTAGGGATAAC | 680 | NoName48_m2 | GTGACTGGAGTTCA | 772 |
| | AGGGTAATCACTGTT | | | GACGTGTGCTCTTC | |
| | GGGGTGACTAACTGT | | | CGATCTGACTAACT | |
| | | | | GTCATGGTTTTCCC | |
| | | | | ACG | |
| NoName48_p1 | CCCCTTAGGGATAAC | 681 | NoName48_p2 | GTGACTGGAGTTCA | 773 |
| | AGGGTAATCTTGCTA | | | GACGTGTGCTCTTC | |
| | ACAGTGGTGAGTTGT | | | CGATCTAACAGTGG | |
| | AATA | | | TGAGTTGTAATACT | |
| | | | | AGCT | |
| NoName49_p1 | CCCCTTAGGGATAAC | 682 | NoName49_p2 | GTGACTGGAGTTCA | 774 |
| | AGGGTAATCAGTTCC | | | GACGTGTGCTCTTC | |
| | TGATCCGGCTCTGGA | | | CGATCTTCCGGCTC | |
| | | | | TGGATTTGTGCACA | |
| | | | | G | |
| NoName50_m1 | CCCCTTAGGGATAAC | 683 | NoName50_m2 | GTGACTGGAGTTCA | 775 |
| | AGGGTAATCCGAGA | | | GACGTGTGCTCTTC | |
| | GGCTCCAGGACCATG | | | CGATCTGCGCTGCA | |
| | ACT | | | CGGCCTCCAC | |
| NoName50_p1 | CCCCTTAGGGATAAC | 684 | NoName50_p2 | GTGACTGGAGTTCA | 776 |
| | AGGGTAATCGGGCTG | | | GACGTGTGCTCTTC | |
| | GCGGGGTGGGAA | | | CGATCTGGGTGGGA | |
| | | | | AGGGAGGGTCAG | |
| NoName51_m1 | CCCCTTAGGGATAAC | 685 | NoName51_m2 | GTGACTGGAGTTCA | 777 |
| | AGGGTAATCGTGCTG | | | GACGTGTGCTCTTC | |
| | GCTGAATTAATAGGA | | | CGATCTAATAGGAG | |
| | GGCA | | | GCACATCTCATCCA | |
| | | | | TTGC | |
| NoName51_p1 | CCCCTTAGGGATAAC | 686 | NoName51_p2 | GTGACTGGAGTTCA | 778 |
| | AGGGTAATCCAAGGT | | | GACGTGTGCTCTTC | |
| | CTTTCAACTTGGGCC | | | CGATCTGAGCACTG | |
| | AGAT | | | CAGGACGTTCAGCA | |
| NoName52_m1 | CCCCTTAGGGATAAC | 687 | NoName52_m2 | GTGACTGGAGTTCA | 779 |
| | AGGGTAATCCCTTGG | | | GACGTGTGCTCTTC | |
| | GTCCTGTCCTGGCA | | | CGATCTTGCTATGA | |
| | | | | GCTGCCCCTGGGT | |
| NoName52_p1 | CCCCTTAGGGATAAC | 688 | NoName52_p2 | GTGACTGGAGTTCA | 780 |
| | AGGGTAATCCGGGGT | | | GACGTGTGCTCTTC | |
| | TCACTGGCCCAGA | | | CGATCTTTCACTGG | |
| | | | | CCCAGAGCTGTGC | |
| NoName6_m1 | CCCCTTAGGGATAAC | 689 | NoName6_2 | GTGACTGGAGTTCA | 781 |
| | AGGGTAATCAAGGG | | | GACGTGTGCTCTTC | |
| | AGCGGGGATTATGGC | | | CGATCTAGGACCAG | |
| | | | | GGTCATGACTAGCT | |
| | | | | AAA | |
| NoName6_p1 | CCCCTTAGGGATAAC | 690 | NoName6_p2 | GTGACTGGAGTTCA | 782 |
| | AGGGTAATCGATCAT | | | GACGTGTGCTCTTC | |
| | GCACCCCGTCCTGAC | | | CGATCTGTCCTGAC | |
| | | | | CCTGACGCTGCAC | |
| NoName7_m1 | CCCCTTAGGGATAAC | 691 | NoName7_m2 | GTGACTGGAGTTCA | 783 |
| | AGGGTAATCCAGACC | | | GACGTGTGCTCTTC | |
| | TGCCGTGGACCTT | | | CGATCTGCCGTGGA | |
| | | | | CCTTGGCTTCC | |
| NoName7_p1 | CCCCTTAGGGATAAC | 692 | NoName7_p2 | GTGACTGGAGTTCA | 784 |
| | AGGGTAATCAGCCGG | | | GACGTGTGCTCTTC | |
| | CGCTAAGAGCAG | | | CGATCTGGCGCTAA | |
| | | | | GAGCAGCTGACC | |
| NoName8_m1 | CCCCTTAGGGATAAC | 693 | NoName8_m2 | GTGACTGGAGTTCA | 785 |
| | AGGGTAATCGCCTGG | | | GACGTGTGCTCTTC | |
| | ATCCCACCCTTGC | | | CGATCTGTGTGGCA | |
| | | | | CAGTGAGGGGTGT | |
| NoName8_p1 | CCCCTTAGGGATAAC | 694 | NoName8_p2 | GTGACTGGAGTTCA | 786 |
| | AGGGTAATCCTGGTC | | | GACGTGTGCTCTTC | |
| | CCGCCGCAGCCT | | | CGATCTCCGCCGCA | |
| | | | | GCCTCGCAGA | |
| NoName9_m1 | CCCCTTAGGGATAAC | 695 | NoName9_m2 | GTGACTGGAGTTCA | 787 |
| | AGGGTAATCGCCCTG | | | GACGTGTGCTCTTC | |
| | GCTATTTGCAAACTG | | | CGATCTATGCTGTC | |
| | CAT | | | CCAGTTCTCTCACC | |
| | | | | ACT | |
| NoName9_p1 | CCCCTTAGGGATAAC | 696 | NoName9_p2 | GTGACTGGAGTTCA | 788 |
| | AGGGTAATCACAGA | | | GACGTGTGCTCTTC | |
| | GATGCAGATAGCCAG | | | CGATCTGGCAGGGA | |
| | GTTAGA | | | TAGGTGAGCTTCAA | |
| | | | | A | |
| PD1_m1 | CCCCTTAGGGATAAC | 697 | PD1_m2 | GTGACTGGAGTTCA | 789 |
| | AGGGTAATCGGGTGG | | | GACGTGTGCTCTTC | |
| | AAGGTCCCTCCAG | | | CGATCTCCCTGGCT | |
| | | | | CTGGGACACCT | |
| PD1_p1 | CCCCTTAGGGATAAC | 698 | PD1_p2 | GTGACTGGAGTTCA | 790 |
| | AGGGTAATCAGTGGA | | | GACGTGTGCTCTTC | |
| | GAAGGCGGCACTC | | | CGATCTACTCTGGT | |
| | | | | GGGGCTGCTCCA | |

TABLE_7
Sequences of anchored primers for TRAC

| First PCR | | SEQ | Second PCR | | SEQ |
|---|---|---|---|---|---|
| primer | | ID | primer | | ID |
| name | Sequence | NO: | name | Sequence | NO: |
| NoName1_m1 | CCCCTTAGGGATAAC | 791 | NoName1_m2 | GTGACTGGAGTTCA | 876 |
| | AGGGTAATCAAGTAG | | | GACGTGTGCTCTTC | |
| | GGCTCAGGGTCGAA | | | CGATCTGGCTCAGG | |
| | GG | | | GTCGAAGGCTCACT | |
| NoName1_p1 | CCCCTTAGGGATAAC | 792 | NoName1_p2 | GTGACTGGAGTTCA | 877 |
| | AGGGTAATCGCAATG | | | GACGTGTGCTCTTC | |
| | GCCGCTGGGAAAAA | | | CGATCTTCAAACCA | |
| | T | | | TCGGGGGAAAAAT | |
| | | | | GACAA | |
| NoName10_m1 | CCCCTTAGGGATAAC | 793 | NoName10_m2 | GTGACTGGAGTTCA | 878 |
| | AGGGTAATCCTATCA | | | GACGTGTGCTCTTC | |
| | TTGTAGATGGGGCCG | | | CGATCTGTAGATGG | |
| | GAAA | | | GGCCGGAAAGTAG | |
| | | | | AAAAG | |
| NoName10_p1 | CCCCTTAGGGATAAC | 794 | NoName10_p2 | GTGACTGGAGTTCA | 879 |
| | AGGGTAATCGCCACT | | | GACGTGTGCTCTTC | |
| | GCCACTGTAGCCT | | | CGATCTCCCAGCTC | |
| | | | | CAAGTCCATCTGG | |
| NoName12_m1 | CCCCTTAGGGATAAC | 795 | NoName12_m2 | GTGACTGGAGTTCA | 880 |
| | AGGGTAATCCAACTC | | | GACGTGTGCTCTTC | |
| | CAGGGCTCAAGCAA | | | CGATCTGCTACCAA | |
| | TCG | | | GCCCCACCCT | |
| NoName12_p1 | CCCCTTAGGGATAAC | 796 | NoName12_p2 | GTGACTGGAGTTCA | 881 |
| | AGGGTAATCGCAGAC | | | GACGTGTGCTCTTC | |
| | ATTTGACCACCCTAT | | | CGATCTCACCCTATA | |
| | ACCC | | | CCCACCATACTCAC | |
| | | | | GTT | |
| NoName13_m1 | CCCCTTAGGGATAAC | 797 | NoName13_m2 | GTGACTGGAGTTCA | 882 |
| | AGGGTAATCGCAGTA | | | GACGTGTGCTCTTC | |
| | GGGAAGGGGCAACT | | | CGATCTAGGGAAGG | |
| | | | | GGCAACTTTTCAAA | |
| | | | | ATCT | |
| NoName13_p1 | CCCCTTAGGGATAAC | 798 | NoName13_p2 | GTGACTGGAGTTCA | 883 |
| | AGGGTAATCGTCTTT | | | GACGTGTGCTCTTC | |
| | CTCTGGCACCAAGCT | | | CGATCTGCACCAAG | |
| | TTTG | | | CTTTTGTGATGCTC | |
| | | | | CAAC | |
| NoName14_m1 | CCCCTTAGGGATAAC | 799 | NoName14_m2 | GTGACTGGAGTTCA | 884 |
| | AGGGTAATCTGGCAC | | | GACGTGTGCTCTTC | |
| | CTGCAGGAAACGGT | | | CGATCTCACCTGCA | |
| | | | | GGAAACGGTTGCGT | |
| | | | | TC | |
| NoName14_p1 | CCCCTTAGGGATAAC | 800 | NoName14_p2 | GTGACTGGAGTTCA | 885 |
| | AGGGTAATCCTGGGC | | | GACGTGTGCTCTTC | |
| | CACCTGGTGTCG | | | CGATCTGCTGGGCC | |
| | | | | GCCTGATCTACC | |
| NoName15_m1 | CCCCTTAGGGATAAC | 801 | NoName15_m2 | GTGACTGGAGTTCA | 886 |
| | AGGGTAATCCCTTGG | | | GACGTGTGCTCTTC | |
| | GCCAGTCACTGCA | | | CGATCTGCCAGTCA | |
| | | | | CTGCAGCTCTCT | |
| NoName15_p1 | CCCCTTAGGGATAAC | 802 | NoName15_p2 | GTGACTGGAGTTCA | 887 |
| | AGGGTAATCTGACCA | | | GACGTGTGCTCTTC | |
| | CATGTCCACCGTTCA | | | CGATCTACATGTCC | |
| | G | | | ACCGTTCAGACACA | |
| | | | | GC | |
| NoName16_m1 | CCCCTTAGGGATAAC | 803 | NoName16_m2 | GTGACTGGAGTTCA | 888 |
| | AGGGTAATCAGCTTG | | | GACGTGTGCTCTTC | |
| | GGAGGCTGGTACTAC | | | CGATCTGGGAGGCT | |
| | TG | | | GGTACTACTGGGCA | |
| | | | | TC | |
| NoName16_p1 | CCCCTTAGGGATAAC | 804 | NoName16_p2 | GTGACTGGAGTTCA | 889 |
| | AGGGTAATCCCCAGA | | | GACGTGTGCTCTTC | |
| | CACTGCTTCCCTGGT | | | CGATCTAGACACTG | |
| | A | | | CTTCCCTGGTAATG | |
| | | | | GAC | |
| NoName17_p1 | CCCCTTAGGGATAAC | 805 | NoName17_p2 | GTGACTGGAGTTCA | 890 |
| | AGGGTAATCTTCCTC | | | GACGTGTGCTCTTC | |
| | CTGCCAGGGTGCA | | | CGATCTCCTGCCAG | |
| | | | | GGTGCAAGAACT | |
| NoName18_m1 | CCCCTTAGGGATAAC | 806 | NoName18_m2 | GTGACTGGAGTTCA | 891 |
| | AGGGTAATCTGTACC | | | GACGTGTGCTCTTC | |
| | ATGAATGTTGTGGCG | | | CGATCTCCATGAAT | |
| | CAT | | | GTTGTGGCGCATTT | |
| | | | | TCAT | |
| NoName18_p1 | CCCCTTAGGGATAAC | 807 | NoName18_p2 | GTGACTGGAGTTCA | 892 |
| | AGGGTAATCAGTCTG | | | GACGTGTGCTCTTC | |
| | GGTCAAGTGCTGTG | | | CGATCTTGCTGTGG | |
| | G | | | GCTCCTTTGCTT | |
| NoName19_p1 | CCCCTTAGGGATAAC | 808 | NoName19_p2 | GTGACTGGAGTTCA | 893 |
| | AGGGTAATCGGAAC | | | GACGTGTGCTCTTC | |
| | AAAGGACCTACATGT | | | CGATCTCAAAGGAC | |
| | GGCT | | | CTACATGTGGCTCC | |
| | | | | AATT | |
| NoName2_m1 | CCCCTTAGGGATAAC | 809 | NoName2_m2 | GTGACTGGAGTTCA | 894 |
| | AGGGTAATCACATAA | | | GACGTGTGCTCTTC | |
| | GCGAAGGATCAGGA | | | CGATCTGCGAAGGA | |
| | GAGT | | | TCAGGAGAGTACTA | |
| | | | | TTAG | |
| NoName20_m1 | CCCCTTAGGGATAAC | 810 | NoName20_m2 | GTGACTGGAGTTCA | 895 |
| | AGGGTAATCTCTAGA | | | GACGTGTGCTCTTC | |
| | GAACATCCGGCAATG | | | CGATCTAGGGGTGG | |
| | CC | | | GAGAGTGCTACT | |
| NoName21_m1 | CCCCTTAGGGATAAC | 811 | NoName21_m2 | GTGACTGGAGTTCA | 896 |
| | AGGGTAATCCCTTAG | | | GACGTGTGCTCTTC | |
| | GCCAAACATCCTTGA | | | CGATCTTGTATGTTG | |
| | CCATA | | | GTTATGCGGGAAGA | |
| | | | | GAC | |
| NoName21_p1 | CCCCTTAGGGATAAC | 812 | NoName21_p2 | GTGACTGGAGTTCA | 897 |
| | AGGGTAATCTCCCCA | | | GACGTGTGCTCTTC | |
| | AAGTCTAAGGAGGC | | | CGATCTTGGATTTC | |
| | TAAGA | | | CAAAGAGAAGCCC | |
| | | | | TAGTC | |
| NoName22_p1 | CCCCTTAGGGATAAC | 813 | NoName22_p2 | GTGACTGGAGTTCA | 898 |
| | AGGGTAATCCCTGAA | | | GACGTGTGCTCTTC | |
| | AAACGGATGAGACT | | | CGATCTACGGATGA | |
| | TCAG | | | GACTTCAGTGAGTA | |
| | | | | C | |
| NoName23_p1 | CCCCTTAGGGATAAC | 814 | NoName23_p2 | GTGACTGGAGTTCA | 899 |
| | AGGGTAATCATTGTG | | | GACGTGTGCTCTTC | |
| | CTTCAGATCCCGTGA | | | CGATCTTGCTTCAG | |
| | CAT | | | ATCCCGTGACATCA | |
| | | | | GTGT | |
| NoName24_m1 | CCCCTTAGGGATAAC | 815 | NoName24_m2 | GTGACTGGAGTTCA | 900 |
| | AGGGTAATCGTGGGG | | | GACGTGTGCTCTTC | |
| | ACTTGCTGCTGGT | | | CGATCTAGTGGGGA | |
| | | | | CTTGCTGCTGGTAT | |
| | | | | CTAC | |
| NoName24_p1 | CCCCTTAGGGATAAC | 816 | NoName24_p2 | GTGACTGGAGTTCA | 901 |
| | AGGGTAATCAGCTCT | | | GACGTGTGCTCTTC | |
| | GCTACATTCAGGTAA | | | CGATCTGCTACATT | |
| | CAT | | | CAGGTAACATGTTT | |
| | | | | CTGC | |
| NoName25_m1 | CCCCTTAGGGATAAC | 817 | NoName25_m2 | GTGACTGGAGTTCA | 902 |
| | AGGGTAATCTCCCTC | | | GACGTGTGCTCTTC | |
| | TTTAGCATCGCCAAA | | | CGATCTGCCAAATC | |
| | TCC | | | CTCCCAGGTGCA | |
| NoName25_p1 | CCCCTTAGGGATAAC | 818 | NoName25_p2 | GTGACTGGAGTTCA | 903 |
| | AGGGTAATCTTGGTG | | | GACGTGTGCTCTTC | |
| | GCCACAACTTAGGTG | | | CGATCTGGCCACAA | |
| | AGA | | | CTTAGGTGAGAGTG | |
| | | | | ACGA | |
| NoName26_m1 | CCCCTTAGGGATAAC | 819 | NoName26_m2 | GTGACTGGAGTTCA | 904 |
| | AGGGTAATCCCCAGG | | | GACGTGTGCTCTTC | |
| | TGTTGCTCATCAGTT | | | CGATCTCCTCTGAA | |
| | CCTCT | | | CTAAGTGGGAGTTT | |
| | | | | GGC | |
| NoName26_p1 | CCCCTTAGGGATAAC | 820 | NoName26_p2 | GTGACTGGAGTTCA | 905 |
| | AGGGTAATCATCACT | | | GACGTGTGCTCTTC | |
| | TTCTCAAGGGACATG | | | CGATCTTGCCATTTC | |
| | CCAT | | | TCTAATCAAGGGGT | |
| | | | | GTG | |
| NoName27_m1 | CCCCTTAGGGATAAC | 821 | NoName27_m2 | GTGACTGGAGTTCA | 906 |
| | AGGGTAATCGTCTCA | | | GACGTGTGCTCTTC | |
| | CAACTCCCAGTCTTG | | | CGATCTTCCCAGTC | |
| | CTTTA | | | TTGCTTTATACTGTG | |
| | | | | CCT | |
| NoName27_p1 | CCCCTTAGGGATAAC | 822 | NoName27_p2 | GTGACTGGAGTTCA | 907 |
| | AGGGTAATCAACTGG | | | GACGTGTGCTCTTC | |
| | GCTCGTTGGTTACCC | | | CGATCTCTGGGCTC | |
| | T | | | GTTGGTTACCCTATT | |
| | | | | CCT | |
| NoName28_m1 | CCCCTTAGGGATAAC | 823 | NoName28_m2 | GTGACTGGAGTTCA | 908 |
| | AGGGTAATCTTTGGT | | | GACGTGTGCTCTTC | |
| | TTGGTTGCTTTGCAG | | | CGATCTGAGGAGCT | |
| | ACTAC | | | ACCAGGGCCCTA | |
| NoName3_m1 | CCCCTTAGGGATAAC | 824 | NoName3_m2 | GTGACTGGAGTTCA | 909 |
| | AGGGTAATCCTTTTC | | | GACGTGTGCTCTTC | |
| | TGCTGTCACCCTCAA | | | CGATCTACCTCATC | |
| | GGAT | | | ATTTCTCAGGCGAA | |
| | | | | AGG | |
| NoName3_p1 | CCCCTTAGGGATAAC | 825 | NoName3_p2 | GTGACTGGAGTTCA | 910 |
| | AGGGTAATCGAGTGA | | | GACGTGTGCTCTTC | |
| | ATGCATGATTGTGTG | | | CGATCTGCATGATT | |
| | ACCGA | | | GTGTGACCGAATGC | |
| | | | | CTCA | |
| NoName30_m1 | CCCCTTAGGGATAAC | 826 | NoName30_m2 | GTGACTGGAGTTCA | 911 |
| | AGGGTAATCTGCTAG | | | GACGTGTGCTCTTC | |
| | TGTCGAGGTTTGCA | | | CGATCTGGTTTGCA | |
| | | | | CCATAGAAAGCTGA | |
| | | | | G | |
| NoName30_p1 | CCCCTTAGGGATAAC | 827 | NoName30_p2 | GTGACTGGAGTTCA | 912 |
| | AGGGTAATCGTGGAG | | | GACGTGTGCTCTTC | |
| | AAAGTGCTAAACAA | | | CGATCTGGTAAACC | |
| | GAAAA | | | AGAACTATCTTTCT | |
| | | | | CTCC | |
| NoName31_m1 | CCCCTTAGGGATAAC | 828 | NoName31_m2 | GTGACTGGAGTTCA | 913 |
| | AGGGTAATCCTCCAG | | | GACGTGTGCTCTTC | |
| | AGTCTATGCTCAACT | | | CGATCTGAACTTGA | |
| | GAA | | | AATGCTTACAGCCA | |
| | | | | GAAT | |
| NoName32_m1 | CCCCTTAGGGATAAC | 829 | NoName32_m2 | GTGACTGGAGTTCA | 914 |
| | AGGGTAATCATGGCC | | | GACGTGTGCTCTTC | |
| | ATAAGTTGAAATTTG | | | CGATCTCCATAAGT | |
| | CGT | | | TGAAATTTGCGTTT | |
| | | | | CGGT | |
| NoName33_m1 | CCCCTTAGGGATAAC | 830 | NoName33_m2 | GTGACTGGAGTTCA | 915 |
| | AGGGTAATCGGGACC | | | GACGTGTGCTCTTC | |
| | TCAGGTGCTGCTT | | | CGATCTCCTCAGGT | |
| | | | | GCTGCTTCCTCAA | |
| NoName33_p1 | CCCCTTAGGGATAAC | 831 | NoName33_p2 | GTGACTGGAGTTCA | 916 |
| | AGGGTAATCTGATTC | | | GACGTGTGCTCTTC | |
| | AATCTTACATGCGAC | | | CGATCTCATGCGAC | |
| | AGCCT | | | AGCCTGATCCGTTT | |
| | | | | CT | |
| NoName34_m1 | CCCCTTAGGGATAAC | 832 | NoName34_m2 | GTGACTGGAGTTCA | 917 |
| | AGGGTAATCAGAGA | | | GACGTGTGCTCTTC | |
| | AGCCTGTCAGGACC | | | CGATCTGCCTGTCA | |
| | AT | | | GGACCATACAAATC | |
| | | | | TTAC | |
| NoName34_p1 | CCCCTTAGGGATAAC | 833 | NoName34_p2 | GTGACTGGAGTTCA | 918 |
| | AGGGTAATCTCACCG | | | GACGTGTGCTCTTC | |
| | TCTACTTCTCTTGTGT | | | CGATCTTTCTCTTGT | |
| | G | | | GTGATCCAGAGTTG | |
| | | | | ACA | |
| NoName35_m1 | CCCCTTAGGGATAAC | 834 | NoName35_m2 | GTGACTGGAGTTCA | 919 |
| | AGGGTAATCCCACAT | | | GACGTGTGCTCTTC | |
| | GCAAATGAACGACA | | | CGATCTAACGACAC | |
| | CTGAC | | | TGACAGAAAACAC | |
| | | | | TCACG | |
| NoName36_m1 | CCCCTTAGGGATAAC | 835 | NoName36_m2 | GTGACTGGAGTTCA | 920 |
| | AGGGTAATCGCAGCA | | | GACGTGTGCTCTTC | |
| | ATTTGGTCCCCCATG | | | CGATCTAGCAATTT | |
| | G | | | GGTCCCCCATGGAG | |
| | | | | AGAC | |
| NoName36_p1 | CCCCTTAGGGATAAC | 836 | NoName36_p2 | GTGACTGGAGTTCA | 921 |
| | AGGGTAATCTCAGAC | | | GACGTGTGCTCTTC | |
| | CGTGACTCAGTATGT | | | CGATCTAAAACTTG | |
| | TG | | | ACTGTTCATTGGGT | |
| | | | | TCAA | |
| NoName37_m1 | CCCCTTAGGGATAAC | 837 | NoName37_m2 | GTGACTGGAGTTCA | 922 |
| | AGGGTAATCAGGCCC | | | GACGTGTGCTCTTC | |
| | CTGTCTCTACCATCC | | | CGATCTCCCCTGTC | |
| | | | | TCTACCATCCTAGA | |
| | | | | CACC | |
| NoName37_p1 | CCCCTTAGGGATAAC | 838 | NoName37_p2 | GTGACTGGAGTTCA | 923 |
| | AGGGTAATCGTGGAG | | | GACGTGTGCTCTTC | |
| | AAGGCAGCCTCCCA | | | CGATCTAGAAGGCA | |
| | A | | | GCCTCCCAAAGCAC | |
| | | | | T | |
| NoName38_m1 | CCCCTTAGGGATAAC | 839 | NoName38_m2 | GTGACTGGAGTTCA | 924 |
| | AGGGTAATCTGCCTG | | | GACGTGTGCTCTTC | |
| | GAGTGGTGTCTGGT | | | CGATCTGCCTGGAG | |
| | | | | TGGTGTCTGGTACA | |
| | | | | ATGA | |
| NoName38_p1 | CCCCTTAGGGATAAC | 840 | NoName38_p2 | GTGACTGGAGTTCA | 925 |
| | AGGGTAATCACAGAC | | | GACGTGTGCTCTTC | |
| | CTCAGAGCCCAGTCC | | | CGATCTGTCCCTGG | |
| | | | | CCTTAAAGAAATGA | |
| | | | | CAGA | |
| NoName39_m1 | CCCCTTAGGGATAAC | 841 | NoName39_m2 | GTGACTGGAGTTCA | 926 |
| | AGGGTAATCGCACAC | | | GACGTGTGCTCTTC | |
| | AGCCAACAAGATGA | | | CGATCTAGCCTTGA | |
| | CTCA | | | TTACTGTTCCCACT | |
| | | | | AGC | |
| NoName39_p1 | CCCCTTAGGGATAAC | 842 | NoName39_p2 | GTGACTGGAGTTCA | 927 |
| | AGGGTAATCCCCCTG | | | GACGTGTGCTCTTC | |
| | TTTTTACCTCAACCT | | | CGATCTGGGCTTCC | |
| | TAGGG | | | TTGCTTTGGTTACT | |
| | | | | GT | |
| NoName4_m1 | CCCCTTAGGGATAAC | 843 | NoName4_m2 | GTGACTGGAGTTCA | 928 |
| | AGGGTAATCTCACTG | | | GACGTGTGCTCTTC | |
| | CTGCCCCCACAAG | | | CGATCTCACTGCTG | |
| | | | | CCCCCACAAGCTTA | |
| | | | | AC | |
| NoName4_p1 | CCCCTTAGGGATAAC | 844 | NoName4_p2 | GTGACTGGAGTTCA | 929 |
| | AGGGTAATCGGCCAG | | | GACGTGTGCTCTTC | |
| | GCCGGAGTCAGG | | | CGATCTGCCGGAGT | |
| | | | | CAGGGGCATC | |
| NoName40_m1 | CCCCTTAGGGATAAC | 845 | NoName40_m2 | GTGACTGGAGTTCA | 930 |
| | AGGGTAATCTTGGAA | | | GACGTGTGCTCTTC | |
| | TGGCAATCCGTTGGA | | | CGATCTATGGCAAT | |
| | AATG | | | CCGTTGGAAATGTC | |
| | | | | TTCT | |
| NoName40_p1 | CCCCTTAGGGATAAC | 846 | NoName40_p2 | GTGACTGGAGTTCA | 931 |
| | AGGGTAATCTGGAAC | | | GACGTGTGCTCTTC | |
| | TGTGGGCATAAGCAT | | | CGATCTCCCATACC | |
| | ATGTC | | | CCACTCCCACTACT | |
| NoName41_m1 | CCCCTTAGGGATAAC | 847 | NoName41_m2 | GTGACTGGAGTTCA | 932 |
| | AGGGTAATCACAGGT | | | GACGTGTGCTCTTC | |
| | TTCAGGCGGAGTGG | | | CGATCTCAGGTTTC | |
| | A | | | AGGCGGAGTGGAA | |
| | | | | GAAGT | |
| NoName41_p1 | CCCCTTAGGGATAAC | 848 | NoName41_p2 | GTGACTGGAGTTCA | 933 |
| | AGGGTAATCAGGAG | | | GACGTGTGCTCTTC | |
| | GAATTAACCCTGTGA | | | CGATCTAACCCTGT | |
| | ACATCG | | | GAACATCGTGATTC | |
| | | | | CAG | |
| NoName42_p1 | CCCCTTAGGGATAAC | 849 | NoName42_p2 | GTGACTGGAGTTCA | 934 |
| | AGGGTAATCTTTCAC | | | GACGTGTGCTCTTC | |
| | AAGAACGGTACTGG | | | CGATCTCGGTACTG | |
| | CCAAT | | | GCCAATGAAATTTT | |
| | | | | CCCA | |
| NoName43_m1 | CCCCTTAGGGATAAC | 850 | NoName43_m2 | GTGACTGGAGTTCA | 935 |
| | AGGGTAATCATAAGA | | | GACGTGTGCTCTTC | |
| | GGTGAACTAGCAAG | | | CGATCTTTGGCTCT | |
| | CAGAGC | | | CTGGATTGTTCCTC | |
| | | | | TAAA | |
| NoName43_p1 | CCCCTTAGGGATAAC | 851 | NoName43_p2 | GTGACTGGAGTTCA | 936 |
| | AGGGTAATCAGAGTG | | | GACGTGTGCTCTTC | |
| | TAAGCTCACCCTACA | | | CGATCTCACCCTAC | |
| | GTCT | | | AGTCTATGTTCCAG | |
| | | | | GTCA | |
| NoName44_m1 | CCCCTTAGGGATAAC | 852 | NoName44_m2 | GTGACTGGAGTTCA | 937 |
| | AGGGTAATCGACAGC | | | GACGTGTGCTCTTC | |
| | AAGTCCAGACTAAG | | | CGATCTCCAGACTA | |
| | GCA | | | AGGCAAGCAACTG | |
| | | | | TAACA | |
| NoName44_p1 | CCCCTTAGGGATAAC | 853 | NoName44_p2 | GTGACTGGAGTTCA | 938 |
| | AGGGTAATCGAGGTA | | | GACGTGTGCTCTTC | |
| | GGGTTCTTCGTGTTG | | | CGATCTCTTCGTGT | |
| | GC | | | TGGCCAGGTGGGT | |
| NoName45_m1 | CCCCTTAGGGATAAC | 854 | NoName45_m2 | GTGACTGGAGTTCA | 939 |
| | AGGGTAATCCCTAAG | | | GACGTGTGCTCTTC | |
| | TGGAGTTGACCTGTA | | | CGATCTTGAAGCTG | |
| | CAAGG | | | AGTTACCTGGGAGC | |
| | | | | TC | |
| NoName45_p1 | CCCCTTAGGGATAAC | 855 | NoName45_p2 | GTGACTGGAGTTCA | 940 |
| | AGGGTAATCCTTCAG | | | GACGTGTGCTCTTC | |
| | CCACTCCCTTATGAG | | | CGATCTCTTACGGG | |
| | GTAG | | | AAAGCAAGTTGACT | |
| | | | | TTGC | |
| NoName46_m1 | CCCCTTAGGGATAAC | 856 | NoName46_m2 | GTGACTGGAGTTCA | 941 |
| | AGGGTAATCACACCA | | | GACGTGTGCTCTTC | |
| | GGCTACAAGTCTCCT | | | CGATCTACAAAACA | |
| | GA | | | AAACCCTCCGGATG | |
| | | | | GTCT | |
| NoName46_p1 | CCCCTTAGGGATAAC | 857 | NoName46_p2 | GTGACTGGAGTTCA | 942 |
| | AGGGTAATCCCCTGC | | | GACGTGTGCTCTTC | |
| | TCCTGTCTGCCTGAT | | | CGATCTTGCTCCTG | |
| | TA | | | TCTGCCTGATTACTT | |
| | | | | ACT | |
| NoName47_m1 | CCCCTTAGGGATAAC | 858 | NoName47_m2 | GTGACTGGAGTTCA | 943 |
| | AGGGTAATCAAGGCT | | | GACGTGTGCTCTTC | |
| | TGTTCACCCTGAGGA | | | CGATCTGGTCATGC | |
| | G | | | CTCCAACCTGCA | |
| NoName47_p1 | CCCCTTAGGGATAAC | 859 | NoName47_p2 | GTGACTGGAGTTCA | 944 |
| | AGGGTAATCGGAAA | | | GACGTGTGCTCTTC | |
| | GCTAAAAGATTTGCG | | | CGATCTTTGCGTTG | |
| | TTGACT | | | ACTTAAATGAAAGT | |
| | | | | GTCC | |
| NoName48_p1 | CCCCTTAGGGATAAC | 860 | NoName48_p2 | GTGACTGGAGTTCA | 945 |
| | AGGGTAATCTCCTTC | | | GACGTGTGCTCTTC | |
| | CACGGAGTTCACTG | | | CGATCTACTGTCGG | |
| | AGT | | | GAGAAGGCGTCT | |
| NoName49_m1 | CCCCTTAGGGATAAC | 861 | NoName49_m2 | GTGACTGGAGTTCA | 946 |
| | AGGGTAATCAGCTTT | | | GACGTGTGCTCTTC | |
| | GGCCCCTAGGATTCT | | | CGATCTTGATCTGTT | |
| | G | | | TGTGAATGGCTCAG | |
| | | | | ACA | |
| NoName49_p1 | CCCCTTAGGGATAAC | 862 | NoName49_p2 | GTGACTGGAGTTCA | 947 |
| | AGGGTAATCCTCTGG | | | GACGTGTGCTCTTC | |
| | GTGCGGGGGAACT | | | CGATCTACTCTGGG | |
| | | | | TGCGGGGGAACTTA | |
| | | | | TTTG | |
| NoName5_m1 | CCCCTTAGGGATAAC | 863 | NoName5_m2 | GTGACTGGAGTTCA | 948 |
| | AGGGTAATCTCCAGT | | | GACGTGTGCTCTTC | |
| | GATCTAGTAACTCCG | | | CGATCTCCGTGGTG | |
| | TGGT | | | GATTTAACTCCCCT | |
| | | | | ATTG | |
| NoName5_p1 | CCCCTTAGGGATAAC | 864 | NoName5_p2 | GTGACTGGAGTTCA | 949 |
| | AGGGTAATCCCTTCA | | | GACGTGTGCTCTTC | |
| | GAAACTAGTTAGCCC | | | CGATCTAGCATTCT | |
| | TGT | | | GCCTCTGACAGG | |
| NoName50_m1 | CCCCTTAGGGATAAC | 865 | NoName50_m2 | GTGACTGGAGTTCA | 950 |
| | AGGGTAATCATGGTC | | | GACGTGTGCTCTTC | |
| | CAAGGTCAGCTGGC | | | CGATCTTCCAAGGT | |
| | GGACA | | | CAGCTGGCGGACA | |
| NoName50_p1 | CCCCTTAGGGATAAC | 866 | NoName50_p2 | GTGACTGGAGTTCA | 951 |
| | AGGGTAATCAGGACC | | | GACGTGTGCTCTTC | |
| | CACCACGGATTCCT | | | CGATCTACGGATTC | |
| | | | | CTGCTGTACTGGCT | |
| | | | | AAAG | |
| NoName6_m1 | CCCCTTAGGGATAAC | 867 | NoName6_m2 | GTGACTGGAGTTCA | 952 |
| | AGGGTAATCACTGCC | | | GACGTGTGCTCTTC | |
| | TCCTCCTTAGTCGAT | | | CGATCTTGCCTCCT | |
| | | | | CCTTAGTCGATTCTT | |
| | | | | ACC | |
| NoName6_p1 | CCCCTTAGGGATAAC | 868 | NoName6_p2 | GTGACTGGAGTTCA | 953 |
| | AGGGTAATCGCTGTA | | | GACGTGTGCTCTTC | |
| | GACAGATTGGCCTCA | | | CGATCTAACAAGTG | |
| | GTT | | | TCCCTGGCAAATGT | |
| | | | | GA | |
| NoName7_m1 | CCCCTTAGGGATAAC | 869 | NoName7_m2 | GTGACTGGAGTTCA | 954 |
| | AGGGTAATCCCAAGG | | | GACGTGTGCTCTTC | |
| | TATGGGGGCTAACCA | | | CGATCTGGGGGCTA | |
| | TT | | | ACCATTGGCAATTG | |
| | | | | AA | |
| NoName7_p1 | CCCCTTAGGGATAAC | 870 | NoName7_p2 | GTGACTGGAGTTCA | 955 |
| | AGGGTAATCTTCTGG | | | GACGTGTGCTCTTC | |
| | AAATTCGTCGAAGG | | | CGATCTTTCGTCGA | |
| | ATGGTC | | | AGGATGGTCTCTCT | |
| | | | | GTTG | |
| NoName8_m1 | CCCCTTAGGGATAAC | 871 | NoName8_m2 | GTGACTGGAGTTCA | 956 |
| | AGGGTAATCAGCTGT | | | GACGTGTGCTCTTC | |
| | GCTCTTCCGTTTCAG | | | CGATCTTGTGCTCT | |
| | TG | | | TCCGTTTCAGTGTG | |
| | | | | AAAA | |
| NoName8_p1 | CCCCTTAGGGATAAC | 872 | NoName8_p2 | GTGACTGGAGTTCA | 957 |
| | AGGGTAATCCCACGA | | | GACGTGTGCTCTTC | |
| | GGCGTATTCATCTGC | | | CGATCTATCTGCATG | |
| | AT | | | CATGAGTCCTGACT | |
| | | | | TC | |
| NoName9_m1 | CCCCTTAGGGATAAC | 873 | NoName9_m2 | GTGACTGGAGTTCA | 958 |
| | AGGGTAATCAATGGA | | | GACGTGTGCTCTTC | |
| | ACCACACTACATCAA | | | CGATCTATCAAGTT | |
| | GTTA | | | ACATAGAAATGGGG | |
| | | | | AGGT | |
| TRAC_m1 | CCCCTTAGGGATAAC | 874 | TRAC_m2 | GTGACTGGAGTTCA | 959 |
| | AGGGTAATCCCTGAC | | | GACGTGTGCTCTTC | |
| | CCTGCCGTGTACCAG | | | CGATCTCCTGCCGT | |
| | | | | GTACCAGCTGAGAG | |
| | | | | AC | |
| TRAC_p1 | CCCCTTAGGGATAAC | 875 | TRAC_p2 | GTGACTGGAGTTCA | 960 |
| | AGGGTAATCCCTGCG | | | GACGTGTGCTCTTC | |
| | AAGGCACCAAAGC | | | CGATCTGCTGTTGT | |
| | | | | TGAAGGCGTTTGCA | |

Referring now to FIG. 4C and FIG. 4D. Chart 411 and chart 412 in FIG. 4C shows off-targets in the iPSC in Example 6 at GAPDH and HBB sites, respectively. Chart 421 and chart 422 in FIG. 4D show off-targets in the T-cell in example 6 at TRAC and PD-1 sites, respectively. As shown in charts 411, 412, 421 and 422, there were 10-26 sites identified as off-targets through fusion detection, while 10%-40% of which were also confirmed by Indel detection. In addition, several sites were validated with Indel frequencies below 0.1%, while translocation could still be detected. Generally, the on-target sites accounted for 7%-20% gene fusions, except HBB locus fetching no fusion partner, as shown in chart 412 (FIG. 4C). It indicated that the sequence contexts flanking DSB end might impact translocation frequency.

### Example 13. Off-Target Profiling and Translocation Dynamics In Vivo

EDITED-Seq was further used to scan off-targets in CRISPR-edited mouse which was edited according to Example 7. Referring to FIGS. 5B and 5C, charts 520 and 530 show off-targets in a mouse at ALB site after 15 or 60 days, respectively.

### Example 14. Summary of Results

In summary, the above results showed that EDITED-Seq can capture all types of off-target events by using an anchored multiplex enrichment of several in-silico predicted genomic loci. Using human tumor-, immune-, and induced pluripotent stem cells and mouse in vivo experiments, the present disclosure showed that EDITED-Seq can identify novel (translocations) off-target sites and quantify editing efficiencies of known off-target sites (InDels), and is compatible with therapeutics pipelines without the need for extra cell manipulations. Most off-target sites (about 90%) that were confirmed by InDels also presented in the form of translocations by EDITED-Seq, albeit translocation frequencies varied in different cell types and genomic contexts. In addition, there were 30%-60% of novel off-target sites that never been detected previously by other existing methods such as DISCOVER-Seq or GUIDE-Seq. The present disclosure demonstrates that EDITED-Seq is sensitive and versatile methods for the detection and evaluation of CRISPR editing efficiency and off-target events and would be compatible with future CRISPR based gene therapy of various genetic diseases.

### Example 15. Discussion

DSBs within genome that created by Cas9 can activate DNA repair pathways, thus resulting in three major kinds of sealed DNA strand formed between different types of double strand breaks (DSBs), including on-target sites, off-target sites, and background: unchanged, mutation (insertion/deletion (Indels) and base mutation), and translocation. Directed by single protospacer RNA, in principle, Cas9 can just make two DSBs at the on-target locus in a diploid human cell. If there is no other unwanted cut, it is unlikely to detect gene fusion. From this view, gene fusion or chromosome arrangement could be observed at undesired cutting site (i.e., off-target). In the example embodiments as described above, the performance of EDITED-Seq, DISCOVER-Seq and GUIDE-Seq in detection of off-targets were compared.

GUIDE-Seq requires an extra double-strand oligonucleotide (dsODN) during wet lab process to generate dsODN insertions at CRISPR editing sites in the genome, which is incompatible with in vivo editing scenarios, and is an undesired extra step for ex vivo editing scenarios. ODN-inserted genome is actually artifact genome derivation, not the nature status of edited one created by nuclease.

DISCOVER-Seq snapshots the intermediate status of MER11, one of key components of the onset double-stranded break (DSB) repair, bound to DSB end to capture genome-wide cutting lesions created by Cas9. Therefore, the sensitivity and specificity of DISCOVER-Seq highly depends on the quality of MER11 antibody, implying uncontrollable fluctuations in outcome as well as a time-consuming procedure if a validation should be conducted via amplicon Next Generation Sequencing (NGS).

In contrast with the two methods above, EDITED-Seq is a versatile approach to detect genome-wide in situ edited off-targets without any artificial perturbation during the mutagenesis (e.g., mutation and translocation) progression induced by genome-editing nucleases. There might be a concern that gene translocation/arrangement just accounts for a small proportion of nuclease-induced mutagenesis, thus potentially limiting the sensitivity of EDITED-Seq. The two steps can significantly improve such potential limitation. Most off-target sites (about 90%) that were confirmed by InDels also presented in the form of translocations by EDITED-Seq, albeit translocation frequencies varied in different cell types and genomic contexts.

There are considerable differences in outcome off-target between repairing DSB and post-repair. Some sites identified by DISCOVER-Seq actually showed few final mutagenesis edit (FIG. 2A and FIG. 2B), indicating biased DSB repair levels at distinguished off-target sites. EDITED-Seq can directly readout the sequence-altered off-targets post DSB repair, representing a clinically useful approach as the most critical concern during gene editing is how many genomic loci as well as genomes are altered in a biopsy pool rather than which locus is cleaved or bound by Cas-nuclease. In this view, EDITED-Seq provides the genome-wide bona fide information of in situ sequence alternation induced by CRISPR, with an economical and straightforward fashion unlike whole genome sequencing. The performance of EDITED-Seq in iPSC and in vivo further extend its application as a parallel quality control step for clinical gene therapy bioproduct.

The exemplary embodiments of the present disclosure are thus fully described. Although the description referred to particular embodiments, it will be clear to one skilled in the art that the present disclosure may be practiced with variation of these specific details. The methods/steps discussed in one figure can be added to or exchanged with methods/steps in other figures. Hence this disclosure should not be construed as limited to the embodiments set forth herein.

What is claimed is:

1. A method of enriching at least one target nucleic acid from a sample comprising a plurality of single-strand nucleic acid fragments, the method comprising:
(a) contacting a universal oligonucleotide adaptor with the sample to produce a ligation product, wherein the universal oligonucleotide adaptor is configured for ligating to a 5′ end of the single-strand nucleic acid fragments;
(b) amplifying the ligation product by a first PCR with a first target-specific primer and optionally a first universal oligonucleotide adaptor primer to form a first PCR product; and
(c) amplifying the first PCR product by a second PCR with a second target-specific primer and a second universal oligonucleotide adaptor primer to form a second PCR product, wherein the second target-specific primer is nested relative to the first target-specific primer.

2. The method of claim 1, wherein prior to (a), the method further comprises at least one of: blocking a 3′ end of the single-strand nucleic acid fragments; phosphorylating the 5′ end of the single-strand nucleic acid fragments; or adenylating the nucleic acid to produce a 3′-adenosine overhang on the single-strand nucleic acid fragments.

3. The method of claim 1, wherein the first PCR is a linear amplification of the ligation product with the first target-specific primer to obtain a nascent primer extension duplex.

4. The method of claim 3, wherein (c) further comprises performing a nested amplification of the nascent primer extension duplex.

5. The method of claim 1, wherein the first PCR is an exponential amplification of the targeted nucleic acid with the first target-specific primer and the first universal oligonucleotide adaptor primer.

6. The method of claim 1, wherein the universal oligonucleotide adaptor comprises: a 3′ recessive end, the 3′ recessive end is configured for ligating to the 5′ end of the single-strand nucleic acid fragments; and/or a 5′ protrude end comprising three to twenty bases of random or degenerate nucleotides; wherein a duplex portion of the universal oligonucleotide adaptor is of sufficient length to remain in duplex form in (a).

7. The method of claim 6, wherein the universal oligonucleotide adaptor comprises a hairpin loop connecting a portion of the duplex form.

8. The method of claim 1, wherein the universal oligonucleotide adaptor comprises a single strand segment comprising three to twenty random nucleotides as a unique molecular index (UMI) for tracing individual original molecules.

9. The method of claim 1, wherein (c) further comprises forming a sequencing library with a sequencing specific adaptor pair.

10. The method of claim 9, wherein the method, after (c), further comprises sequencing the sequencing library using a sequencing primer pair, wherein the sequencing primer pair is at least partially complementary to opposite strands of the second PCR product, respectively.

11. A method of identifying genome-wide gene editing off-targets from a sample comprising a plurality of single-strand nucleic acid fragments, comprising:
(a) contacting a universal oligonucleotide adaptor with the sample to produce a ligation product, wherein the universal oligonucleotide adaptor is configured for ligating to a 5′ end of the single-strand nucleic acid fragments;
(b) amplifying the ligation product by performing a first PCR with a first target-specific primer to form a first PCR product;
(c) amplifying the first PCR product by a second PCR with a sequencing specific adaptor primer and a second target-specific primer nested relative to the first target-specific primer, to form a sequencing library;
(d) quantifying and reading the sequencing library to obtain sequencing results; and
(e) mapping the sequencing results to a reference genome and evaluating the gene editing off-targets.

12. The method of claim 11, wherein prior to (a), the method further comprises at least one of: blocking a 3′ end of the single-strand nucleic acid fragments; phosphorylating the 5′ end of the single-strand nucleic acid fragments; or adenylating the nucleic acid to produce a 3′-adenosine overhang on the single-strand nucleic acid fragments.

13. The method of claim 11, wherein the first PCR is a linear amplification of the ligation product with the first target-specific primer to obtain a nascent primer extension duplex.

14. The method of claim 13, wherein (c) further comprises performing a nested amplification of the nascent primer extension duplex.

15. The method of claim 11, wherein the first PCR is an exponential amplification of the targeted nucleic acid with the first target-specific primer and a first universal oligonucleotide adaptor primer.

16. The method of claim 11, wherein the universal oligonucleotide adaptor comprises: a 3′ recessive end, the 3′ recessive end is configured for ligating to the 5′ end of the single-strand nucleic acid fragments; and/or a 5′ protrude end comprising three to twenty bases of random or degenerate nucleotides; wherein a duplex portion of the universal oligonucleotide adaptor is of sufficient length to remain in duplex form in (a).

17. The method of claim 16, wherein the universal oligonucleotide adaptor comprises a hairpin loop connecting a portion of the duplex form.

18. The method of claim 11, wherein the universal oligonucleotide adaptor comprises a single strand segment comprising three to twenty random nucleotides as a unique molecular index (UMI) for tracing individual original molecules.

19. The method of claim 11, wherein (c) further comprises forming a sequencing library with a sequencing specific adaptor pair.

20. The method of claim 19, wherein the method, after (c), further comprises sequencing the sequencing library using a sequencing primer pair, wherein the sequencing primer pair is at least partially complementary to opposite strands of the second PCR product, respectively.
